(19) United States
(12) Patent Application Publication
(10) Pub. No.: US 2026/0216198 A1
(43) Pub. Date: Jul. 30, 2026

(54) GPR35 MODULATORS

(71) Applicant: ThirtyFiveBio Limited, Milton, Abingdon (GB)

(72) Inventors: Christopher Andrew LUCKHURST, Milton, Abingdon (GB); Roger John BUTLIN, Milton, Abingdon (GB); Monica Jade CLEMENTS, Milton, Abingdon (GB); Timothy HUXFORD, Milton, Abingdon (GB); Ageo MICCOLI, Milton, Abingdon (GB); Gianna TOSCHI, Milton, Abingdon (GB)

(21) Appl. No.: 19/143,724

(22) PCT Filed: Jan. 2, 2024
(86) PCT No.: PCT/GB2024/050005

§ 371 Date: Jun. 26, 2025

(30) Foreign Application Priority Data
Jan. 3, 2023 (GB) 2300021.9

(51) Int. Cl. A61K 31/5377; A61K 31/192; A61K 31/4412; A61K 31/4439; A61K 31/444; A61K 31/497; A61K 31/501; A61K 31/506; C07C 237/40; C07D 213/55; C07D 237/24; C07D 241/28; C07D 265/30; C07D 401/04; C07D 403/04; C07D 413/04; C07D 417/04

(52) U.S. Cl. CPC A61K 31/5377; A61K 31/192; A61K 31/4412; A61K 31/4439; A61K 31/444; A61K 31/497; A61K 31/501; A61K 31/506; C07C 237/40; C07D 213/55; C07D 237/24; C07D 241/28; C07D 265/30; C07D 401/04; C07D 403/04; C07D 413/04; C07D 417/04

(57) ABSTRACT

One aspect of the invention relates to a compound of formula (I), or a pharmaceutically acceptable salt or solvate thereof, X is NH and Y is CO ring C is a 6-membered heteroaryl or aryl group, or a partially or fully unsaturated 6-membered heterocyclic group containing at least one N, and optionally comprising at least one CO group, and wherein said aryl, heteroaryl or heterocyclic group is optionally further substituted by one or more substituents selected from alkyl, haloalkyl, alkoxy, haloalkoxy, halo, cyano, NR₉SO₂R₁₁, NR₉COR₁₂, NR₁₃R₁₄, OH, CO₂R₁₅, SO₂NR₁₆R₁₇, CONR₁₈R₁₉, cycloalkyl and (CH₂)_{q}-heterocycloalkyl; ring A is a group: wherein A is phenyl group or a 6-membered heteroaryl group; or ring A is a group: wherein A is a 5-membered heteroaryl group; wherein in each case the wavy lines indicate the point of attachment to Y and ring C respectively; ring B is a phenyl group or a 6-membered heteroaryl group; R₂ is (CR₃₃R₃₄)ₘCOOH; each R₆ is independently selected from alkyl, halo, haloalkyl, alkoxy, haloalkoxy, cyano, NR₁₀COR₂₀, NR₁₀SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl; each R₈ is independently selected from alkyl, halo, haloalkyl, alkoxy, haloalkoxy, cyano, NR₁₀COR₂₀, NR₁₀SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl; R₇ is selected from H and alkyl; each R₉ is independently selected from H and alkyl; each R₁₀ is independently selected from H and alkyl; R₁₁-R₃₁ are each independently selected from H, alkyl, haloalkyl, aralkyl, alkoxy, alkoxyalkyl, hydroxyalkyl and cycloalkyl; m, n and p are each independently an integer from 0 to 4; each q is independently an integer from 0 to 4; L is a direct bond or is a group selected from, —SO₂—, —O—SO₂—, —SO₂—O—, —O—, —NR₃₂—SO₂—, —NR₃₂—SO₂-alkylene, —SO₂—NR₃₂—, —SO₂—NR₃₂-alkylene, alkylene, heteroalkylene, cycloalkylene, heterocycloalkylene, alkylene-cycloalkylene, alkylene-SO₂—, —SO₂-alkylene, alkylene-SO—, —SO-alkylene, alkylene-SO₂-alkylene, alkylene-SO-alkylene, cycloalkylene-alkylene, alkylene-heterocycloalkylene, heterocycloalkylene-alkylene, heteroalkylene-heterocycloalkylene, heterocycloalkylene-heteroalkylene, heteroalkylene-cycloalkylene, cycloalkylene-heteroalkylene, wherein the alkylene moiety in the above groups is optionally substituted by one or more substituents selected from halo, alkyl, haloalkyl and cycloalkyl; R₃₂, R₃₃ and R₃₄ are each independently selected from H and alkyl; and Z is a group selected from alkyl, cycloalkyl, aryl, heteroaryl and heterocycloalkyl, each of which is optionally further substituted by one or more groups selected from CN, halo, alkyl, haloalkyl, alkenyl, alkynyl, alkoxy and haloalkoxy. Further aspects of the invention relate to pharmaceutical compositions comprising compounds of formula (I), and the use of said compounds in the treatment of various GPR35-related disorders.

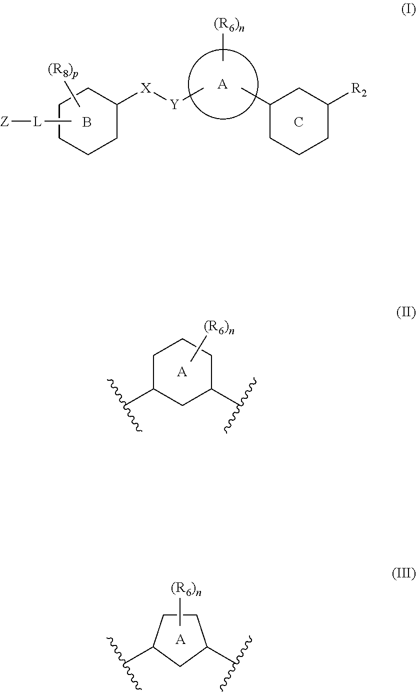

The present invention relates to compounds that are capable of modulating GPR35 function. The compounds have potential therapeutic applications in the treatment of a variety of disorders, including proliferative, immune and inflammatory disorders.

### BACKGROUND TO THE INVENTION

GPR35 is a class A, G protein-coupled receptor.^{1,2} The human gene can be expressed as three variants: Variant 1 encodes a 309 amino acid polypeptide designated GPR35a (short form), while variants 2 and 3 encode a long form, GPR35b, which has a 31 amino acid extension at the N-terminus. Although several ligands have been suggested as endogenous agonists of GPR35, the GPCR officially remains an orphan receptor.³⁻¹¹ Several synthetic and exogenous modulators have also been reported, including agonists¹²⁻¹⁴ and antagonists.¹⁵ The synthetic chemical zaprinast, (5-(2-propoxyphenyl)-1H-[1,2,3]triazolo-[4,5-d]pyrimidin-7(4H)-one) is currently the standard GPR35 agonist used as a reference compound.¹⁸ To date, however, the only GPR35 activator that has advanced into clinical trials is sodium cromoglycate (also known as RVT-1601 or PA101) which was investigated in the treatment of chronic cough in idiopathic pulmonary fibrosis^{1,37}.

Both isoforms of GPR35 display similar pharmacology with respect to reported agonists¹³ and any potential distinctive functions are unknown. GPR35 is primarily expressed throughout the epithelium of the gastrointestinal (GI) tract, including the stomach, gall bladder, duodenum, small intestine and colon^{16,17}, although expression is also prominent in certain macrophages and dendritic cells.^{2,18} Evidence suggests possible links between GPR35 and a range of pathological conditions including inflammation, asthma, cardiovascular disorders, and diabetes.¹⁸ Increased GPR35 expression is also associated with certain cancers.²⁵ High expression of GPR35 in gastric cancer is associated with poorer prognosis of patients. In vitro, GPR35 expression was associated with increased gastric cancer cell viability and proliferation, and reduced apoptosis.⁴⁰ siRNA knockdown of GPR35 in macrophages also reduced M2 markers ARG1 and PPARG, suggesting a role for GPR35 in supporting a pro-cancer macrophage M2 phenotype.

GPR35 signalling, therefore, represents an attractive pathway for therapeutic intervention for the treatment of a range of diseases. Accordingly, there is an ongoing need to develop new small molecule GPR35 modulators.

The present invention seeks to provide compounds that are capable of modulating GPR35. As made clear from the above discussion, such compounds have potential therapeutic applications in the treatment of a variety of disorders, including proliferative disorders and immune disorders, as well as inflammatory disorders.

### STATEMENT OF INVENTION

A first aspect of the invention relates to a compound of formula (I), or a pharmaceutically acceptable salt or solvate thereof,

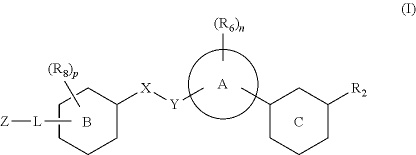

X is NR⁷ and Y is CO;
ring C is a 6-membered heteroaryl or aryl group, or a partially or fully unsaturated 6-membered heterocyclic group containing at least one N, and optionally comprising at least one CO group, and wherein said aryl, heteroaryl or heterocyclic group is optionally further substituted by one or more substituents selected from alkyl, haloalkyl, alkoxy, haloalkoxy, halo, cyano, NR₉SO₂R₁₁, NR₉COR₁₂, NR₁₃R₁₄, OH, CO₂R₁₅, SO₂NR₁₆R₁₇, CONR₁₈R₁₉, cycloalkyl and (CH₂)_{q}-heterocycloalkyl;
ring A is a group:

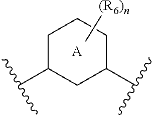

wherein A is a phenyl group or a 6-membered heteroaryl group;
or ring A is a group:

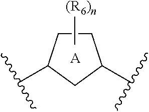

wherein A is a 5-membered heteroaryl group;
wherein in each case the wavy lines indicate the point of attachment to Y and ring C respectively;
ring B is a phenyl group or a 6-membered heteroaryl group; R₂ is (CR₃₃R₃₄)ₘCOOH;
each R₆ is independently selected from alkyl, halo, haloalkyl, alkoxy, haloalkoxy, cyano, NR₁₀COR₂₀, NR₁₀SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl;
each R₈ is independently selected from alkyl, halo, haloalkyl, alkoxy, haloalkoxy, cyano, NR₁₀COR₂₀, NR₁₀—SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl;
R₇ is selected from H and alkyl;
each R₉ is independently selected from H and alkyl;
each R₁₀ is independently selected from H and alkyl;
R₁₁-R₃₁ are each independently selected from H, alkyl, haloalkyl, aralkyl, alkoxy, alkoxyalkyl, hydroxyalkyl and cycloalkyl;
m, n and p are each independently an integer from 0 to 4;
each q is independently an integer from 0 to 4;
L is a direct bond or is a group selected from, —SO₂—, —O—SO₂—, —SO₂—O—, —O—, —NR₃₂—SO₂—, —NR₃₂—SO₂-alkylene, —SO₂—NR₃₂—, —SO₂—NR₃₂-alkylene, alkylene, heteroalkylene, cycloalkylene, heterocycloalkylene, alkylene-cycloalkylene, alkylene-SO₂—, —SO₂-alkylene, alkylene-SO—, —SO-alkylene, alkylene-SO₂-alkylene, alkylene-SO-alkylene, cycloalkylene-alkylene, alkylene-heterocycloalkylene, heterocycloalkylene-alkylene, heteroalkylene-heterocycloalkylene, heterocycloalkylene-heteroalkylene, heteroalkylene-cycloalkylene, cycloalkylene-heteroalkylene, wherein the alkylene moiety in the above groups is optionally substituted by one or more substituents selected from halo, alkyl, haloalkyl and cycloalkyl;
R₃₂, R₃₃ and R₃₄ are each independently selected from H and alkyl; and
Z is a group selected from alkyl, cycloalkyl, aryl, heteroaryl and heterocycloalkyl, each of which is optionally further substituted by one or more groups selected from cyano, halo, alkyl, haloalkyl, alkenyl, alkynyl, alkoxy and haloalkoxy.

Advantageously, the presently claimed compounds are capable of modulating GPR35, thereby rendering the compounds of therapeutic interest in the treatment of various disorders, including oncology applications, inflammatory disorders, and gastrointestinal disorders.

Another aspect of the invention relates to a pharmaceutical composition comprising a compound as described above and a pharmaceutically acceptable diluent, excipient, or carrier.

Another aspect of the invention relates to a pharmaceutical composition as described above for use as a medicament.

Another aspect of the invention relates to a compound as described above for use in treating or preventing a disorder selected from a proliferative disorder, a gastrointestinal disorder, a fibrotic disorder, cardiovascular disease, an immune disorder and an inflammatory disorder.

Another aspect of the invention relates to a pharmaceutical composition as described above for use in treating or preventing a disorder selected from a proliferative disorder, a gastrointestinal disorder, a fibrotic disorder, a cardiovascular disease, an immune disorder and an inflammatory disorder.

Another aspect of the invention relates to a method of treating a disorder, comprising administering to a subject a compound or a pharmaceutical composition as described above.

Another aspect of the invention relates to a compound as defined herein, or a pharmaceutically acceptable salt or solvate thereof, for use in treating or preventing a GPR35-associated disease or disorder.

Another aspect of the invention relates to the use of a compound as defined herein, or a pharmaceutically acceptable salt or solvate thereof, in the preparation of a medicament for treating or preventing a GPR35-associated disease or disorder in a subject.

Another aspect of the invention relates to the use of a compound as defined herein, or a pharmaceutically acceptable salt or solvate thereof, in the preparation of a medicament for treating or preventing a disorder selected from a proliferative disorder, a gastrointestinal disorder, an inflammatory disorder, a fibrotic disorder, an immune disorder and cardiovascular disease.

### DETAILED DESCRIPTION

The present invention relates to compounds that are capable of modulating GPR35.

“Alkyl” is defined herein as a straight-chain or branched alkyl radical, preferably C₁₋₂₀ alkyl, more preferably C₁₋₁₂ alkyl, even more preferably C₁₋₁₀ alkyl or C₁₋₆ alkyl, for example, methyl, ethyl, propyl, isopropyl, butyl, isobutyl, tert-butyl, pentyl, hexyl. More preferably, the alkyl is a C₁₋₃ alkyl.

“Cycloalkyl” is defined herein as a cyclic alkyl ring, preferably, C₃₋₇-cycloalkyl, more preferably C₃₋₆-cycloalkyl. Preferred examples include cyclopropyl, cyclobutyl, cyclopentyl, cyclohexyl or cycloheptyl, or a fused bicyclic ring system such as norbornane.

As used herein, the term “aryl” or “aromatic” refers to a C₆₋₁₂ aromatic group, which may be benzocondensed, for example, phenyl or naphthyl.

“Halogen” or “halo” is defined herein as chloro, fluoro, bromo or iodo.

“Haloalkyl” is defined herein as a straight-chain or branched alkyl radical as defined above, for example, methyl, ethyl, propyl, isopropyl, butyl, isobutyl, tert-butyl, pentyl, hexyl, that is substituted with one or more halogen atoms (that may be the same or different), such as fluorine, chlorine, bromine, and iodine. Preferably the haloalkyl group is a C₁₋₂₀ haloalkyl, more preferably C₁₋₁₂ haloalkyl, even more preferably C₁₋₁₀ haloalkyl or C₁₋₆ haloalkyl. Preferred examples are CF₃ and CHF₂, with CF₃ being particularly preferred.

“Alkoxy” is defined herein as an oxygen atom bonded to an alkyl group as defined above. Preferably the alkoxy group is a C₁₋₂₀ alkoxy, more preferably C₁₋₁₂ alkoxy, even more preferably C₁₋₁₀ alkoxy or C₁₋₆ alkoxy, for example methoxy, ethoxy, propoxy, isopropoxy, butoxy, isobutoxy, tert-butoxy, pentoxy and hexoxy. A particularly preferred example is methoxy (—OCH₃).

“Haloalkoxy” is defined herein as an alkoxy group as described above substituted with one or more halogen atoms (that may be the same or different), such as fluorine, chlorine, bromine, and iodine. Preferably the haloalkoxy group is a C₁₋₂₀ haloalkoxy, more preferably C₁₋₁₂ haloalkoxy, even more preferably C₁₋₁₀ haloalkoxy or C₁₋₆ haloalkoxy A particularly preferred example is OCF₃.

“Heteroaryl” is defined herein as a monocyclic aromatic ring comprising one or more heteroatoms (that may be the same or different), such as oxygen, nitrogen or sulphur. Examples of suitable 6-membered heteroaryl groups include pyridinyl, pyrazinyl, pyrimidinyl, pyridazinyl and triazinyl.

“Aralkyl’ is defined herein as an alkyl group as defined above substituted by one or more aryl groups as defined above.

“Heterocycloalkyl” refers to a cyclic aliphatic group containing one or more heteroatoms selected from nitrogen, oxygen and sulphur, which is optionally interrupted by one or more —(CO)— groups in the ring and/or which optionally contains one or more double bonds in the ring. Preferably, the heterocycloalkyl group is monocyclic or bicyclic. Preferably, the heterocycloalkyl group is a C₃₋₇-heterocycloalkyl, more preferably a C₃₋₆-heterocycloalkyl. Alternatively, the heterocycloalkyl group is a C₄₋₇-heterocycloalkyl, more preferably a C₄₋₆-heterocycloalkyl. Preferred heterocycloalkyl groups include, but are not limited to, piperazinyl, piperidinyl, morpholinyl, thiomorpholinyl, pyrrolidinyl, tetrahydrofuranyl, tetrahydropyranyl, oxetanyl and azetidinyl. Preferably the heterocycloalkyl group is monovalent. Preferably the heterocycloalkyl group is monocyclic.

As used herein, the term “alkenyl” refers to both straight and branched carbon chains which have at least one carbon-carbon double bond. In some embodiments, alkenyl groups may include C₂-C₁₂ alkenyl groups. In other embodiments, alkenyl includes C₂-C₁₀, C₂-C₈, C₂-C₆ or C₂-C₄ alkenyl groups. In one embodiment of alkenyl, the number of double bonds is 1-3; in another embodiment of alkenyl, the number of double bonds is one. Other ranges of carbon-carbon double bonds and carbon numbers are also contemplated depending on the location of the alkenyl moiety on the molecule. “C₂-C₁₀-alkenyl” groups may include more than one double bond in the chain.

As used herein, the term “alkynyl” refers to both straight and branched carbon chains which have at least one carbon-carbon triple bond. In some embodiments, alkynyl groups may include C₂-C₁₂ alkynyl groups. In other embodiments, alkynyl includes C₂-C₁₀, C₂-C₈, C₂-C₆ or C₂-C₄ alkynyl groups. In one embodiment of alkynyl, the number of triple bonds is 1-3; in another embodiment of alkenyl, the number of triple bonds is one. A particularly preferred alkynyl group is —C≡CH.

As used herein, the term “alkylene” refers to a linear or branched saturated divalent hydrocarbon radical. Preferably, the alkylene group is a linear saturated divalent hydrocarbon radical comprising one to six carbon atoms, or a branched saturated divalent hydrocarbon radical comprising three to six carbon atoms.

As used herein, the term “heteroalkylene” refers to a divalent alkylene having one or more carbon atoms replaced with a sulfur, oxygen, or NR′ where R′ is H or alkyl. Preferably, the heteroalkylene group is a divalent alkylene having one or more carbon atoms replaced with an oxygen or a sulphur, more preferably an oxygen.

As used herein, the term “cycloalkylene” refers to a divalent cyclic saturated hydrocarbon radical, preferably comprising three to ten carbon atoms.

As used herein, the term “heterocycloalkylene” refers to a divalent cycloalkylene group as defined above, having one or more carbon atoms replaced with a sulfur, oxygen, or NR′ where R′ is H or alkyl.

As used herein, preferably alkyl is C₁-C₆ alkyl, haloalkyl is C₁-C₆ haloalkyl, haloalkoxy is C₁-C₆ haloalkoxy and alkoxy is C₁-C₆ alkoxy.

### Compounds According to the Invention

Preferred definitions of the various groups apply to all formulae and subformulae described herein.

In one aspect of the invention relates to a compound of formula (I′), or a pharmaceutically acceptable salt or solvate thereof,

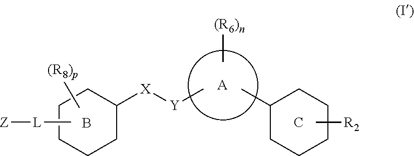

X—Y is —CONR₇— or —NR₇CO—;
ring C is a 6-membered aryl or heteroaryl group, or a partially or fully unsaturated 6-membered heterocyclic group containing at least one N, and optionally comprising at least one CO group, and wherein said aryl, heteroaryl or heterocyclic group is optionally further substituted by one or more substituents selected from alkyl, haloalkyl, alkoxy, haloalkoxy, halo, cyano, NR₉SO₂—R₁₁, NR₉COR₁₂, NR₁₃R₁₄, OH, CO₂R₁₅, SO₂NR₁₆R₁₇, CONR₁₈R₁₉, cycloalkyl and (CH₂)_{q}-heterocycloalkyl;
ring A is a phenyl group or a 5- or 6-membered heteroaryl group;
ring B is a phenyl group or a 6-membered heteroaryl group;
R₂ is (CR₃₃R₃₄)ₘCOOH;
each R₆ is independently selected from alkyl, halo, haloalkyl, alkoxy, haloalkoxy, cyano, NR₁₀COR₂₀, NR₁₀SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl;
each R₈ is independently selected from alkyl, halo, haloalkyl, alkoxy, haloalkoxy, cyano, NR₁₀COR₂₀, NR₁₀—SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl;
R₇ is selected from H and alkyl;
each R₉ is independently selected from H and alkyl;
each R₁₀ is independently selected from H and alkyl;
R₁₁-R₃₁ are each independently selected from H, alkyl, aralkyl, alkoxy, alkoxyalkyl, hydroxyalkyl and cycloalkyl;
m, n and p are each independently an integer from 0 to 4;
each q is independently an integer from 0 to 4;
L is a direct bond or is a group selected from, —SO₂—, —O—SO₂—, —SO₂—O—, —O—, —NR₃₂—SO₂—, —NR₃₂—SO₂-alkylene, —SO₂—NR₃₂—, —SO₂—NR₃₂-alkylene, alkylene, heteroalkylene, cycloalkylene, heterocycloalkylene, alkylene-cycloalkylene, alkylene-SO₂—, —SO₂-alkylene, alkylene-SO—, —SO-alkylene, alkylene-SO₂-alkylene, alkylene-SO-alkylene, cycloalkylene-alkylene, alkylene-heterocycloalkylene, heterocycloalkylene-alkylene, heteroalkylene-heterocycloalkylene, heterocycloalkylene-heteroalkylene, heteroalkylene-cycloalkylene, cycloalkylene-heteroalkylene, wherein the alkylene moiety in the above groups is optionally substituted by one or more substituents selected from halo, alkyl and cycloalkyl;
R₃₂, R₃₃ and R₃₄ are each independently selected from H and alkyl; and
Z is a group selected from alkyl, cycloalkyl, aryl, heteroaryl and heterocycloalkyl, each of which is optionally further substituted by one or more groups selected from CN, halo, alkyl, alkenyl, alkynyl, alkoxy and haloalkoxy.

In one preferred embodiment, X—Y is —NH—CO—.

In one preferred embodiment, the R² group is in the meta-position on the C ring relative to the point of attachment to the A ring.

In one preferred embodiment, R² is in the ortho-position relative to the point of attachment to the A ring.

In one preferred embodiment, R² is in the para-position relative to the point of attachment to the A ring.

In one preferred embodiment, ring A is:

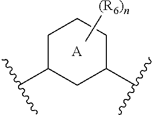

wherein A is phenyl or a 6-membered heteroaryl group, and Z, L, B, X, Y, X₁-X₅, R₂, R₆, R₈, n and p are as defined above.

In one preferred embodiment, A is selected from phenyl, pyridinyl, pyrimidinyl and pyrazinyl, each of which is optionally substituted by one to four R₆ groups.

In another preferred embodiment, ring A is:

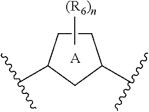

wherein A is a 5-membered heteroaryl group, and Z, L, B, X, Y, X₁-X₅, R₂, R₆, R₈, n and p are as defined above.

In one preferred embodiment, A is selected from pyrrolyl, thiazolyl, oxazolyl, furanyl, thienyl and pyrazolyl, each of which is optionally substituted by one to three R₆ groups.

In one preferred embodiment, the compound is of formula (Ia):

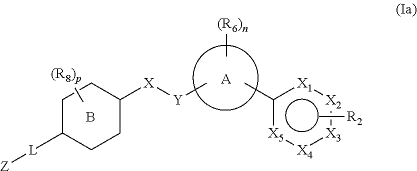

wherein X₁, X₂, X₃, X₄ and X₅ form a 6-membered aryl or heteroaryl group containing at least one N, and wherein said aryl or heteroaryl group is optionally further substituted by one or more substituents selected from alkyl, haloalkyl, alkoxy, haloalkoxy, halo, NR₉SO₂R₁₁, NR₉COR₁₂, NR₁₃R₁₄, OH, CO₂R₁₅, SO₂NR₁₆R₁₇, CONR₁₈R₁₉, cycloalkyl and (CH₂)_{q}-heterocycloalkyl; and A, B, Z, L, X, Y, R₂, R₆, R₈, n and p are as defined above.

In one preferred embodiment, the compound of the invention is of formula (I), or a pharmaceutically acceptable salt or solvate thereof,

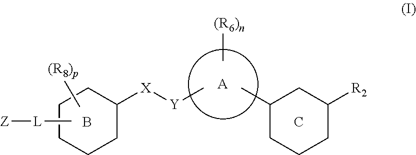

X is NR⁷ and Y is CO;
ring C is a 6-membered aryl or heteroaryl group, or a partially or fully unsaturated 6-membered heterocyclic group containing at least one N, and optionally comprising at least one CO group, and wherein said aryl, heteroaryl or heterocyclic group is optionally further substituted by one or more substituents selected from alkyl, haloalkyl, alkoxy, haloalkoxy, halo, cyano, NR₉SO₂R₁₁, NRCOR₁₂, NR₁₃R₁₄, OH, CO₂R₁₅, SO₂NR₁₆R₁₇, CONR₁₈R₁₉, cycloalkyl and (CH₂)_{q}-heterocycloalkyl;
ring A is a group:

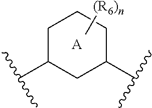

wherein A is a phenyl group or a 6-membered heteroaryl group;
or ring A is a group:

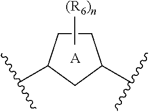

wherein A is a 5-membered heteroaryl group;
wherein in each case the wavy lines indicate the point of attachment to Y and ring C respectively;
ring B is a phenyl group or a 6-membered heteroaryl group;
R₂ is (CR₃₃R₃₄)ₘCOOH;
each R₆ is independently selected from alkyl, halo, haloalkyl, alkoxy, haloalkoxy, cyano, NR₁₀COR₂₀, NR₁₀—SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl;
each R₈ is independently selected from alkyl, halo, haloalkyl, alkoxy, haloalkoxy, cyano, NR₁₀COR₂₀, NR₁₀—SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl;
R₇ is selected from H and alkyl;
each R₉ is independently selected from H and alkyl;
each R₁₀ is independently selected from H and alkyl;
R₁₁-R₃₁ are each independently selected from H, alkyl, haloalkyl, aralkyl, alkoxy, alkoxyalkyl, hydroxyalkyl and cycloalkyl;
m, n and p are each independently an integer from 0 to 4;
each q is independently an integer from 0 to 4;
L is a direct bond or is a group selected from, —SO₂—, —O—SO₂—, —SO₂—O—, —O—, —NR₃₂—SO₂—, —NR₃₂—SO₂-alkylene, —SO₂—NR₃₂—, —SO₂—NR₃₂-alkylene, alkylene, heteroalkylene, cycloalkylene, heterocycloalkylene, alkylene-cycloalkylene, alkylene-SO₂—, —SO₂-alkylene, alkylene-SO—, —SO-alkylene, alkylene-SO₂-alkylene, alkylene-SO-alkylene, cycloalkylene-alkylene, alkylene-heterocycloalkylene, heterocycloalkylene-alkylene, heteroalkylene-heterocycloalkylene, heterocycloalkylene-heteroalkylene, heteroalkylene-cycloalkylene, cycloalkylene-heteroalkylene, wherein the alkylene moiety in the above groups is optionally substituted by one or more substituents selected from halo, alkyl, haloalkyl and cycloalkyl;
R₃₂, R₃₃ and R₃₄ are each independently selected from H and alkyl; and
Z is a group selected from alkyl, cycloalkyl, aryl, heteroaryl and heterocycloalkyl, each of which is optionally further substituted by one or more groups selected from CN, halo, alkyl, haloalkyl, alkenyl, alkynyl, alkoxy and haloalkoxy.

In one preferred embodiment of the invention, R₇ is H, i.e. the group X—Y is —NH—CO—, and the compound is of formula (I-1):

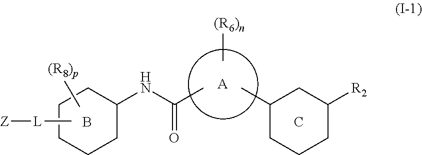

wherein ring A, ring B, ring C, L, Z, R₂, R₆, R₈, n and p are as defined above.

In one preferred embodiment, the compound is of formula (Ie):

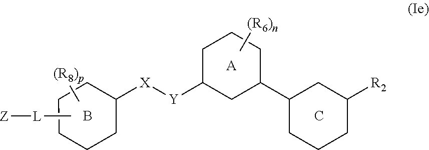

wherein A is phenyl or a 6-membered heteroaryl group, and R₂, R₆, R₈, ring B, ring C, X, Y, Z, L, n and p are as defined above.

In one preferred embodiment of formula (Ie), A is selected from phenyl, pyridinyl, pyrimidinyl, pyradizinyl and pyrazinyl, each of which is optionally substituted by one to four R₆ groups. Preferably, A is an optionally substituted phenyl group. In one preferred embodiment, A is an unsubstituted phenyl group, i.e. n is 0.

In one preferred embodiment, the compound according to claim 1 which is of formula (If):

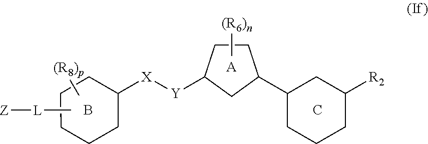

and wherein A is a 5-membered heteroaryl group, and R₂, R₆, R₈, ring B, ring C, X, Y, Z, L, n and p are as defined above. Preferably, n is an integer from 0 to 3.

In one preferred embodiment of formula (If), A is selected from pyrrolyl, thiazolyl, oxazolyl, furanyl, thienyl and pyrazolyl, each of which is optionally substituted by one to three R₆ groups. Preferably, A is a thiazoylyl group optionally substituted by one to three R₆ groups.

In one preferred embodiment, compound is of formula (Ig):

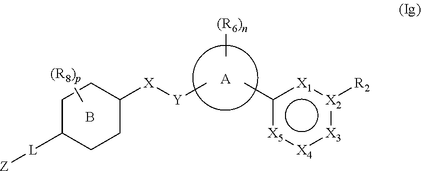

wherein X₁, X₂, X₃, X₄, X₅ form a 6-membered heteroaryl group containing at least one N, or a 6-membered aryl group, and wherein said aryl or heteroaryl group is optionally further substituted by one or more substituents selected from alkyl, haloalkyl, alkoxy, haloalkoxy, halo, NR₉SO₂R₁₁, NRCOR₁₂, NR₁₃R₁₄, OH, CO₂R₁₅, SO₂NR₁₆R₁₇, CONR₁₈R₁₉, cycloalkyl and (CH₂)_{q}-heterocycloalkyl; and
A, B, Z, L, X, Y, R₂, R₆, R₈, n and p are as defined above.

In one preferred embodiment, A is a phenyl group or a 6-membered heteroaryl group, and the compound is of formula (Ig-1):

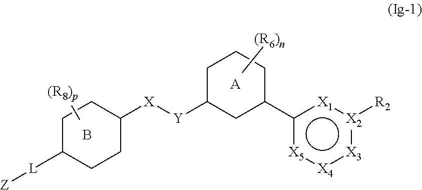

wherein B, Z, L, X, Y, X₁, X₂, X₃, X₄, X₅, R₂, R₆, R₃, n and p are as defined above.

In another preferred embodiment, A is a 5-membered heteroaryl group, and the compound is of formula (Ig-2):

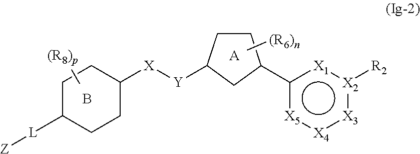

wherein B, Z, L, X, Y, X₁, X₂, X₃, X₄, X₅, R₂, R₆, R₃, n and p are as defined above.

In one preferred embodiment, each R₆ is independently selected from C₁₋₆-alkyl, halo, C₁₋₆-haloalkyl, C₁₋₆-alkoxy, C₁₋₆-haloalkoxy, cyano, NHCO—C₁₋₆-alkyl, NHSO₂—C₁₋₆-alkyl, S—C₁₋₆-alkyl, SO—C₁₋₆-alkyl, CH₂SO₂—C₁₋₆-alkyl, SO₂—C₁₋₆-alkyl, SO₂N(C₁₋₆-alkyl)₂, CH₂OH, CH₂O—C₁₋₆-alkyl, N(C₁₋₆-alkyl)₂, CON(C₁₋₆-alkyl)₂, cycloalkyl, heterocycloalkyl and CH₂-heterocycloalkyl.

In one preferred embodiment, each R₆ is independently selected from C₁₋₆-alkyl, halo, C₁₋₆-haloalkyl, C₁₋₆-alkoxy, C₁₋₆-haloalkoxy, cyano, NHCO—C₁₋₆-alkyl, NHSO₂—C₁₋₆-alkyl, S—C₁₋₆-alkyl, SO—C₁₋₆-alkyl, CH₂SO₂—C₁₋₆-alkyl, SO₂—C₁₋₆-alkyl, SO₂N(C₁₋₆-alkyl)₂, CH₂OH, CH₂O—C₁₋₆-alkyl, CON(C₁₋₆-alkyl)₂, cycloalkyl, heterocycloalkyl and CH₂-heterocycloalkyl.

In one preferred embodiment, each R₆ is independently selected from C₁₋₆-alkyl, halo, C₁₋₆-haloalkyl, C₁₋₆-alkoxy and C₁₋₆-haloalkoxy.

In one preferred embodiment, each R₆ is independently selected from Me, halo, CF₃, OMe, OCF₃, amino, cyano, NHCOMe, NHSO₂Me, S-Me, CH₂SO₂Me, SO₂Me, SO₂NMe₂, CH₂OH, CH₂OMe, cycloalkyl, CH₂—N-morpholinyl and N-morpholinyl.

In one preferred embodiment, each R₈ is independently selected from C₁₋₆-alkyl, halo, C₁₋₆-haloalkyl, C₁₋₆-alkoxy, C₁₋₆-haloalkoxy, cyano, NHCO—C₁₋₆-alkyl, NHSO₂—C₁₋₆-alkyl, S—C₁₋₆-alkyl, SO—C₁₋₆-alkyl, CH₂SO₂—C₁₋₆-alkyl, SO₂—C₁₋₆-alkyl, SO₂N(C₁₋₆-alkyl)₂, CH₂OH, CH₂O—C₁₋₆-alkyl, N(C₁₋₆-alkyl)₂, CON(C₁₋₆-alkyl)₂, cycloalkyl, heterocycloalkyl and CH₂-heterocycloalkyl.

In one preferred embodiment, each R₈ is independently selected from C₁₋₆-alkyl, halo, C₁₋₆-haloalkyl, C₁₋₆-alkoxy and C₁₋₆-haloalkoxy.

In one preferred embodiment, each R₈ is independently selected from Me, halo, CF₃, OMe, OCF₃, amino, cyano, NHCOMe, NHSO₂Me, S-Me, CH₂SO₂Me, SO₂Me, SO₂NMe₂, CH₂OH, CH₂OMe, cycloalkyl, CH₂—N-morpholinyl and N-morpholinyl.

In one preferred embodiment, R₇, each R₉ and each R₁₀ are each independently selected from H and C₁₋₆ alkyl. More preferably, R₇, each R₉ and each R₁₀ are each independently selected from H and Me. Even more preferably, R₇, each R₉ and each R₁₀ are all H.

In one preferred embodiment, R₇ is H, i.e. the group X—Y is —NH—C(═O)— in the general formulae and subformulae described herein, with NH linked to the B ring and the CO linked to the A ring.

In one preferred embodiment, R₁₁-R₃₁ are each independently selected from H, C₁₋₆-alkyl, C₁₋₆-haloalkyl, aralkyl, C₁₋₆-alkoxy, hydroxyl-C₁₋₆-alkyl and C₃₋₆-cycloalkyl, more preferably H and C₁₋₆-alkyl.

In one preferred embodiment, R₁₁-R₃₁ are each independently selected from H, Me, CF₃, cyclopropyl, —CH₂CH₂OH and —CH₂CH₂Me, more preferably, H and Me.

In one preferred embodiment, R₃₃ and R₃₄ are each independently selected from H and alkyl. Preferably, R₃₃ and R₃₄ are both H.

In one preferred embodiment, m is 0 or 1, i.e. R₂ is CH₂COOH or COOH. More preferably, m is 0, i.e. R₂ is COOH.

In one preferred embodiment, the compound is of formula (Ib):

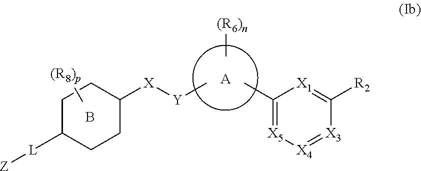

wherein:
X₁ is N or CR₁;
X₃ is N or CR₃;
X₄ is N or CR₄;
X₅ is N or CR₅;
R₁, R₃, R₄ and R₅ are each independently selected from H, alkyl, haloalkyl, alkoxy, haloalkoxy, halo, NR₁₃R₁₄, OH, NR₉SO₂-alkyl, CONR₁₈R₁₉, cycloalkyl and (CH₂)_{q}-heterocycloalkyl; and
wherein R₂, R₆, R₈, n, p, L, A, B, X, Y and Z are as defined above.

For each of the above sub-formula for ring A, each R₆ is independently selected from C₁₋₆-alkyl, halo, C₁₋₆-haloalkyl, C₁₋₆-alkoxy and C₁₋₆-haloalkoxy.

In one preferred embodiment, n is 0.

In one preferred embodiment, X₁, X₂, X₃, X₄ and X₅ form a phenyl group or a 6-membered heteroaryl group comprising at least one nitrogen.

In one preferred embodiment, X₁, X₂, X₃, X₄ and X₅ form a 6-membered heteroaryl group comprising 1, 2 or 3 nitrogens. In one preferred embodiment, the 6-membered heteroaryl group comprises 1 or 2 nitrogens. In another preferred embodiment, the 6-membered heteroaryl group comprises 1 nitrogen.

In one preferred embodiment, X₁, X₂, X₃, X₄ and X₅ form a phenyl, pyridinyl, pyrimidinyl, pyridazinyl or pyrazinyl group.

In one preferred embodiment, X₁, X₂, X₃, X₄ and X₅ form a pyridinyl or pyrazinyl group.

In one preferred embodiment, A is a phenyl group or a 6-membered heteroaryl group.

In one preferred embodiment, the compound is of formula (Ic):

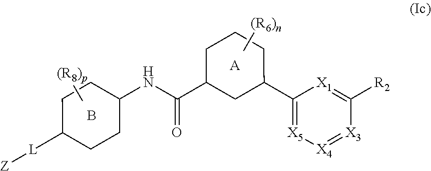

wherein:
X₁ is N or CR₁;
X₃ is N or CR₃;
X₄ is N or CR₄;
X₅ is N or CR₅;
R₁, R₃, R₄ and R₅ are each independently selected from H, alkyl, haloalkyl, alkoxy, haloalkoxy, halo, NR₁₃R₁₄, OH, NR₉SO₂-alkyl, CONR₁₈R₁₉, cycloalkyl and (CH₂)_{q}-heterocycloalkyl; A is a phenyl or pyridinyl group, more preferably phenyl; and
R₂, R₆, R₃, n, p, L, B and Z are as defined above.

Preferably, q is 0 or 1, more preferably 0.

In one preferred embodiment, R₁, R₃, R₄ and R₅ are each independently selected from H, C₁₋₆-alkyl, C₁₋₆-haloalkyl, C₁₋₆-alkoxy, C₁₋₆-haloalkoxy, Cl, F, NH₂, NH—C₁₋₆-alkyl, NH—C₃₋₆-cycloalkyl, N(C₁₋₆-alkyl)₂, OH, NHSO₂—C₁₋₆-alkyl, CONR₁₈R₁₉, C₃₋₆-cycloalkyl, NH-(hydroxy-C₁₋₆-alkyl), NH—(C₁₋₆-alkoxy), CH₂—C₃₋₇-heterocycloalkyl and C₃₋₇-heterocycloalkyl.

In one preferred embodiment, R₃ is selected from H, C₁₋₆-alkyl, C₁₋₆-haloalkyl, C₁₋₆-alkoxy, C₁₋₆-haloalkoxy, F, Cl, NH₂, NH—C₃₋₆-cycloalkyl, NH—C₁₋₆-alkyl, N(C₁₋₆-alkyl)₂, NH—C₁₋₆-alkoxy, NH-(hydroxy-C₁₋₆-alkyl), OH, NHSO₂—C₁₋₆-alkyl, C₀₂C₁₋₆-alkyl, C₃₋₆-cycloalkyl and C₃₋₆-heterocycloalkyl; and R₁, R₄ and R₅ are H.

In one preferred embodiment, R₁, R₃, R₄ and R₅ are each independently selected from H, Me, MeO, CF₃, Cl, F, NH₂, NH-Me, NH-cyclopropyl, NMe₂, OH, NHSO₂Me, CONH₂, cyclopropyl, NHCH₂CH₂OH, NHCH₂CH₂OMe, CH₂—N-morpholinyl and N-morpholinyl.

In one preferred embodiment, R₃ is selected from H, Me, MeO, CF₃, Cl, F, NH₂, NH-Me, NH-cyclopropyl, NMe₂, OH, NHSO₂Me, CONH₂, cyclopropyl, NHCH₂CH₂OH, NHCH₂CH₂OMe, CH₂—N-morpholinyl and N-morpholinyl; and R₁, R₄ and R₅ are H.

In one preferred embodiment:
R₂ is COOH;
X₁ is CR₁;
X₃ is CR₃;
X₄ is N; and
X₅ is CR₅.

Preferably, for this embodiment, X₁ and X₅ are CH, and X₃ is CR₃, wherein R₃ is selected from H, Me, MeO, CF₃, C, F, NH₂, NH-Me, NH-cyclopropyl, NMe₂, OH, NHSO₂Me, CONH₂, cyclopropyl, NHCH₂CH₂OH, NHCH₂CH₂OMe, CH₂—N-morpholinyl and N-morpholinyl.

In one preferred embodiment:
R₂ is COOH;
X₁ is N;
X₃ is CR₃;
X₄ is CR₄; and
X₅ is CR₅.

Preferably, for this embodiment, X₄ and X₅ are CH, and X₃ is CR₃, wherein R₃ is selected from H, Me, MeO, CF₃, Cl, F, NH₂, NH-Me, NH-cyclopropyl, NMe₂, OH, NHSO₂Me, CONH₂, cyclopropyl, NHCH₂CH₂OH, NHCH₂CH₂OMe, CH₂—N-morpholinyl and N-morpholinyl.

In one preferred embodiment:
R₂ is COOH;
X₁ is N;
X₃ is CR₃;
X₄ is N; and
X₅ is CR₅.

Preferably, for this embodiment, X₅ is CH, and X₃ is CR₃, wherein R₃ is selected from H, Me, MeO, CF₃, C, F, NH₂, NH-Me, NH-cyclopropyl, NMe₂, OH, NHSO₂Me, CONH₂, cyclopropyl, NHCH₂CH₂OH, NHCH₂CH₂OMe, CH₂—N-morpholinyl and N-morpholinyl.

In one preferred embodiment:
R₂ is COOH;
X₁ is CH or N, more preferably CH;
X₃ is C-alkyl, preferably Me;
X₄ is N; and
X₅ is CH.

In one preferred embodiment, ring C is:

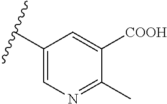 [SMILES: *c1cnc(C)c(C(=O)O)c1]

wherein the wavy line indicates the point of attachment to ring A.

In one preferred embodiment, A is a 5-membered heteroaryl group, and the compound is of formula (Ic′):

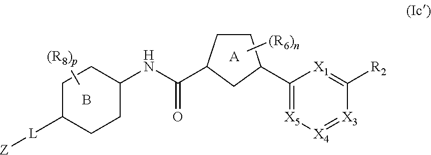

wherein X₁, X₃, X₄, X₅, R₂, R₆, R₈, n, p, L, B and Z are as defined above for formula (Ic).

In one preferred embodiment, ring C is a partially or fully unsaturated 6-membered heterocyclic group containing at least one N, and optionally comprising at least one CO group.

In one preferred embodiment, the compound is of formula (Id)

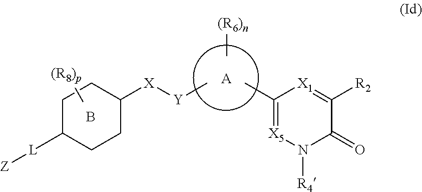

wherein X₁ is N or CR₁ and X₅ is N or CR₅, where R₁, R₂, R₅, R₆, R₈, A, B, X, Y, L, Z, n and p are as defined above, and R₄′ is selected from H, alkyl and cycloalkyl. Preferably, X₁ and X₅ are both CH. Preferably, R₄′ is H.

The skilled person will recognise when R₄′ is H, compounds of formula (Id) can exist as one of two possible tautomers as shown below. For example, where X₁ is CR₁ and X₅ is CR₅:

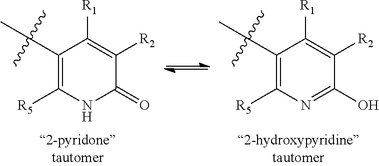

The 2-pyridone tautomer is believed to be the predominant solid state form. In solution, the energy difference between the two tautomeric forms is understood to be very small and is dependent on the polarity of the solvent. The skilled person would appreciate that other hydroxy substituted N-containing heteroaromatic groups (e.g. pyrimidine, other pyridine regioisomers) can be similarly represented in tautomeric form as shown above. The invention encompasses all tautomeric forms of the compounds.

In one preferred embodiment, A is a phenyl group or a 6-membered heteroaryl group, and the compound is of formula (Id-1):

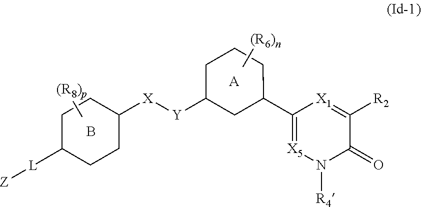

wherein X₁ is N or CR₁ and X_{R} is N or CR₂, where R₁, R₂, R₄, R₅, R₆, R₈, B, X, Y, L, Z, n and p are as defined above.

In one preferred embodiment, A is a 5-membered heteroaryl group and the compound is of formula (Id-2):

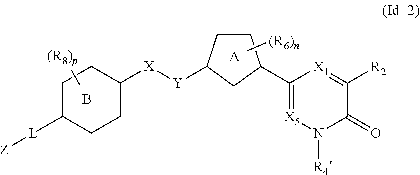

wherein X₁ is N or CR₁ and X₅ is N or CR₅, where R₁, R₂, R₄′, R₅, R₆, R₈, B, X, Y, L, Z, n and p are as defined above.

For all of the above embodiments described herein, B is preferably a phenyl or pyridinyl group, each of which is optionally substituted by one to four R₈ groups as defined above.

Preferably, each R₈ is independently selected from C₁₋₆-alkyl, halo, C₁₋₆-haloalkyl, C₁₋₆-alkoxy and C₁₋₆-haloalkoxy.

In one preferred embodiment, B is a phenyl group, optionally substituted by one or two halo groups.

In one preferred embodiment, p is 0.

For all of the above embodiments described herein, Z is preferably a group selected from C₁₋₆-alkyl, phenyl, C₃₋₆-cycloalkyl and a 5- or 6-membered heterocycloalkyl group, each of which is optionally further substituted by one or more groups selected from alkyl, CN, halo, haloalkyl, alkenyl, alkynyl and alkoxy.

For all of the above embodiments described herein, Z is preferably a group selected from C₁₋₆-alkyl, phenyl, C₃₋₆-cycloalkyl and a 5- or 6-membered heterocycloalkyl group, each of which is optionally further substituted by one or more groups selected from alkyl, alkenyl, alkynyl and alkoxy.

In one preferred embodiment, Z is a group selected from phenyl, pyridinyl, cyclopropyl, piperidinyl and tetrahydropyranyl, more preferably phenyl, each of which is optionally further substituted by one or more groups selected from alkyl, cyano, halo, haloalkyl, alkenyl, alkynyl and alkoxy.

In one preferred embodiment, Z is a group selected from phenyl, cyclopropyl and tetrahydropyranyl, more preferably phenyl.

In the compounds described herein, L is a direct bond or is a group selected from, —SO₂—, —O—SO₂—, —SO₂—O—, —O—, —NR₃₂—SO₂—, —NR₃₂—SO₂-alkylene, —SO₂—NR₃₂—, —SO₂—NR₃₂-alkylene, alkylene, heteroalkylene, cycloalkylene, heterocycloalkylene, alkylene-cycloalkylene, alkylene-SO₂-alkylene, alkylene-SO-alkylene, alkylene-SO₂—, —SO₂-alkylene, alkylene-SO—, —SO-alkylene, cycloalkylene-alkylene, alkylene-heterocycloalkylene, heterocycloalkylene-alkylene, heteroalkylene-heterocycloalkylene, heterocycloalkylene-heteroalkylene, heteroalkylene-cycloalkylene, cycloalkylene-heteroalkylene, wherein the alkylene moiety in the above groups is optionally substituted by one or more substituents selected from halo, alkyl, haloalkyl and cycloalkyl.

As used herein, substitution of an alkylene moiety refers to one or both of the hydrogens of one or more —CH₂— groups in the alkylene (or heteroalkylene) group being replaced by a substituent selected from halo, haloalkyl, alkyl and cycloalkyl.

In one preferred embodiment, the heteroalkylene group is a divalent alkylene group having one or more carbon atoms replaced with a heteroatom independently selected from O and S. More preferably, the heteroalkylene group is a divalent alkylene group having one or more carbon atoms replaced with an oxygen.

In the compounds described herein, L is a direct bond or is a group selected from, —SO₂—, —O—SO₂—, —SO₂—O—, —O—, —NR₃₂—SO₂—, —NR₃₂—SO₂-alkylene, —SO₂—NR₃₂—, —SO₂—NR₃₂-alkylene, alkylene, heteroalkylene, cycloalkylene, heterocycloalkylene, alkylene-cycloalkylene, alkylene-SO₂-alkylene, alkylene-SO-alkylene, alkylene-SO₂—, —SO₂-alkylene, alkylene-SO—, —SO-alkylene, cycloalkylene-alkylene, alkylene-heterocycloalkylene, heterocycloalkylenealkylene, heteroalkylene-heterocycloalkylene, heterocycloalkylene-heteroalkylene, heteroalkylene-cycloalkylene, cycloalkylene-heteroalkylene, wherein the alkylene moiety in the above groups is optionally substituted by one or more substituents selected from halo, alkyl and cycloalkyl.

Preferably, R₃₂ is H or Me, more preferably H.

For all of the above embodiments, L is preferably selected from —O—, —NH—SO₂—, —NH—SO₂—(CR′R″)ₐ—, —(CR′R″)ₐ—SO₂—NH—, —SO₂—NH—, —SO₂—NH—(CR′R″)ₐ—, —(CR′R″)ₐ—NH—SO₂—, —O—SO₂—, —SO₂—O—, —(CR′R″)ₐ—, —(CR′R″)ₐ—O—, O—(CR′R″)ₐ—, —(CR′R″)ₐ—O—(CR′R″)_{b}—, —(CR′R″)ₐ—S—(CR′R″)_{b}—, —(CR′R″)ₐ—SO—(CR′R″)_{b}—, —(CR′R″)ₐ—SO₂—, —SO₂—(CR′R″)_{b}—, —(CR′R″)ₐ—SO—, —SO—(CR′R″)_{b}—, —(CR′R″)ₐ—SO₂—(CR′R″)_{b}—, —(CR′R″)ₐ—S—(CR′R″)_{b}—O—, —O—(CR′R″)ₐ—S—(CR′R″)_{b}—, —(CR′R″)ₐ—O—(CR′R″)_{b}—S—, —S—(CR′R″)ₐ—O—(CR′R″)_{b}—, heterocycloalkylene-(CR′R″)ₐ—O—, —O—(CR′R″)ₐ-heterocycloalkylene-, —(CR′R″)ₐ—O-heterocycloalkylene, heterocycloalkylene-O—(CR′R″)ₐ—, —O-heterocycloalkylene-(CR′R″)ₐ— and —(CR′R″)ₐ-heterocycloalkylene-O—, wherein a and b are each independently an integer from 1 to 6, and each R′ and each R″ is independently selected from H, alkyl and haloalkyl.

In one preferred embodiment, a and b are each independently an integer from 1 to 3.

In one preferred embodiment, each R′ and each R″ is independently selected from H, Me and CF₃.

For all of the above embodiments, L is preferably selected from from —O—, —NH—SO₂—, —NH—SO₂—(CH₂)ₐ—, —SO₂—NH—, —SO₂—NH—(CH₂)ₐ—, —(CH₂)ₐ—NH—SO₂—, —O—SO₂—, —SO₂—O—, —(CH₂)ₐ—, —(CH₂)ₐ—O—, O—(CH₂)ₐ—, —(CH₂)ₐ—O—(CH₂)_{b}—, —(CH₂)ₐ—S—(CH₂)_{b}—, —(CH₂)ₐ—SO—(CH₂)_{b}—, —(CH₂)ₐ—SO₂—, —SO₂—(CH₂)_{b}—, —(CH₂)ₐ—SO—, —SO—(CH₂)_{b}—, —(CH₂)ₐ—SO₂—(CH₂)_{b}—, —(CH₂)ₐ—S—(CH₂)_{b}—O—, —O—(CH₂)ₐ—S—(CH₂)_{b}—, —(CH₂)ₐ—O—(CH₂)_{b}—S—, —S—(CH₂)ₐ—O—(CH₂)_{b}—, heterocycloalkylene-(CH₂)ₐ—O—, —O—(CH₂)ₐ-heterocycloalkylene-, —(CH₂)ₐ—O-heterocycloalkylene, heterocycloalkylene-O—(CH₂)ₐ—, —O-heterocycloalkylene-(CH₂)ₐ— and —(CH₂)ₐ-heterocycloalkylene-O—, wherein a and b are each independently an integer from 1 to 6.

In one preferred embodiment, L is selected from —O—, —NH—SO₂—, —NH—SO₂—(CH₂)ₐ—, —SO₂—NH—, —SO₂—NH—(CH₂)ₐ—, —O—SO₂—, —SO₂—O—, —(CH₂)ₐ—, —(CH₂)ₐ—O—, O—(CH₂)ₐ—, —(CH₂)ₐ—O—(CH₂)_{b}—, —(CH₂)ₐ—S—(CH₂)_{b}—, —(CH₂)ₐ—SO—(CH₂)_{b}—, —(CH₂)ₐ—SO₂—, —SO₂—(CH₂)_{b}—, —(CH₂)ₐ—SO—, —SO—(CH₂)_{b}—, —(CH₂)ₐ—SO₂—(CH₂)_{b}—, —(CH₂)ₐ—S—(CH₂)_{b}—O—, —O—(CH₂)ₐ—S—(CH₂)_{b}—, —(CH₂)ₐ—O—(CH₂)_{b}—S—, —S—(CH₂)ₐ—O—(CH₂)_{b}—, heterocycloalkylene-(CH₂)ₐ—O—, —O—(CH₂)ₐ-heterocycloalkylene-, —(CH₂)ₐ—O-heterocycloalkylene, heterocycloalkylene-O—(CH₂)ₐ—, —O-heterocycloalkylene-(CH₂)ₐ— and —(CH₂)ₐ-heterocycloalkylene-O—, wherein a and b are each independently an integer from 1 to 6.

More preferably, L is selected from —O—, —O—SO₂—, —SO₂—O—, —(CH₂)ₐ—, —(CH₂)ₐ—O—, O—(CH₂)ₐ—, —(CH₂)ₐ—O—(CH₂)_{b}—, —(CH₂)ₐ—S—(CH₂)_{b}—, —(CH₂)ₐ—S—(CH₂)_{b}—O—, —O—(CH₂)ₐ—S—(CH₂)_{b}—, —(CH₂)ₐ—O—(CH₂)_{b}—S—, —S—(CH₂)ₐ—O—(CH₂)_{b}—, heterocycloalkylene-(CH₂)ₐ—O— and —(CH₂)ₐ—O-heterocycloalkylene, wherein a and b are each independently an integer from 1 to 6.

In one preferred embodiment, L is selected from —CH₂—, —CH₂CH₂—, —CH₂CH₂CH₂—, —CH₂CH₂O—, —OCH₂CH₂—, —CH₂O—, —OCH₂—, —CH(Me)OCH₂, —CH₂OCH(Me)-, —CH(CF₃)OCH₂—, —CH₂OCH(CF₃)—, —NH—SO₂—, —NH—SO₂—CH₂—, —CH₂—SO₂NH—, —SO₂—NH—, —SO₂—NH—CH₂—, —CH₂—NH—SO₂—, —CH₂CH₂CH₂O—, —OCH₂CH₂CH₂—, —CH₂SO₂CH₂—, —CH₂SO₂—, —SO₂CH₂—, —CH₂SO—, —SOCH₂—, —CH₂SOCH₂—, —CH₂SCH₂—, —CH₂CH₂CH₂CH₂O—, —OCH₂CH₂CH₂CH₂—, —CH₂OCH₂, —CH(Me)O—, —OCH(Me)-, —CH(CF₃)O—, —OCH(CF₃)—, —CH₂SCH₂O— and —O—CH₂SCH₂—.

In one preferred embodiment, L is selected from —CH₂—, —CH₂CH₂CH₂—, —CH₂CH₂O—, —NH—SO₂—, —NH—SO₂—CH₂—, —SO₂—NH—, —SO₂—NH—CH₂—, —OCH₂CH₂—, —CH₂CH₂CH₂O—, —OCH₂CH₂CH₂—, —CH₂SO₂CH₂—, —CH₂SO₂—, —SO₂CH₂—, —CH₂SO—, —SOCH₂—, —CH₂SOCH₂—, —CH₂SCH₂—, —CH₂CH₂CH₂CH₂O—, —OCH₂CH₂CH₂CH₂—, —CH₂OCH₂ and —CH₂SCH₂O—.

In one preferred embodiment, L-Z is —OCH₂CH₂Ph, —OCH₂Ph, —OCH₂CH₂CH(Me)₂, —OCH₂CH(Me)₂, —OSO₂-(4-methylphenyl), —CH₂SO₂CH₂-Ph, —CH₂SO₂C-Ph, —OCH₂-cyclopropyl, —OCH₂CH₂CH₂CH₃, —OCH₂CH₂CH₂CH₂Ph, —CH₂OCH₂Ph, —OCH₂SCH₂Ph, —CH₂OCH₂Ph, —CH₂CH₂Ph, —CH₂SO₂-(4-methylphenyl), —CH₂SO₂-(4-methoxyphenyl), —CH₂SO₂-(4-chlorophenyl), —CH₂SO₂-(3-chlorophenyl), —CH₂SO₂-(2-chlorophenyl), —CH₂OCH(Me)-Ph, —CH₂OCH(CF₃)-Ph, —CH₂OCH₂-cyclopropyl, —CH₂O-cyclopropyl, —OCH(Me)-cyclopropyl, —CH(Me)O-cyclopropyl, —OCH(CF₃)-cyclopropyl, —CH(CF₃)O-cyclopropyl, CH₂OCH₂-(pyridin-2-yl), —CH₂OCH₂(4-methoxyphenyl), CH₂OCH₂(3-methoxyphenyl), —CH₂SO₂-(4-methylpyridin-3-yl)-chlorophenyl),

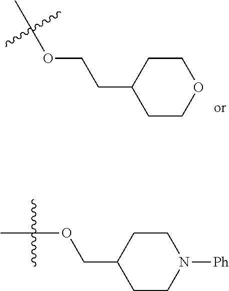 [SMILES: *OCCC1CCOCC1 ; *OCC1CCN(c2ccccc2)CC1]

In one preferred embodiment, L-Z is —OCH₂CH₂Ph, —OCH₂Ph, —OCH₂CH₂CH(Me)₂, —OCH₂CH(Me)₂, —OSO₂-(4-methylphenyl), —CH₂SO₂CH₂-Ph, —OCH₂-cyclopropyl, —OCH₂CH₂CH₂CH₃, —CH₂OCH₂Ph,

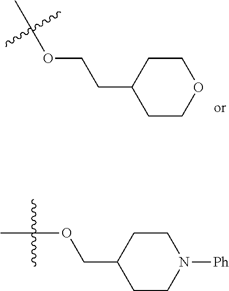 [SMILES: *OCCC1CCOCC1 ; *OCC1CCN(c2ccccc2)CC1]

In one highly preferred embodiment, L-Z is —OCH₂CH₂Ph, —OCH₂CH₂CH₂CH₂Ph or —CH₂OCH₂Ph. More preferably, L-Z is —OCH₂CH₂Ph.

In one preferred embodiment, the compound is selected from the following:

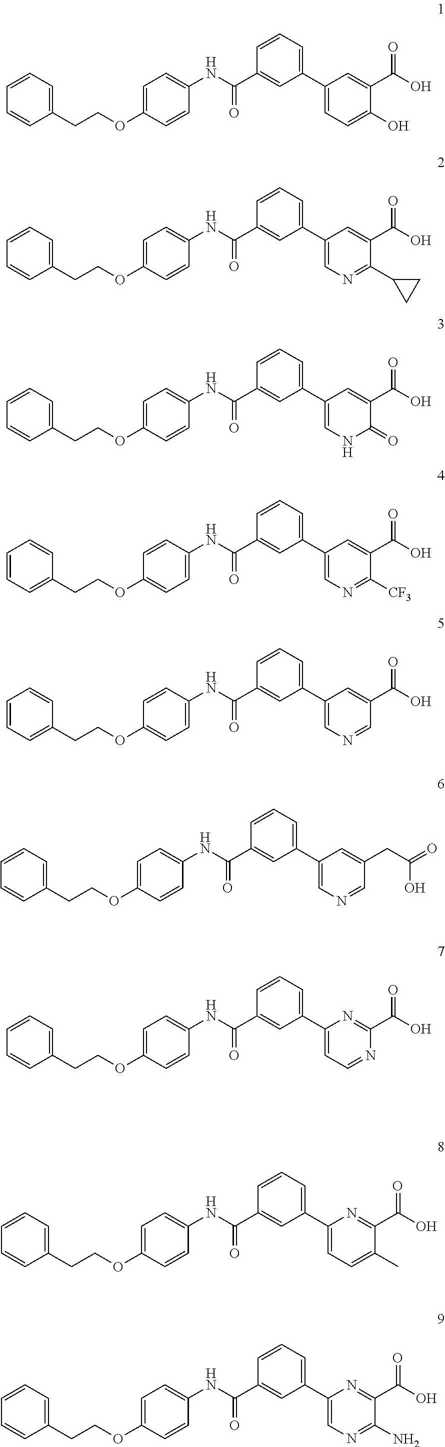

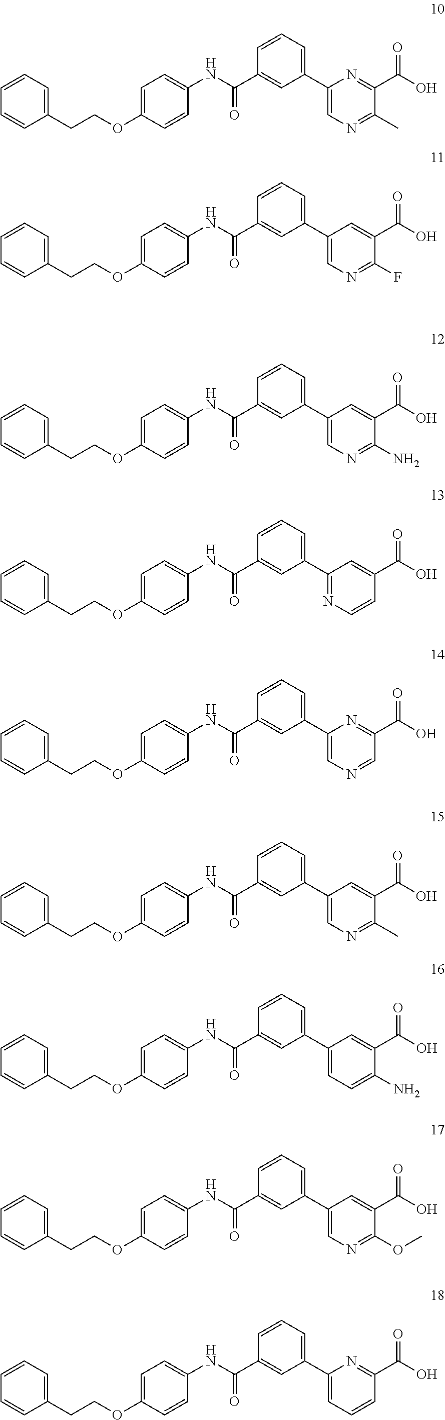

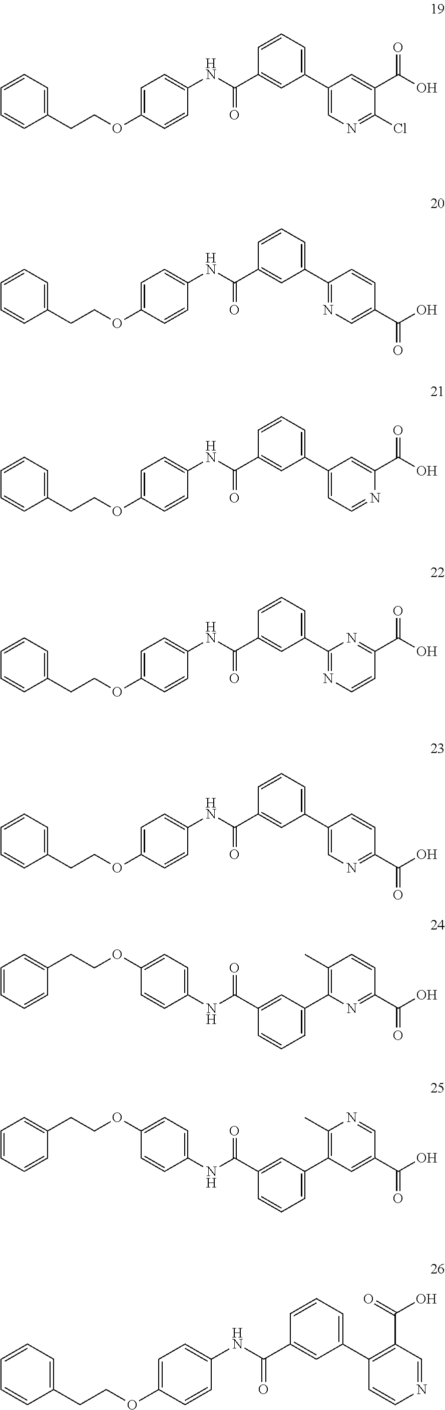

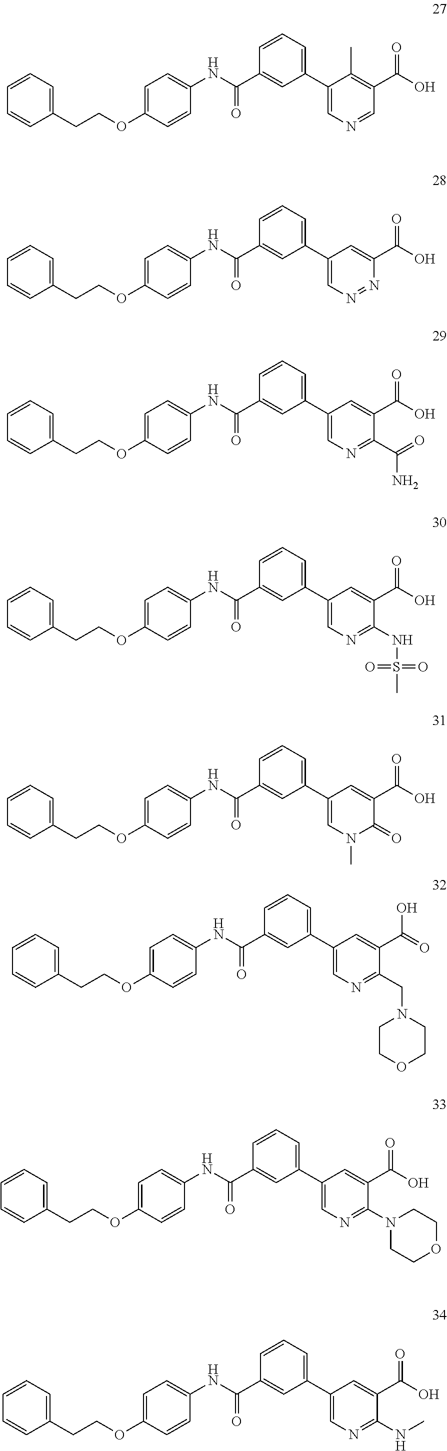

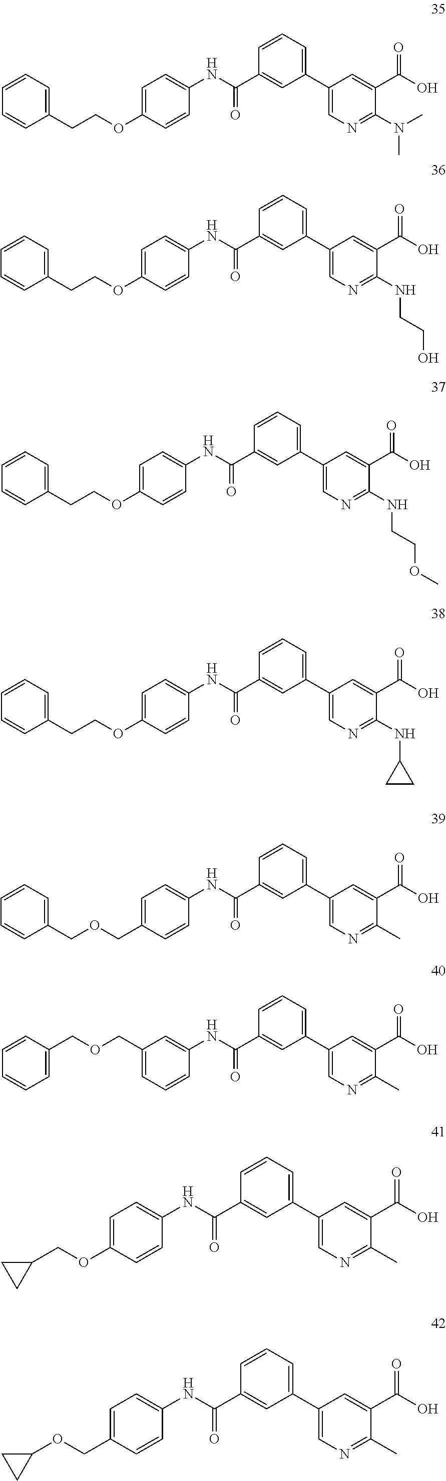

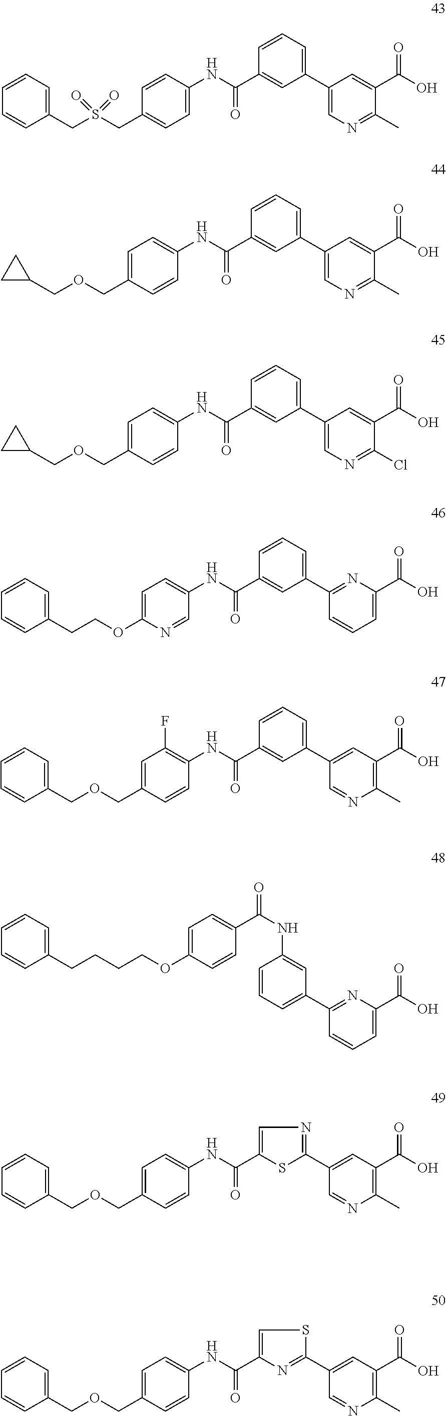

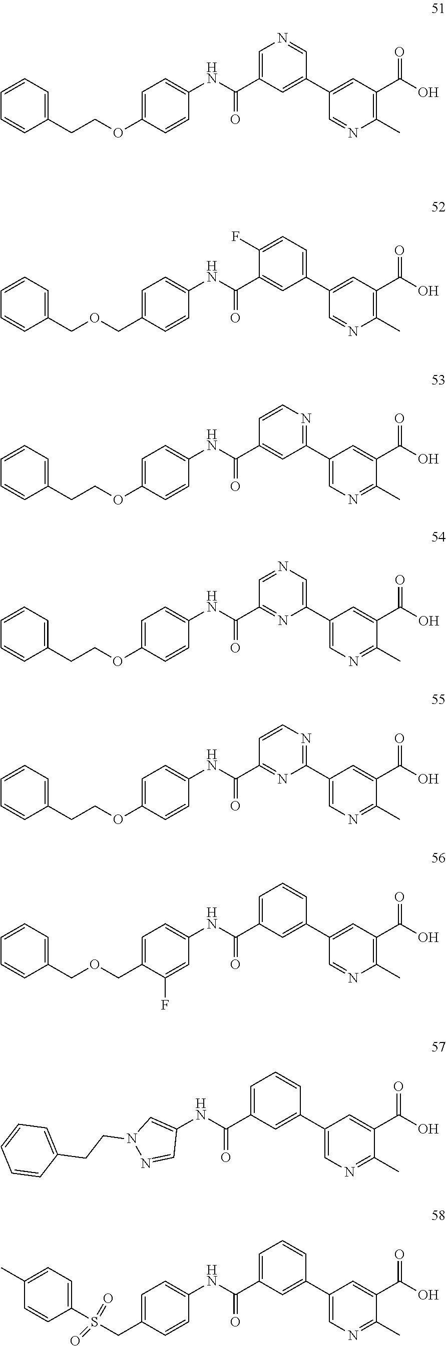

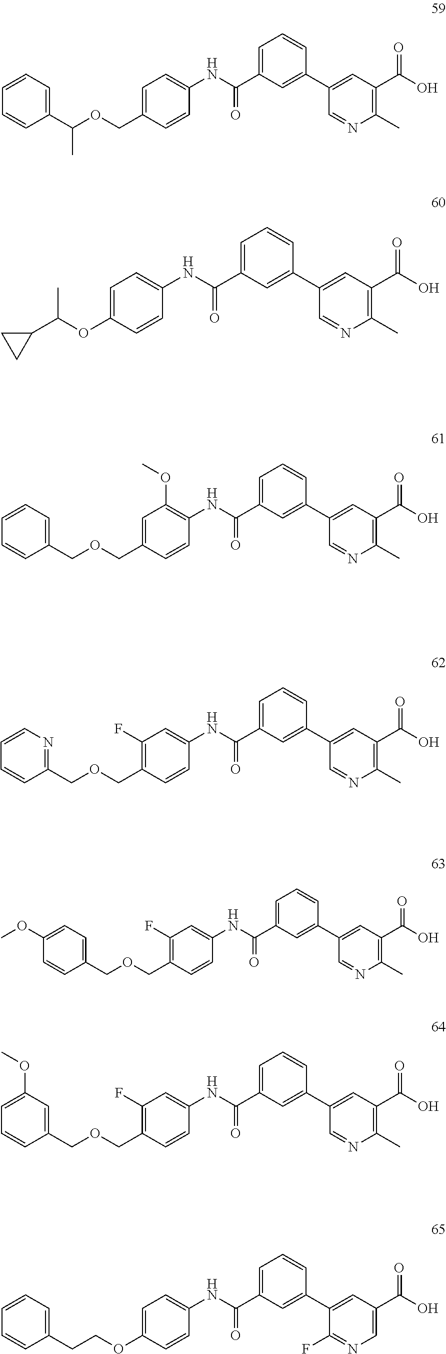

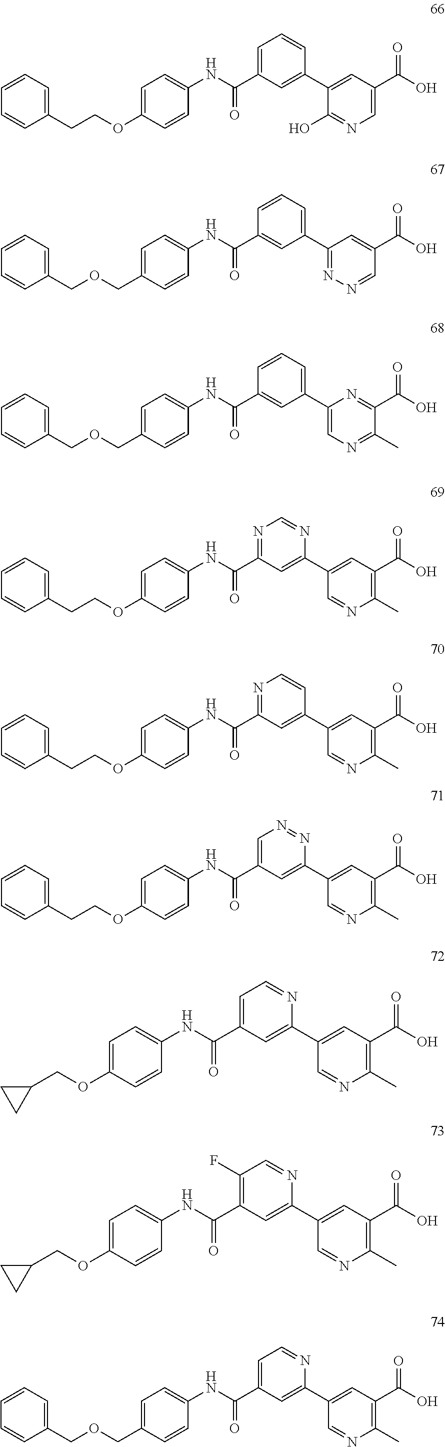

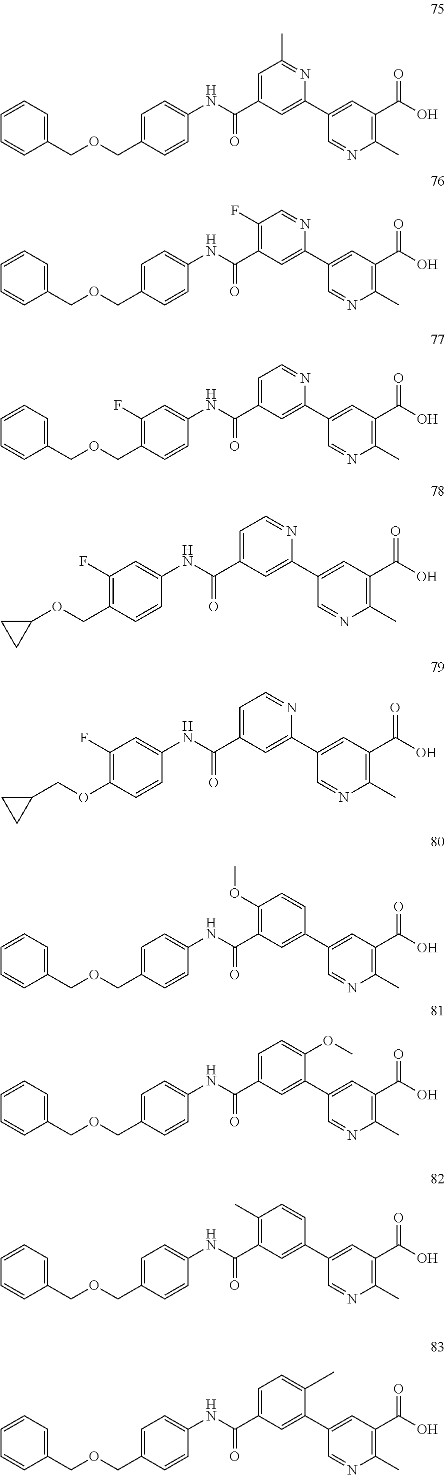

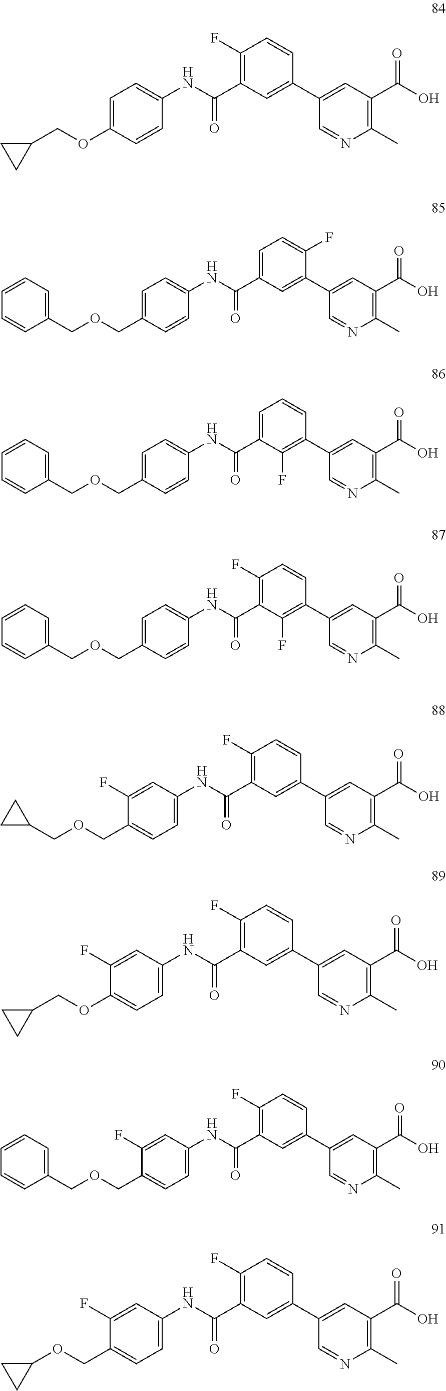

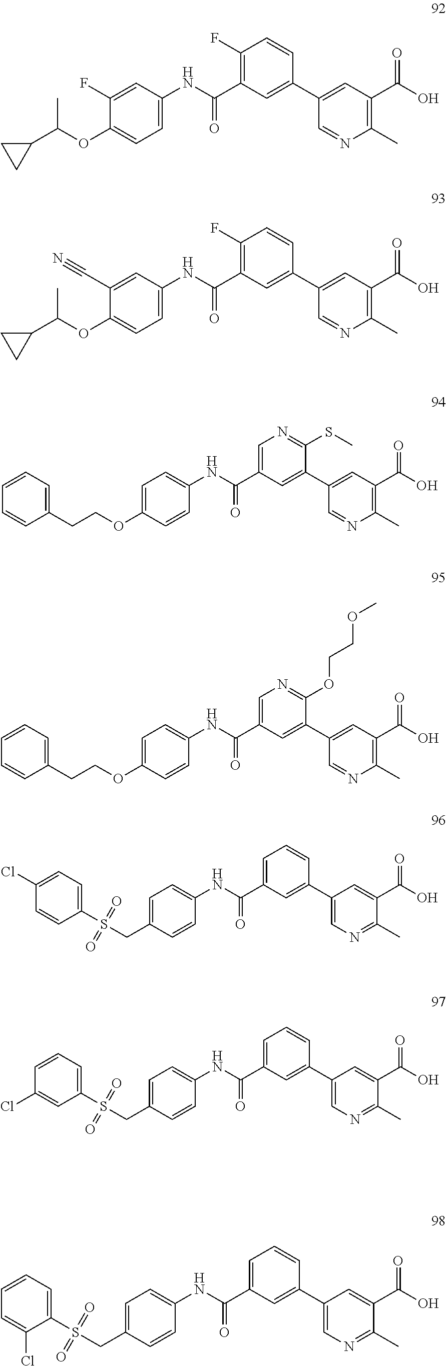

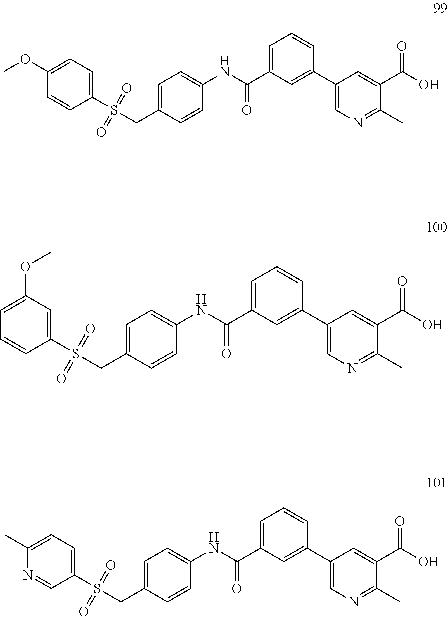 [SMILES: COc1ccc(S(=O)(=O)Cc2ccc(NC(=O)c3cccc(-c4cnc(C)c(C(=O)O)c4)c3)cc2)cc1 ; COc1cccc(S(=O)(=O)Cc2ccc(NC(=O)c3cccc(-c4cnc(C)c(C(=O)O)c4)c3)cc2)c1 ; Cc1ccc(S(=O)(=O)Cc2ccc(NC(=O)c3cccc(-c4cnc(C)c(C(=O)O)c4)c3)cc2)cn1]

and pharmaceutically acceptable salts and solvates thereof.

In one preferred embodiment, the compound of formula (m) is selected from the following compounds as shown herein: 1-20, 22, 24, 28, 30, 33-39, 48, 52, 53, 56-60, 63-65, 67-70, 74-77, 80, 81, 83, 85, 88-90, 92 and 96, and pharmaceutically acceptable salts and solvates thereof.

In another preferred embodiment, the compound of formula (I) is selected from the following:

| | |
|---|---|
| 1 | 4-Hydroxy-3′-((4-phenethoxyphenyl)carbamoyl)-[1,1′-biphenyl]-3-carboxylic acid |
| 2 | 2-Cyclopropyl-5-(3-((4-phenethoxyphenyl)carbamoyl)phenyl)nicotinic acid |
| 3 | 2-Oxo-5-(3-((4-phenethoxyphenyl)carbamoyl)phenyl)-1,2-dihydropyridine-3- |
| | carboxylic acid |
| 4 | 5-(3-((4-Phenethoxyphenyl)carbamoyl)phenyl)-2-(trifluoromethyl)nicotinic acid |
| 5 | 5-(3-((4-Phenethoxyphenyl)carbamoyl)phenyl)nicotinic acid |
| 6 | 2-(5-(3-((4-Phenethoxyphenyl)carbamoyl)phenyl)pyridin-3-yl)acetic acid |
| 7 | 4-(3-((4-Phenethoxyphenyl)carbamoyl)phenyl)pyrimidine-2-carboxylic acid |
| 8 | 3-Methyl-6-(3-((4-phenethoxyphenyl)carbamoyl)phenyl)picolinic acid |
| 9 | 3-Amino-6-(3-((4-phenethoxyphenyl)carbamoyl)phenyl)pyrazine-2-carboxylic acid |
| 10 | 3-Methyl-6-(3-((4-phenethoxyphenyl)carbamoyl)phenyl)pyrazine-2-carboxylic acid |
| 11 | 2-Fluoro-5-(3-((4-phenethoxyphenyl)carbamoyl)phenyl)nicotinic acid |
| 12 | 2-Amino-5-(3-((4-phenethoxyphenyl)carbamoyl)phenyl)nicotinic acid |
| 13 | 2-(3-((4-Phenethoxyphenyl)carbamoyl)phenyl)isonicotinic acid |
| 14 | 6-(3-((4-Phenethoxyphenyl)carbamoyl)phenyl)pyrazine-2-carboxylic acid |
| 15 | 2-Methyl-5-(3-((4-phenethoxyphenyl)carbamoyl)phenyl)nicotinic acid |
| 16 | 4-Amino-3′-((4-phenethoxyphenyl)carbamoyl)-[1,1′-biphenyl]-3-carboxylic acid |
| 17 | 2-Methoxy-5-(3-((4-phenethoxyphenyl)carbamoyl)phenyl)nicotinic acid |
| 18 | 6-(3-((4-Phenethoxyphenyl)carbamoyl)phenyl)picolinic acid |
| 19 | 2-Chloro-5-(3-((4-phenethoxyphenyl)carbamoyl)phenyl)nicotinic acid |
| 20 | 6-(3-((4-Phenethoxyphenyl)carbamoyl)phenyl)nicotinic acid |
| 21 | 4-(3-((4-Phenethoxyphenyl)carbamoyl)phenyl)picolinic acid |
| 22 | 2-(3-((4-Phenethoxyphenyl)carbamoyl)phenyl)pyrimidine-4-carboxylic acid |
| 23 | 5-(3-((4-Phenethoxyphenyl)carbamoyl)phenyl)picolinic acid |
| 24 | 5-Methyl-6-(3-((4-phenethoxyphenyl)carbamoyl)phenyl)picolinic acid |
| 25 | 6-Methyl-5-(3-((4-phenethoxyphenyl)carbamoyl)phenyl)nicotinic acid |
| 26 | 4-(3-((4-Phenethoxyphenyl)carbamoyl)phenyl)nicotinic acid |
| 27 | 4-Methyl-5-(3-((4-phenethoxyphenyl)carbamoyl)phenyl)nicotinic acid |
| 28 | 5-(3-((4-Phenethoxyphenyl)carbamoyl)phenyl)pyridazine-3-carboxylic acid |
| 29 | 2-Carbamoyl-5-(3-((4-phenethoxyphenyl)carbamoyl)phenyl)nicotinic acid |
| 30 | 2-(Methylsulfonamido)-5-(3-((4-phenethoxyphenyl)carbamoyl)phenyl)nicotinic acid |
| 31 | 1-Methyl-2-oxo-5-(3-((4-phenethoxyphenyl)carbamoyl)phenyl)-1,2- |
| | dihydropyridine-3-carboxylic acid |
| 32 | 2-(Morpholinomethyl)-5-(3-((4-phenethoxyphenyl)carbamoyl)phenyl)nicotinic acid |
| 33 | 2-Morpholino-5-(3-((4-phenethoxyphenyl)carbamoyl)phenyl)nicotinic acid |
| 34 | 2-(Methylamino)-5-(3-((4-phenethoxyphenyl)carbamoyl)phenyl)nicotinic acid |
| 35 | 2-(Dimethylamino)-5-(3-((4-phenethoxyphenyl)carbamoyl)phenyl)nicotinic acid |
| 36 | 2-((2-Hydroxyethyl)amino)-5-(3-((4-phenethoxyphenyl)carbamoyl)phenyl)nicotinic |
| | acid |
| 37 | 2-((2-Methoxyethyl)amino)-5-(3-((4-phenethoxyphenyl)carbamoyl)phenyl)nicotinic |
| | acid |
| 38 | 2-(Cyclopropylamino)-5-(3-((4-phenethoxyphenyl)carbamoyl)phenyl)nicotinic acid |
| 39 | 5-(3-((4-((Benzyloxy)methyl)phenyl)carbamoyl)phenyl)-2-methylnicotinic acid |
| 40 | 5-(3-((3-((Benzyloxy)methyl)phenyl)carbamoyl)phenyl)-2-methylnicotinic acid |
| 41 | 5-(3-((4-(Cyclopropylmethoxy)phenyl)carbamoyl)phenyl)-2-methylnicotinic acid |
| 42 | 5-(3-((4-(Cyclopropoxymethyl)phenyl)carbamoyl)phenyl)-2-methylnicotinic acid |
| 43 | 5-(3-((4-((Benzylsulfonyl)methyl)phenyl)carbamoyl)phenyl)-2-methylnicotinic acid |
| 44 | 5-(3-((4-((Cyclopropylmethoxy)methyl)phenyl)carbamoyl)phenyl)-2-methylnicotinic |
| | acid |
| 45 | 2-Chloro-5-(3-((4-((cyclopropylmethoxy)methyl)phenyl)carbamoyl)phenyl)nicotinic |
| | acid |
| 46 | 6-(3-((6-Phenethoxypyridin-3-yl)carbamoyl)phenyl)picolinic acid |
| 47 | 5-(3-((4-((Benzyloxy)methyl)-2-fluorophenyl)carbamoyl)phenyl)-2-methylnicotinic |
| | acid |
| 48 | 6-(3-(4-(4-Phenylbutoxy)benzamido)phenyl)picolinic acid |
| 49 | 5-(5-((4-((Benzyloxy)methyl)phenyl)carbamoyl)thiazol-2-yl)-2-methylnicotinic acid |
| 50 | 5-(4-((4-((Benzyloxy)methyl)phenyl)carbamoyl)thiazol-2-yl)-2-methylnicotinic acid |
| 51 | 6-Methyl-5′-((4-phenethoxyphenyl)carbamoyl)-[3,3′-bipyridine]-5-carboxylic acid |
| 52 | 5-(3-((4-((Benzyloxy)methyl)phenyl)carbamoyl)-4-fluorophenyl)-2-methylnicotinic |
| | acid |
| 53 | 6′-Methyl-4-((4-phenethoxyphenyl)carbamoyl)-[2,3′-bipyridine]-5′-carboxylic acid |
| 54 | 2-Methyl-5-(6-((4-phenethoxyphenyl)carbamoyl)pyrazin-2-yl)nicotinic acid |
| 55 | 2-Methyl-5-(4-((4-phenethoxyphenyl)carbamoyl)pyrimidin-2-yl)nicotinic acid |
| 56 | 5-[3-[[4-(Benzyloxymethyl)-3-fluoro-phenyl]carbamoyl]phenyl]-2-methyl-pyridine-3- |
| | carboxylic acid |
| 57 | 2-Methyl-5-[3-[[1-(2-phenylethyl)pyrazol-4-yl]carbamoyl]phenyl]pyridine-3- |
| | carboxylic acid |
| 58 | 2-Methyl-5-[3-[[4-(p-tolylsulfonylmethyl)phenyl]carbamoyl]phenyl]pyridine-3- |
| | carboxylic acid |
| 59 | 2-Methyl-5-[3-[[4-(1-phenylethoxymethyl)phenyl]carbamoyl]phenyl]pyridine-3- |
| | carboxylic acid |
| 60 | 5-[3-[[4-(1-Cyclopropylethoxy)phenyl]carbamoyl]phenyl]-2-methyl-pyridine-3- |
| | carboxylic acid |
| 61 | 5-[3-[[4-(Benzyloxymethyl)-2-methoxy-phenyl]carbamoyl]phenyl]-2-methyl- |
| | pyridine-3-carboxylic acid |
| 62 | 5-[3-[[3-Fluoro-4-(2-pyridylmethoxymethyl)phenyl]carbamoyl]phenyl]-2-methyl- |
| | pyridine-3-carboxylic acid |
| 63 | 5-[3-[[3-Fluoro-4-[(4-methoxyphenyl)methoxymethyl]phenyl]carbamoyl]phenyl]-2- |
| | methyl-pyridine-3-carboxylic acid |
| 64 | 5-[3-[[3-Fluoro-4-[(3-methoxyphenyl)methoxymethyl]phenyl]carbamoyl]phenyl]-2- |
| | methyl-pyridine-3-carboxylic acid |
| 65 | 6-Fluoro-5-[3-[[4-(2-phenylethoxy)phenyl]carbamoyl]phenyl]pyridine-3-carboxylic |
| | acid |
| 66 | 6-Hydroxy-5-[3-[[4-(2-phenylethoxy)phenyl]carbamoyl]phenyl]pyridine-3-carboxylic |
| | acid |
| 67 | 6-[3-[[4-(Benzyloxymethyl)phenyl]carbamoyl]phenyl]pyridazine-4-carboxylic acid |
| 68 | 6-[3-[[4-(Benzyloxymethyl)phenyl]carbamoyl]phenyl]-3-methyl-pyrazine-2- |
| | carboxylic acid |
| 69 | 2-Methyl-5-[6-[[4-(2-phenylethoxy)phenyl]carbamoyl]pyrimidin-4-yl]pyridine-3- |
| | carboxylic acid |
| 70 | 2-Methyl-5-[2-[[4-(2-phenylethoxy)phenyl]carbamoyl]-4-pyridyl]pyridine-3- |
| | carboxylic acid |
| 71 | 2-Methyl-5-[5-[[4-(2-phenylethoxy)phenyl]carbamoyl]pyridazin-3-yl]pyridine-3- |
| | carboxylic acid |
| 72 | 5-[4-[[4-(Cyclopropylmethoxy)phenyl]carbamoyl]-2-pyridyl]-2-methyl-pyridine-3- |
| | carboxylic acid |
| 73 | 5-[4-[[4-(Cyclopropylmethoxy)phenyl]carbamoyl]-5-fluoro-2-pyridyl]-2-methyl- |
| | pyridine-3-carboxylic acid |
| 74 | 5-[4-[[4-(Benzyloxymethyl)phenyl]carbamoyl]-2-pyridyl]-2-methyl-pyridine-3- |
| | carboxylic acid |
| 75 | 5-[4-[[4-(Benzyloxymethyl)phenyl]carbamoyl]-6-methyl-2-pyridyl]-2-methyl- |
| | pyridine-3-carboxylic acid |
| 76 | 5-[4-[[4-(Benzyloxymethyl)phenyl]carbamoyl]-5-fluoro-2-pyridyl]-2-methyl-pyridine- |
| | 3-carboxylic acid |
| 77 | 5-[4-[[4-(Benzyloxymethyl)-3-fluoro-phenyl]carbamoyl]-2-pyridyl]-2-methyl- |
| | pyridine-3-carboxylic acid |
| 78 | 5-[4-[[4-(Cyclopropoxymethyl)-3-fluoro-phenyl]carbamoyl]-2-pyridyl]-2-methyl- |
| | pyridine-3-carboxylic acid |
| 79 | 5-[4-[[4-(Cyclopropylmethoxy)-3-fluoro-phenyl]carbamoyl]-2-pyridyl]-2-methyl- |
| | pyridine-3-carboxylic acid |
| 80 | 5-[3-[[4-(Benzyloxymethyl)phenyl]carbamoyl]-4-methoxy-phenyl]-2-methyl- |
| | pyridine-3-carboxylic acid |
| 81 | 5-[5-[[4-(Benzyloxymethyl)phenyl]carbamoyl]-2-methoxy-phenyl]-2-methyl- |
| | pyridine-3-carboxylic acid |
| 82 | 5-[3-[[4-(Benzyloxymethyl)phenyl]carbamoyl]-4-methyl-phenyl]-2-methyl-pyridine- |
| | 3-carboxylic acid |
| 83 | 5-[5-[[4-(Benzyloxymethyl)phenyl]carbamoyl]-2-methyl-phenyl]-2-methyl-pyridine- |
| | 3-carboxylic acid |
| 84 | 5-[3-[[4-(Cyclopropylmethoxy)phenyl]carbamoyl]-4-fluoro-phenyl]-2-methyl- |
| | pyridine-3-carboxylic acid |
| 85 | 5-[5-[[4-(Benzyloxymethyl)phenyl]carbamoyl]-2-fluoro-phenyl]-2-methyl-pyridine-3- |
| | carboxylic acid |
| 86 | 5-[3-[[4-(Benzyloxymethyl)phenyl]carbamoyl]-2-fluoro-phenyl]-2-methyl-pyridine-3- |
| | carboxylic acid |
| 87 | 5-[3-[[4-(Benzyloxymethyl)phenyl]carbamoyl]-2,4-difluoro-phenyl]-2-methyl- |
| | pyridine-3-carboxylic acid |
| 88 | 5-[3-[[4-(Cyclopropylmethoxymethyl)-3-fluoro-phenyl]carbamoyl]-4-fluoro-phenyl]- |
| | 2-methyl-pyridine-3-carboxylic acid |
| 89 | 5-[3-[[4-(Cyclopropylmethoxy)-3-fluoro-phenyl]carbamoyl]-4-fluoro-phenyl]-2- |
| | methyl-pyridine-3-carboxylic acid |
| 90 | 5-[3-[[4-(Benzyloxymethyl)-3-fluoro-phenyl]carbamoyl]-4-fluoro-phenyl]-2-methyl- |
| | pyridine-3-carboxylic acid |
| 91 | 5-[3-[[4-(Cyclopropoxymethyl)-3-fluoro-phenyl]carbamoyl]-4-fluoro-phenyl]-2- |
| | methyl-pyridine-3-carboxylic acid |
| 92 | 5-[3-[[4-(1-Cyclopropylethoxy)-3-fluoro-phenyl]carbamoyl]-4-fluoro-phenyl]-2- |
| | methyl-pyridine-3-carboxylic acid |
| 93 | 5-[3-[[3-Cyano-4-(1-cyclopropylethoxy)phenyl]carbamoyl]-4-fluoro-phenyl]-2- |
| | methyl-pyridine-3-carboxylic acid |
| 94 | 2-Methyl-5-[2-methylsulfanyl-5-[[4-(2-phenylethoxy)phenyl]carbamoyl]-3- |
| | pyridyl]pyridine-3-carboxylic acid |
| 95 | 5-[2-(2-Methoxyethoxy)-5-[[4-(2-phenylethoxy)phenyl]carbamoyl]-3-pyridyl]-2- |
| | methyl-pyridine-3-carboxylic acid |
| 96 | 5-[3-[[4-[(4-Chlorophenyl)sulfonylmethyl]phenyl]carbamoyl]phenyl]-2-methyl- |
| | pyridine-3-carboxylic acid |
| 97 | 5-[3-[[4-[(3-Chlorophenyl)sulfonylmethyl]phenyl]carbamoyl]phenyl]-2-methyl- |
| | pyridine-3-carboxylic acid |
| 98 | 5-[3-[[4-[(2-Chlorophenyl)sulfonylmethyl]phenyl]carbamoyl]phenyl]-2-methyl- |
| | pyridine-3-carboxylic acid |
| 99 | 5-[3-[[4-[(4-Methoxyphenyl)sulfonylmethyl]phenyl]carbamoyl]phenyl]-2-methyl- |
| | pyridine-3-carboxylic acid |
| 100 | 5-[3-[[4-[(3-Methoxyphenyl)sulfonylmethyl]phenyl]carbamoyl]phenyl]-2-methyl- |
| | pyridine-3-carboxylic acid |
| 101 | 2-Methyl-5-[3-[[4-[(6-methyl-3- |
| | pyridyl)sulfonylmethyl]phenyl]carbamoyl]phenyl]pyridine-3-carboxylic acid |

and pharmaceutically acceptable salts and solvates thereof.

### Process

A further aspect of the invention relates to processes for preparing a compound as defined herein.

In one embodiment, the process comprises reacting a dioxaborolane intermediate with a halo intermediate (preferably a bromo intermediate) as shown below to form a compound of formula (I):

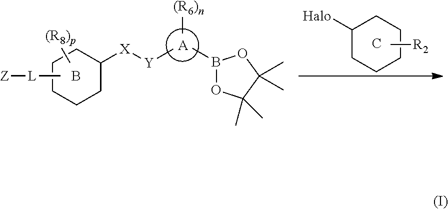

In an alternative embodiment, the process comprises reacting a dioxaborolane intermediate with a halo intermediate (preferably a bromo intermediate) as shown below to form a compound of formula (I):

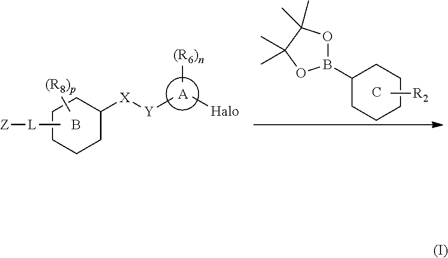

In another embodiment, X—Y is NH—CO and the process comprises coupling an amine intermediate with a carboxylic acid as shown below:

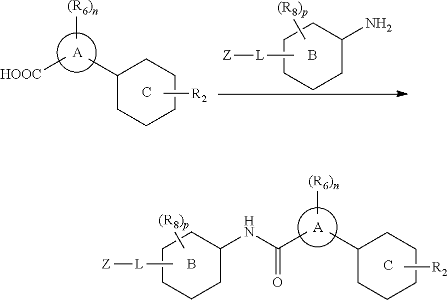

Further details of the synthetic processes are set forth in the accompanying examples section.

### Therapeutic Applications

A further aspect of the invention relates to compounds as described herein for use in medicine. The compounds have particular use in the field of oncology, gastrointestinal disorders, and inflammatory disorders as described in more detail below. In a preferred embodiment, the compound of the invention modulates GPR35 function.

One aspect of the invention therefore relates to compounds as described herein for use as a medicament.

Preferably, the compound of formula (I) is for use in treating or preventing a disorder selected from a proliferative disorder, a fibrotic disorder, a gastrointestinal disorder, a cardiovascular disease, an immune disorder and an inflammatory disorder.

In one preferred embodiment, the compounds have applications in the field of oncology. For example, in one preferred embodiment, the compounds are for use in treating a proliferative disorder, preferably a cancer or leukemia. GPR35 expression is known to be associated with cancer. More specifically, GPR35 expression is commonly upregulated in cancers of the GI tract, relative to normal tissue.²⁴ GPR35 expression is capable of transforming NIH3T3 murine fibroblast cells and is expressed in gastric cancer cells.²⁵ GPR35b is expressed by colon cancer cell lines and primary colon tumours, while involved patient lymph nodes can express high levels of GPR35b.²⁶ High expression of GPR35b in lymph nodes of colon cancer patients is a marker for poor prognosis. Also, high expression of GPR35 in primary gastric tumours is associated with poor prognosis.⁴⁰ Similarly, above median expression of GPR35 in primary tumours was seen as a poor prognostic marker in males with colorectal cancer, though the opposite effect was reported in females.²⁷ siRNA knockdown of GPR35 reduces viability and proliferation of human gastric cancer cells, while also reversing pro-tumour M2 macrophage phenotype.⁴⁰ Murine Gpr35 was shown to promote glycolysis, proliferation and oncogenic signalling by engaging with the sodium potassium pump (Na/K-ATPase).²¹ Deletion of Gpr35 promoted Na/K-ATPase-mediated ion transport and reduced Src kinase activation and overall metabolic activity in both macrophages and intestinal epithelial cells. Gpr35 deletion or inhibition, with a specific anti-Gpr35 peptide (pepducin),²⁸ prevented inflammation-associated and spontaneous intestinal tumorigenesis in mice. Furthermore, activation of human GPR35 in human inducible pluripotent stem cell (iPSC)-derived macrophages, by way of expression of T108M hypermorphic variant, has been shown to promote angiogenic tube formation by enhanced release of pro-angiogenic factors.²⁸ Finally, selective deletion of Gpr35 in macrophages profoundly reduced tumour growth in inflammation-associated and mutant (hypomorphic) tumour suppressor adenomatous polyposis coli (Apc^{min}), spontaneous tumour models.

In one preferred embodiment, the cancer is selected from cancers of the gastrointestinal tract (e.g. colon, rectum, colorectal, stomach, oesophagus, colorectal adenocarcinoma, oesophageal adenocarcinoma, gastric/stomach cancer/adenocarcinoma) and associated tissues (e.g. pancreas, gall bladder and bile duct, liver, intra- and extra-hepatic, perihilar bile duct cancer/adenocarcinoma, cholangiocarcinoma), and also lung, kidney, gynaecological, breast, testicular, skin, prostate, central nervous system and brain.

In one preferred embodiment, the compounds have applications in the field of immune-oncology and the treatment of immune disorders. Thus, in one preferred embodiment, the compound of formula (I) is for use in treating an immune disorder. In another preferred embodiment, the compound of formula (I) is for use in immunotherapy for the treatment of cancer.

In one preferred embodiment, the immune disorder is an autoimmune disorder. Thus, in one preferred embodiment, the compounds of the invention have applications in treating or preventing multiple sclerosis (MS). Recent studies have implicated the gut microbiota in the pathogenesis of MS. In particular, gut microbiota-induced kynurenic acid recruits GPR35-positive macrophages to promote experimental autoimmune encephalitis, which is an established animal model for MS.⁴¹

In one preferred embodiment, the disorder is fibrosis or a fibrotic disorder. Fibrosis is defined by the excessive accumulation of fibrous connective tissue (components of the extracellular matrix (ECM) such as collagen and fibronectin) in and around inflamed or damaged tissue, which can lead to permanent scarring and organ malfunction.

In one preferred embodiment, the compounds have applications in treating or preventing inflammatory disorders/diseases and/or inflammation.

In one preferred embodiment, the disorder is a gastrointestinal disorder, preferably selected from inflammatory bowel disease, ulcerative colitis, primary sclerosing cholangitis and Crohn's disease.⁴²⁻⁴⁵ Single nucleotide polymorphisms (SNPs) of human GPR35 have been investigated in genome wide association studies.¹⁸, Six of these SNPs have been associated with inflammatory diseases of the GI tract. Types of inflammatory bowel disease (IBD) include ulcerative colitis, Crohn's disease and primary sclerosing cholangitis.^{19,20} SNP rs3749171 is synonymous with a coding variant T108M (GPR35a amino acid sequence numbering) and has been associated with IBD. Research has shown that this variant is hypermorphic, leading to activation of GPR35 and increased proliferation and metabolism in bone marrow-derived macrophages.²¹ Furthermore, expression of T108M GPR35 leads to increased production of VEGF and CXCL8 by macrophages compared to the reference allele, with reduced production in GPR35-deficient cells.²² It has also been suggested that the hypermorphic nature of T108M contributes to pathogenesis in IBD patients and could act as a biomarker for patients that respond better to TNF blockers.^{2,23}

In one preferred embodiment, the disorder is a cardiovascular disease, preferably selected from hypertension, heart failure, atherosclerosis, peripheral vascular disease and stroke. A number of recent publications have suggested a role for GPR35 in hypertension and the pathology of both heart failure and atherosclerosis. For example, a S294R SNP within GPR35 was shown to have significant association with coronary artery calcification in a patient cohort²⁹. Further studies demonstrated a correlation between GPR35 upregulation and traditional heart failure biomarkers such as plasma brain natriuretic peptide, ejection fraction, and pulmonary arterial pressure, thereby implying that GPR35 plays a role in heart failure and hypertension³⁰.

Another aspect relates to a compound as described herein for use in treating or preventing a disorder caused by, associated with or accompanied by abnormal activity of GPR35.

Another aspect relates to a compound as described herein for use in treating or preventing a GPR35-associated disease or disorder.

Another aspect of the invention relates to a method of treating a disorder as described above comprising administering a compound as described herein to a subject.

Another aspect of the invention relates to a method of treating a GPR35-associated disease or disorder in a subject. The method according to this aspect of the present invention is effected by administering to a subject in need thereof a therapeutically effective amount of a compound of the present invention, as described hereinabove, either per se, or, more preferably, as a part of a pharmaceutical composition, mixed with, for example, a pharmaceutically acceptable carrier, as is detailed hereinafter.

Yet another aspect of the invention relates to a method of treating a subject having a disease state alleviated by modulation of GPR35 wherein the method comprises administering to the subject a therapeutically effective amount of a compound according to the invention.

Another aspect relates to a method of treating a disease state alleviated by modulation of GPR35, wherein the method comprises administering to a subject a therapeutically effective amount of a compound according to the invention.

In one preferred embodiment, the compound inhibits GPR35 activity, for example, as demonstrated in the functional GPR35 assay described in the accompanying examples section.

In one preferred embodiment, the compound is a GPR35 antagonist or inverse agonist.

In one preferred embodiment, the compound is a GPR35 antagonist, which reverses the agonist-driven function of a receptor.

In another preferred embodiment, the compound is an inverse agonist of GPR35. Inverse agonists are compounds that interact with receptor-signal transduction systems that have a constitutive level of activity, and, through interaction with the receptor, reduce the activity in the direction opposite of that of a pure agonist.

In one preferred embodiment, the compound of the invention is an allosteric modulator of GPR35, more preferably a negative allosteric modulator. As used herein, a negative allosteric modulator antagonises the agonist activation of a receptor via binding to a different site to that of the agonist. Accordingly, a negative allosteric modulator reduces the affinity or efficacy of an agonist for a receptor. This is in contrast to an orthosteric antagonist that blocks the agonist activation of a receptor via binding at the same site as the agonist.

Preferably, the subject is a mammal, more preferably a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₉₀ as determined in cell culture. Such information can be used to more accurately to determine useful doses in humans. Initial dosages can also be estimated from in vivo data. Using these initial guidelines one of ordinary skill in the art could determine an effective dosage in humans.

Moreover, toxicity and therapeutic efficacy of the compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see, e.g., Fingl et al, 1975, The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/day, commonly from about 100-1000 mg/day, preferably from about 150-700 mg/day and most preferably from 50-150 mg/day. Preferably, therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

As used herein, “GPR35-related disease or disorder” refers to a disease or disorder characterized by inappropriate GPR35 activity. Inappropriate GPR35 activity refers to either an increase or decrease in GPR35 activity as measured by enzyme or cellular assays, for example, compared to the activity in a healthy subject. Inappropriate activity could also be due to overexpression of GPR35 in diseased tissue compared with healthy adjacent tissue where GPR35 expression is lower.

Preferred diseases or disorders that the compounds described herein may be useful in preventing include those described hereinbefore.

Thus, the present invention further provides use of compounds as defined herein in the preparation of a medicament for the treatment of a disease where it is desirable to modulate GPR35. Such diseases include proliferative disorders, gastrointestinal disorders, fibrotic disorders, cardiovasular diseases, immune disorders and inflammatory disorders. Proliferative disorders preferably include therapeutic applications in the field of oncology.

As used herein the phrase “preparation of a medicament” includes the use of the components of the invention directly as the medicament in addition to their use in any stage of the preparation of such a medicament.

The functional GPR35 assay as described in the accompanying examples measures the ability of GPR35 modulators to inhibit a GPR35 agonist-induced phospho-ERK signal. This is expressed as the concentration of modulator required to reduce the phospho-ERK signal by 50 percent, i.e. the IC₅₀; the signal window being defined as the difference between agonist plus modulator vehicle (no modulator) and agonist vehicle (no agonist) controls.

In one preferred embodiment, the compound exhibits an IC₅₀ value in the aforementioned GPR35 assay of less than about 50 μM. More preferably, the compound exhibits an IC₅₀ value in the GPR35 assay of less than about 10 μM, more preferably, less than about 1 μM.

In one preferred embodiment, the compound according to the invention exhibits an IC₅₀ of <10 μM in the aforementioned GPR35 assay. In one preferred embodiment, the compound is selected from those denoted “A” or “B” in Table 1.

In one preferred embodiment, the compound according to the invention exhibits an IC₅₀ of >1 μM and <10 μM in the aforementioned GPR35 assay. In one preferred embodiment, the compound is selected from those denoted “B” in Table 1.

In a more preferred embodiment, the compound according to the invention exhibits an IC₅₀ of <1 μM in the aforementioned assay. In one preferred embodiment, the compound is selected from those denoted “A” in Table 1.

### Pharmaceutical Compositions

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers, excipients or diluents therefor and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller. The carrier, or, if more than one be present, each of the carriers, must be acceptable in the sense of being compatible with the other ingredients of the formulation and not deleterious to the recipient.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet.

Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound as described herein into conjunction or association with a pharmaceutically or veterinarily acceptable carrier or vehicle.

### Salts/Esters

The compounds of the invention can be present as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetra phthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

The present invention also includes all suitable isotopic variations of the compound or a pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹¹C, ¹³C, ¹⁴C, ¹⁵N, ¹⁷O, ¹⁸O, ³¹P, ³²P ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formula (I) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Atropisomers

Some of the compounds of the invention may exist as atropisomers. Atropisomers are stereoisomers arising because of hindered rotation about a single bond, where energy differences due to steric strain or other contributors create a barrier to rotation that is high enough to allow for isolation of individual conformers. The invention encompasses all such atropisomers.

### Prodrugs

The invention further includes the compounds of the present invention in prodrug form, i.e. covalently bonded compounds which release the active parent drug in vivo. Such prodrugs are generally compounds of the invention wherein one or more appropriate groups have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Solvates

The present invention also includes solvate forms of the compounds of the present invention. The terms used in the claims encompass these forms. Preferably, the solvate is a hydrate.

### Combinations

A further aspect of the invention relates to a combination comprising a compound as described herein and one or more additional active agents. In a particularly preferred embodiment, the one or more compounds of the invention are administered in combination with one or more additional active agents, for example, existing drugs available on the market. In such cases, the compounds of the invention may be administered consecutively, simultaneously or sequentially with the one or more other active agents.

Drugs in general can be more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance.

Beneficial combinations may be suggested by studying the activity of the test compounds with agents known or suspected of being valuable in the treatment of a particular disorder. This procedure can also be used to determine the order of administration of the agents, i.e. before, simultaneously, or after delivery. Such scheduling may be a feature of all the active agents identified herein.

In the context of cancer, compounds of the invention can be used in combination with immunotherapies such as cancer vaccines and/or with other immune-modulators. Thus, in one preferred embodiment, the additional active agent is an immunotherapy agent, more preferably a cancer immunotherapy agent. An “immunotherapy agent” refers to a treatment that uses the subject's own immune system to fight diseases such as cancer. For other disorders the compounds of the invention can be used in combination with agents that block or decrease inflammation such as antibodies that target pro-inflammatory cytokines. The compounds of the invention can also be used in combination with other chemotherapy agents and/or in conjunction with radiotherapy.

### Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient, and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

The dosage amount will further be modified according to the mode of administration of the compound. For example, to achieve an “effective amount” for acute therapy, parenteral administration of a compound is typically preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg; preferably between 0.1 and 20 mg, in a manner to maintain the concentration of drug in the plasma at a concentration effective to modulate GPR35. The compounds may be administered one to four times daily at a level to achieve a total daily dose of about 0.4 to about 400 mg. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 500 mg or about 0.1 to about 50 mg in a manner consistent with the condition of the patient. Preferably the oral dose would be about 0.5 to about 50 mg or about 0.5 to about 20 mg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

The invention is further described by way of the following non-limiting examples.

### EXAMPLES

Where the preparation of starting materials is not described, these are commercially available, known in the literature, or readily obtainable by those skilled in the art using standard procedures. Where it is indicated that compounds were prepared analogously to earlier examples or intermediates, it will be appreciated by the skilled person that the reaction time, number of equivalents of reagents, solvent, concentration and temperature can be modified for each specific reaction and that it may be necessary or desirable to employ different work-up or purification techniques.

### General Schemes

Abbreviations

| | |
|---|---|
| AcOH | Acetic acid |
| aq. | Aqueous |
| Ar | Aryl |
| C18 | Octadecyl carbon |
| Celite ® | Diatomaceous earth |
| d | Days |
| d | Doublet (NMR spectrum) |
| DCM | Dichloromethane |
| Dioxane | 1,4-Dioxane |
| DIPEA | N,N-Diisopropylethylamine |
| DMAP | 4-Dimethylaminopyridine |
| DME | 1,2-Dimethoxyethane |
| DMF | N,N-Dimethylformamide |
| DMSO | Dimethyl sulphoxide |
| Et₂O | Diethyl ether |
| EtOAc | Ethyl acetate |
| EtOH | Ethanol |
| eq. | Molar equivalents |
| h | Hour(s) |
| HATU | 1-[bis(Dimethylamino)methylene]-1H-1,2,3-triazolo[4,5-b]pyridinium 3- |
| | oxid hexafluorophosphate |
| het | Heterocycle |
| iPnONO | Isopentyl/isoamyl nitrite |
| LCMS | Liquid chromatography mass spectrometry |
| m | Multiplet |
| mCPBA | 3-Chloroperbenzoic acid |
| MeCN | Acetonitrile |
| MeOH | Methanol |
| min | Minute(s) |
| mol. eq. | Molar equivalent |
| Ms | Methanesulfonyl |
| NaBH(OAc)₃ | Sodium triacetoxyborohydride |
| NEt₃ | Triethylamine |
| Pd-118 | [1,1′-bis(Di-tert-butylphosphino)ferrocene]dichloropalladium(II) |
| Pd(dppf)Cl₂ | [1,1′-bis(diphenylphosphino)ferrocene]dichloropalladium(II) |
| q | Quartet |
| rt | Room temperature |
| RP | Reverse phase |
| s | Singlet |
| Sat. | Saturated |
| SCX | SiliaBond ® Propylsulfonic Acid (SiliaBond SCX-2) |
| t | Triplet |
| tBuOK | Potassium tert-butoxide |
| T3P | 2,4,6-tripropyl-1,3,5,2,4,6-trioxatriphosphinane 2,4,6-trioxide |
| TBME | tert-Butyl methyl ether |
| THF | Tetrahydrofuran |
| Tr | Trityl |
| wt. | Weight |
| X | Halogen atom (bromine or chlorine) |

Other abbreviations are intended to convey their generally accepted meaning.

### General Experimental Conditions

All starting materials and solvents were obtained either from commercial sources or prepared according to literature methods.

Normal phase (“flash”) chromatography and RP (“flash”) chromatography were performed on a CombiFlash Companion automated flash chromatography system, using either RediSep® Silver (230-400 mesh, 40-63 μm irregular) or Silicycle, SiliaSep C18 (230-400 mesh, 40-63 μm irregular) pre-packed silica cartridges respectively. RP flash chromatography was performed using basic modifier (C18, 0-100% MeCN in 0.1% aq. NH₄OH) except for Examples 15, 17, 39-42 and 49-51 where acidic modifier (C18, 0-100% MeCN with 0.1% HCO₂H in 0.1% aq. HCO₂H) was used.

Analytical UPLC-MS experiments to determine retention times and associated mass ions were performed using a Waters ACQUITY UPLC® H-Class system, equipped with ACQUITY PDA Detector and ACQUITY QDa mass spectrometer or Waters SQD mass spectrometer, running the analytical method described below.

Analytical LC-MS experiments to determine retention times and associated mass ions were performed using an Agilent 1200 series HPLC system coupled to an Agilent 1956, 6100 or 6120 series single quadrupole mass spectrometer running one of the analytical methods described below.

Method A - LCMS Acidic Method

| Mobile phase | (A) | 0.1% Formic acid in water | |
|---|---|---|---|
| | (B) | 100% Acetonitrile | |
| Instrument | | Agilent 1260; Quaternary Pump, HiP | |
| | | Sampler, Column Compartment, DAD, | |
| | | G6150 MSD | |
| Column | | Waters Cortecs C18, 30 × 2.1 mm, 2.7 μm | |
| Column oven temperature | | 40° C. | |
| Flow rate | | 1.35 mL/min | |
| Run Time | | 3 min | |
| Detection | | UV at 260 nm +/− 90 nm unless otherwise | |
| | | indicated, MS by electrospray ionisation | |
| Gradient | | | |
| Time | Flow Rate | | |
| (min) | (mL/min) | % A | % B |
| 0.00 | 1.35 | 98 | 2 |
| 2.5 | 1.35 | 0 | 100 |
| 3 | 1.35 | 0 | 100 |

Method B - LCMS Basic Method 10 min

| Mobile phase | (A) | 0.1% Ammonia in water | |
|---|---|---|---|
| | (B) | 100% Acetonitrile | |
| Instrument | | Agilent 1260; Quaternary Pump, HiP | |
| | | Sampler, Column Compartment, DAD, | |
| | | G6150 MSD | |
| Column | | Phenomenex Evo C18, 30 × 2.1 mm, | |
| | | 2.6 μm | |
| Column oven temperature | | 40° C. | |
| Flow rate | | 1.35 mL/min | |
| Run Time | | 3 min | |
| Detection | | UV at 260 nm +/− 90 nm unless otherwise | |
| | | indicated, MS by electrospray ionisation | |
| Gradient | | | |
| Time | Flow Rate | | |
| (min) | (mL/min) | % A | % B |
| 0.00 | 1.35 | 98 | 2 |
| 2.5 | 1.35 | 0 | 100 |
| 3 | 1.35 | 0 | 100 |

Method C - LCMS Acidic Method (1 min)

| Mobile phase | (A) | 0.1% Formic acid in water | |
|---|---|---|---|
| | (B) | 100% Acetonitrile | |
| Instrument | | Agilent 1260; Quaternary Pump, HiP | |
| | | Sampler, Column Compartment, DAD: | |
| | | G6150 MSD | |
| Column | | Waters Cortecs C18, 30 × 2.1 mm, 2.7 μm | |
| Column oven temperature | | 40° C. | |
| Flow rate | | 1.80 mL/min | |
| Run Time | | 1.49 min | |
| Detection | | UV at 260 nm +/− 90 nm unless otherwise | |
| | | indicated, MS by electrospray ionisation | |
| Gradient | | | |
| Time | Flow Rate | | |
| (min) | (mL/min) | % A | % B |
| 0.00 | 1.80 | 95 | 5 |
| 1.00 | 1.80 | 0 | 100 |
| 1.49 | 1.80 | 0 | 100 |

Preparative HPLC purifications were performed using a Waters X-Bridge BEH C18 column conducted as detailed below.

Prep Method A - Preparative HPLC Acidic Method (x-y %)

| Mobile phase | (A) | 0.1% Formic acid in water | |
|---|---|---|---|
| | (B) | 100% Acetonitrile | |
| Instrument | | 1260 Infinity II Preparative LC/MSD | |
| | | System | |
| Column | | Waters XBridge BEH C18 ODB prep | |
| | | column, 130 Å, 5 μm, 30 mm × 100 mm | |
| Column oven temperature | | rt | |
| Flow rate | | 42 mL/min | |
| Run Time | | 13 min | |
| Detection | | UV at 190-400 nm unless otherwise | |
| | | indicated, MS by electrospray ionisation | |
| Gradient | | | |
| Time | Flow Rate | | |
| (min) | (mL/min) | % A | % B |
| 0.00 | 42 | 100-x | x |
| 1.40 | 42 | 100-x | x |
| 10.50 | 42 | 100-y | y |

Prep Method B - Preparative HPLC Basic Method (x-y %)

| Mobile phase | (A) | 0.1% Ammonia in water | |
|---|---|---|---|
| | (B) | 100% Acetonitrile | |
| Instrument | | 1260 Infinity II Preparative LC/MSD | |
| | | System | |
| Column | | Waters XBridge BEH C18 ODB prep | |
| | | column, 130 Å, 5 μm, 30 mm × 100 mm | |
| Column oven temperature | | rt | |
| Flow rate | | 42 mL/min | |
| Run Time | | 13 min | |
| Detection | | UV at 190-400 nm unless otherwise | |
| | | indicated, MS by electrospray ionisation | |
| Gradient | | | |
| Time | Flow Rate | | |
| (min) | (mL/min) | % A | % B |
| 0.00 | 42 | 100-x | x |
| 1.40 | 42 | 100-x | x |
| 10.50 | 42 | 100-y | y |

NMR spectra were recorded using a Bruker 500 MHz Avance III HO spectrometer equipped with a Bruker 5 mm SmartProbe™. Spectra were measured at 298 K, unless indicated otherwise, and were referenced relative to the solvent resonance. The chemical shifts are reported in parts per million. Data were acquired using Bruker TopSpin software and processed using MestreNova software.

Reactions were conducted at ambient temperature (typically ca. 20° C.) unless stated otherwise.

All yields are adjusted for purity.

### Intermediates

Where procedures are not given for intermediates identified in synthetic schemes these compounds were purchased.

### Intermediate 1

### N-(4-Phenethoxyphenyl)-3-(4,4,5,5-tetramethyl-1,3,2-dioxaborolan-2-yl)benzamide (I-1)

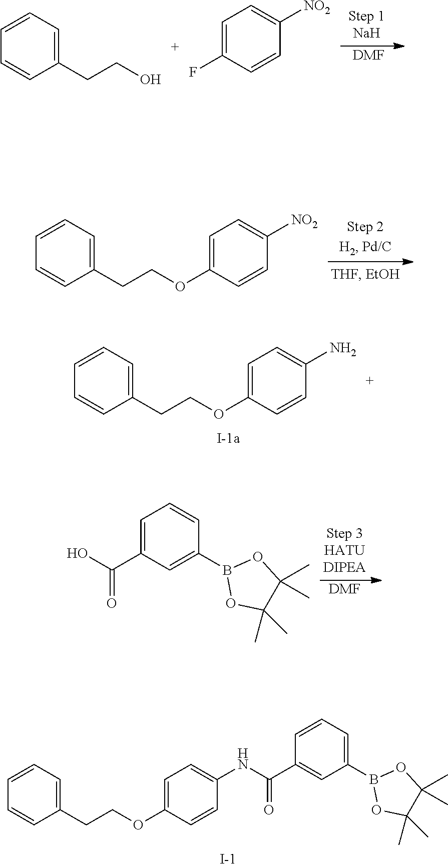

### Step 1: 1-Nitro-4-phenethoxybenzene

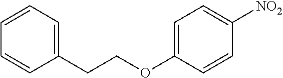 [SMILES: O=[N+]([O-])c1ccc(OCCc2ccccc2)cc1]

To a solution of 2-phenylethan-1-ol (CAS 60-12-8, Fluorochem, 3.38 g, 27.6 mmol) in DMF (20 mL) was added NaH (60% dispersion in oil, 1.02 g, 25.5 mmol) and the mixture stirred at rt for 30 min. 1-Fluoro-4-nitrobenzene (CAS 350-46-9, Alfa Aesar, 3.00 g, 21.3 mmol) was added, the reaction stirred at rt for 18 h then poured into ice-water (400 mL) and the product was extracted with EtOAc (3×100 mL). The organic extracts were washed with brine (50 mL), dried over MgSO₄ and the filtrate adsorbed onto silica gel. Purification by flash chromatography (silica gel, 0-30% EtOAc in isohexane) afforded 1-nitro-4-phenethoxybenzene (4.80 g, 93%).

LCMS: Method A, 2.00 min, MS: ES⁺ 244.0.

### Step 2: 4-Phenethoxyaniline (I-1a)

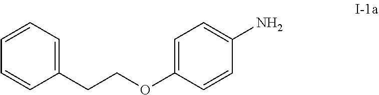 [SMILES: Nc1ccc(OCCc2ccccc2)cc1]

To a solution of 1-nitro-4-phenethoxybenzene (4.50 g, 18.5 mmol) in EtOH (55 mL) and THF (30 mL) was added 10% Pd/C (wet with 50% water, 0.984 g, 9.25 mmol) and the mixture was stirred at rt under hydrogen (3 bar) for 18 h. The mixture was filtered through Celite® and concentrated under reduced pressure to afford 4-phenethoxyaniline (3.50 g, 86%).

LCMS: Method A, 0.99 min, MS: ES⁺ 214.2.

### Step 3: N-(4-Phenethoxyphenyl)-3-(4,4,5,5-tetramethyl-1,3,2-dioxaborolan-2-yl)benzamide (I-1)

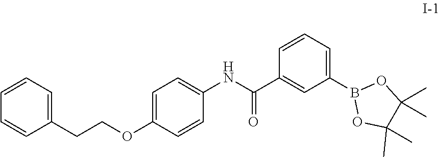 [SMILES: CC1(C)OB(c2cccc(C(=O)Nc3ccc(OCCc4ccccc4)cc3)c2)OC1(C)C]

To a stirred solution of 3-(4,4,5,5-tetramethyl-1,3,2-dioxaborolan-2-yl)benzoic acid (CAS 269409-73-6, BLD, 4.48 g, 18.1 mmol), and DIPEA (8.60 mL, 49.2 mmol) in DMF (40 mL), was added HATU (7.49 g, 20 mmol). After 30 min 4-phenethoxyaniline (3.50 g, 16.4 mmol) was added. The mixture was stirred at 40° C. for 18 h then cooled, poured into ice-water (500 mL) and extracted with EtOAc (3×120 mL). The combined organic phases were washed with brine (50 mL), dried over MgSO₄ and adsorbed onto silica gel, then purified by flash chromatography (silica gel, 0-50% EtOAc in isohexane) to afford N-(4-phenethoxyphenyl)-3-(4,4,5,5-tetramethyl-1,3,2-dioxaborolan-2-yl)benzamide (7.10 g, 87%).

LCMS: Method A, 2.27 min, MS: ES⁺ 444.2.

### Intermediate 2

### 2-Chloro-5-(3-((4-phenethoxyphenyl)carbamoyl)phenyl)nicotinate (I-2)

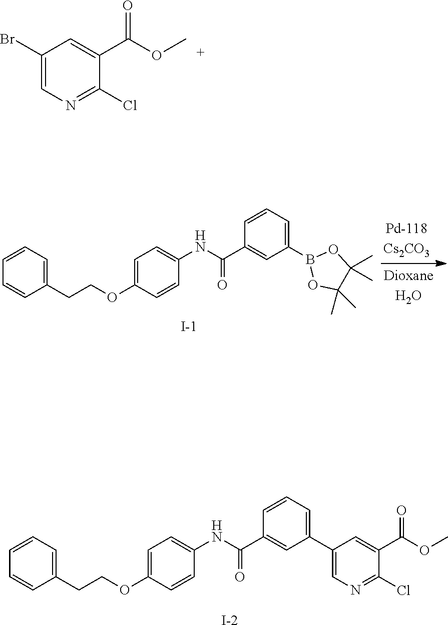

To a stirred solution of methyl 5-bromo-2-chloronicotinate (CAS 78686-79-0, Fluorochem 237 mg, 0.95 mmol) and N-(4-phenethoxyphenyl)-3-(4,4,5,5-tetramethyl-13,2-dioxaborolan-2-yl)benzamide (I-1, 350 mg, 0.79 mmol) in dioxane (4 ml) and water (1 mL), Cs₂O₃ (1000 mg, 3.16 mmol) was added and the reaction mixture was degassed with a flow of nitrogen at rt for 5 min. Pd(dppf)Cl₂ (87 mg, 0.12 mmol) was added and the mixture degassed for a further 2 min. The reaction mixture was stirred at 80° C. for 18 h then cooled to rt, acidified with aq. HCl (1M, 5 mL) and extracted with EtOAc (3×10 mL). The combined organic phases were dried over Na₂SO₄ and adsorbed onto silica gel then purified by flash chromatography (silica gel, 0-100% EtOAc in isohexane) to afford 2-chloro-5-(3-((4-phenethoxyphenyl)carbamoyl)phenyl)nicotinate (300 mg, 67%).

LCMS: Method A, 2.14 min MS: ES⁺ 487.2.

### Intermediate 3

### 4-((Benzyloxy)methyl)aniline (I-3)

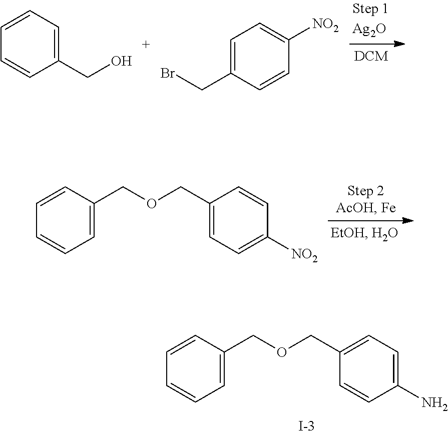

### Step 1: 1-((Benzyloxy)methyl)-4-nitrobenzene

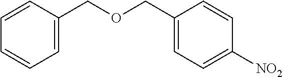 [SMILES: O=[N+]([O-])c1ccc(COCc2ccccc2)cc1]

Phenylmethanol (CAS 100-51-6, Alfa Aesar, 3.60 g, 33.3 mmol) was added to a stirred suspension of 1-(bromomethyl)-4-nitrobenzene (CAS 100-11-8, Combi-Blocks, 6.00 g, 27.8 mmol) and Ag₂O (9.65 g, 41.7 mmol) in DCM (60 mL) and the reaction stirred at 45° C. for 18 h. The mixture was filtered through Celite® and the filtrate was adsorbed onto silica gel then purified by flash chromatography (silica gel, 0-30% TBME in heptane) to afford 4-((benzyloxy)methyl)aniline (6.33 g, 94%).

LCMS: Method A, 1.96 min, MS: ES⁺ 244.1

### Step 2: 4-((Benzyloxy)methyl)aniline (I-3)

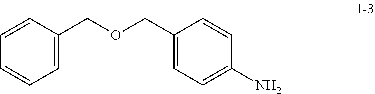 [SMILES: Nc1ccc(COCc2ccccc2)cc1]

AcOH (1.65 mL, 28.8 mmol) was added to a mixture of 1-((benzyloxy)methyl)-4-nitrobenzene (3.50 g, 14.4 mmol) and iron (4.02 g, 71.9 mmol) in EtOH (25 mL) and water (25 mL) and the reaction mixture was stirred at rt for 72 h. The mixture was filtered through Celite® and the crude concentrate was captured on SCX, washed with MeOH and eluted with 10% of 0.7M ammonia/MeOH solution in DCM. Further purification by chromatography on silica gel (80 g cartridge, 0-10% (0.7M ammonia/MeOH) in DCM) afforded 4-((benzyloxy)methyl)aniline (2.58 g, 79%).

LCMS: Method A, 1.09 min, MS: ES⁺ 214.1

### Intermediate 4

### 3-((Benzyloxy)methyl)aniline (I-4)

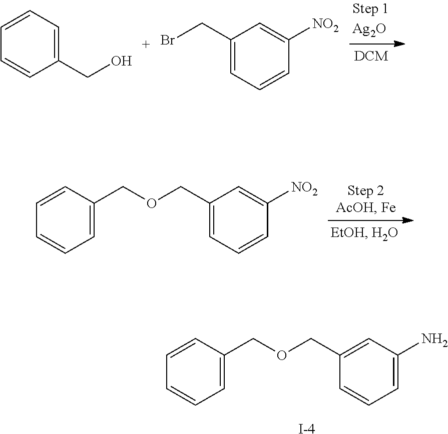

### Step 1: 1-((Benzyloxy)methyl)-3-nitrobenzene

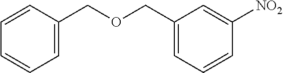 [SMILES: O=[N+]([O-])c1cccc(COCc2ccccc2)c1]

Following the procedure of Intermediate 3 Step 1, using 1-(bromomethyl)-3-nitrobenzene (CAS 3958-57-4, Thermo Scientific) in place of 1-(bromomethyl)-4-nitrobenzene, was obtained 1-((benzyloxy)methyl)-3-nitrobenzene in 76% yield.

¹H NMR (500 MHz, DMSO) δ ppm: 8.21 (s, 1H), 8.19-8.14 (m, 1H), 7.83 (d, J=7.6 Hz, 1H), 7.68 (t, J=7.9 Hz, 1H), 7.41-7.35 (m, 4H), 7.35-7.28 (m, 1H), 4.69 (s, 2H), 4.61 (s, 2H).

### Step 2: 3-((Benzyloxy)methyl)aniline (I-4)

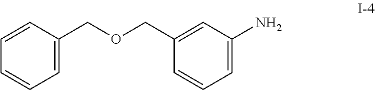 [SMILES: Nc1cccc(COCc2ccccc2)c1]

Following the procedure of Intermediate 3 Step 2, using 1-((benzyloxy)methyl)-3-nitrobenzene in place of 1-((benzyloxy)methyl)-4-nitrobenzene and without purification of crude product, was obtained 3-((benzyloxy)methyl)aniline in 75% yield.

LCMS: Method A, 1.14 min, MS ES⁺ 214.2.

### Intermediate 5

### N-(4-((Cyclopropylmethoxy)methyl)phenyl)-3-(4,4,5,5-tetramethyl-1,3,2-dioxaborolan-2-yl)benzamide (I-5)

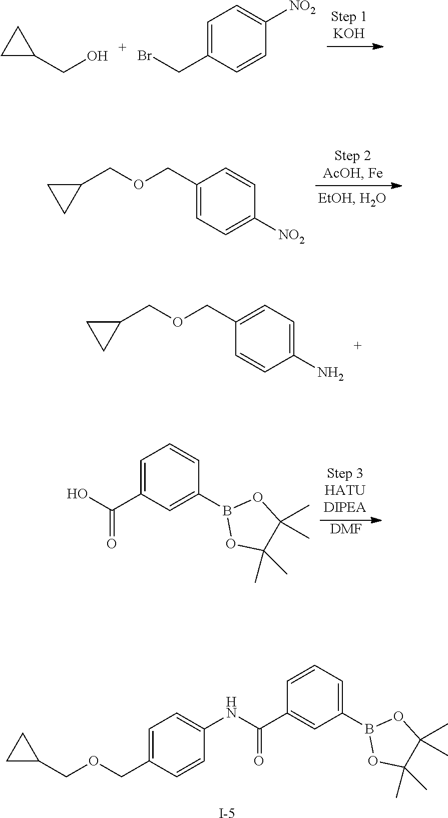

### Step 1: 1-((Cyclopropylmethoxy)methyl)-4-nitrobenzene

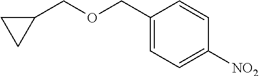 [SMILES: O=[N+]([O-])c1ccc(COCC2CC2)cc1]

A solution of 1-(bromomethyl)-4-nitrobenzene (CAS 100-11-8, Thermo Scientific, 500 mg, 2.31 mmol) in cyclopropylmethanol (CAS 2516-33-8, Fluorochem, 1.9 mL, 23.1 mmol) was treated with KOH (156 mg, 2.78 mmol). The mixture was stirred at rt for 18 h. Further KOH (156 mg, 2.78 mmol) was added, and the mixture stirred for a further 3 h, then diluted with water (200 mL) and the product was extracted EtOAc (3×50 mL). The combined organic fractions were washed with brine (3×50 mL), dried over Na₂SO₄, and the filtrate adsorbed onto silica gel. Purification by flash chromatography A (silica gel, 0-10% EtOAc in isohexane) afforded 1-((cyclopropylmethoxy)methyl)-4-nitrobenzene (400 mg, 83%).

LCMS: Method A, 1.76 min, MS: ES⁺ 208.2

### Step 2: 4-((Cyclopropylmethoxy)methyl)aniline

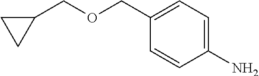 [SMILES: Nc1ccc(COCC2CC2)cc1]

Following the procedure of Intermediate 3 Step 2, using 1-((benzyloxy)methyl)-3-nitrobenzene in place of 1-((benzyloxy)methyl)-4-nitrobenzene, was obtained 4-((cyclopropylmethoxy)methyl)aniline in 58% yield.

LCMS: Method A, 0.42 min, MS ES⁺ 178.2.

### Step 3: N-(4-((Cyclopropylmethoxy)methyl)phenyl)-3-(4,4,5,5-tetramethyl-1,3,2-dioxaborolan-2-yl)benzamide (I-5)

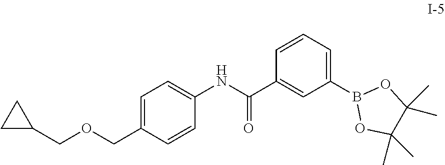 [SMILES: CC1(C)OB(c2cccc(C(=O)Nc3ccc(COCC4CC4)cc3)c2)OC1(C)C]

Following the procedure of Intermediate 1 Step 3, using 3-(4,4,5,5-tetramethyl-1,3,2-dioxaborolan-2-yl)benzoic acid in place of 3-(4,4,5,5-tetramethyl-1,3,2-dioxaborolan-2-yl)benzoic acid and without purification of crude product, was obtained N-(4-((cyclopropylmethoxy)methyl)phenyl)-3-(4,4,5,5-tetramethyl-1,3,2-dioxaborolan-2-yl)benzamide in 52% yield.

LCMS: Method A, 2.13 min, MS ES⁺ 408.2.

### Intermediate 6

### 4-(Cyclopropylmethoxy)aniline (I-6)

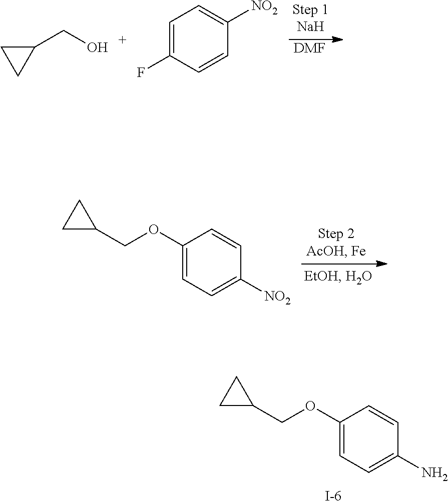

### Step 1: 1-(Cyclopropylmethoxy)-4-nitrobenzene

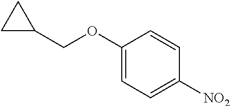 [SMILES: O=[N+]([O-])c1ccc(OCC2CC2)cc1]

Following the procedure of Intermediate 1 Step 1, using cyclopropylmethanol (CAS 2516-33-8, Fluorochem) in place of 2-phenylethan-1-ol, was obtained 1-(cyclopropylmethoxy)-4-nitrobenzene in 71% yield.

LCMS: Method A, 1.79 min, MS ES⁺ 194.2.

### Step 2: 4-(Cyclopropylmethoxy)aniline (I-6)

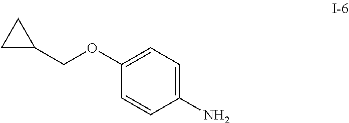 [SMILES: Nc1ccc(OCC2CC2)cc1]

Following the procedure of Intermediate 3 Step 2, using 1-(cyclopropylmethoxy)-4-nitrobenzene in place of 1-((benzyloxy)methyl)-4-nitrobenzene and without purification of crude product, was obtained 4-(cyclopropylmethoxy)aniline in 60% yield.

LCMS: Method A, 0.22 min, MS ES⁺ 164.2.

### Intermediate 7

### 4-(Cyclopropoxymethyl)aniline (I-7)

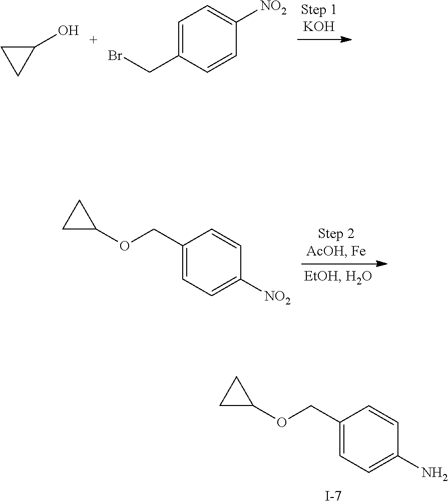

### Step 1: 1-(Cyclopropoxymethyl)-4-nitrobenzene

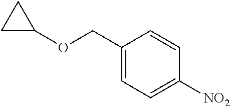 [SMILES: O=[N+]([O-])c1ccc(COC2CC2)cc1]

Following the procedure of Intermediate 5 Step 1, using cyclopropanol (CAS 16545-68-9, Combi-Blocks) in place of cyclopropylmethanol, was obtained 1-(cyclopropoxymethyl)-4-nitrobenzene in 89% yield.

LCMS: Method A, 1.70 min, MS ES⁺ 194.2.

### Step 2: 4-(Cyclopropoxymethyl)aniline (I-7)

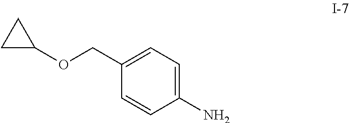 [SMILES: Nc1ccc(COC2CC2)cc1]

Following the procedure of Intermediate 3 Step 2, using 1-(cyclopropoxymethyl)-4-nitrobenzene in place of 1-((benzyloxy)methyl)-4-nitrobenzene, was obtained 4-(cyclopropoxymethyl)aniline in 60% yield.

LCMS: Method A, 0.22 min, MS ES⁺ 164.2.

### Intermediate 8

### N-(6-Phenethoxypyridin-3-yl)-3-(4,4,5,5-tetramethyl-1,3,2-dioxaborolan-2-yl)benzamide (I-8)

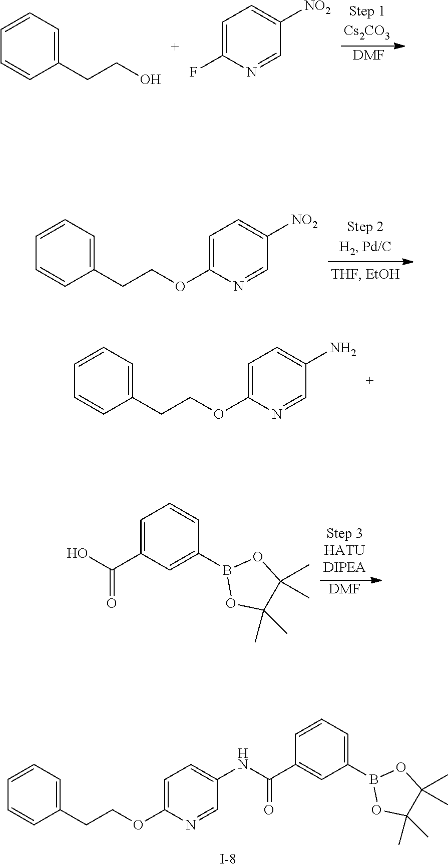

### Step 1: 5-Nitro-2-phenethoxypyridine

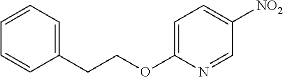 [SMILES: O=[N+]([O-])c1ccc(OCCc2ccccc2)nc1]

To a solution of 2-phenylethan-1-ol (CAS 60-12-8, Fluorochem, 0.55 mL, 4.7 mmol) in DMF (7 mL) were added 2-fluoro-5-nitropyridine (CAS 456-24-6, Activate Scientific, 0.50 g, 3.5 mmol) and Cs₂CO₃ (2.29 g, 7.0 mmol). The mixture was stirred at 40° C. for 18 h then cooled to rt, diluted with brine (30 mL) and extracted with EtOAc (3×30 mL). The combined organic fractions were washed with brine (3×30 mL), dried over MgSO₄, filtered, and adsorbed onto silica gel. Purification by flash chromatography (silica gel, 0-30% EtOAc in isohexane) afforded 5-nitro-2-phenethoxypyridine (0.70 g, 80%).

LCMS: Method A, 1.96 min, MS: ES⁺ 245.0.

### Step: 6-Phenethoxypyridin-3-amine

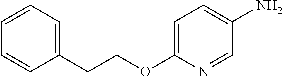 [SMILES: Nc1ccc(OCCc2ccccc2)nc1]

A solution of methyl 5-nitro-2-phenethoxypyridine (700 mg, 2.9 mmol) in MeOH (25 mL) was passed through a 10% Pd/C cartridge under hydrogen at 30° C. at 1 bar (H-Cube®) for 1 h. The reaction mixture was concentrated under reduced pressure to give 6-phenethoxypyridin-3-amine (550 mg, 82%), used without any purification.

LCMS: Method A, 1.12 min, MS: ES⁺ 215.2.

### Step 3: N-(6-Phenethoxypyridin-3-yl)-3-(4,4,5,5-tetramethyl-1,3,2-dioxaborolan-2-yl)benzamide (I-8)

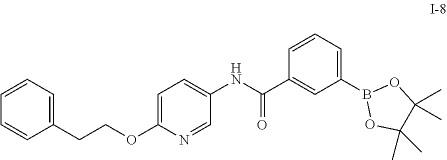 [SMILES: CC1(C)OB(c2cccc(C(=O)Nc3ccc(OCCc4ccccc4)nc3)c2)OC1(C)C]

Following the procedure of Intermediate 1 Step 3, using 6-phenethoxypyridin-3-amine in place of 4-phenethoxyaniline, was obtained N-(6-phenethoxypyridin-3-yl)-3-(4,4,5,5-tetramethyl-1,3,2-dioxaborolan-2-yl)benzamide in 48% yield.

LCMS: Method A, 2.18 min, MS ES⁺ 445.2.

### Intermediate 9

### 4-((Benzylsulfonyl)methyl)aniline (I-9)

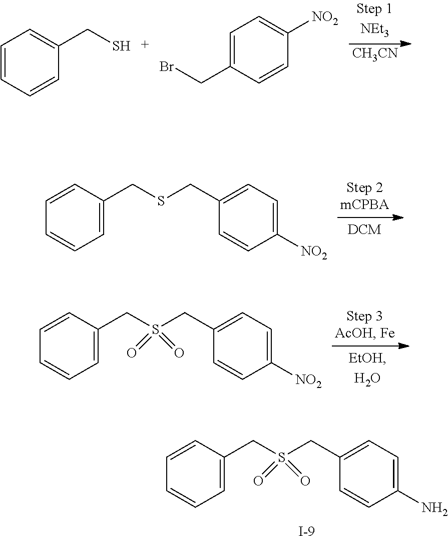

### Step 1: Benzyl(4-nitrobenzyl)sulfane

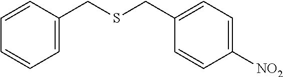 [SMILES: O=[N+]([O-])c1ccc(CSCc2ccccc2)cc1]

To a solution of phenylmethanethiol (CAS 100-53-8, Merck, 0.22 mL, 1.9 mmol) and triethylamine (0.26 mL, 1.9 mmol) in MeCN (5 mL), 1-(bromomethyl)-4-nitrobenzene (CAS 100-11-8, Apollo, 200 mg, 0.9 mmol) was added at 0° C. The mixture was stirred at rt for 8 h, diluted with sat. aq. Na₂CO₃ (20 mL) and extracted with EtOAc (3×10 mL). The combined organic phases were dried over Na₂SO₄ and concentrated under reduced pressure. Purification by flash chromatography (silica gel, 0-50% EtOAc in isohexane) afforded benzyl(4-nitrobenzyl)sulfane (150 mg, 62%).

¹H NMR (500 MHz, DMSO) δ ppm: 8.23-8.15 (d, J=8.5 Hz, 2H), 7.57 (d, J=8.5 Hz, 2H), 7.35-7.22 (m, 5H), 3.81 (s, 2H), 3.69 (s, 2H).

### Step 2: 1-((Benzylsulfonyl)methyl)-4-nitrobenzene

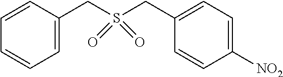 [SMILES: O=[N+]([O-])c1ccc(CS(=O)(=O)Cc2ccccc2)cc1]

To a solution of benzyl(4-nitrobenzyl)sulfane (150 mg, 0.58 mmol) in DCM (4 mL) was added mCPBA (314 mg, 70%, 1.3 mmol) slowly at 0° C. The reaction was allowed to warm to rt for 4 h, then quenched with aq. Na₂S₂O₅ solution (10% by wt, 20 mL) and extracted with ethyl acetate (3×15 mL). The combined organic phases were washed with sat. aq. NaHCO₃ (20 mL), dried over Na₂SO₄, and concentrated under reduced pressure to afford 1-((benzylsulfonyl)methyl)-4-nitrobenzene (157 mg, 89%).

¹H NMR (500 MHz, DMSO) δ ppm: 8.27 (d, J=8.3 Hz, 2H), 7.67 (d, J=8.3 Hz, 2H), 7.46-7.36 (m, 5H), 4.70 (s, 2H), 4.55 (s, 2H).

### Step 3: 4-((Benzylsulfonyl)methyl)aniline (I-9)

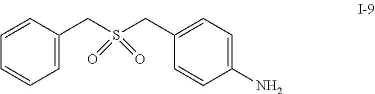 [SMILES: Nc1ccc(CS(=O)(=O)Cc2ccccc2)cc1]

Following the procedure of Intermediate 3 Step 2, using 1-((benzylsulfonyl)methyl)-4-nitrobenzene in place of 1-((benzyloxy)methyl)-4-nitrobenzene and without purification of crude product, was obtained 4-((benzylsulfonyl)methyl)aniline in 64% yield.

LCMS: Method A, 0.22 min, MS ES⁺ 164.2.

### Intermediate 10

### N-(4-((Benzyloxy)methyl)-2-fluorophenyl)-3-(4,4,5,5-tetramethyl-1,3,2-dioxaborolan-2-yl)benzamide (I-10)

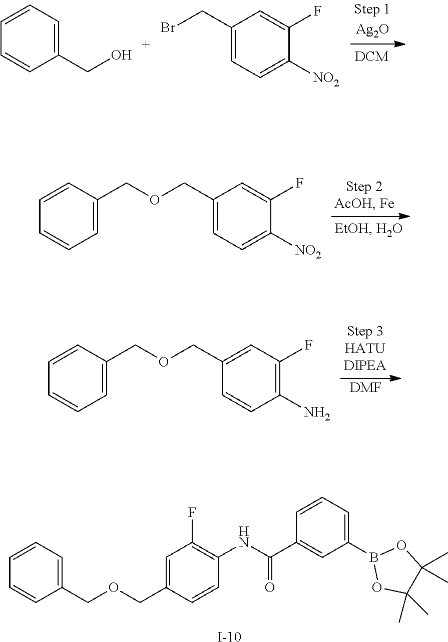

### Step 1: 4-((Benzyloxy)methyl)-2-fluoro-1-nitrobenzene

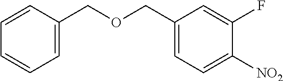 [SMILES: O=[N+]([O-])c1ccc(COCc2ccccc2)cc1F]

Following the procedure of Intermediate 3 Step 1, using 4-(bromomethyl)-2-fluoro-1-nitrobenzene (CAS 131858-37-2, Apollo) in place of 1-(bromomethyl)-4-nitrobenzene, there was thus obtained 4-((benzyloxy)methyl)-2-fluoro-1-nitrobenzene in 96% yield.

LCMS: Method A, 1.94 min, MS: ES⁺ 261.1.

### Step 2: 4-((Benzyloxy)methyl)-2-fluoroaniline

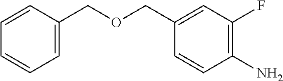 [SMILES: Nc1ccc(COCc2ccccc2)cc1F]

Following the procedure of Intermediate 3 Step 2, using 4-((benzyloxy)methyl)-2-fluoro-1-nitrobenzene in place of 1-((benzyloxy)methyl)-4-nitrobenzene and with purification by flash chromatography (silica gel, 0-50% TBME in isohexane), there was thus obtained 4-((benzyloxy)methyl)-2-fluoroaniline in 75% yield.

LCMS: Method A, 1.64 min, MS ES⁺ 232.1.

### Step 3: N-(4-((Benzyloxy)methyl)-2-fluorophenyl)-3-(4,4,5,5-tetramethyl-1,3,2-dioxaborolan-2-yl)benzamide (I-10)

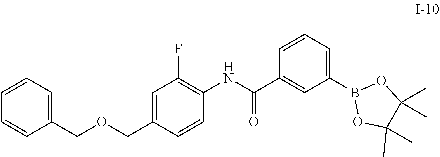 [SMILES: CC1(C)OB(c2cccc(C(=O)Nc3ccc(COCc4ccccc4)cc3F)c2)OC1(C)C]

Following the procedure of Intermediate 1 Step 3, using 4-((benzyloxy)methyl)-2-fluoroaniline in place of 4-phenethoxyaniline, was thus obtained N-(4-((benzyloxy)methyl)-2-fluorophenyl)-3-(4,4,5,5-tetramethyl-1,3,2-dioxaborolan-2-yl)benzamide in 46% yield.

LCMS: Method A, 2.26 min, MS ES⁺ 462.2.

### Intermediate 11

### 3-(5-(Methoxycarbonyl)-6-methylpyridin-3-yl)benzoic acid (I-11)

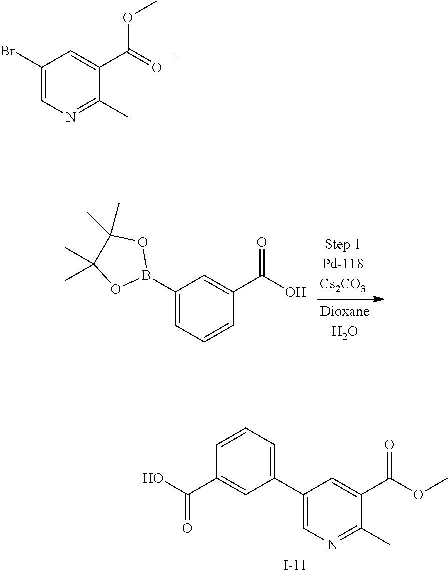

Following the procedure of Intermediate 2, using methyl 5-bromo-2-methylnicotinate (CAS 1215916-40-7, BLD) instead of methyl 5-bromo-2-chloronicotinate and 3-(4,4,5,5-tetramethyl-1,3,2-dioxaborolan-2-yl)benzoic acid (CAS 269409-73-6, BLD) instead of N-(4-phenethoxyphenyl)-3-(4,4,5,5-tetramethyl-1,3,2-dioxaborolan-2-yl)benzamide, and with reaction at 80° C. for 2 h and a final trituration with Et₂O, was obtained 3-(5-(methoxycarbonyl)-6-methylpyridin-3-yl)benzoic acid in 98% yield.

LCMS: Method A, 1.30 min, MS: ES⁺ 272.1.

### Intermediate 12

### N-(4-((benzyloxy)methyl)-2-fluorophenyl)-5-(4,4,5,5-tetramethyl-1,3,2-dioxaborolan-2-yl)benzamide (I-12)

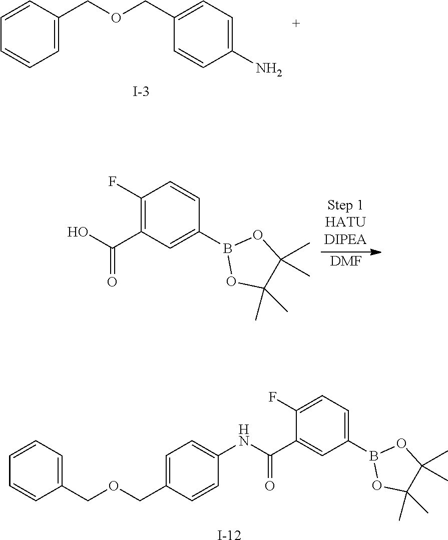

Following the procedure of Intermediate 1 Step 3, using 4-((benzyloxy)methyl)aniline (I-3) in place of 4-phenethoxyaniline and 2-fluoro-5-(4,4,5,5-tetramethyl-1,3,2-dioxaborolan-2-yl)benzoic acid (CAS 882679-10-9, Fluorochem) in place of 3-(4,4,5,5-tetramethyl-1,3,2-dioxaborolan-2-yl)benzoic acid (CAS 269409-73-6, BLD), was obtained N-(4-((benzyloxy)methyl)-2-fluorophenyl)-5-(4,4,5,5-tetramethyl-1,3,2-dioxaborolan-2-yl)benzamide in 46% yield.

LCMS: Method A, 2.26 min, MS: ES⁺ 462.2.

### Intermediate 13

### 2-Bromo-N-(4-phenethoxyphenyl)isonicotinamide (I-13)

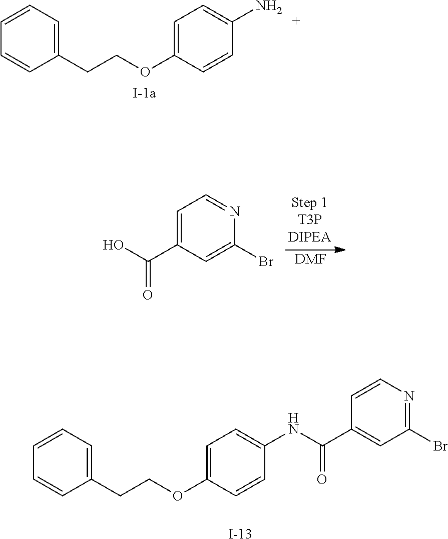

To a solution of 2-bromoisonicotinic acid (CAS 66572-56-3, Apollo, 417 mg, 2.06 mmol) and 4-phenethoxyaniline (I-1a, 500 mg, 1.88 mmol) in DMF (2.00 mL) was added DIPEA (0.98 mL, 5.63 mmol) and T3P (50% by wt. in EtOAc, 1.22 mL, 2.06 mmol) at rt. The mixture was stirred for 18 h then diluted with water (10 mL) and extracted with EtOAc (3×20 mL). The combined organic phases were dried over Na₂SO₄ and adsorbed onto silica gel. Purification by flash chromatography (silica gel, 0-100% EtOAc in isohexane) afforded 2-bromo-N-(4-phenethoxyphenyl)isonicotinamide (705 mg, 87%).

LCMS: Method C, 1.10 min, MS: ES⁺ 397.0 and 399.0.

### Intermediate 14

### 6-Chloro-N-(4-phenethoxyphenyl)pyrazine-2-carboxamide (I-14)

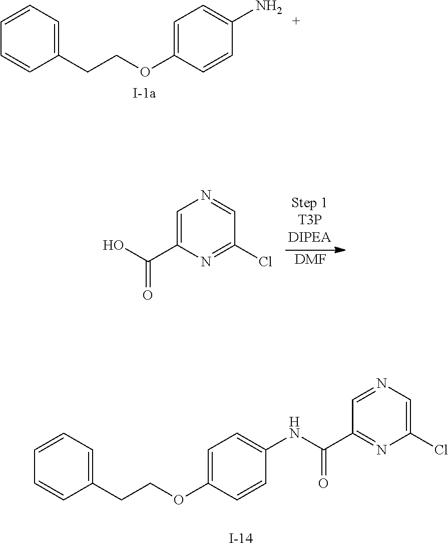

Following the procedure of Intermediate 13, using 6-chloropyrazine-2-carboxylic acid (CAS 23688-89-3, Apollo) in place of 2-bromoisonicotinic acid (CAS 66572-56-3, Apollo), was obtained 6-chloro-N-(4-phenethoxyphenyl)pyrazine-2-carboxamide in 75% yield.

LCMS: Method C, 1.11 min, MS: ES⁺ 354.2.

### Intermediate 15

### 2-Chloro-N-(4-phenethoxyphenyl)pyrimidine-4-carboxamide (I-15)

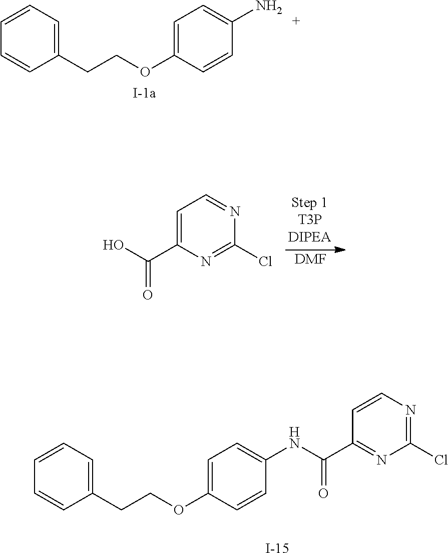

Following the procedure of Intermediate 13, using 2-chloropyrimidine-4-carboxylic acid (CAS 149849-92-3, BLD) in place of 2-bromoisonicotinic acid (CAS 66572-56-3, Apollo), was obtained 2-chloro-N-(4-phenethoxyphenyl)pyrimidine-4-carboxamide in 84% yield.

LCMS: Method C, 1.1 min, MS: ES⁺ 354.0.

### Intermediate 16

### N-(4-((Benzyloxy)methyl)-3-fluorophenyl)-3-(4,4,5,5-tetramethyl-1,3,2-dioxaborolan-2-yl)benzamide (I-16)

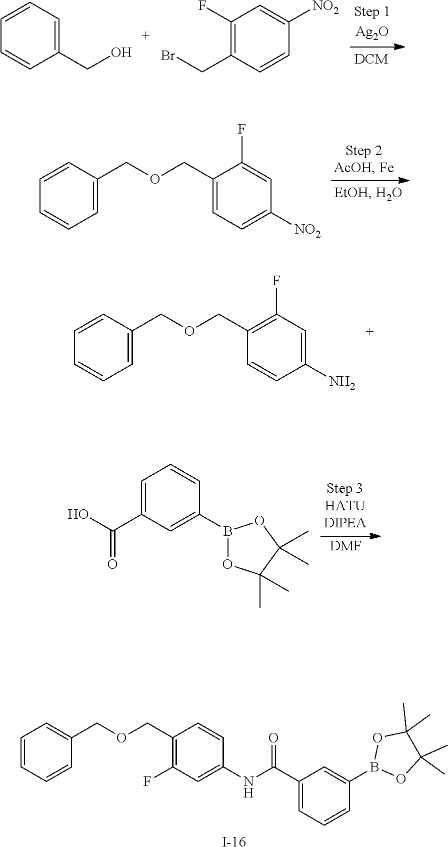

### Step 1: 1-((Benzyloxy)methyl)-2-fluoro-4-nitrobenzene

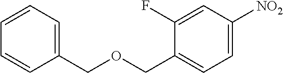 [SMILES: O=[N+]([O-])c1ccc(COCc2ccccc2)c(F)c1]

Following the procedure of Intermediate 3 Step 1, using 1-(bromomethyl)-2-fluoro-4-nitrobenzene [CAS 127349-56-8, Apollo] in place of 1-(bromomethyl)-4-nitrobenzene was obtained 1-((benzyloxy)methyl)-2-fluoro-4-nitrobenzene in 95% yield.

LCMS: Method A: 1.98 min, MS: ES⁺ mass ion not observed

### Step 2: 4-((Benzyloxy)methyl)-3-fluoroaniline

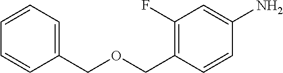 [SMILES: Nc1ccc(COCc2ccccc2)c(F)c1]

Following the procedure of Intermediate 3 Step 2, using 1-((benzyloxy)methyl)-2-fluoro-4-nitrobenzene in place of 1-((benzyloxy)methyl)-4-nitrobenzene, at 45° C. for 18 h, and without purification of crude product, was obtained 4-((benzyloxy)methyl)-3-fluoroaniline in 67% yield.

LCMS: Method A, 1.56 min, MS ES⁺ 232.1

### Step 3: N-(4-((Benzyloxy)methyl)-3-fluorophenyl)-3-(4,4,5,5-tetramethyl-1,3,2-dioxaborolan-2-yl)benzamide (I-16)

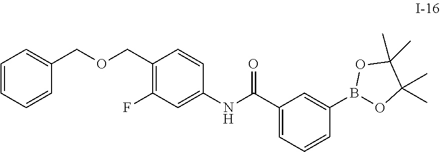 [SMILES: CC1(C)OB(c2cccc(C(=O)Nc3ccc(COCc4ccccc4)c(F)c3)c2)OC1(C)C]

To a mixture of 3-(4,4,5,5-tetramethyl-1,3,2-dioxaborolan-2-yl)benzoic acid (CAS 269409-73-6, BLD, 0.51 g, 2.1 mmol) and DIPEA (0.42 mL, 2.4 mmol) in DMF (2 mL) was added HATU (0.79 g, 2.1 mmol). After 10 min, 4-((benzyloxy)methyl)-3-fluoroaniline (0.40 g, 1.7 mmol) was added and the mixture stirred at 40° C. for 18 hours. The mixture was cooled, poured into ice-water (30 mL) and extracted with EtOAc (2×50 mL). The combined organics were dried over Na₂SO₄ and concentrated under reduced pressure. Purification by flash chromatography (silica gel, 0-100% EtOAc in isohexane) afforded N-(4-((benzyloxy)methyl)-3-fluorophenyl)-3-(4,4,5,5-tetramethyl-1,3,2-dioxaborolan-2-yl)benzamide (360 mg, 34%).

LCMS: Method A: 2.31 min, MS: ES⁺ 462.2

### Intermediate 17

### N-(4-(1-Cyclopropylethoxy)-3-fluorophenyl)-2-fluoro-5-(4,4,5,5-tetramethyl-1,3,2-dioxaborolan-2-yl)benzamide (I-17)

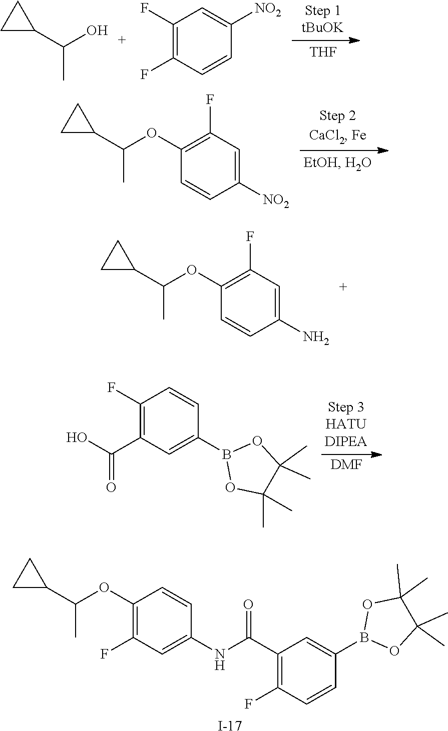

### Step 1: 1-(1-Cyclopropylethoxy)-2-fluoro-4-nitrobenzene

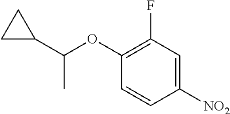 [SMILES: CC(Oc1ccc([N+](=O)[O-])cc1F)C1CC1]

To 1-cyclopropylethan-1-ol (CAS 765-42-4, Fluorochem, 0.62 mL, 6.29 mmol) in THF (150 mL) at 0° C. was added tBuOK (776 mg, 6.91 mmol) in one portion. After 15 min, 1,2-difluoro-4-nitrobenzene (CAS 369-34-6, Fluorochem, 1.00 g, 6.29 mmol) in THF (40 mL) was added over 10 min and the ice-bath was removed. The mixture was stirred at room temperature for 1 h. Water (150 mL) was added and the mixture extracted with EtOAc (3×200 mL). The combined organics were washed with water (200 mL) and brine (200 mL), dried over Na₂SO₄ and concentrated under reduced pressure to afford 1-(1-cyclopropylethoxy)-2-fluoro-4-nitrobenzene (1.20 g, 79%).

LCMS: Method A: 1.90 min, MS: ES⁺ 226.1

### Step 2: 4-(1-Cyclopropylethoxy)-3-fluoroaniline

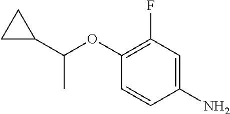

Following the procedure of Intermediate 3 Step 2, using 1-(1-cyclopropylethoxy)-2-fluoro-4-nitrobenzene in place of 1-((benzyloxy)methyl)-4-nitrobenzene, CaCl₂ (4.5 eq) in place of AcOH, at 80° C. for 18 h, and without purification of crude product, was obtained 4-(1-cyclopropylethoxy)-3-fluoroaniline in 37% yield.

LCMS: Method A, 0.97 min, MS ES⁺ 196.2

### Step 3: N-(4-(1-Cyclopropylethoxy)-3-fluorophenyl)-2-fluoro-5-(4,4,5,5-tetramethyl-1,3,2-dioxaborolan-2-yl)benzamide (I-17)

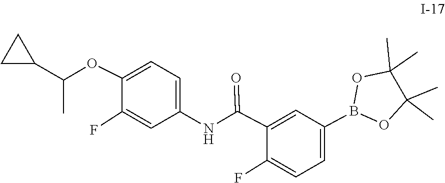 [SMILES: CC(Oc1ccc(NC(=O)c2cc(B3OC(C)(C)C(C)(C)O3)ccc2F)cc1F)C1CC1]

Following the procedure of Intermediate 16 Step 3, at rt rather than 40° C., using 2-fluoro-5-(4,4,5,5-tetramethyl-1,3,2-dioxaborolan-2-yl)benzoic acid [CAS 882679-10-9, BLD] in place of 3-(4,4,5,5-tetramethyl-1,3,2-dioxaborolan-2-yl)benzoic acid, and 4-(1-Cyclopropylethoxy)-3-fluoroaniline in place of 4-((benzyloxy)methyl)-3-fluoroaniline was obtained N-(4-(1-cyclopropylethoxy)-3-fluorophenyl)-2-fluoro-5-(4,4,5,5-tetramethyl-1,3,2-dioxaborolan-2-yl)benzamide in 27% yield.

LCMS: Method A: 2.33 min, MS: ES⁺ 444.2

### Intermediate 18

### N-(3-Cyano-4-(1-cyclopropylethoxy)phenyl)-2-fluoro-5-(4,4,5,5-tetramethyl-1,3,2-dioxaborolan-2-yl)benzamide (I-18)

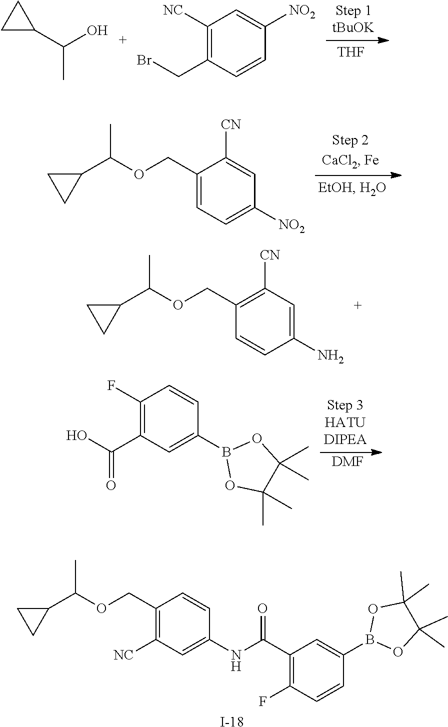

### Step 1: 2-(1-Cyclopropylethoxy)-5-nitrobenzonitrile

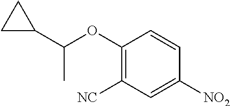 [SMILES: CC(Oc1ccc([N+](=O)[O-])cc1C#N)C1CC1]

To 1-cyclopropylethan-1-ol (CAS 765-42-4, Fluorochem, 0.59 mL, 6.02 mmol) in THF (150 mL) at 0° C. was added tBuOK (743 mg, 6.62 mmol) in one portion. After 15 minutes, 2-fluoro-5-nitrobenzonitrile (CAS 17417-09-3, Fluorochem, 1.00 g, 6.02 mmol) in THF (40 mL) was added over 10 min and the ice-bath was removed. The mixture was stirred at rt for 1 h. Water (150 mL) was added and the mixture extracted with EtOAc (3×200 mL). The combined organics were washed with water (200 mL) and brine (200 mL), dried over Na₂SO₄ and concentrated under reduced pressure to afford 2-(1-cyclopropylethoxy)-5-nitrobenzonitrile (1.15 g, 76%).

LCMS: Method A: 1.76 min, MS: ES⁺ 233.1

### Step 2: 5-Amino-2-(1-cyclopropylethoxy)benzonitrile

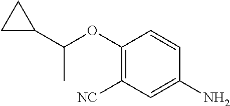

Following the procedure of Intermediate 3 Step 2, using 2-(1-cyclopropylethoxy)-5-nitrobenzonitrile in place of 1-((benzyloxy)methyl)-4-nitrobenzene, CaCl₂ (4.5 eq) in place of AcOH, at 80° C. for 18 h, and without purification of crude product, was obtained 5-amino-2-(1-cyclopropylethoxy)benzonitrile in 25% yield.

LCMS: Method A, 1.24 min, MS ES⁺ 203.2

### Step 3: N-(3-Cyano-4-(1-cyclopropylethoxy)phenyl)-2-fluoro-5-(4,4,5,5-tetramethyl-1,3,2-dioxaborolan-2-yl)benzamide (I-18)

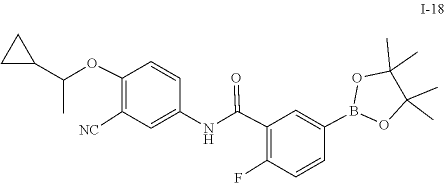 [SMILES: CC(Oc1ccc(NC(=O)c2cc(B3OC(C)(C)C(C)(C)O3)ccc2F)cc1C#N)C1CC1]

Following the procedure of Intermediate 16 Step 3, using 2-fluoro-5-(4,4,5,5-tetramethyl-1,3,2-dioxaborolan-2-yl)benzoic acid [CAS 882679-10-9, BLD] in place of 3-(4,4,5,5-tetramethyl-1,3,2-dioxaborolan-2-yl)benzoic acid, and 5-amino-2-(1-cyclopropylethoxy)benzonitrile in place of 4-((benzyloxy)methyl)-3-fluoroaniline, and at rt rather than 40° C., was obtained N-(3-cyano-4-(1-cyclopropylethoxy)phenyl)-2-fluoro-5-(4,4,5,5-tetramethyl-1,3,2-dioxaborolan-2-yl)benzamide in 33% yield, which was used in the next step without purification or analysis.

### Intermediate 19

### N-(4-((1-Phenylethoxy)methyl)phenyl)-3-(4,4,5,5-tetramethyl-1,3,2-dioxaborolan-2-yl)benzamide (I-19)

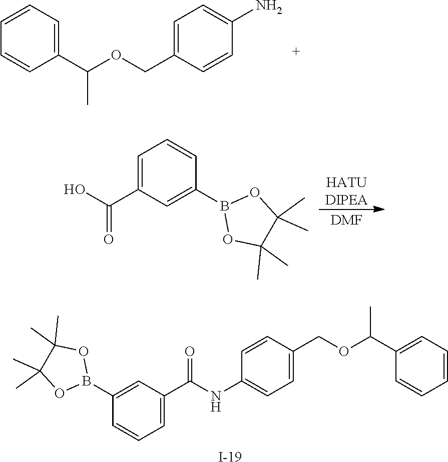

Following the procedure of Intermediate 16 Step 3, using 4-((1-phenylethoxy)methyl)aniline [CAS 2743-01-3, Fluorochem] in place of 4-((benzyloxy)methyl)-3-fluoroaniline was obtained N-(4-((1-phenylethoxy)methyl)phenyl)-3-(4,4,5,5-tetramethyl-1,3,2-dioxaborolan-2-yl)benzamide in 17% yield.

LCMS: Method A: 2.3 min, MS: ES⁺ 458.2

### Intermediate 20

### N-(4-(1-Cyclopropylethoxy)phenyl)-3-(4,4,5,5-tetramethyl-1,3,2-dioxaborolan-2-yl)benzamide (I-20)

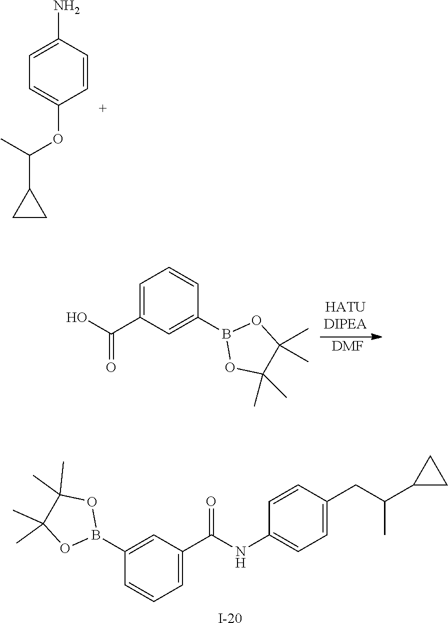

Following the procedure of Intermediate 16 Step 3, using 4-(1-cyclopropylethoxy)aniline [CAS 2168664-20-6, Enamine] in place of 4-((benzyloxy)methyl)-3-fluoroaniline was obtained N-(4-(1-cyclopropylethoxy)phenyl)-3-(4,4,5,5-tetramethyl-1,3,2-dioxaborolan-2-yl)benzamide in 23% yield.

LCMS: Method A: 2.17 min, MS: ES⁺ 408.2

### Intermediate 21

### N-(4-((Benzyloxy)methyl)-2-methoxyphenyl)-3-(4,4,5,5-tetramethyl-1,3,2-dioxaborolan-2-yl)benzamide (I-21)

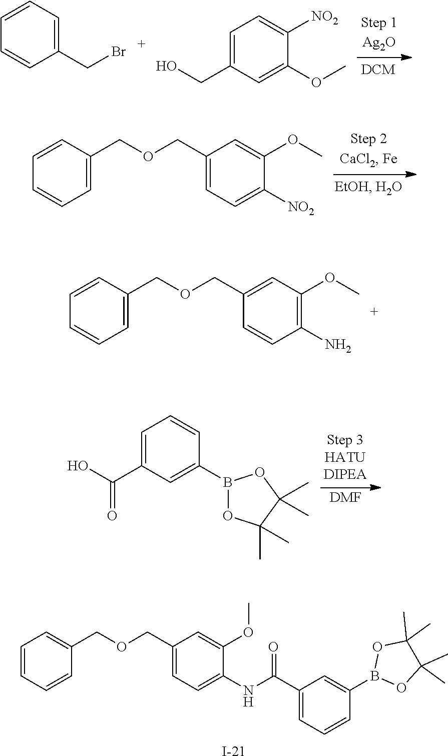

### Step 1: 4-((Benzyloxy)methyl)-2-methoxy-1-nitrobenzene

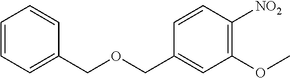 [SMILES: COc1cc(COCc2ccccc2)ccc1[N+](=O)[O-]]

Following the procedure of Intermediate 3 Step 1, using (3-methoxy-4-nitrophenyl)methanol [CAS 80866-88-2, BLD] in place of phenylmethanol, and benzyl bromide [CAS 100-39-0, Merck] in place of 1-(bromomethyl)-4-nitrobenzene, with LiOH (1 eq) and purification by flash chromatography (silica gel, 0-10% (0.7M ammonia/MeOH) in DCM), was obtained 4-((benzyloxy)methyl)-2-methoxy-1-nitrobenzene in 85% yield.

LCMS: Method A: 1.94 min, MS: ES⁺ 274.2

### Step 2: 4-((Benzyloxy)methyl)-2-methoxyaniline

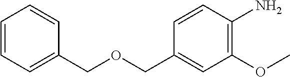 [SMILES: COc1cc(COCc2ccccc2)ccc1N]

Following the procedure of Intermediate 3 Step 2, using 4-((benzyloxy)methyl)-2-methoxy-1-nitrobenzene in place of 1-((benzyloxy)methyl)-4-nitrobenzene, CaCl₂ (4.5 eq) in place of AcOH, at 40° C. for 18 h, and without purification of crude product, was obtained 4-((benzyloxy)methyl)-2-methoxyaniline in 73% yield.

LCMS: Method A, 1.27 min, MS ES⁺ 244.2

### Step 3: N-(4-((Benzyloxy)methyl)-2-methoxyphenyl)-3-(4,4,5,5-tetramethyl-1,3,2-dioxaborolan-2-yl)benzamide (I-21)

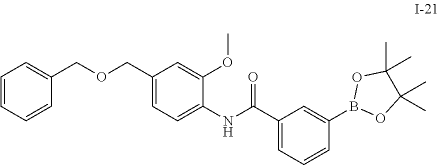 [SMILES: COc1cc(COCc2ccccc2)ccc1NC(=O)c1cccc(B2OC(C)(C)C(C)(C)O2)c1]

Following the procedure of Intermediate 16 Step 3, using 4-((benzyloxy)methyl)-2-methoxyaniline in place of 4-((benzyloxy)methyl)-3-fluoroaniline was obtained N-(4-((benzyloxy)methyl)-2-methoxyphenyl)-3-(4,4,5,5-tetramethyl-1,3,2-dioxaborolan-2-yl)benzamide in 30% yield.

LCMS: Method A: 2.38 min, MS: ES⁺ 474.2

### Intermediate 22

### N-(3-Fluoro-4-((pyridin-2-ylmethoxy)methyl)phenyl)-3-(4,4,5,5-tetramethyl-1,3,2-dioxaborolan-2-yl)benzamide (I-22)

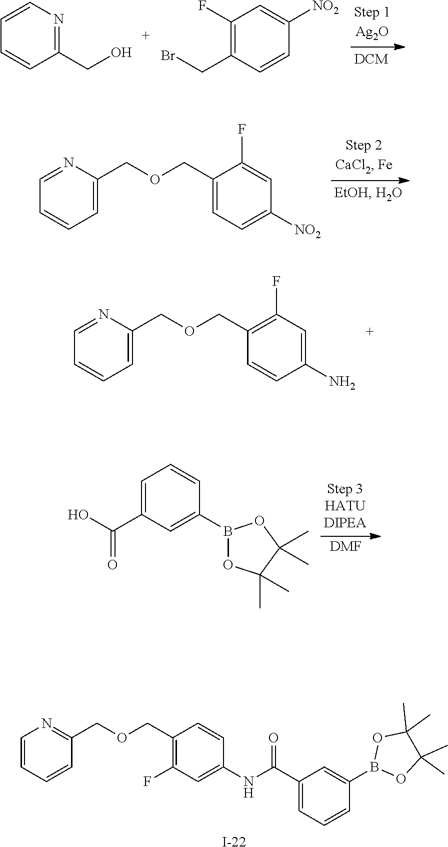

### Step 1: 2-(((2-Fluoro-4-nitrobenzyl)oxy)methyl)pyridine

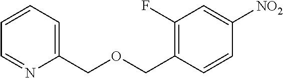 [SMILES: O=[N+]([O-])c1ccc(COCc2ccccn2)c(F)c1]

Following the procedure of Intermediate 3 Step 1, using pyridin-2-ylmethanol [CAS 586-98-1, BLD] in place of phenylmethanol, and 1-(bromomethyl)-2-fluoro-4-nitrobenzene [CAS 127349-56-8, BLD] in place of 1-(bromomethyl)-4-nitrobenzene, and purification by flash chromatography (silica gel, 0-100% EtOAc in isohexane), was obtained 2-(((2-fluoro-4-nitrobenzyl)oxy)methyl)pyridine in 53% yield.

LCMS: Method A: 1.16 min, MS: ES⁺ 263.1

### Step 2: 3-Fluoro-4-((pyridin-2-ylmethoxy)methyl)aniline

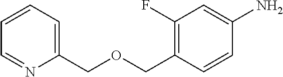 [SMILES: Nc1ccc(COCc2ccccn2)c(F)c1]

Following the procedure of Intermediate 3 Step 2, using 2-(((2-fluoro-4-nitrobenzyl)oxy)methyl)pyridine in place of 1-((benzyloxy)methyl)-4-nitrobenzene, CaCl₂ (4.5 eq) in place of AcOH, at 45° C. for 18 h, and without purification of crude product, was obtained 3-fluoro-4-((pyridin-2-ylmethoxy)methyl)aniline in 80% yield.

LCMS: Method A, 0.62 min, MS ES⁺ 233.2

### Step 3: N-(3-Fluoro-4-((pyridin-2-ylmethoxy)methyl)phenyl)-3-(4,4,5,5-tetramethyl-1,3,2-dioxaborolan-2-yl)benzamide (I-22)

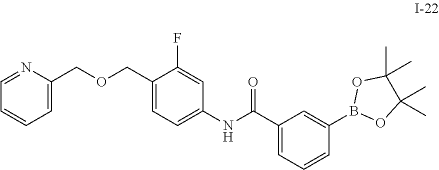 [SMILES: CC1(C)OB(c2cccc(C(=O)Nc3ccc(COCc4ccccn4)c(F)c3)c2)OC1(C)C]

Following the procedure of Intermediate 16 Step 3, using 3-fluoro-4-((pyridin-2-ylmethoxy)methyl)aniline in place of 4-((benzyloxy)methyl)-3-fluoroaniline was obtained N-(3-fluoro-4-((pyridin-2-ylmethoxy)methyl)phenyl)-3-(4,4,5,5-tetramethyl-1,3,2-dioxaborolan-2-yl)benzamide in 72% yield.

LCMS: Method A: 1.43 min, MS: ES⁺ 463.2

### Intermediate 23

### N-(3-Fluoro-4-(((4-methoxybenzyl)oxy)methyl)phenyl)-3-(4,4,5,5-tetramethyl-1,3,2-dioxaborolan-2-yl)benzamide (I-23)

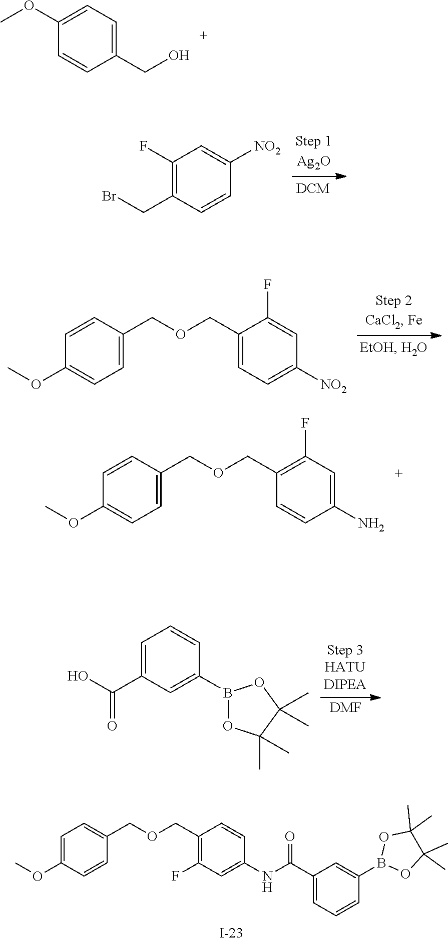

### Step 1: 2-Fluoro-1-(((4-methoxybenzyl)oxy)methyl)-4-nitrobenzene

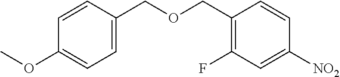 [SMILES: COc1ccc(COCc2ccc([N+](=O)[O-])cc2F)cc1]

Following the procedure of Intermediate 3 Step 1, using (4-methoxyphenyl)methanol [CAS 105-13-5, Apollo] in place of phenylmethanol, and 1-(bromomethyl)-2-fluoro-4-nitrobenzene [CAS 127349-56-8, BLD] in place of 1-(bromomethyl)-4-nitrobenzene, and purification by flash chromatography (silica gel, 0-100% EtOAc in isohexane), was obtained 2-fluoro-1-(((4-methoxybenzyl)oxy)methyl)-4-nitrobenzene in 73% yield.

LCMS: Method A: 1.66 min, MS: mass ion not observed

### Step 2: 3-Fluoro-4-(((4-methoxybenzyl)oxy)methyl)aniline

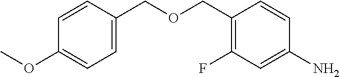 [SMILES: COc1ccc(COCc2ccc(N)cc2F)cc1]

Following the procedure of Intermediate 3 Step 2, using 2-fluoro-1-(((4-methoxybenzyl)oxy)methyl)-4-nitrobenzene in place of 1-((benzyloxy)methyl)-4-nitrobenzene, CaCl₂ (4.5 eq) in place of AcOH, at 45° C. for 18 h, and without purification of crude product, was obtained 3-fluoro-4-(((4-methoxybenzyl)oxy)methyl)aniline in 91% yield.

LCMS: Method A, 1.24 min, MS ES⁺ 262.2

### Step 3: N-(3-Fluoro-4-(((4-methoxybenzyl)oxy)methyl)phenyl)-3-(4,4,5,5-tetramethyl-1,3,2-dioxaborolan-2-yl)benzamide (I-23)

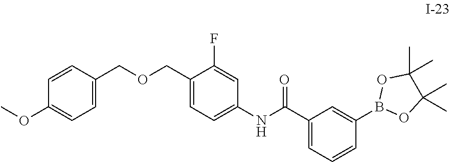

Following the procedure of Intermediate 16 Step 3, using 3-fluoro-4-(((4-methoxybenzyl)oxy)methyl)aniline in place of 4-((benzyloxy)methyl)-3-fluoroaniline was obtained N-(3-fluoro-4-(((4-methoxybenzyl)oxy)methyl)phenyl)-3-(4,4,5,5-tetramethyl-1,3,2-dioxaborolan-2-yl)benzamide in 49% yield.

LCMS: Method A: 1.97 min, MS: ES⁺ 492.2

### Intermediate 24

### N-(3-Fluoro-4-(((3-methoxybenzyl)oxy)methyl)phenyl)-3-(4,4,5,5-tetramethyl-1,3,2-dioxaborolan-2-yl)benzamide (I-24)

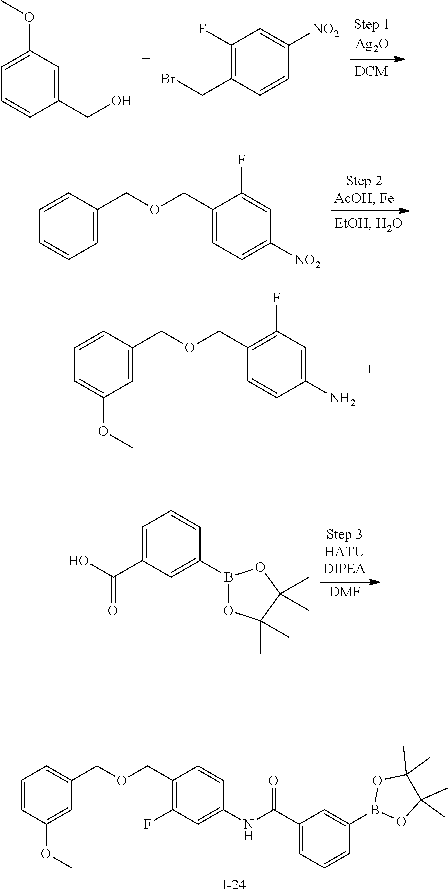

### Step 1: 2-Fluoro-1-(((3-methoxybenzyl)oxy)methyl)-4-nitrobenzene

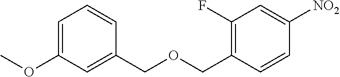 [SMILES: COc1cccc(COCc2ccc([N+](=O)[O-])cc2F)c1]

Following the procedure of Intermediate 3 Step 1, using (3-methoxyphenyl)methanol [CAS 6971-51-3, Fluorochem] in place of phenylmethanol, and 1-(bromomethyl)-2-fluoro-4-nitrobenzene [CAS 127349-56-8, BLD] in place of 1-(bromomethyl)-4-nitrobenzene, and purification by flash chromatography (silica gel, 0-100% EtOAc in isohexane), was obtained 2-fluoro-1-(((3-methoxybenzyl)oxy)methyl)-4-nitrobenzene in 81% yield.

LCMS: Method A: 1.67 min, MS: ES⁺ 309.1 (M+NH4)+

### Step 2: 3-Fluoro-4-(((3-methoxybenzyl)oxy)methyl)aniline

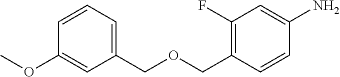 [SMILES: COc1cccc(COCc2ccc(N)cc2F)c1]

Following the procedure of Intermediate 3 Step 2, using 2-fluoro-1-(((3-methoxybenzyl)oxy)methyl)-4-nitrobenzene in place of 1-((benzyloxy)methyl)-4-nitrobenzene, CaCl₂ (4.5 eq) in place of AcOH, at 45° C. for 18 h, and without purification of crude product, was obtained 3-fluoro-4-(((3-methoxybenzyl)oxy)methyl)aniline in 75% yield.

LCMS: Method A, 1.28 min, MS ES⁺ 262.1

### Step 3: N-(3-Fluoro-4-(((3-methoxybenzyl)oxy)methyl)phenyl)-3-(4,4,5,5-tetramethyl-1,3,2-dioxaborolan-2-yl)benzamide (I-24)

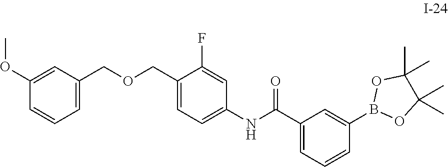

Following the procedure of Intermediate 16 Step 3, using 3-fluoro-4-(((3-methoxybenzyl)oxy)methyl)aniline in place of 4-((benzyloxy)methyl)-3-fluoroaniline was obtained N-(3-fluoro-4-(((3-methoxybenzyl)oxy)methyl)phenyl)-3-(4,4,5,5-tetramethyl-1,3,2-dioxaborolan-2-yl)benzamide in 56% yield.

LCMS: Method A: 1.99 min, MS: ES⁺ 492.2

### Intermediate 25

### N-(4-((Benzyloxy)methyl)phenyl)-3-(4,4,5,5-tetramethyl-1,3,2-dioxaborolan-2-yl)benzamide (I-25)

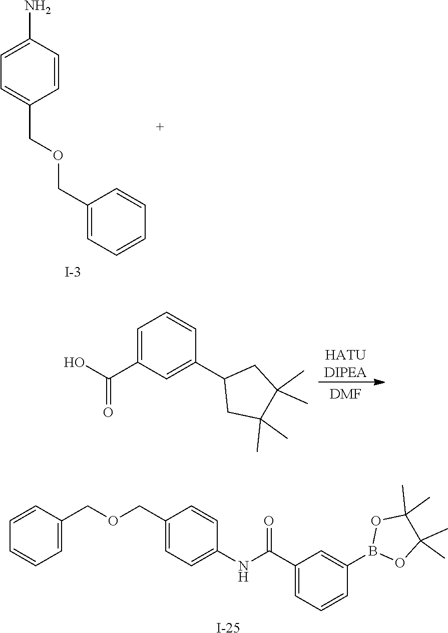

Following the procedure of Intermediate 16 Step 3, using 4-((benzyloxy)methyl)aniline (I-3) in place of 4-((benzyloxy)methyl)-3-fluoroaniline was obtained N-(4-((benzyloxy)methyl)phenyl)-3-(4,4,5,5-tetramethyl-1,3,2-dioxaborolan-2-yl)benzamide in 70% yield.

LCMS: Method A: 2.23 min, MS: ES⁺ 444.2

### Intermediate 26

### 6-Chloro-N-(4-phenethoxyphenyl)pyrimidine-4-carboxamide (I-26)

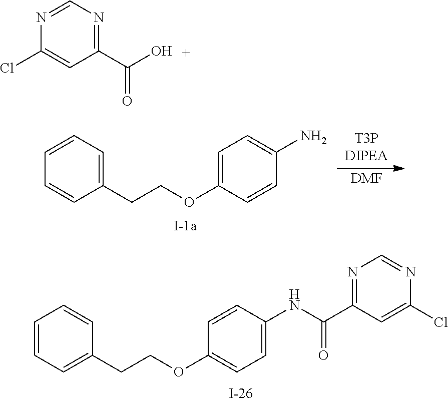

Following the procedure of Intermediate 16 Step 3, using T3P (50% in EtOAc, 1.1 eq) in place of HATU, 6-chloro-4-pyrimidinecarboxylic acid [CAS 37131-91-2, BLD] in place of 3-(4,4,5,5-tetramethyl-1,3,2-dioxaborolan-2-yl)benzoic acid, and 4-phenethoxyaniline (I-1a) in place of 4-((benzyloxy)methyl)-3-fluoroaniline was obtained 6-chloro-N-(4-phenethoxyphenyl)pyrimidine-4-carboxamide in 46% yield.

LCMS: Method C: 1.12 min, MS: ES⁺ 354

### Intermediate 27

### 4-Bromo-N-(4-phenethoxyphenyl)picolinamide (I-27)

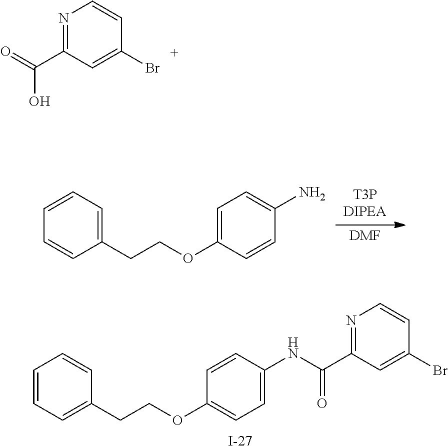

Following the procedure of Intermediate 16 Step 3, using T3P (50% in EtOAc, 1.1 eq) in place of HATU, 4-bromopicolinic acid [CAS 30766-03-1, BLD] in place of 3-(4,4,5,5-tetramethyl-1,3,2-dioxaborolan-2-yl)benzoic acid, and 4-phenethoxyaniline (I-1a) in place of 4-((benzyloxy)methyl)-3-fluoroaniline was obtained 4-bromo-N-(4-phenethoxyphenyl)picolinamide in 74% yield.

LCMS: Method C: 1.18 min, MS: ES⁺ 397.0/399.0

### Intermediate 28

### 6-Chloro-N-(4-phenethoxyphenyl)pyridazine-4-carboxamide (I-28)

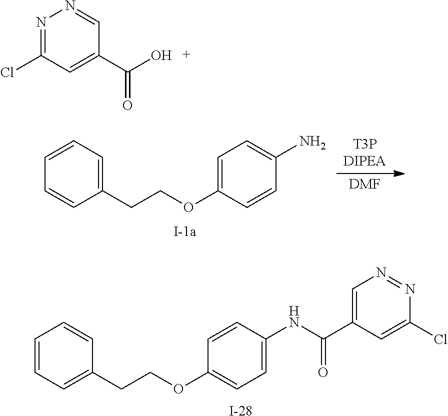

Following the procedure of Intermediate 16 Step 3, using T3P (50% in EtOAc, 1.1 eq) in place of HATU, 3-chloropyridazine-5-carboxylic acid [CAS 1256794-24-7, BLD] in place of 3-(4,4,5,5-tetramethyl-1,3,2-dioxaborolan-2-yl)benzoic acid, and 4-phenethoxyaniline (I-1a) in place of 4-((benzyloxy)methyl)-3-fluoroaniline was obtained 6-chloro-N-(4-phenethoxyphenyl)pyridazine-4-carboxamide in 81% yield.

LCMS: Method C: 1.04 min, MS: ES⁺ 354

### Intermediate 29

### 2-Bromo-N-(4-(cyclopropylmethoxy)phenyl)isonicotinamide (I-29)

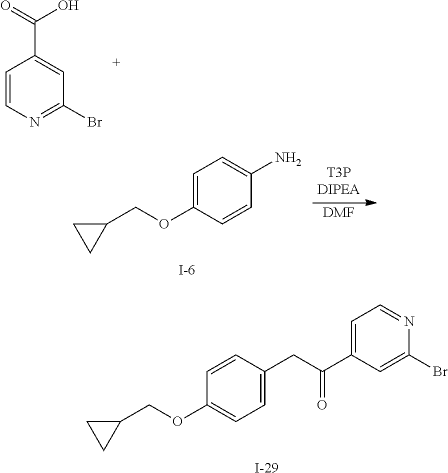

Following the procedure of Intermediate 16 Step 3, using T3P (50% in EtOAc, 1.1 eq) in place of HATU, 2-bromoisonicotinic acid [CAS 66572-56-3, Apollo] in place of 3-(4,4,5,5-tetramethyl-1,3,2-dioxaborolan-2-yl)benzoic acid, and 4-(cyclopropylmethoxy)aniline (I-6) in place of 4-((benzyloxy)methyl)-3-fluoroaniline was obtained 2-bromo-N-(4-(cyclopropylmethoxy)phenyl)isonicotinamide in 50% yield.

LCMS: Method C: 1.02 min, MS: ES⁺ 347.0/349.0

### Intermediate 30

### 2-Bromo-N-(4-(cyclopropylmethoxy)phenyl)-5-fluoroisonicotinamide (I-30)

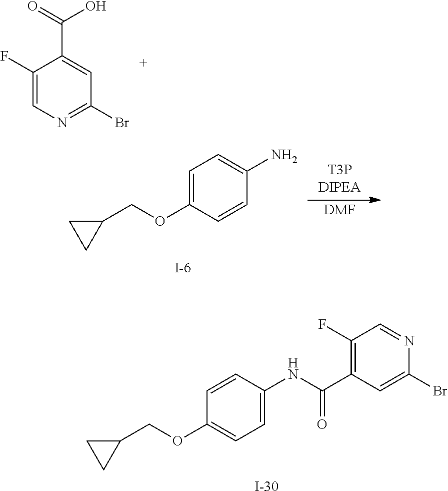

Following the procedure of Intermediate 16 Step 3, using T3P (50% in EtOAc, 1.1 eq) in place of HATU, 2-bromo-5-fluoroisonicotinic acid [CAS 885588-12-5, BLD] in place of 3-(4,4,5,5-tetramethyl-1,3,2-dioxaborolan-2-yl)benzoic acid, and 4-(cyclopropylmethoxy)aniline (I-6) in place of 4-((benzyloxy)methyl)-3-fluoroaniline was obtained 2-bromo-N-(4-(cyclopropylmethoxy)phenyl)-5-fluoroisonicotinamide in 68% yield.

LCMS: Method C: 1.04 min, MS: ES⁺ 365.0/367.0

### Intermediate 31

### N-(4-((Benzyloxy)methyl)phenyl)-2-bromoisonicotinamide (I-31)

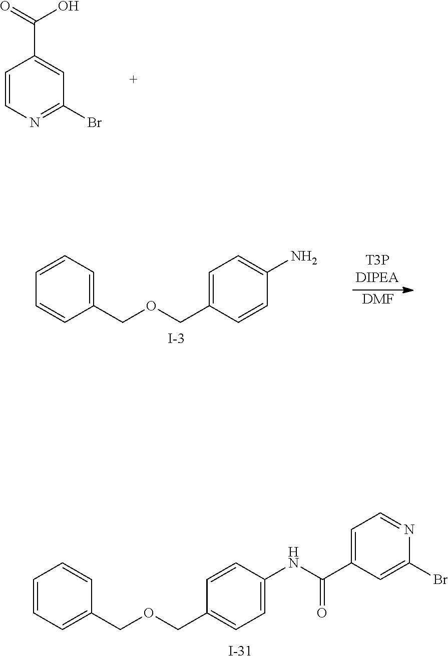

Following the procedure of Intermediate 16 Step 3, using T3P (50% in EtOAc, 1.1 eq) in place of HATU, 2-bromoisonicotinic acid [CAS 66572-56-3, Apollo] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline was obtained N-(4-((benzyloxy)methyl)phenyl)-2-bromoisonicotinamide in 53% yield.

LCMS: Method A: 1.97 min, MS: ES⁺ 397.0/399.0

### Intermediate 32

### N-(4-((Benzyloxy)methyl)phenyl)-2-chloro-6-methylisonicotinamide (I-32)

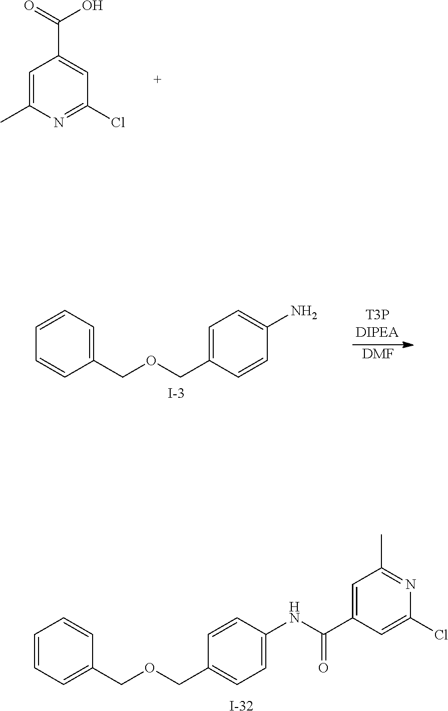

Following the procedure of Intermediate 16 Step 3, using T3P (50% in EtOAc, 1.1 eq) in place of HATU, 2-chloro-6-methylisonicotinic acid [CAS 25462-85-5, Fluorochem] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline was obtained N-(4-((benzyloxy)methyl)phenyl)-2-chloro-6-methylisonicotinamide in 52% yield.

LCMS: Method C: 0.84 min, MS: ES⁺ 367.2

### Intermediate 33

### N-(4-((Benzyloxy)methyl)phenyl)-2-bromo-5-fluoroisonicotinamide (I-33)

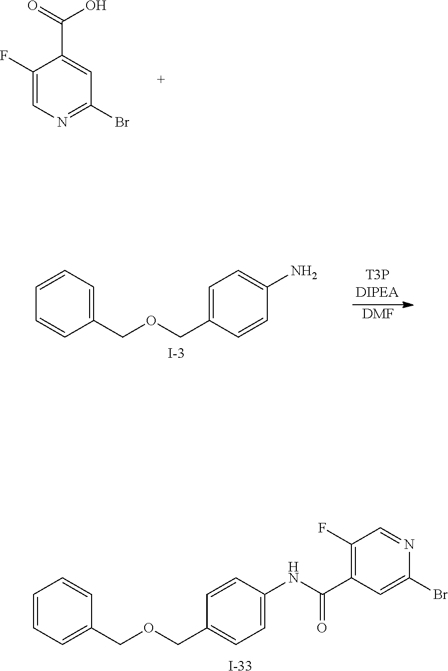

Following the procedure of Intermediate 16 Step 3, using T3P (50% in EtOAc, 1.1 eq) in place of HATU, 2-bromo-5-fluoroisonicotinic acid [CAS 885588-12-5, BLD] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline was obtained N-(4-((benzyloxy)methyl)phenyl)-2-bromo-5-fluoroisonicotinamide in 63% yield.

LCMS: Method C: 1.1 min, MS: ES⁺ 415.0/417.0

### Intermediate 34

### N-(4-((Benzyloxy)methyl)-3-fluorophenyl)-2-bromoisonicotinamide (I-34)

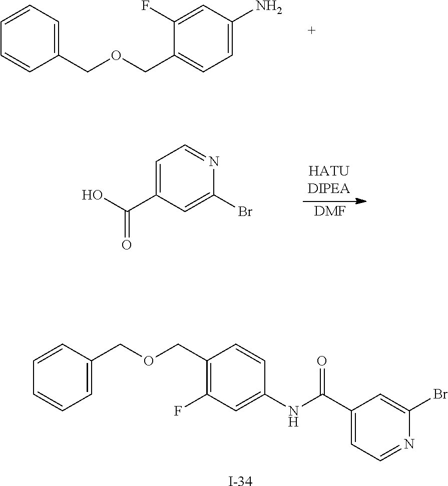

Following the procedure of Intermediate 16 Step 3, using 2-bromoisonicotinic acid [CAS 66572-56-3, Apollo] in place of 3-(4,4,5,5-tetramethyl-1,3,2-dioxaborolan-2-yl)benzoic acid, and at rt rather than 40° C., was obtained N-(4-((benzyloxy)methyl)-3-fluorophenyl)-2-bromoisonicotinamide in 41% yield.

LCMS: Method A: 2 min, MS: ES⁺ 415.1/417.0

### Intermediate 35

### 2-Bromo-N-(4-(cyclopropoxymethyl)-3-fluorophenyl)isonicotinamide (I-35)

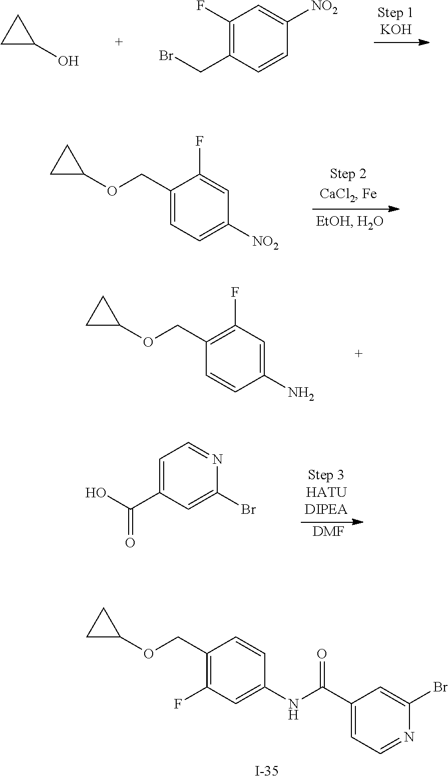

### Step 1: 1-(Cyclopropoxymethyl)-2-fluoro-4-nitrobenzene

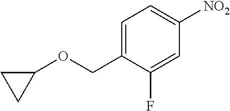 [SMILES: O=[N+]([O-])c1ccc(COC2CC2)c(F)c1]

To a mixture of cyclopropanol (CAS 16545-68-9, BLD, 2.71 mL, 42.7 mmol) and 1-(bromomethyl)-2-fluoro-4-nitrobenzene (CAS 127349-56-8, BLD, 1.00 g, 4.27 mmol) was added KOH (288 mg, 5.13 mmol). The mixture was stirred at rt for 18 h, poured into ice-water (~400 mL) and extracted with EtOAc (3×100 mL). The organics were washed with brine (50 mL), dried over MgSO₄, and adsorbed onto silica gel. Purification by flash chromatography (silica gel, 0-10% EtOAc in isohexane) afforded 1-(cyclopropoxymethyl)-2-fluoro-4-nitrobenzene (300 mg, 32%).

LCMS: Method A: 1.71 min, MS: ES⁺ 212.1

### Step 2: 4-(Cyclopropoxymethyl)-3-fluoroaniline

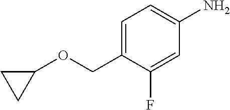 [SMILES: Nc1ccc(COC2CC2)c(F)c1]

Following the procedure of Intermediate 3 Step 2, using 1-(cyclopropoxymethyl)-2-fluoro-4-nitrobenzene in place of 1-((benzyloxy)methyl)-4-nitrobenzene, CaCl₂ (4.5 eq) in place of AcOH, at 80° C. for 18 h, and without purification of crude product, was obtained 4-(cyclopropoxymethyl)-3-fluoroaniline in 58% yield.

LCMS: Method A, 1.13 min, MS: ES⁺ 182.2

### Step 3: 2-Bromo-N-(4-(cyclopropoxymethyl)-3-fluorophenyl)isonicotinamide (I-35)

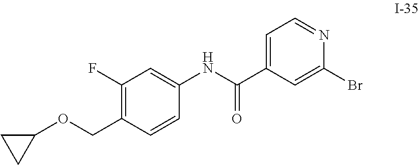 [SMILES: O=C(Nc1ccc(COC2CC2)c(F)c1)c1ccnc(Br)c1]

Following the procedure of Intermediate 16 Step 3, using 2-bromoisonicotinic acid [CAS 66572-56-3, Apollo] in place of 3-(4,4,5,5-tetramethyl-1,3,2-dioxaborolan-2-yl)benzoic acid, and 4-(cyclopropoxymethyl)-3-fluoroaniline in place of 4-((benzyloxy)methyl)-3-fluoroaniline and at rt rather than 40° C., was obtained 2-bromo-N-(4-(cyclopropoxymethyl)-3-fluorophenyl)isonicotinamide in 65% yield.

LCMS: Method A: 1.78 min, MS: ES⁺ 365.0/367.0

### Intermediate 36

### 2-Bromo-N-(4-(cyclopropylmethoxy)-3-fluorophenyl)isonicotinamide (I-36)

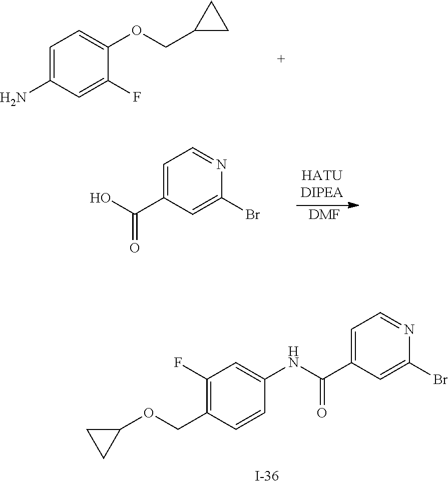

Following the procedure of Intermediate 16 Step 3, using 2-bromoisonicotinic acid [CAS 66572-56-3, Apollo] in place of 3-(4,4,5,5-tetramethyl-1,3,2-dioxaborolan-2-yl)benzoic acid, and 4-(cyclopropylmethoxy)-3-fluoroaniline [CAS 937598-42-0, Enamine] in place of 4-((benzyloxy)methyl)-3-fluoroaniline, and at rt rather than 40° C., was obtained 2-bromo-N-(4-(cyclopropylmethoxy)-3-fluorophenyl)isonicotinamide in 74% yield.

LCMS: Method A: 1.82 min, MS: ES⁺ 365.0/367.0

### Intermediate 37

### (3-((4-((Benzyloxy)methyl)phenyl)carbamoyl)-4-methoxyphenyl)boronic acid (I-37)

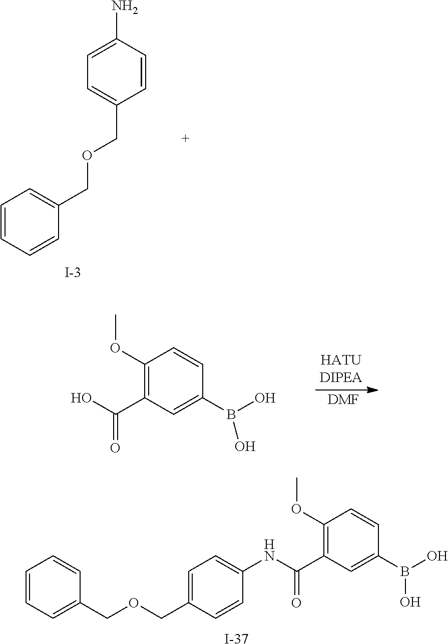

Following the procedure of Intermediate 16 Step 3, using 5-borono-2-methoxybenzoic acid [CAS 913836-12-1, BLD] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline was obtained (3-((4-((benzyloxy)methyl)phenyl)carbamoyl)-4-methoxyphenyl)boronic acid in 87% yield.

LCMS: Method A: 1.74 min, MS: ES⁺ 392.2

### Intermediate 38

### N-(4-((Benzyloxy)methyl)phenyl)-4-methoxy-3-(4,4,5,5-tetramethyl-1,3,2-dioxaborolan-2-yl)benzamide (I-38)

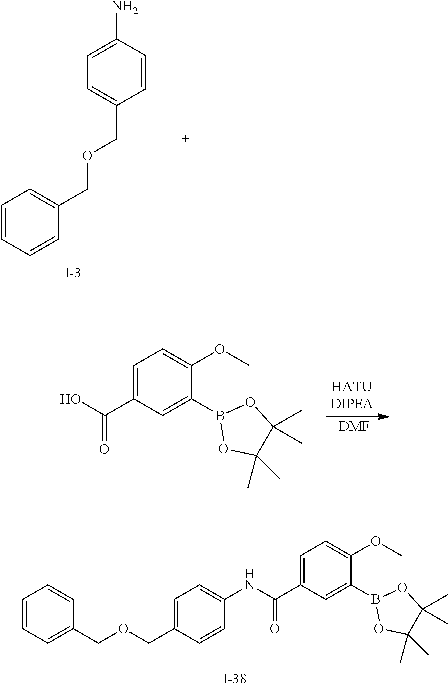

Following the procedure of Intermediate 16 Step 3, using 4-methoxy-3-(4,4,5,5-tetramethyl-1,3,2-dioxaborolan-2-yl)benzoic acid [CAS 269409-71-4, Manchester Organics] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline was obtained N-(4-((benzyloxy)methyl)phenyl)-4-methoxy-3-(4,4,5,5-tetramethyl-1,3,2-dioxaborolan-2-yl)benzamide in 52% yield.

LCMS: Method A: 2.12 min, MS: ES⁺ 474.2

### Intermediate 39

### (3-((4-((Benzyloxy)methyl)phenyl)carbamoyl)-4-methylphenyl)boronic acid (I-39)

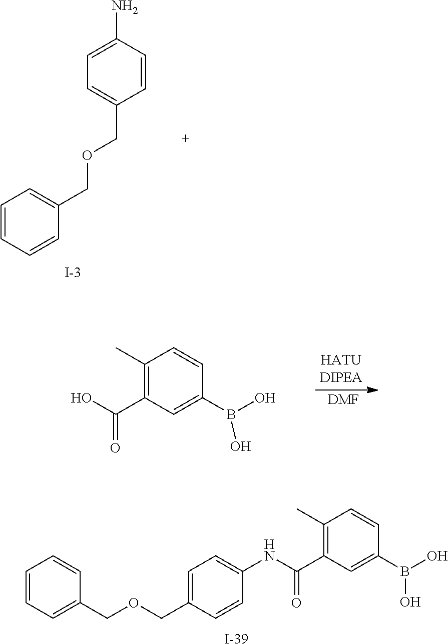

Following the procedure of Intermediate 16 Step 3, using 5-borono-2-methylbenzoic acid [CAS 1256346-18-5, Combi-Blocks] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline was obtained (3-((4-((benzyloxy)methyl)phenyl)carbamoyl)-4-methylphenyl)boronic acid in 34% yield.

LCMS: Method A: 1.71 min, MS: ES⁺ 376.2

### Intermediate 40

### N-(4-((Benzyloxy)methyl)phenyl)-4-methyl-3-(4,4,5,5-tetramethyl-1,3,2-dioxaborolan-2-yl)benzamide (I-40)

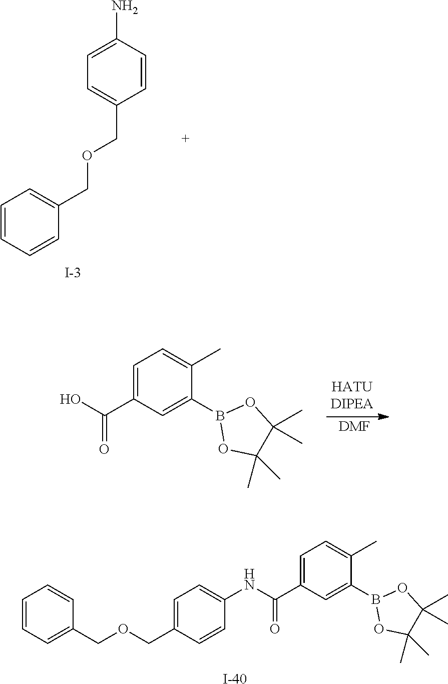

Following the procedure of Intermediate 16 Step 3, using 4-methyl-3-(4,4,5,5-tetramethyl-1,3,2-dioxaborolan-2-yl)benzoic acid [CAS 515131-35-8, BLD] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline was obtained N-(4-((benzyloxy)methyl)phenyl)-4-methyl-3-(4,4,5,5-tetramethyl-1,3,2-dioxaborolan-2-yl)benzamide in 57% yield.

LCMS: Method A: 2.34 min, MS: ES⁺ 458.3

### Intermediate 41

### N-(4-(Cyclopropylmethoxy)phenyl)-2-fluoro-5-(4,4,5,5-tetramethyl-1,3,2-dioxaborolan-2-yl)benzamide (I-41)

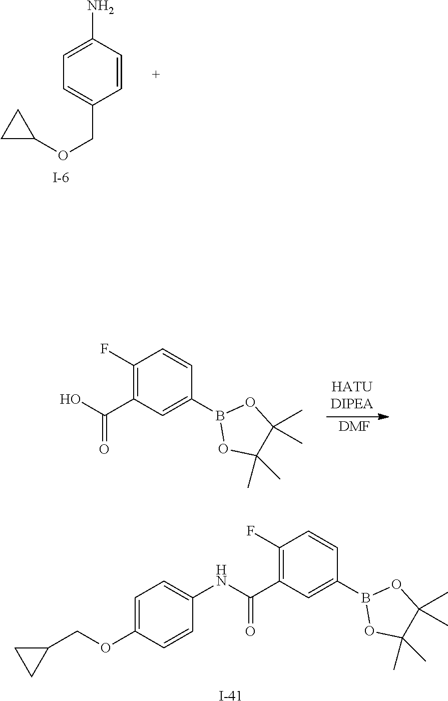

Following the procedure of Intermediate 16 Step 3, using 2-fluoro-5-(4,4,5,5-tetramethyl-1,3,2-dioxaborolan-2-yl)benzoic acid [CAS 882679-10-9, BLD] in place of 3-(4,4,5,5-tetramethyl-1,3,2-dioxaborolan-2-yl)benzoic acid, and 4-(cyclopropylmethoxy)aniline (I-6) in place of 4-((benzyloxy)methyl)-3-fluoroaniline, and purification by flash chromatography (silica gel, 0-10% MeOH in DCM), was obtained N-(4-(cyclopropylmethoxy)phenyl)-2-fluoro-5-(4,4,5,5-tetramethyl-1,3,2-dioxaborolan-2-yl)benzamide in 42% yield.

LCMS: Method A: 2.16 min, MS: ES⁺ 412.2

### Intermediate 42

### (5-((4-((Benzyloxy)methyl)phenyl)carbamoyl)-2-fluorophenyl)boronic acid (I-42)

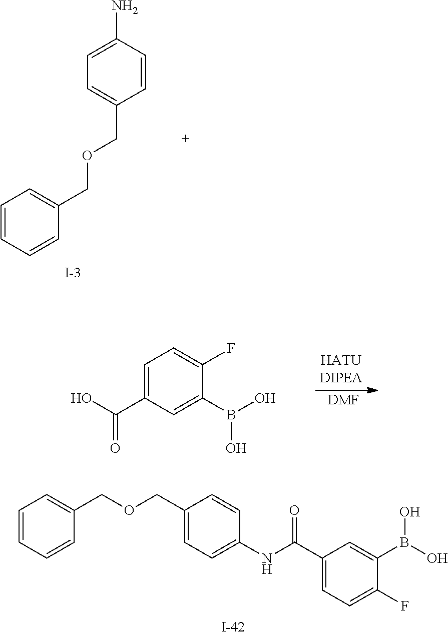

Following the procedure of Intermediate 16 Step 3, using 3-borono-4-fluorobenzoic acid [CAS 874219-59-7, BLD] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline was obtained (5-((4-((benzyloxy)methyl)phenyl)carbamoyl)-2-fluorophenyl)boronic acid in 91% yield.

LCMS: Method A: 1.67 min, MS: ES⁺ 380.2

### Intermediate 43

### (3-((4-((Benzyloxy)methyl)phenyl)carbamoyl)-2-fluorophenyl)boronic acid (I-43)

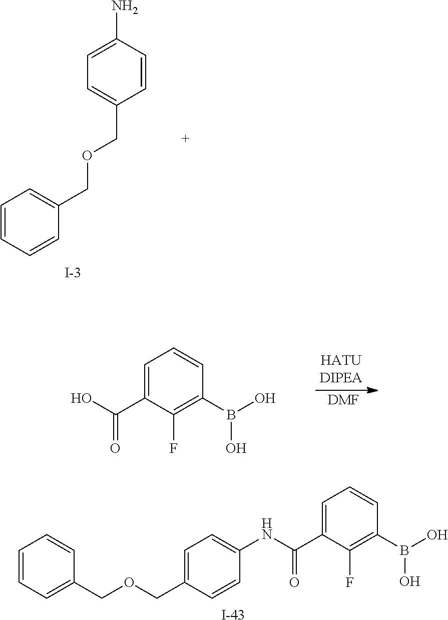

Following the procedure of Intermediate 16 Step 3, using 3-borono-2-fluorobenzoic acid [CAS 1072952-09-0, Combi-Blocks] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline was obtained (3-((4-((benzyloxy)methyl)phenyl)carbamoyl)-2-fluorophenyl)boronic acid in 57% yield.

LCMS: Method A: 1.67 min, MS: ES⁺ 380.2

### Intermediate 44

### (3-((4-((Benzyloxy)methyl)phenyl)carbamoyl)-2,4-difluorophenyl)boronic acid (I-44)

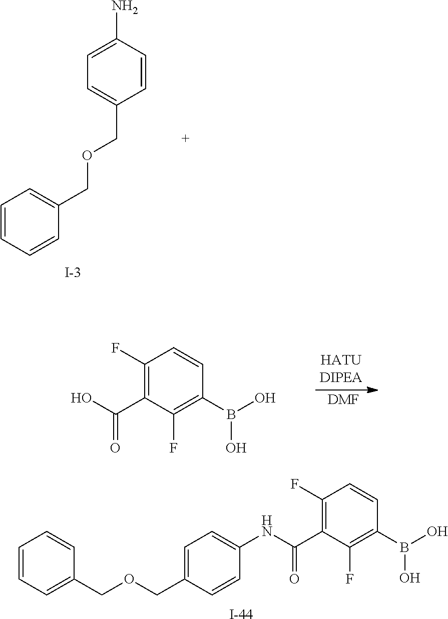

Following the procedure of Intermediate 16 Step 3, using 3-borono-2,6-difluorobenzoic acid [CAS 1451393-05-7, Combi-Blocks] in place of 3-(4,4,5,5-tetramethyl-1,3,2-dioxaborolan-2-yl)benzoic acid, and 4-((benzyloxy)methyl)aniline (I-3) in place of 4-((benzyloxy)methyl)-3-fluoroaniline, and purification by flash chromatography (silica gel, 0-10% MeOH in DCM), was obtained (3-((4-((benzyloxy)methyl)phenyl)carbamoyl)-2,4-difluorophenyl)boronic acid in 35% yield.

LCMS: Method A: 1.68 min, MS: ES⁺ 398.1

### Intermediate 45

### N-(4-((Cyclopropylmethoxy)methyl)-3-fluorophenyl)-2-fluoro-5-(4,4,5,5-tetramethyl-1,3,2-dioxaborolan-2-yl)benzamide (I-45)

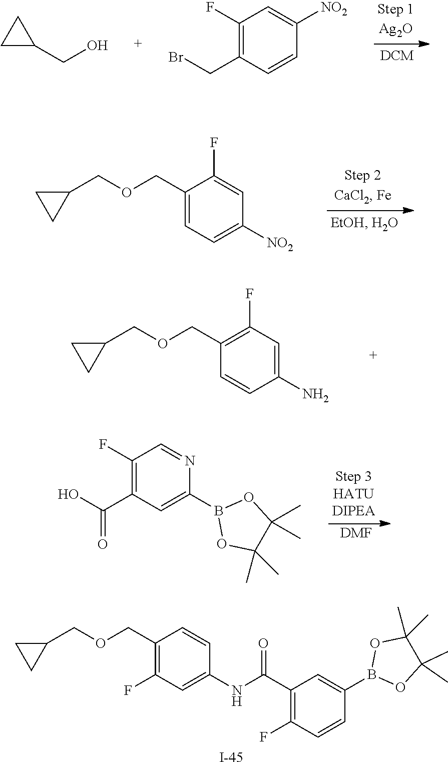

### Step 1: 1-((Cyclopropylmethoxy)methyl)-2-fluoro-4-nitrobenzene

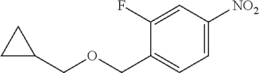 [SMILES: O=[N+]([O-])c1ccc(COCC2CC2)c(F)c1]

Following the procedure of Intermediate 3 Step 1, using cyclopropylmethanol [CAS 2516-33-8, Fluorochem] in place of phenylmethanol, and 1-(bromomethyl)-2-fluoro-4-nitrobenzene [CAS 127349-56-8, Apollo] in place of 1-(bromomethyl)-4-nitrobenzene was obtained 1-((cyclopropylmethoxy)methyl)-2-fluoro-4-nitrobenzene in 82% yield.

LCMS: Method A: 1.82 min, MS: ES⁺ 226.1

### Step 2: 4-((Cyclopropylmethoxy)methyl)-3-fluoroaniline

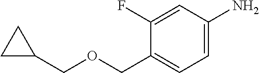 [SMILES: Nc1ccc(COCC2CC2)c(F)c1]

Following the procedure of Intermediate 3 Step 2, using 1-((cyclopropylmethoxy)methyl)-2-fluoro-4-nitrobenzene in place of 1-((benzyloxy)methyl)-4-nitrobenzene, CaCl₂ (4.5 eq) in place of AcOH, at 80° C. for 18 h, and without purification of crude product, was obtained 4-((cyclopropylmethoxy)methyl)-3-fluoroaniline in 67% yield.

LCMS: Method A, 1.23 min, MS ES⁺ 196.2

### Step 3: N-(4-((Cyclopropylmethoxy)methyl)-3-fluorophenyl)-2-fluoro-5-(4,4,5,5-tetramethyl-1,3,2-dioxaborolan-2-yl)benzamide (I-45)

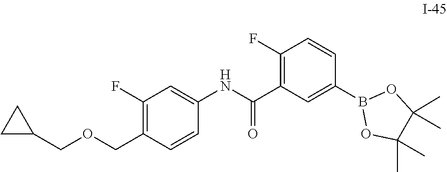 [SMILES: CC1(C)OB(c2ccc(F)c(C(=O)Nc3ccc(COCC4CC4)c(F)c3)c2)OC1(C)C]

Following the procedure of Intermediate 16 Step 3, using 2-fluoro-5-(4,4,5,5-tetramethyl-1,3,2-dioxaborolan-2-yl)benzoic acid [CAS 882679-10-9, BLD] in place of 3-(4,4,5,5-tetramethyl-1,3,2-dioxaborolan-2-yl)benzoic acid, and 4-((cyclopropylmethoxy)methyl)-3-fluoroaniline in place of 4-((benzyloxy)methyl)-3-fluoroaniline, and at rt rather than 40° C., was obtained N-(4-((cyclopropylmethoxy)methyl)-3-fluorophenyl)-2-fluoro-5-(4,4,5,5-tetramethyl-1,3,2-dioxaborolan-2-yl)benzamide in 60% yield.

LCMS: Method A: 2.22 min, MS: ES⁺ 444.2

### Intermediate 46

### N-(4-(Cyclopropylmethoxy)-3-fluorophenyl)-2-fluoro-5-(4,4,5,5-tetramethyl-1,3,2-dioxaborolan-2-yl)benzamide (I-46)

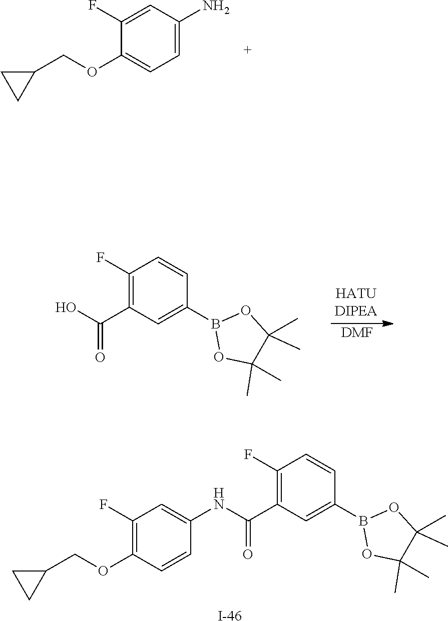

Following the procedure of Intermediate 16 Step 3, using 2-fluoro-5-(4,4,5,5-tetramethyl-1,3,2-dioxaborolan-2-yl)benzoic acid [CAS 882679-10-9, BLD] in place of 3-(4,4,5,5-tetramethyl-1,3,2-dioxaborolan-2-yl)benzoic acid, and 4-(cyclopropylmethoxy)-3-fluoroaniline [CAS 937598-42-0, Enamine] in place of 4-((benzyloxy)methyl)-3-fluoroaniline, and at rt rather than 40° C., was obtained N-(4-(cyclopropylmethoxy)-3-fluorophenyl)-2-fluoro-5-(4,4,5,5-tetramethyl-1,3,2-dioxaborolan-2-yl)benzamide in 71% yield.

LCMS: Method A: 2.2 min, MS: ES⁺ 430.2

### Intermediate 47

### N-(4-((Benzyloxy)methyl)-3-fluorophenyl)-2-fluoro-5-(4,4,5,5-tetramethyl-1,3,2-dioxaborolan-2-yl)benzamide (I-47)

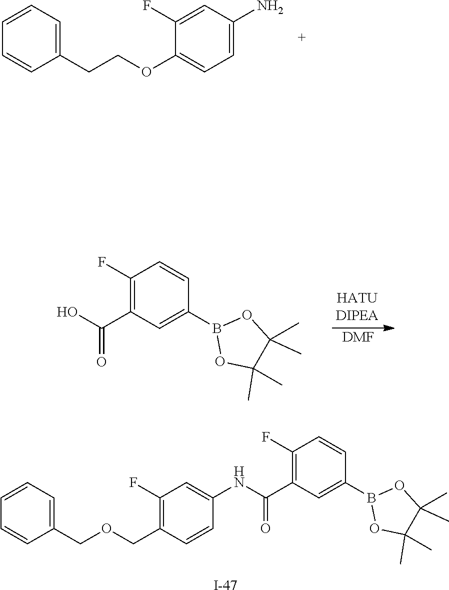

Following the procedure of Intermediate 16 Step 3, using 2-fluoro-5-(4,4,5,5-tetramethyl-1,3,2-dioxaborolan-2-yl)benzoic acid [CAS 882679-10-9, BLD] in place of 3-(4,4,5,5-tetramethyl-1,3,2-dioxaborolan-2-yl)benzoic acid, and at rt rather than 40° C., was obtained N-(4-((benzyloxy)methyl)-3-fluorophenyl)-2-fluoro-5-(4,4,5,5-tetramethyl-1,3,2-dioxaborolan-2-yl)benzamide in 57% yield.

LCMS: Method A: 2.34 min, MS: ES⁺ 480.2

### Intermediate 48

### N-(4-(Cyclopropoxymethyl)-3-fluorophenyl)-2-fluoro-5-(4,4,5,5-tetramethyl-1,3,2-dioxaborolan-2-yl)benzamide (I-48)

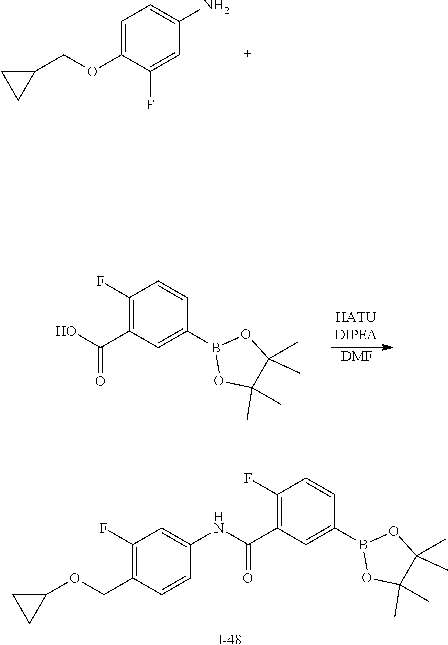

Following the procedure of Intermediate 16 Step 3, using 2-fluoro-5-(4,4,5,5-tetramethyl-1,3,2-dioxaborolan-2-yl)benzoic acid [CAS 882679-10-9, BLD] in place of 3-(4,4,5,5-tetramethyl-1,3,2-dioxaborolan-2-yl)benzoic acid, and 4-(cyclopropoxymethyl)-3-fluoroaniline (Intermediate 35 Step 2) in place of 4-((benzyloxy)methyl)-3-fluoroaniline, and at rt rather than 40° C., was obtained N-(4-(cyclopropoxymethyl)-3-fluorophenyl)-2-fluoro-5-(4,4,5,5-tetramethyl-1,3,2-dioxaborolan-2-yl)benzamide in 35% yield.

LCMS: Method A: 2.19 min, MS: ES⁺ 430.2

### Intermediate 49

### 3-Bromo-N-(1-phenethyl-1H-pyrazol-4-yl)benzamide (I-49)

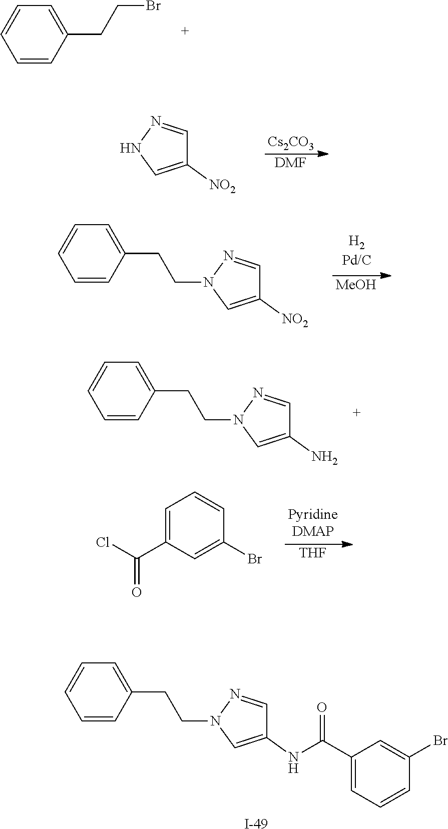

### Step 1: 4-Nitro-1-phenethyl-1H-pyrazole

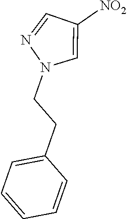 [SMILES: O=[N+]([O-])c1cnn(CCc2ccccc2)c1]

A mixture of 4-nitro-1H-pyrazole (CAS 2075-46-9, Fluorochem, 300 mg, 2.65 mmol), Cs₂CO₃ (1.73 g, 5.31 mmol) and (2-bromoethyl)benzene (CAS 103-63-9, Combi-Blocks, 0.722 mL, 5.31 mmol) in DMF (2 mL) was stirred at 60° C. for 16 h. Water (30 mL) was added and the mixture extracted with EtOAc (3×20 mL). The organics were dried over Na₂SO₄ and concentrated under reduced pressure. Purification by flash chromatography (silica gel, 0-50% EtOAc in isohexane) afforded 4-nitro-1-phenethyl-1H-pyrazole (620 mg, 100%).

LCMS: Method A: 1.56 min, MS: ES⁺ 218.1

### Step: 1-Phenethyl-1H-pyrazol-4-amine

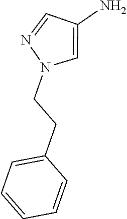 [SMILES: Nc1cnn(CCc2ccccc2)c1]

A solution of 4-nitro-1-phenethyl-1H-pyrazole (300 mg, 1.30 mmol) in MeOH (15.0 mL) was passed through a Pd/C 10% cartridge under hydrogen at rt at 18 bar (H-Cube®). The mixture was concentrated under reduced pressure to afford 1-phenethyl-1H-pyrazol-4-amine (238 mg, 89%).

LCMS: Method A: 0.14 min, MS: ES⁺ 188.2

### Step 3: 3-Bromo-N-(1-phenethyl-1H-pyrazol-4-yl)benzamide (I-49)

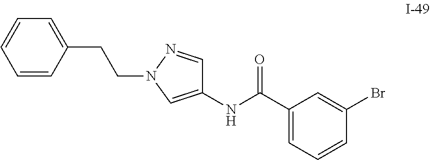 [SMILES: O=C(Nc1cnn(CCc2ccccc2)c1)c1cccc(Br)c1]

To 3-bromobenzoyl chloride (1711-09-7, BLD, 254 mg, 1.16 mmol), pyridine (0.233 mL, 2.89 mmol) in THF (8 mL) was added 1-phenethyl-1H-pyrazol-4-amine (238 mg, 1.16 mmol) and then DMAP (28 mg, 0.231 mmol). The mixture was stirred at 40° C. for 2 h, cooled to rt. Water (2 mL) was added and the mixture extracted with DCM (30 mL). The organic phase was dried under reduced pressure and the residue was triturated with Et₂O (2×10 mL) to afford 3-bromo-N-(1-phenethyl-1H-pyrazol-4-yl)benzamide (400 mg, 93%).

LCMS: Method A: 1.79 min, MS: ES⁺ 370.0/372.0

### Intermediate 50

### 3-Bromo-N-(4-(tosylmethyl)phenyl)benzamide (I-50)

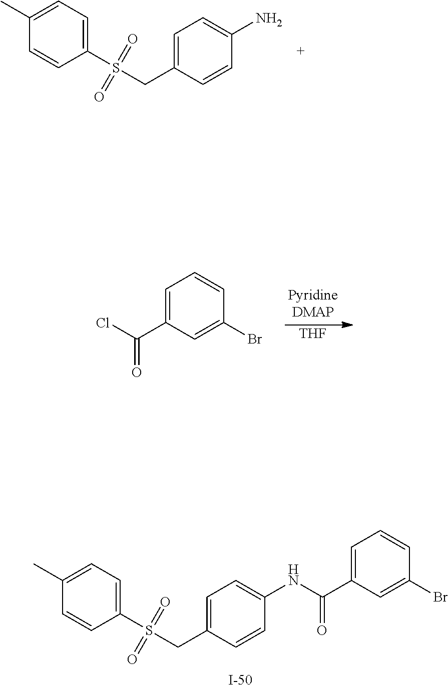

Following the procedure of Intermediate 49 Step 3, using (4-{[(4-methylphenyl)sulfonyl]methyl}phenyl)amine [54306-15-9, Combi-Blocks] in place of 1-phenethyl-1H-pyrazol-4-amine was obtained 3-bromo-N-(4-(tosylmethyl)phenyl)benzamide in 85% yield.

LCMS: Method A: 1.89 min, MS: ES⁺ 461.1/463.1 (M+17)

### Intermediate 51

### Methyl 5-(3-(chlorocarbonyl)phenyl)-2-methylnicotinate, HCl (I-51)

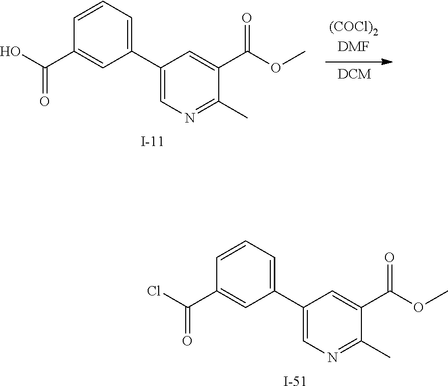

To 3-(5-(methoxycarbonyl)-6-methylpyridin-3-yl)benzoic acid (I-11, 790 mg, 2.91 mmol) in DCM (12 mL) at 0° C. was added oxalyl chloride (2.55 mL, 29.1 mmol) and DMF (0.023 mL, 0.291 mmol). The mixture was stirred at rt for 18 h and concentrated under reduced pressure to afford methyl 5-(3-(chlorocarbonyl)phenyl)-2-methylnicotinate, HCl (986 mg, 85%).

LCMS: Method A: 1.28 min, MS: ES⁺ 286.1 (in MeOH)

### Intermediate 52

### 5-Bromo-6-chloro-N-(4-phenethoxyphenyl)nicotinamide (I-52)

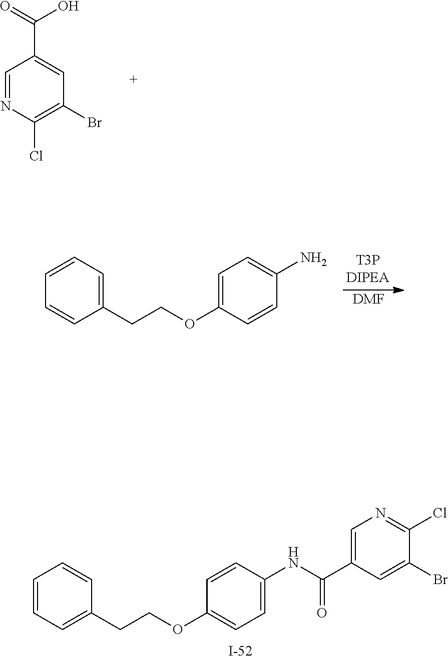

Following the procedure of Intermediate 16 Step 3, using T3P (50% in EtOAc, 1.1 eq) in place of HATU, 5-bromo-6-chloronicotinic acid [CAS 29241-62-1, Fluorochem] in place of 3-(4,4,5,5-tetramethyl-1,3,2-dioxaborolan-2-yl)benzoic acid, and 4-phenethoxyaniline (I-1a) in place of 4-((benzyloxy)methyl)-3-fluoroaniline was obtained 5-bromo-6-chloro-N-(4-phenethoxyphenyl)nicotinamide in 76% yield.

LCMS: Method C: 1.16 min, MS: ES⁺ 431.0/433.0

### Intermediate 53

### Methyl 5-(3-((4-(((4-chlorophenyl)sulfonyl)methyl)phenyl)carbamoyl)phenyl)-2-methylnicotinate (I-53)

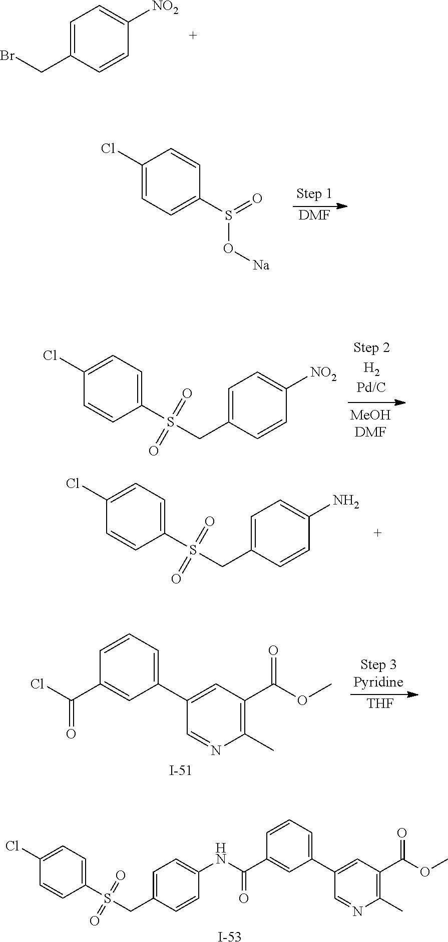

### Step 1: 1-Chloro-4-((4-nitrobenzyl)sulfonyl)benzene

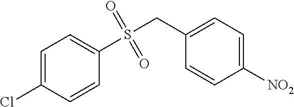 [SMILES: O=[N+]([O-])c1ccc(CS(=O)(=O)c2ccc(Cl)cc2)cc1]

To a vial containing sodium 4-chlorobenzenesulfinate (101 mg, 0.509 mmol) in DMF (2.00 mL) was added 1-(bromomethyl)-4-nitrobenzene (100 mg, 0.463 mmol) and the mixture was stirred for 2 h at rt to give 1-chloro-4-((4-nitrobenzyl)sulfonyl)benzene. This was used directly in the subsequent step, assuming 100% yield.

LCMS: Method A: 1.69 min, MS: ES⁻ 310.0

### Step 2: 4-(((4-Chlorophenyl)sulfonyl)methyl)aniline

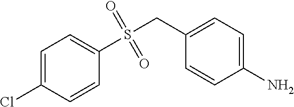 [SMILES: Nc1ccc(CS(=O)(=O)c2ccc(Cl)cc2)cc1]

The mixture from Step 1 was diluted with MeOH (8 mL) and passed through a Pd/C 10% cartridge under hydrogen at rt for 1 h at 1 bar (H-Cube®). The mixture was concentrated under reduced pressure to afford 4-(((4-chlorophenyl)sulfonyl)methyl)aniline (120 mg, 92% over 2 steps) which was used without purification.

LCMS: Method A: 1.11 min, MS: ES⁺ 282.1

### Step 3: Methyl 5-(3-((4-(((4-chlorophenyl)sulfonyl)methyl)phenyl)carbamoyl)phenyl)-2-methylnicotinate (I-53)

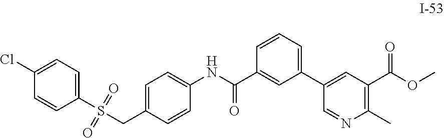 [SMILES: COC(=O)c1cc(-c2cccc(C(=O)Nc3ccc(CS(=O)(=O)c4ccc(Cl)cc4)cc3)c2)cnc1C]

To methyl 5-(3-(chlorocarbonyl)phenyl)-2-methylnicotinate·HCl (I-51, 77 mg, 0.176 mmol) and pyridine (0.047 mL, 0.586 mmol) in THF (4 mL) was added 4-(((4-chlorophenyl)sulfonyl)methyl)aniline (55 mg, 0.195 mmol). The mixture was stirred at rt for 2 h, quenched with water (2 mL) and extracted with DCM (30 mL). The organics were dried under reduced pressure, and the residue was triturated with Et₂O (2×10 mL) to afford methyl 5-(3-((4-(((4-chlorophenyl)sulfonyl)methyl)phenyl)carbamoyl)phenyl)-2-methylnicotinate (70 mg, 54%).

LCMS: Method A: 1.87 min, MS: ES⁺ 535.1

### Intermediate 54

### Methyl 5-(3-((4-(((3-chlorophenyl)sulfonyl)methyl)phenyl)carbamoyl)phenyl)-2-methylnicotinate (I-54)

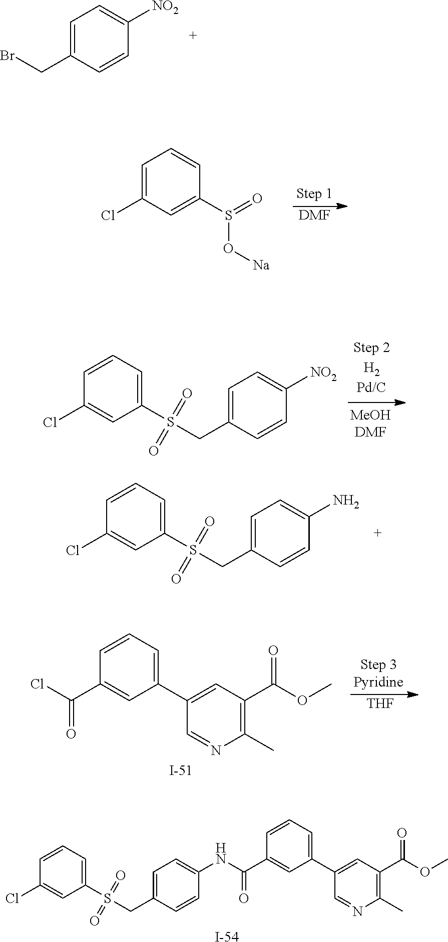

### Step 1: 1-Chloro-3-((4-nitrobenzyl)sulfonyl)benzene

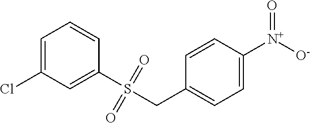 [SMILES: O=[N+]([O-])c1ccc(CS(=O)(=O)c2cccc(Cl)c2)cc1]

Following the procedure of Intermediate 53 Step 1, using sodium 3-chlorobenzenesulfinate [CAS 15946-37-9, Fluorochem] in place of sodium 4-chlorobenzenesulfinate, with isolation by addition of water and collecting the solid by filtration, was obtained 1-chloro-3-((4-nitrobenzyl)sulfonyl)benzene in 71% yield.

LCMS: Method A: 1.28 min, MS: ES⁻ 310

### Step 2: 4-(((3-Chlorophenyl)sulfonyl)methyl)aniline

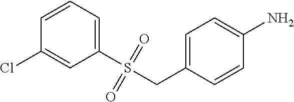 [SMILES: Nc1ccc(CS(=O)(=O)c2cccc(Cl)c2)cc1]

Following the procedure of Intermediate 53 Step 2, using 1-chloro-3-((4-nitrobenzyl)sulfonyl)benzene in place of 1-chloro-4-((4-nitrobenzyl)sulfonyl)benzene was obtained 4-(((3-chlorophenyl)sulfonyl)methyl)aniline in 93% yield.

LCMS: Method A: 0.87 min, MS: ES⁺ 282.1

### Step 3: Methyl 5-(3-((4-(((3-chlorophenyl)sulfonyl)methyl)phenyl)carbamoyl)phenyl)-2-methylnicotinate (I-54)

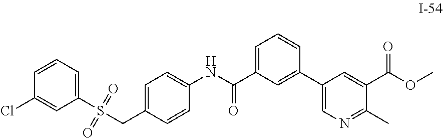 [SMILES: COC(=O)c1cc(-c2cccc(C(=O)Nc3ccc(CS(=O)(=O)c4cccc(Cl)c4)cc3)c2)cnc1C]

Following the procedure of Intermediate 53 Step 3, using 4-(((3-chlorophenyl)sulfonyl)methyl)aniline in place of 4-(((4-chlorophenyl)sulfonyl)methyl)aniline was obtained methyl 5-(3-((4-(((3-chlorophenyl)sulfonyl)methyl)phenyl)carbamoyl)phenyl)-2-methylnicotinate (I-54) in 67% yield.

LCMS: Method A: 1.57 min, MS: ES⁺ 535.1

### Intermediate 55

### Methyl 5-(3-((4-(((2-chlorophenyl)sulfonyl)methyl)phenyl)carbamoyl)phenyl)-2-methylnicotinate (I-55)

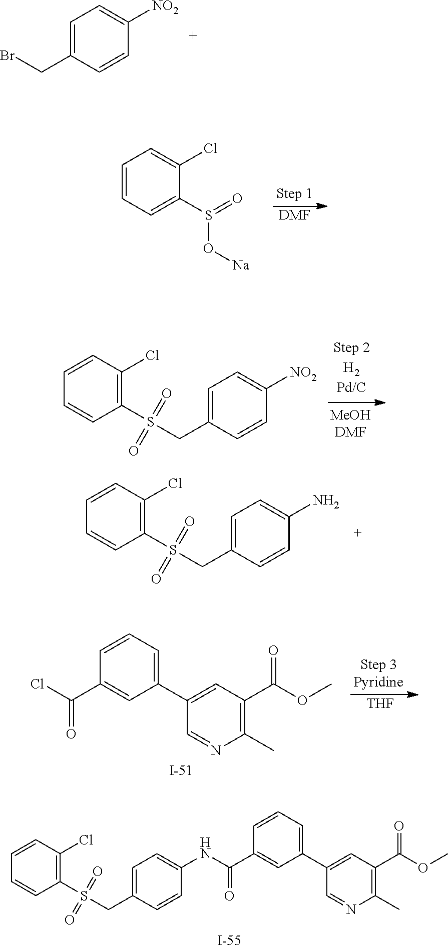

### Step 1: 1-Chloro-2-((4-nitrobenzyl)sulfonyl)benzene

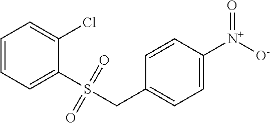 [SMILES: O=[N+]([O-])c1ccc(CS(=O)(=O)c2ccccc2Cl)cc1]

Following the procedure of Intermediate 53 Step 1, using sodium 2-chlorobenzenesulfinate [CAS 15946-36-8, Fluorochem] in place of sodium 4-chlorobenzenesulfinate, with isolation by addition of water and collecting the solid by filtration, was obtained 1-chloro-2-((4-nitrobenzyl)sulfonyl)benzene in 82% yield.

LCMS: Method A: 0.16 min, MS: ES⁺ mass ion not observed

### Step 2: 4-(((2-Chlorophenyl)sulfonyl)methyl)aniline

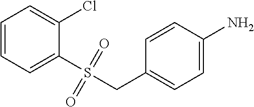 [SMILES: Nc1ccc(CS(=O)(=O)c2ccccc2Cl)cc1]

Following the procedure of Intermediate 53 Step 2, using 1-chloro-2-((4-nitrobenzyl)sulfonyl)benzene in place of 1-chloro-4-((4-nitrobenzyl)sulfonyl)benzene MeOH/THF (1:3) was obtained 4-(((2-chlorophenyl)sulfonyl)methyl)aniline in 79% yield.

LCMS: Method A: 0.79 min, MS: ES⁺ 282.1

### Step 3: Methyl 5-(3-((4-(((2-chlorophenyl)sulfonyl)methyl)phenyl)carbamoyl)phenyl)-2-methylnicotinate (I-55)

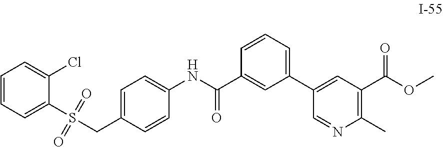 [SMILES: COC(=O)c1cc(-c2cccc(C(=O)Nc3ccc(CS(=O)(=O)c4ccccc4Cl)cc3)c2)cnc1C]

Following the procedure of Intermediate 53 Step 3, using 4-(((2-chlorophenyl)sulfonyl)methyl)aniline in place of 4-(((4-chlorophenyl)sulfonyl)methyl)aniline was obtained methyl 5-(3-((4-(((2-chlorophenyl)sulfonyl)methyl)phenyl)carbamoyl)phenyl)-2-methylnicotinate in 79% yield.

LCMS: Method A: 0.79 min, MS: ES⁺ 535

### Intermediate 56

### Methyl 5-(3-((4-(((4-methoxyphenyl)sulfonyl)methyl)phenyl)carbamoyl)phenyl)-2-methylnicotinate (I-56)

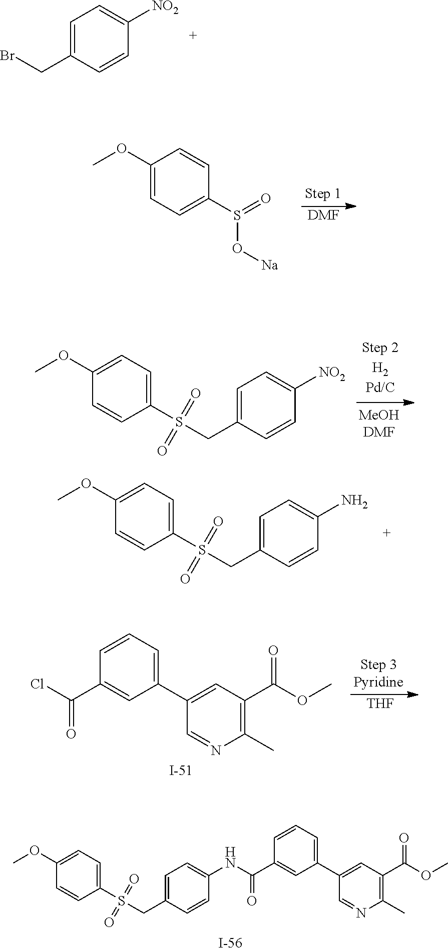

### Step 1: 1-Methoxy-4-((4-nitrobenzyl)sulfonyl)benzene

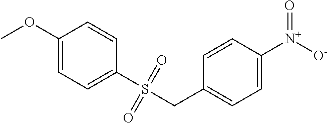 [SMILES: COc1ccc(S(=O)(=O)Cc2ccc([N+](=O)[O-])cc2)cc1]

Following the procedure of Intermediate 53 Step 1, using sodium 4-methoxybenzenesulfinate [CAS 6462-50-6, BLD] in place of sodium 4-chlorobenzenesulfinate, with isolation by addition of water and collecting the solid by filtration, was obtained 1-methoxy-4-((4-nitrobenzyl)sulfonyl)benzene in 31% yield.

LCMS: Method A: 1.25 min, MS: ES⁺ 308

### Step 2: 4-(((4-Methoxyphenyl)sulfonyl)methyl)aniline

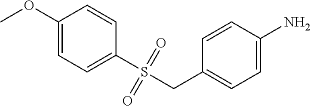 [SMILES: COc1ccc(S(=O)(=O)Cc2ccc(N)cc2)cc1]

A mixture of 1-methoxy-4-((4-nitrobenzyl)sulfonyl)benzene (250 mg, 0.496 mmol), zinc (195 mg, 2.98 mmol) and ammonium formate (188 mg, 2.98 mmol) in EtOH (8 mL) was stirred at 80° C. for 4 h. The mixture was diluted with DCM (40 mL) and the organics decanted, and dried under reduced pressure to afford 4-(((4-methoxyphenyl)sulfonyl)methyl)aniline (95 mg, 43%).

LCMS: Method A: 0.68 min, MS: ES⁺ 278.1

### Step 3: Methyl 5-(3-((4-(((4-methoxyphenyl)sulfonyl)methyl)phenyl)carbamoyl)phenyl)-2-methylnicotinate (I-56)

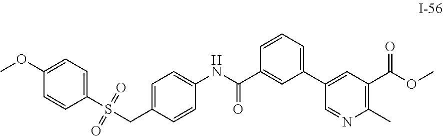

Following the procedure of Intermediate 53 Step 3, using 4-(((4-methoxyphenyl)sulfonyl)methyl)aniline in place of 4-(((4-chlorophenyl)sulfonyl)methyl)aniline was obtained methyl 5-(3-((4-(((4-methoxyphenyl)sulfonyl)methyl)phenyl)carbamoyl)phenyl)-2-methylnicotinate in 34% yield.

LCMS: Method A: 1.47 min, MS: ES⁺ 531.2

### Intermediate 57

### Methyl 5-(3-((4-(((3-methoxyphenyl)sulfonyl)methyl)phenyl)carbamoyl)phenyl)-2-methylnicotinate (I-57)

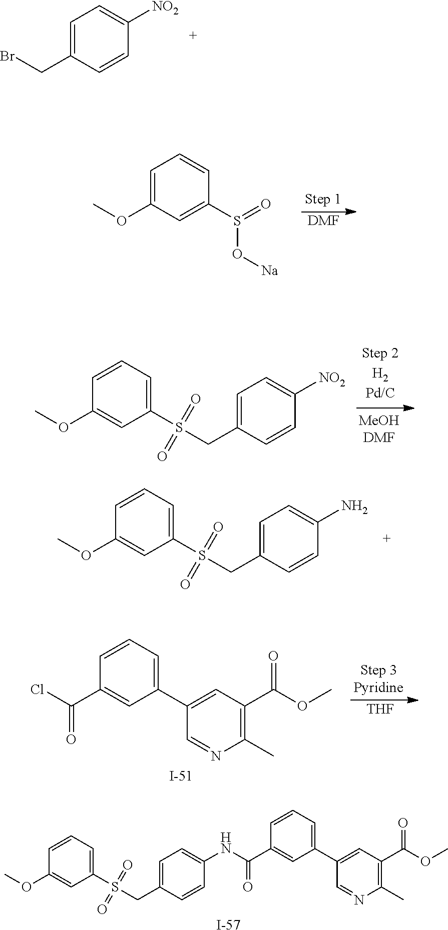

### Step 1: 1-Methoxy-3-((4-nitrobenzyl)sulfonyl)benzene

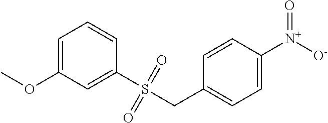 [SMILES: COc1cccc(S(=O)(=O)Cc2ccc([N+](=O)[O-])cc2)c1]

Following the procedure of Intermediate 53 Step 1, using sodium 2-methoxybenzenesulfinate [CAS 15898-41-6, BLD] in place of sodium 4-chlorobenzenesulfinate, with isolation by addition of water and collecting the solid by filtration, was obtained 1-methoxy-3-((4-nitrobenzyl)sulfonyl)benzene in 77% yield.

LCMS: Method A: 1.28 min, MS: ES⁺ 325.1 (M+NH₄⁺)⁺

### Step: 4-(((3-Methoxyphenyl)sulfonyl)methyl)aniline

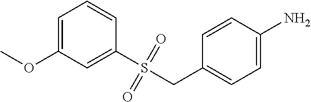 [SMILES: COc1cccc(S(=O)(=O)Cc2ccc(N)cc2)c1]

A mixture of 1-methoxy-3-((4-nitrobenzyl)sulfonyl)benzene (385 mg, 1.25 mmol), zinc (491 mg, 7.52 mmol) and ammonium formate (474 mg, 7.52 mmol) in ethanol (8 mL) was stirred at 80° C. for 4 h. Additional Zn (200 mg) and ammonium formate (500 mg) were added and the mixture stirred at 80° C. for a further 14 h. The mixture was diluted with THF/MeOH (30 mL, 3:1) and the solids removed by filtration. Purification by capturing on SCX, washing with MeOH and eluting with NH₃ in MeOH (2M) afforded 4-(((3-methoxyphenyl)sulfonyl)methyl)aniline in 57% yield.

LCMS: Method A: 0.72 min, MS: ES⁺ 278.1

### Step 3: Methyl 5-(3-((4-(((3-methoxyphenyl)sulfonyl)methyl)phenyl)carbamoyl)phenyl)-2-methylnicotinate (I-57)

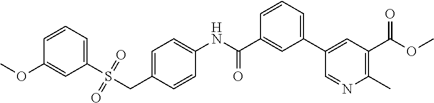

Following the procedure of Intermediate 53 Step 3, using 4-(((3-methoxyphenyl)sulfonyl)methyl)aniline in place of 4-(((4-chlorophenyl)sulfonyl)methyl)aniline was obtained methyl 5-(3-((4-(((3-methoxyphenyl)sulfonyl)methyl)phenyl)carbamoyl)phenyl)-2-methylnicotinate in 43% yield.

LCMS: Method A: 1.49 min, MS: ES⁺ 531.2

### Intermediate 58

### Methyl 2-methyl-5-(3-((4-(((6-methylpyridin-3-yl)sulfonyl)methyl)phenyl)carbamoyl)phenyl)nicotinate (I-58)

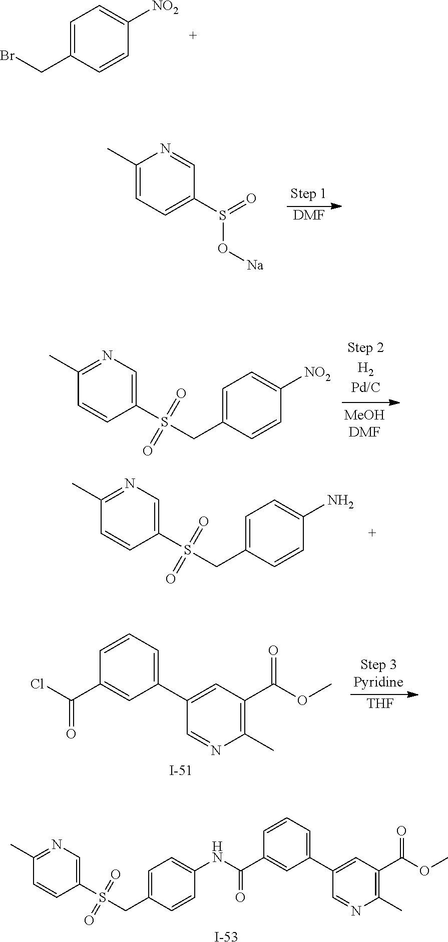

### Step 1: 2-Methyl-5-((4-nitrobenzyl)sulfonyl)pyridine

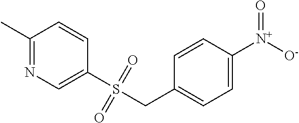 [SMILES: Cc1ccc(S(=O)(=O)Cc2ccc([N+](=O)[O-])cc2)cn1]

Following the procedure of Intermediate 53 Step 1, using sodium 6-methylpyridine-3-sulfinate [CAS 1138034-14-6, Biosynth] in place of sodium 4-chlorobenzenesulfinate, with isolation by addition of water and collecting the solid by filtration, was obtained 2-methyl-5-((4-nitrobenzyl)sulfonyl)pyridine in 69% yield.

LCMS: Method A: 1.01 min, MS: ES⁺ 293.1

### Step: 4-(((6-Methylpyridin-3-yl)sulfonyl)methyl)aniline

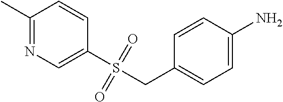 [SMILES: Cc1ccc(S(=O)(=O)Cc2ccc(N)cc2)cn1]

A mixture of 2-methyl-5-((4-nitrobenzyl)sulfonyl)pyridine (510 mg, 1.74 mmol), zinc (1.14 g, 17.4 mmol) and ammonium formate (1.10 g, 17.4 mmol) in EtOH (8 mL) was stirred at 80° C. for 4 h. The mixture was concentrated under reduced pressure. THF/MeOH (30 mL, 3:1) was added and the mixture was filtered. The filtrate was concentrated under reduced pressure to afford 4-(((6-methylpyridin-3-yl)sulfonyl)methyl)aniline (540 mg, 50%).

LCMS: Method A: 0.38 min, MS: ES⁺ 263.1

### Step 3: Methyl 2-methyl-5-(3-((4-(((6-methylpyridin-3-yl)sulfonyl)methyl)phenyl)carbamoyl)phenyl)nicotinate (I-58)

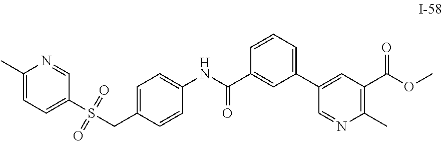

Following the procedure of Intermediate 53 Step 3, using 4-(((6-methylpyridin-3-yl)sulfonyl)methyl)aniline in place of 4-(((4-chlorophenyl)sulfonyl)methyl)aniline was obtained methyl 2-methyl-5-(3-((4-(((6-methylpyridin-3-yl)sulfonyl)methyl)phenyl)carbamoyl)phenyl)nicotinate in 23% yield.

LCMS: Method A: 1.44 min, MS: ES⁺ 516.1

### Intermediate 59

### 5-Bromo-6-(methylthio)-N-(4-phenethoxyphenyl)nicotinamide (I-59)

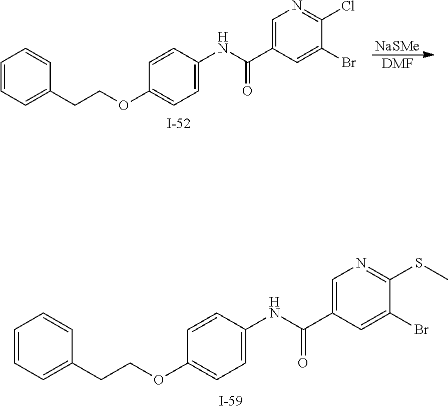

To 5-bromo-6-chloro-N-(4-phenethoxyphenyl)nicotinamide (I-52, 319 mg, 0.724 mmol) in dry DMF (2 mL) was added NaSMe (51 mg, 0.724 mmol). The mixture was stirred at rt for 18 h and diluted with water (10 mL). The solid was collected by filtration, and washed with MeOH and acetone to give 5-bromo-6-(methylthio)-N-(4-phenethoxyphenyl)nicotinamide (213 mg, 66%).

LCMS: Method A: 0.94 min, MS: ES⁺ 443.0/445.0

### Intermediate 60

### 5-Bromo-6-(2-methoxyethoxy)-N-(4-phenethoxyphenyl)nicotinamide (I-60)

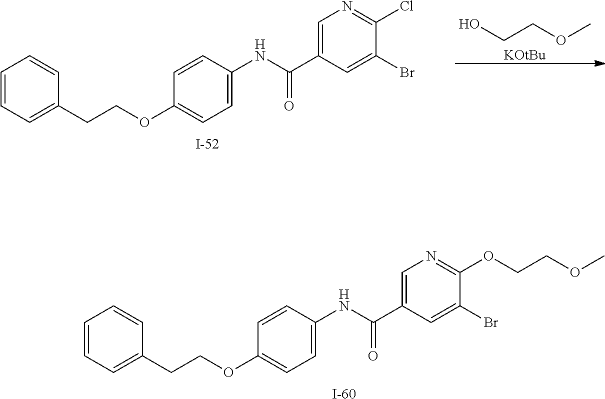

A mixture of 2-methoxyethanol (0.627 mL, 7.95 mmol) and KOtBu (446 mg, 3.97 mmol) was stirred at rt for 10 min and 5-bromo-6-chloro-N-(4-phenethoxyphenyl)nicotinamide (I-52, 350 mg, 0.795 mmol) was added. The mixture was stirred at rt for 1 h and 50° C. for 4 h. The mixture was cooled to rt and diluted with water (10 mL). The solid was collected by filtration, washing with water to give 5-bromo-6-(2-methoxyethoxy)-N-(4-phenethoxyphenyl)nicotinamide (249 mg, 63%).

LCMS: Method C: 0.88 min, MS: ES⁺ 471.0/473.0

### EXAMPLES

### Example 1

### 4-Hydroxy-3′-((4-phenethoxyphenyl)carbamoyl)-[1,1′-biphenyl]-3-carboxylic acid

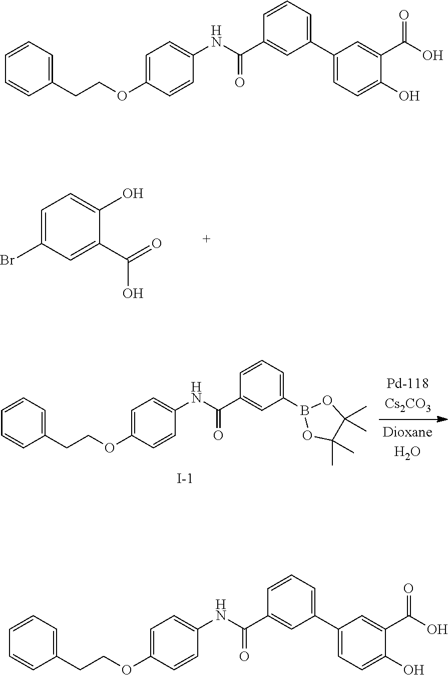 [SMILES: O=C(Nc1ccc(OCCc2ccccc2)cc1)c1cccc(-c2ccc(O)c(C(=O)O)c2)c1 ; O=C(O)c1cc(Br)ccc1O ; CC1(C)OB(c2cccc(C(=O)Nc3ccc(OCCc4ccccc4)cc3)c2)OC1(C)C ; [I-] ; O=C(Nc1ccc(OCCc2ccccc2)cc1)c1cccc(-c2ccc(O)c(C(=O)O)c2)c1]

To a stirred mixture of 5-bromo-2-hydroxybenzoic acid (CAS 89-55-4, Acros, 50 mg, 0.23 mmol), and N-(4-phenethoxyphenyl)-3-(4,4,5,5-tetramethyl-1,3,2-dioxaborolan-2-yl)benzamide (I-1, 123 mg, 0.28 mmol) in dioxane (4 mL) and water (1 mL), was added Cs₂CO₃ (300 mg, 0.92 mmol) and the reaction mixture was degassed with a flow of nitrogen at rt for 5 min. Pd-118 (0.03 g, 0.04 mmol) was added and the mixture degassed for a further 2 min. The reaction mixture was stirred at 80° C. for 18 h then cooled to rt, acidified with aq. HCl (1M, 5 mL), and extracted with EtOAc (3×10 mL). The combined organic phases were dried over Na₂SO₄ and concentrated under reduced pressure. The crude product was purified by RP flash chromatography (C18, 0-100% MeCN in 0.1% aq. NH₄OH) to afford 4-hydroxy-3′-((4-phenethoxyphenyl)carbamoyl)-[1,1′-biphenyl]-3-carboxylic acid (40 mg, 36%).

LCMS: Method A, 2.12 min, MS: ES⁺ 454.2; ¹H NMR (500 MHz, DMSO) δ ppm: 10.23 (s, 1H), 8.14 (s, 1H), 8.12 (d, J=2.6 Hz, 1H), 7.85 (d, J=7.7 Hz, 1H), 7.83-7.78 (m, 2H), 7.68 (d, J=9.0 Hz, 2H), 7.57 (t, J=7.7 Hz, 1H), 7.38-7.29 (m, 4H), 7.27-7.20 (m, 1H), 6.95 (d, J=9.0 Hz, 2H), 4.19 (t, J=6.9 Hz, 2H), 3.04 (t, J=6.9 Hz, 2H). 3H were obscured/not observed. The isolated compound contained up to 1 mol. eq. of ammonia.

### Examples 2-29

The following Examples 2-29 were prepared analogously to Example 1, substituting 5-bromo-2-hydroxybenzoic acid (CAS 89-55-4) with the appropriate starting material and with any minor modifications described. In some cases, an ester was used in place of a carboxylic acid, and under the reaction conditions partial or complete hydrolysis to the acid product occurred. Examples 15 and 17 were purified with acidic modifier.

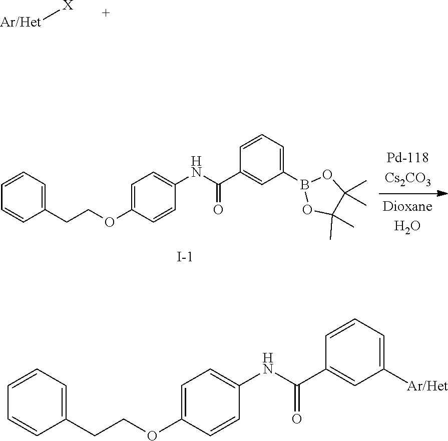

### Example 2

### 2-Cyclopropyl-5-(3-((4-phenethoxyphenyl)carbamoyl)phenyl)nicotinic acid

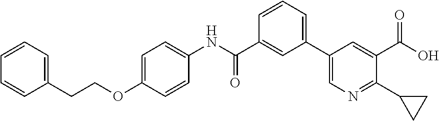 [SMILES: O=C(Nc1ccc(OCCc2ccccc2)cc1)c1cccc(-c2cnc(C3CC3)c(C(=O)O)c2)c1]

From the starting material 5-bromo-2-cyclopropylnicotinic acid (CAS 1601026-05-4, Enamine). Reaction at 90° C. for 18 h afforded 2-cyclopropyl-5-(3-((4-phenethoxyphenyl)carbamoyl)phenyl)nicotinic acid in 62% yield.

LCMS: Method A, 2.05 min MS: ES⁺ 479.2; ¹H NMR (500 MHz, DMSO) δ ppm: 13.57 (s, 1H), 10.22 (s, 1H), 8.93 (s, 1H), 8.38 (s, 1H), 8.26 (d, J=1.9 Hz, 1H), 7.99-7.91 (m, 2H), 7.68-7.63 (m, 3H), 7.37-7.29 (m, 4H), 7.27-7.20 (m, 1H), 6.98-6.93 (m, 2H), 4.19 (t, J=6.9 Hz, 2H), 3.12-3.07 (m, 1H), 3.04 (t, J=6.9 Hz, 2H), 1.11-1.06 (m, 2H), 1.05-1.00 (m, 2H). The isolated compound contained up to 1 mol. eq. of ammonia

### Example 3

### 2-oxo-5-(3-((4-Phenethoxyphenyl)carbamoyl)phenyl)-1,2-dihydropyridine-3-carboxylic acid

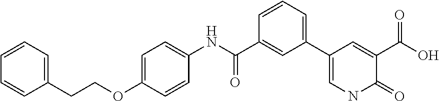 [SMILES: O=C(Nc1ccc(OCCc2ccccc2)cc1)c1cccc(-c2c[nH]c(=O)c(C(=O)O)c2)c1]

From the starting material methyl 5-bromo-2-hydroxynicotinate (CAS 120034-05-1, BLD). Reaction at 90° C. for 18 h afforded 2-oxo-5-(3-((4-phenethoxyphenyl)carbamoyl)phenyl)-1,2-dihydropyridine-3-carboxylic acid in 10% yield.

LCMS: Method B, 1.23 min, MS: ES⁺ 455.2; ¹H NMR (500 MHz, DMSO) δ ppm: 13.74 (s, 1H), 10.24 (s, 1H), 8.66 (d, J=2.8 Hz, 1H), 8.44 (d, J=2.8 Hz, 1H), 8.21-8.17 (m, 1H), 7.91 (d, J=7.7 Hz, 1H), 7.88 (d, J=7.7 Hz, 1H), 7.70-7.63 (m, 2H), 7.60 (t, J=7.7 Hz, 1H), 7.38-7.29 (m, 4H), 7.27-7.20 (m, 1H), 6.99-6.92 (m, 2H), 4.19 (t, J=6.9 Hz, 2H), 3.05 (t, J=6.9 Hz, 2H). 1H obscured/not observed. The isolated compound contained up to 1 mol. eq. of ammonia.

### Example 4

### 5-(3-((4-Phenethoxyphenyl)carbamoyl)phenyl)-2-(trifluoromethyl)nicotinic acid

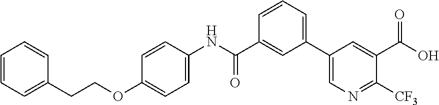 [SMILES: O=C(Nc1ccc(OCCc2ccccc2)cc1)c1cccc(-c2cnc(C(F)(F)F)c(C(=O)O)c2)c1]

From the starting material 5-bromo-2-(trifluoromethyl)nicotinic acid (CAS 436799-36-9, Combi-Blocks). Reaction at 90° C. for 18 h afforded 5-(3-((4-phenethoxyphenyl)carbamoyl)phenyl)-2-(trifluoromethyl)nicotinic acid in 46% yield.

LCMS: Method A, 2.03 min, MS: ES⁺ 507.1; ¹H NMR (500 MHz, DMSO) δ ppm: 10.21 (s, 1H), 8.92 (s, 1H), 8.27 (d, J=1.9 Hz, 1H), 8.19 (s, 1H), 7.97-7.92 (m, 2H), 7.61-7.58 (m, 2H), 7.29-7.23 (m, 4H), 7.17-7.12 (m, 2H), 6.90-6.87 (m, 2H), 4.12 (t, J=6.9 Hz, 2H), 2.97 (t, J=6.9 Hz, 2H). 1H obscured/not observed. The isolated compound contained up to 1 mol. eq. of ammonia.

### Example 5

### 5-(3-((4-Phenethoxyphenyl)carbamoyl)phenyl)nicotinic acid

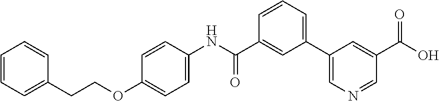 [SMILES: O=C(O)c1cncc(-c2cccc(C(=O)Nc3ccc(OCCc4ccccc4)cc3)c2)c1]

From the starting material methyl 5-bromonicotinate (CAS 29681-44-5, Fluorochem). Reaction at 85° C. for 18 h afforded 5-(3-((4-phenethoxyphenyl)carbamoyl)phenyl)nicotinic acid in 35% yield.

LCMS: Method A, 1.85 min, MS: ES⁺ 439.2; ¹H NMR (500 MHz, DMSO) δ ppm: 10.27 (s, 1H), 9.15-9.12 (m, 1H), 9.11-9.05 (m, 1H), 8.59-8.54 (m, 1H), 8.34-8.30 (m, 1H), 8.03-7.96 (m, 2H), 7.69-7.63 (m, 3H), 7.37-7.29 (m, 4H), 7.26-7.19 (m, 1H), 6.99-6.93 (m, 2H), 4.19 (t, J=6.9 Hz, 2H), 3.04 (t, J=6.9 Hz, 2H). 1H obscured/not observed.

The isolated compound contained up to 1 mol. eq. of ammonia.

### Example 6

### 2-(5-(3-((4-Phenethoxyphenyl)carbamoyl)phenyl)pyridin-3-yl)acetic acid

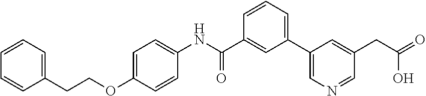 [SMILES: O=C(O)Cc1cncc(-c2cccc(C(=O)Nc3ccc(OCCc4ccccc4)cc3)c2)c1]

From the starting material 2-(5-bromopyridin-3-yl)acetic acid (CAS 39891-12-839, Thermo Scientific). Reaction at 80° C. for 18 h afforded 2-(5-(3-((4-phenethoxyphenyl)carbamoyl)-phenyl)pyridin-3-yl)acetic acid in 45% yield.

LCMS: Method A, 1.66 min, MS: ES⁺ 453.2; ¹H NMR (500 MHz, DMSO) δ ppm: 10.18 (s, 1H), 8.75 (d, J=2.1 Hz, 1H), 8.39 (d, J=2.1 Hz, 1H), 8.17 (s, 1H), 7.96 (s, 1H), 7.89 (d, J=7.7 Hz, 1H), 7.83 (d, J=7.7 Hz, 1H), 7.64-7.55 (m, 3H), 7.30-7.22 (m, 4H), 7.20-7.13 (m, 1H), 6.88 (d, J=8.6 Hz, 2H), 4.11 (t, J=6.9 Hz, 2H), 3.53 (s, 2H), 2.97 (t, J=6.9 Hz, 2H). 1H obscured/not observed. The isolated compound contained up to 1 mol. eq. of ammonia.

### Example 7

### 4-(3-((4-Phenethoxyphenyl)carbamoyl)phenyl)pyrimidine-2-carboxylic acid

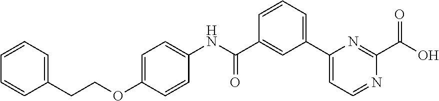 [SMILES: O=C(Nc1ccc(OCCc2ccccc2)cc1)c1cccc(-c2ccnc(C(=O)O)n2)c1]

From the starting material methyl 4-bromopyrimidine-2-carboxylate (CAS 1206250-40-9, BLD). Reaction at 90° C. for 18 h afforded 4-(3-((4-phenethoxyphenyl)carbamoyl)phenyl)pyrimidine-2-carboxylic acid in 30% yield.

LCMS: Method A, 1.24 min, MS: ES⁺ 440.2; ¹H NMR (500 MHz, DMSO) δ ppm: 10.35 (s, 1H), 8.77 (d, J=5.3 Hz, 1H), 8.68 (d, J=5.3 Hz, 1H), 8.35 (d, J=7.8 Hz, 1H), 8.08 (d, J=7.8 Hz, 1H), 7.98 (d, J=5.3 Hz, 1H), 7.70-7.66 (m, 3H), 7.36-7.28 (m, 4H), 7.26-7.20 (m, 1H), 6.95 (d, J=8.5 Hz, 2H), 4.19 (t, J=6.9 Hz, 2H), 3.04 (t, J=6.9 Hz, 2H). 1H obscured/not observed. The isolated compound contained up to 1 mol. eq. of ammonia.

### Example 8

### 3-Methyl-6-(3-((4-phenethoxyphenyl)carbamoyl)phenyl)picolinic acid

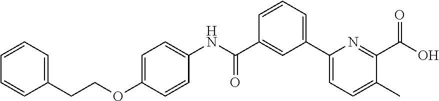 [SMILES: Cc1ccc(-c2cccc(C(=O)Nc3ccc(OCCc4ccccc4)cc3)c2)nc1C(=O)O]

From the starting material 6-bromo-3-methylpyridine-2-carboxylic acid (CAS 1211516-18-5, BLD). Reaction at 90° C. for 18 h afforded 3-methyl-6-(3-((4-phenethoxyphenyl)carbamoyl)phenyl)picolinic acid in 34% yield.

LCMS: Method A, 2.03 min, MS: ES⁺ 453.2; ¹H NMR (500 MHz, DMSO) δ ppm: 10.29 (s, 1H), 8.64-8.59 (m, 1H), 8.29 (d, J=7.8 Hz, 1H), 8.03 (d, J=8.1 Hz, 1H), 7.97 (d, J=7.8 Hz, 1H), 7.81 (d, J=8.1 Hz, 1H), 7.74-7.67 (m, 2H), 7.63 (t, J=7.7 Hz, 1H), 7.37-7.29 (m, 4H), 7.27-7.21 (m, 1H), 7.04-6.91 (m, 2H), 4.19 (t, J=6.9 Hz, 2H), 3.05 (t, J=6.9 Hz, 2H), 2.42 (s, 3H). 1H obscured/not observed. The isolated compound contained up to 1 mol. eq. of ammonia.

### Example 9

### 3-Amino-6-(3-((4-phenethoxyphenyl)carbamoyl)phenyl)pyrazine-2-carboxylic acid

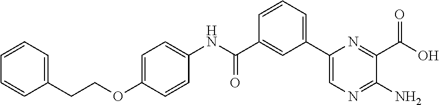 [SMILES: Nc1ncc(-c2cccc(C(=O)Nc3ccc(OCCc4ccccc4)cc3)c2)nc1C(=O)O]

From the starting material methyl 3-amino-6-bromopyrazine-2-carboxylate (CAS 6966-01-4, Fluorochem). Reaction at 80° C. for 18 h afforded 3-amino-6-(3-((4-phenethoxyphenyl)carbamoyl)phenyl)pyrazine-2-carboxylic acid in 8% yield.

LCMS: Method A, 1.87 min, MS: ES⁺ 455.2; ¹H NMR (500 MHz, DMSO) δ ppm: 10.20 (s, 1H), 8.98 (s, 1H), 8.56 (s, 1H), 8.27 (d, J=7.9 Hz, 1H), 7.91 (d, J=7.9 Hz, 1H), 7.68 (d, J=8.8 Hz, 2H), 7.65-7.54 (m, 3H), 7.37-7.29 (m, 4H), 7.24 (d, J=6.7 Hz, 1H), 6.96 (d, J=8.8 Hz, 2H), 4.19 (t, J=6.9 Hz, 2H), 3.05 (t, J=6.9 Hz, 2H). 1H obscured/not observed. The isolated compound contained up to 1 mol. eq. of ammonia.

### Example 10

### 3-Methyl-6-(3-((4-phenethoxyphenyl)carbamoyl)phenyl)pyrazine-2-carboxylic acid

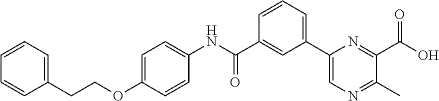 [SMILES: Cc1ncc(-c2cccc(C(=O)Nc3ccc(OCCc4ccccc4)cc3)c2)nc1C(=O)O]

From the starting material methyl 6-chloro-3-methylpyrazine-2-carboxylate (CAS 1166831-45-3, Combi-Blocks). Reaction at 90° C. for 18 h afforded 3-methyl-6-(3-((4-phenethoxy-phenyl)carbamoyl)phenyl)pyrazine-2-carboxylic acid in 55% yield.

LCMS: Method A, 1.92 min, MS: ES⁺ 454.2; ¹H NMR (500 MHz, DMSO) δ ppm: ¹H NMR (500 MHz, DMSO) δ 13.81 (s, 1H), 10.27 (s, 1H), 9.25 (s, 1H), 8.64 (s, 1H), 8.32 (d, J=7.8 Hz, 1H), 8.03 (d, J=7.7 Hz, 1H), 7.71-7.64 (m, 3H), 7.37-7.28 (m, 4H), 7.27-7.20 (m, 1H), 6.99-6.92 (m, 2H), 4.19 (t, J=6.9 Hz, 2H), 3.04 (t, J=6.9 Hz, 2H), 2.66 (s, 3H). The isolated compound contained up to 1 mol. eq. of ammonia.

### Example 11

### 2-Fluoro-5-(3-((4-phenethoxyphenyl)carbamoyl)phenyl)nicotinic acid

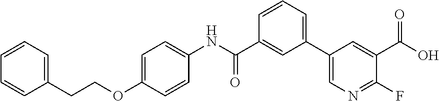 [SMILES: O=C(Nc1ccc(OCCc2ccccc2)cc1)c1cccc(-c2cnc(F)c(C(=O)O)c2)c1]

From the starting material 5-bromo-2-fluoronicotinic acid (CAS 29241-66-5, Fluorochem). Reaction at 80° C. for 9 h afforded 2-cyclopropyl-5-(3-((4-phenethoxyphenyl)carbamoyl)-phenyl)nicotinic acid in 40% yield.

LCMS: Method A, 1.93 min, MS: ES⁺ 457.2; ¹H NMR (500 MHz, DMSO) δ ppm: 10.27 (s, 1H), 8.46 (s, 1H), 8.40-8.35 (m, 1H), 8.25 (s, 1H), 7.95 (d, J=7.7 Hz, 1H), 7.90 (d, J=7.7 Hz, 1H), 7.70-7.59 (m, 3H), 7.38-7.29 (m, 3H), 7.24 (t, J=6.9 Hz, 1H), 6.98-6.93 (m, 2H), 4.19 (t, J=6.9 Hz, 2H), 3.05 (t, J=6.9 Hz, 2H). 2H obscured/not observed. The isolated compound contained up to 1 mol. eq. of ammonia.

### Example 12

### 2-Amino-5-(3-((4-phenethoxyphenyl)carbamoyl)phenyl)nicotinic acid

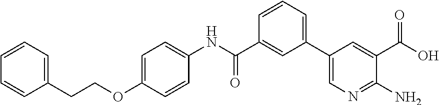 [SMILES: Nc1ncc(-c2cccc(C(=O)Nc3ccc(OCCc4ccccc4)cc3)c2)cc1C(=O)O]

From the starting material methyl 2-amino-5-bromonicotinate (CAS 50735-34-7, Fluorochem). Reaction at 90° C. for 18 h afforded 2-amino-5-(3-((4-phenethoxyphenyl)carbamoyl)phenyl)nicotinic acid in 29% yield.

LCMS: Method A, 1.63 min, MS: ES⁺ 454.2; ¹H NMR (500 MHz, DMSO) δ ppm: 10.21 (s, 1H), 8.64 (d, J=2.7 Hz, 1H), 8.40 (d, J=2.6 Hz, 1H), 8.15 (s, 1H), 7.87 (d, J=7.7 Hz, 1H), 7.83 (d, J=7.7 Hz, 1H), 7.67 (d, J=8.7 Hz, 2H), 7.58 (t, J=7.7 Hz, 1H), 7.38-7.29 (m, 4H), 7.24 (t, J=6.9 Hz, 1H), 6.95 (d, J=8.9 Hz, 2H), 4.19 (t, J=6.9 Hz, 2H), 3.04 (t, J=6.9 Hz, 2H). 3H obscured/not observed. The isolated compound contained up to 1 mol. eq. of ammonia.

### Example 13

### 2-(3-((4-Phenethoxyphenyl)carbamoyl)phenyl)isonicotinic acid

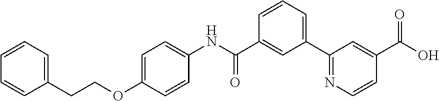 [SMILES: O=C(O)c1ccnc(-c2cccc(C(=O)Nc3ccc(OCCc4ccccc4)cc3)c2)c1]

From the starting material methyl-2-bromoisonicotinate (CAS 26156-48-9, Fluorochem). Reaction at 80° C. for 18 h afforded 2-(3-((4-phenethoxyphenyl)carbamoyl)phenyl)-isonicotinic acid in 11% yield.

LCMS: Method A, 1.91 min, MS: ES⁺ 439.2; ¹H NMR (500 MHz, DMSO) δ ppm: 10.32 (s, 1H), 8.83 (d, J=4.9 Hz, 1H), 8.64 (s, 1H), 8.41 (s, 1H), 8.32 (d, J=7.8 Hz, 1H), 8.02 (d, J=7.8 Hz, 1H), 7.79 (d, J=4.9 Hz, 1H), 7.71-7.64 (m, 3H), 7.36-7.30 (m, 4H), 7.22 (t, J=6.8 Hz, 1H), 6.95 (d, J=8.6 Hz, 2H), 4.19 (t, J=6.9 Hz, 2H), 3.04 (t, J=6.9 Hz, 2H). 1H obscured/not observed. The isolated compound contained up to 1 mol. eq. of ammonia.

### Example 14

### 6-(3-((4-Phenethoxyphenyl)carbamoyl)phenyl)pyrazine-2-carboxylic acid

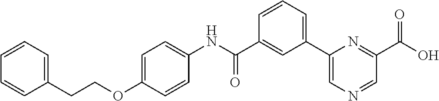 [SMILES: O=C(Nc1ccc(OCCc2ccccc2)cc1)c1cccc(-c2cncc(C(=O)O)n2)c1]

From the starting material methyl 6-bromopyrazine-2-carboxylate (CAS 40155-34-8, BLD). Reaction at 90° C. for 18 h afforded 6-(3-((4-phenethoxyphenyl)carbamoyl)phenyl)pyrazine-2-carboxylic acid in 17% yield.

LCMS: Method A, 1.86 min, MS: ES⁺ 440.0; ¹H NMR (500 MHz, DMSO) δ ppm: 10.32 (s, 1H), 9.32 (s, 1H), 9.01 (s, 1H), 8.69 (t, J=1.9 Hz, 1H), 8.35 (d, J=7.7 Hz, 1H), 8.05 (d, J=7.7 Hz, 1H), 7.73-7.65 (m, 3H), 7.36-7.30 (m, 4H), 7.27-7.22 (m, 1H), 6.98-6.94 (m, 2H), 4.20 (t, J=6.9 Hz, 2H), 3.05 (t, J=6.9 Hz, 2H). 1H obscured/not observed. The isolated compound contained up to 1 mol. eq. of ammonia.

### Example 15

### 2-Methyl-5-(3-((4-phenethoxyphenyl)carbamoyl)phenyl)nicotinic acid

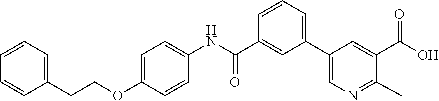 [SMILES: Cc1ncc(-c2cccc(C(=O)Nc3ccc(OCCc4ccccc4)cc3)c2)cc1C(=O)O]

From the starting material methyl 5-bromo-2-methylnicotinate (CAS 1215916-40-7, BLD). Reaction at 80° C. for 9 h afforded 2-methyl-5-(3-((4-phenethoxyphenyl)carbamoyl)phenyl)-nicotinic acid in 40% yield.

LCMS: Method A, 1.73 min, MS: ES⁺ 453.2; ¹H NMR (500 MHz, DMSO) δ ppm: 13.44 (s, 1H), 10.24 (s, 1H), 9.01 (d, J=2.4 Hz, 1H), 8.48 (d, J=2.4 Hz, 1H), 8.27 (s, 1H), 7.97 (t, J=7.5 Hz, 2H), 7.69-7.64 (m, 3H), 7.35-7.29 (m, 4H), 7.25-7.20 (m, 1H), 6.97-6.93 (m, 2H), 4.19 (t, J=6.9 Hz, 2H), 3.04 (t, J=6.9 Hz, 2H), 2.77 (s, 3H).

### Example 16

### 4-amino-3′-((4-phenethoxyphenyl)carbamoyl)-[1,1′-biphenyl]-3-carboxylic acid

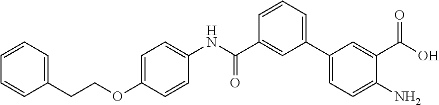 [SMILES: Nc1ccc(-c2cccc(C(=O)Nc3ccc(OCCc4ccccc4)cc3)c2)cc1C(=O)O]

From the starting material methyl 2-amino-5-bromobenzoate (CAS 1215916-40-7, Combi-Blocks). Reaction at 90° C. for 18 h afforded 4-amino-3′-((4-phenethoxyphenyl)carbamoyl)-[1,1′-biphenyl]-3-carboxylic acid in 55% yield.

LCMS: Method A, 1.97 min, MS: ES⁺ 453.2; ¹H NMR (500 MHz, DMSO) δ ppm: 10.20 (s, 1H), 8.11-8.07 (m, 2H), 7.81 (d, J=7.7 Hz, 1H), 7.75 (d, J=7.8 Hz, 1H), 7.71-7.65 (m, 3H), 7.54 (t, J=7.7 Hz, 1H), 7.38-7.29 (m, 4H), 7.27-7.20 (m, 1H), 6.97-6.91 (m, 2H), 6.89 (d, J=8.7 Hz, 1H), 4.19 (t, J=6.9 Hz, 2H), 3.04 (t, J=6.9 Hz, 2H). 3H obscured/not observed. The isolated compound contained up to 1 mol. eq. of ammonia.

### Example 17

### 2-Methoxy-5-(3-((4-phenethoxyphenyl)carbamoyl)phenyl)nicotinic acid

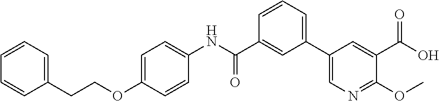 [SMILES: COc1ncc(-c2cccc(C(=O)Nc3ccc(OCCc4ccccc4)cc3)c2)cc1C(=O)O]

From the starting material methyl 5-bromo-2-methoxynicotinate (CAS 122433-41-4, BLD). Reaction at 90° C. for 18 h afforded 2-methoxy-5-(3-((4-phenethoxyphenyl)carbamoyl)-phenyl)nicotinic acid in 42% yield.

LCMS: Method A, 1.96 min, MS: ES⁺ 469.2; ¹H NMR (500 MHz, DMSO) δ ppm: 13.18 (s, 1H), 10.22 (s, 1H), 8.76 (d, J=2.6 Hz, 1H), 8.46 (d, J=2.6 Hz, 1H), 8.24 (d, J=2.0 Hz, 1H), 7.93 (t, J=9.2 Hz, 2H), 7.70-7.64 (m, 2H), 7.63 (t, J=7.7 Hz, 1H), 7.38-7.29 (m, 4H), 7.27-7.20 (m, 1H), 6.98-6.92 (m, 2H), 4.19 (t, J=6.9 Hz, 2H), 3.99 (s, 3H), 3.05 (t, J=6.9 Hz, 2H).

### Example 18

### 6-(3-((4-Phenethoxyphenyl)carbamoyl)phenyl)picolinic acid

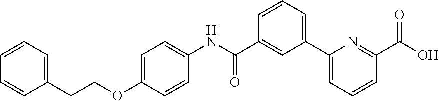 [SMILES: O=C(Nc1ccc(OCCc2ccccc2)cc1)c1cccc(-c2cccc(C(=O)O)n2)c1]

From the starting material methyl 6-bromopicolinate (CAS 26218-75-7, Fluorochem). Reaction at 90° C. for 18 h afforded 6-(3-((4-phenethoxyphenyl)carbamoyl)phenyl)picolinic acid in 30% yield.

LCMS: Method A, 1.92 min, MS: ES⁺ 439.2; ¹H NMR (500 MHz, DMSO) δ ppm: 10.31 (s, 1H), 8.67 (d, J=1.9 Hz, 1H), 8.38-8.34 (m, 1H), 8.23 (d, J=7.8 Hz, 1H), 8.06 (t, J=7.8 Hz, 1H), 8.01 (d, J=7.7 Hz, 1H), 7.98 (d, J=7.7 Hz, 1H), 7.72-7.69 (m, 2H), 7.66 (d, J=7.7 Hz, 1H), 7.37-7.30 (m, 4H), 7.26-7.21 (m, 1H), 6.97-6.94 (m, 2H), 4.20 (t, J=6.9 Hz, 2H), 3.05 (t, J=6.9 Hz, 2H). 1H obscured/not observed. The isolated compound contained up to 1 mol. eq. of ammonia.

### Example 19

### 2-Chloro-5-(3-((4-phenethoxyphenyl)carbamoyl)phenyl)nicotinic acid

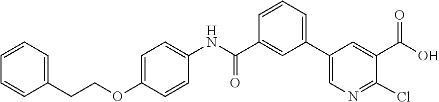 [SMILES: O=C(Nc1ccc(OCCc2ccccc2)cc1)c1cccc(-c2cnc(Cl)c(C(=O)O)c2)c1]

From the starting material methyl 5-bromo-2-chloronicotinate (CAS 78686-79-0, Fluorochem). Reaction at 80° C. for 18 h afforded 2-chloro-5-(3-((4-phenethoxyphenyl)-carbamoyl)phenyl)nicotinic acid in 35% yield.

LCMS: Method A, 1.95 min, MS: ES⁺ 473.1; ¹H NMR (500 MHz, DMSO) δ ppm: 10.26 (s, 1H), 8.70 (d, J=2.6 Hz, 1H), 8.28 (s, 1H), 8.22 (s, 1H), 7.98 (d, J=7.7 Hz, 1H), 7.95 (d, J=7.7 Hz, 1H), 7.70-7.61 (m, 3H), 7.38-7.29 (m, 4H), 7.27-7.21 (m, 1H), 6.99-6.92 (m, 2H), 4.19 (t, J=6.9 Hz, 2H), 3.04 (t, J=6.9 Hz, 2H). 1H obscured/not observed. The isolated compound contained up to 1 mol. eq. of ammonia.

### Example 20

### 6-(3-((4-Phenethoxyphenyl)carbamoyl)phenyl)nicotinic acid

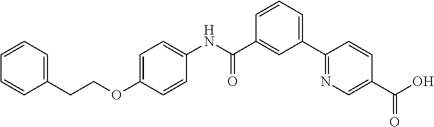 [SMILES: O=C(O)c1ccc(-c2cccc(C(=O)Nc3ccc(OCCc4ccccc4)cc3)c2)nc1]

From the starting material 6-bromonicotinic acid (CAS 6311-35-9, Fluorochem). Reaction at 90° C. for 18 h afforded 6-(3-((4-phenethoxyphenyl)carbamoyl)phenyl)nicotinic acid in 21% yield.

LCMS: Method B, 1.30 min, MS: ES⁺ 439.2; ¹H NMR (500 MHz, DMSO) δ ppm: δ 13.49 (s, 1H), 10.29 (s, 1H), 9.18 (d, J=1.9 Hz, 1H), 8.69 (d, J=1.9 Hz, 1H), 8.40-8.33 (m, 2H), 8.22 (d, J=8.3 Hz, 1H), 8.05 (d, J=7.7 Hz, 1H), 7.71-7.65 (m, 3H), 7.36-7.29 (m, 4H), 7.26-7.21 (m, 1H), 6.98-6.92 (m, 2H), 4.19 (t, J=6.9 Hz, 2H), 3.04 (t, J=6.9 Hz, 2H). The isolated compound contained up to 1 mol. eq. of ammonia.

### Example 21

### 4-(3-((4-Phenethoxyphenyl)carbamoyl)phenyl)picolinic acid

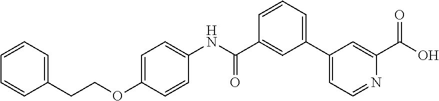 [SMILES: O=C(Nc1ccc(OCCc2ccccc2)cc1)c1cccc(-c2ccnc(C(=O)O)c2)c1]

From the starting material methyl 4-bromopicolinate (CAS 29681-42-3, Fluorochem). Reaction at 90° C. for 18 h afforded 4-(3-((4-phenethoxyphenyl)carbamoyl)phenyl)picolinic acid in 9% yield.

LCMS: Method B, 1.22 min, MS: ES⁺ 439.2; ¹H NMR (500 MHz, DMSO) δ ppm 10.31 (s, 1H), 8.64 (d, J=4.9 Hz, 1H), 8.33 (s, 1H), 8.27 (s, 1H), 8.04-7.97 (m, 2H), 7.81-7.76 (m, 1H), 7.70-7.64 (m, 3H), 7.36-7.28 (m, 4H), 7.25-7.20 (m, 1H), 6.98-6.92 (m, 2H), 4.19 (t, J=6.9 Hz, 2H), 3.05 (d, J=6.9 Hz, 2H). 1H obscured/not observed. The isolated compound contained up to 1 mol. eq. of ammonia.

### Example 22

### 2-(3-((4-Phenethoxyphenyl)carbamoyl)phenyl)pyrimidine-4-carboxylic acid

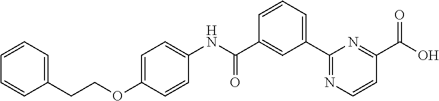 [SMILES: O=C(Nc1ccc(OCCc2ccccc2)cc1)c1cccc(-c2nccc(C(=O)O)n2)c1]

From the starting material methyl 2-bromopyrimidine-4-carboxylate (CAS 1209459-78-8, BLD). Reaction at 90° C. for 18 h afforded 2-(3-((4-phenethoxyphenyl)carbamoyl)phenyl)-pyrimidine-4-carboxylic acid in 36% yield.

LCMS: Method B, 1.24 min, MS: ES⁺ 440.0; ¹H NMR (500 MHz, DMSO) δ ppm 10.34 (s, 1H), 9.11 (d, J=4.9 Hz, 1H), 8.97-8.94 (m, 1H), 8.62 (d, J=7.8 Hz, 1H), 8.10 (d, J=7.8 Hz, 1H), 7.85 (d, J=4.9 Hz, 1H), 7.72-7.67 (m, 3H), 7.36-7.29 (m, 4H), 7.26-7.21 (m, 1H), 6.97-6.92 (m, 2H), 4.19 (t, J=6.9 Hz, 2H), 3.04 (t, J=6.9 Hz, 2H). 1H obscured/not observed. The isolated compound contained up to 1 mol. eq. of ammonia.

### Example 23

### 5-(3-((4-Phenethoxyphenyl)carbamoyl)phenyl)picolinic acid

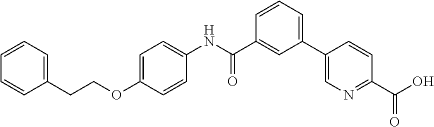 [SMILES: O=C(Nc1ccc(OCCc2ccccc2)cc1)c1cccc(-c2ccc(C(=O)O)nc2)c1]

From the starting material methyl 5-bromopicolinate (CAS 29682-15-3, Fluorochem). Reaction at 90° C. for 18 h afforded 5-(3-((4-phenethoxyphenyl)carbamoyl)phenyl)picolinic acid in 36% yield.

LCMS: Method A, 1.79 min, MS: ES⁺ 439.2; ¹H NMR (500 MHz, DMSO) δ ppm 10.36 (s, 1H), 8.94-8.90 (m, 1H), 8.33-8.29 (m, 1H), 8.18-8.12 (m, 1H), 7.99-7.92 (m, 3H), 7.73-7.61 (m, 3H), 7.37-7.29 (m, 4H), 7.23 (t, J=6.8 Hz, 1H), 6.97-6.91 (m, 2H), 4.19 (t, J=6.9 Hz, 2H), 3.04 (t, J=6.9 Hz, 2H). 1H obscured/not observed. The isolated compound contained up to 1 mol. eq. of ammonia.

### Example 24

### 5-Methyl-6-(3-((4-phenethoxyphenyl)carbamoyl)phenyl)picolinic acid

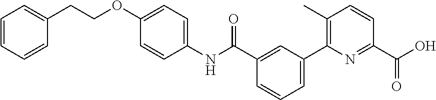 [SMILES: Cc1ccc(C(=O)O)nc1-c1cccc(C(=O)Nc2ccc(OCCc3ccccc3)cc2)c1]

From the starting material methyl 6-chloro-5-methylpicolinate (CAS 178421-22-2, Fluorochem). Reaction at 90° C. for 18 h afforded 5-methyl-6-(3-((4-phenethoxyphenyl)-carbamoyl)phenyl)picolinic acid in 51% yield.

LCMS: Method A, 1.91 min, MS: ES⁺ 453.2; ¹H NMR (500 MHz, DMSO) δ ppm 10.20 (s, 1H), 8.11 (s, 1H), 8.00 (d, J=7.8 Hz, 1H), 7.84 (d, J=7.8 Hz, 1H), 7.79-7.71 (m, 2H), 7.68 (d, J=8.9 Hz, 2H), 7.62 (t, J=7.8 Hz, 1H), 7.37-7.29 (m, 4H), 7.27-7.20 (m, 1H), 6.96-6.91 (m, 2H), 4.18 (t, J=6.9 Hz, 2H), 3.04 (t, J=6.9 Hz, 2H), 2.35 (s, 3H). 1H obscured/not observed. The isolated compound contained up to 1 mol. eq. of ammonia.

### Example 25

### 6-Methyl-5-(3-((4-phenethoxyphenyl)carbamoyl)phenyl)nicotinic acid

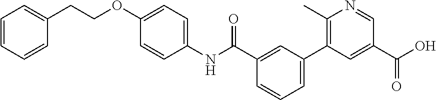 [SMILES: Cc1ncc(C(=O)O)cc1-c1cccc(C(=O)Nc2ccc(OCCc3ccccc3)cc2)c1]

From the starting material 5-bromo-6-methylnicotinic acid (CAS 1190862-72-6, Apollo). Reaction at 90° C. for 18 h afforded 6-methyl-5-(3-((4-phenethoxyphenyl)carbamoyl)phenyl)-nicotinic acid in 43% yield.

LCMS: Method A, 1.76 min, MS: ES⁺ 453.2; ¹H NMR (500 MHz, DMSO) δ ppm 13.53 (s, 1H), 10.18 (s, 1H), 8.99 (d, J=2.1 Hz, 1H), 8.09 (d, J=2.1 Hz, 1H), 8.04-7.98 (m, 2H), 7.71-7.61 (m, 4H), 7.37-7.29 (m, 4H), 7.27-7.20 (m, 1H), 6.98-6.91 (m, 2H), 4.18 (t, J=6.9 Hz, 2H), 3.04 (t, J=6.9 Hz, 2H), 2.53 (s, 3H). The isolated compound contained up to 1 mol. eq. of ammonia.

### Example 26

### 4-(3-((4-Phenethoxyphenyl)carbamoyl)phenyl)nicotinic acid

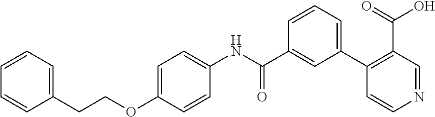 [SMILES: O=C(Nc1ccc(OCCc2ccccc2)cc1)c1cccc(-c2ccncc2C(=O)O)c1]

From the starting material 4-chloronicotinic acid (CAS 10177-29-4, Fluorochem). Reaction at 90° C. for 18 h afforded 4-(3-((4-phenethoxyphenyl)carbamoyl)phenyl)nicotinic acid in 24% yield.

LCMS: Method A, 1.66 min, MS: ES⁺ 439.2; ¹H NMR (500 MHz, DMSO) δ ppm 10.18 (s, 1H), 8.70 (s, 1H), 8.58 (d, J=5.1 Hz, 1H), 8.03 (d, J=1.9 Hz, 1H), 7.95 (d, J=7.6 Hz, 1H), 7.71-7.64 (m, 3H), 7.55 (t, J=7.7 Hz, 1H), 7.42 (d, J=5.1 Hz, 1H), 7.37-7.29 (m, 4H), 7.27-7.20 (m, 1H), 6.97-6.90 (m, 2H), 4.18 (t, J=6.9 Hz, 2H), 3.04 (t, J=6.9 Hz, 2H). 1H obscured/not observed. The isolated compound contained up to 1 mol. eq. of ammonia.

### Example 27

### 4-Methyl-5-(3-((4-phenethoxyphenyl)carbamoyl)phenyl)nicotinic acid

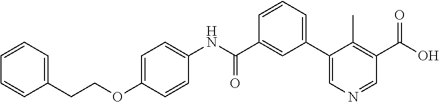 [SMILES: Cc1c(C(=O)O)cncc1-c1cccc(C(=O)Nc2ccc(OCCc3ccccc3)cc2)c1]

From the starting material 5-bromo-4-methylnicotinic acid (CAS 677702-58-8, Combi-Blocks). Reaction at 90° C. for 18 h afforded 4-methyl-5-(3-((4-phenethoxyphenyl)-carbamoyl)phenyl)nicotinic acid in 63% yield.

LCMS: Method A, 1.75 min, MS: ES⁺ 453.2; ¹H NMR (500 MHz, DMSO) δ ppm 13.46 (s, 1H), 10.15 (s, 1H), 8.89 (s, 1H), 8.56 (s, 1H), 8.02 (d, J=7.5 Hz, 1H), 7.97 (s, 1H), 7.69-7.60 (m, 4H), 7.37-7.29 (m, 4H), 7.27-7.20 (m, 1H), 6.98-6.91 (m, 2H), 4.18 (t, J=6.9 Hz, 2H), 3.04 (t, J=6.9 Hz, 2H), 2.43 (s, 3H). The isolated compound contained up to 1 mol. eq. of ammonia.

### Example 28

### 5-(3-((4-Phenethoxyphenyl)carbamoyl)phenyl)pyridazine-3-carboxylic acid

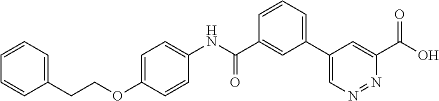 [SMILES: O=C(Nc1ccc(OCCc2ccccc2)cc1)c1cccc(-c2cnnc(C(=O)O)c2)c1]

From the starting material 5-chloropyridazine-3-carboxylic acid (CAS 1211587-01-7, BLD). Reaction at 90° C. for 18 h afforded 5-(3-((4-phenethoxyphenyl)carbamoyl)phenyl)-pyridazine-3-carboxylic acid in 19% yield.

LCMS: Method B, 1.25 min, MS: ES⁺ 440.2; ¹H NMR (500 MHz, DMSO) δ ppm 10.32 (s, 1H), 9.59 (s, 1H), 8.45 (s, 1H), 8.30-8.26 (m, 1H), 8.11 (d, J=7.8 Hz, 1H), 8.06 (d, J=7.8 Hz, 1H), 7.74-7.65 (m, 3H), 7.37-7.29 (m, 4H), 7.24 (t, J=7.0 Hz, 1H), 7.11-6.89 (m, 2H), 4.20 (t, J=6.9 Hz, 2H), 3.05 (t, J=6.9 Hz, 2H). 1H obscured/not observed. The isolated compound contained up to 1 mol. eq. of ammonia.

### Example 29

### 2-Carbamoyl-5-(3-((4-phenethoxyphenyl)carbamoyl)phenyl)nicotinic acid

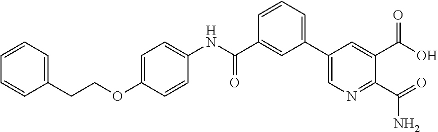 [SMILES: NC(=O)c1ncc(-c2cccc(C(=O)Nc3ccc(OCCc4ccccc4)cc3)c2)cc1C(=O)O]

From the starting material 5-bromo-2-cyanonicotinic acid (CAS 914637-97-1, BLD). Reaction at 90° C. for 18 h afforded 2-carbamoyl-5-(3-((4-phenethoxyphenyl)carbamoyl)-phenyl)nicotinic acid in 5% yield.

LCMS: Method A, 1.78 min, MS: ES⁺ 482.2; ¹H NMR (500 MHz, DMSO) δ ppm 13.40 (s, 1H), 10.26 (s, 1H), 9.03 (s, 1H), 8.34 (s, 1H), 8.22 (s, 1H), 8.02 (t, J=8.6 Hz, 2H), 7.72-7.64 (m, 3H), 7.38-7.29 (m, 4H), 7.27-7.21 (m, 1H), 6.99-6.93 (m, 2H), 4.20 (t, J=6.9 Hz, 2H), 3.05 (t, J=6.9 Hz, 2H). 2H obscured/not observed. The isolated compound contained up to 1 mol. eq. of ammonia.

### Example 30

### 2-(Methylsulfonamido)-5-(3-((4-phenethoxyphenyl)carbamoyl)phenyl)nicotinic acid

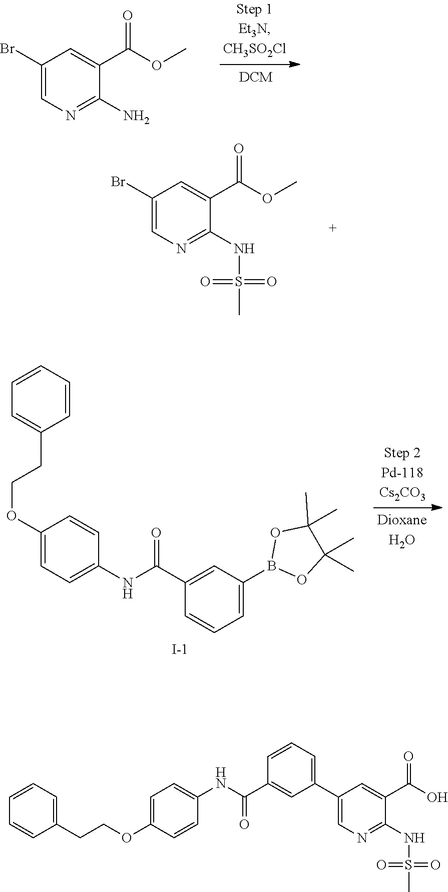 [SMILES: COC(=O)c1cc(Br)cnc1N ; COC(=O)c1cc(Br)cnc1NS(C)(=O)=O ; CC1(C)OB(c2cccc(C(=O)Nc3ccc(OCCc4ccccc4)cc3)c2)OC1(C)C ; [I-] ; CS(=O)(=O)Nc1ncc(-c2cccc(C(=O)Nc3ccc(OCCc4ccccc4)cc3)c2)cc1C(=O)O]

### Step 1: Methyl 5-bromo-2-(methylsulfonamido)nicotinate

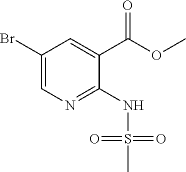 [SMILES: COC(=O)c1cc(Br)cnc1NS(C)(=O)=O]

To an ice-cold solution of methyl 2-amino-5-bromonicotinate (CAS 50735-34-7, Fluorochem, 50 mg, 0.22 mmol) and Et₃N (0.15 mL, 1.08 mmol) in DCM (4 mL) was added methanesulfonyl chloride (0.084 mL, 1.08 mmol) dropwise at 0° C. The reaction mixture was stirred at 40° C. for 72 h, then cooled to rt, treated with sat. aq. NH₄Cl (5 mL) and extracted with EtOAc (3×10 mL). The combined organic phases were washed with water (2 mL), dried over Na₂SO₄ and concentrated under reduced pressure to give methyl 5-bromo-2-(methylsulfonamido)nicotinate (70 mg, 86%).

LCMS: Method A, 1.43 min, MS: ES⁺ 309.0 and 311.0.

### Step 2: 2-(Methylsulfonamido)-5-(3-((4-phenethoxyphenyl)carbamoyl)phenyl)nicotinic acid (Example 30)

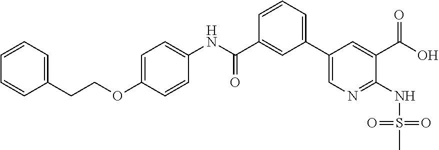 [SMILES: CS(=O)(=O)Nc1ncc(-c2cccc(C(=O)Nc3ccc(OCCc4ccccc4)cc3)c2)cc1C(=O)O]

Following the procedure of Example 1, using methyl 5-bromo-2-(methylsulfonamido)nicotinate in place of 5-bromo-2-hydroxybenzoic acid (CAS 89-55-4, Acros) and with reaction at 80° C. for 9 h, was obtained 2-(methylsulfonamido)-5-(3-((4-phenethoxyphenyl)carbamoyl)phenyl)nicotinic acid in 35% yield.

LCMS: Method B, 1.24 min, MS: ES⁺ 532.0; ¹H NMR (500 MHz, DMSO) δ ppm: 10.24 (s, 1H), 8.81 (s, 1H), 8.58 (s, 1H), 8.24 (s, 1H), 7.96-7.89 (m, 2H), 7.70-7.66 (m, 2H), 7.63 (t, J=7.7 Hz, 1H), 7.37-7.29 (m, 4H), 7.27-7.22 (m, 1H), 6.98-6.93 (m, 2H), 4.19 (t, J=6.9 Hz, 2H), 3.39 (s, 3H), 3.05 (t, J=6.9 Hz, 2H). 2H were obscured/not observed. The isolated compound contained up to 1 mol. eq. of ammonia.

### Example 31

### 1-Methyl-2-oxo-5-(3-((4-phenethoxyphenyl)carbamoyl)phenyl)-1,2-dihydropyridine-3-carboxylic acid

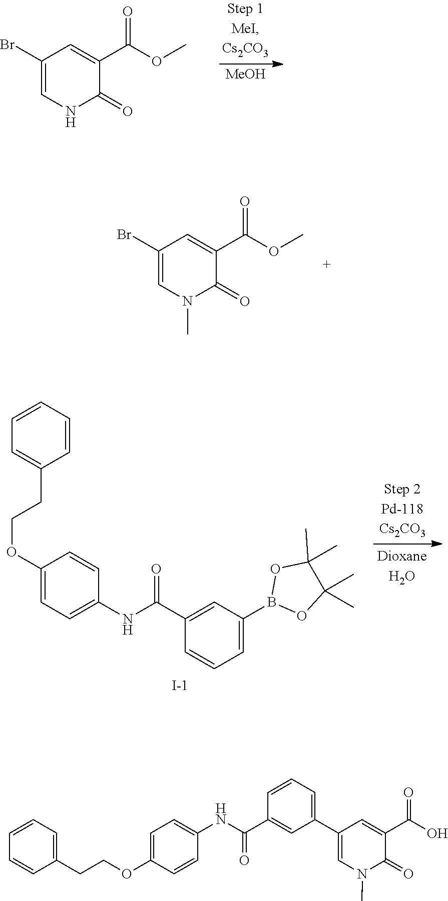 [SMILES: "MeOH" ; COC(=O)c1cc(Br)c[nH]c1=O ; COC(=O)c1cc(Br)cn(C)c1=O ; CC1(C)OB(c2cccc(C(=O)Nc3ccc(OCCc4ccccc4)cc3)c2)OC1(C)C ; [I-] ; Cn1cc(-c2cccc(C(=O)Nc3ccc(OCCc4ccccc4)cc3)c2)cc(C(=O)O)c1=O]

### Step 1: Methyl 5-bromo-1-methyl-2-oxo-1,2-dihydropyridine-3-carboxylate

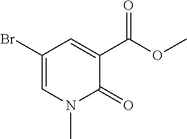

To a slurry of methyl 5-bromo-2-hydroxynicotinate (CAS 120034-05-1, BLD, 100 mg, 0.43 mmol) and Cs₂CO₃ (280 mg, 0.86 mmol) in MeOH (3 mL) was added iodomethane (80 μL, 1.3 mmol), and the mixture was stirred under microwave irradiation at 80° C. for 2 h. The mixture was concentrated under reduced pressure, diluted with EtOAc (30 mL) and filtered through Celite® to give methyl 5-bromo-1-methyl-2-oxo-1,2-dihydropyridine-3-carboxylate (60 mg, 56%).

LCMS: Method A, 0.93 min, MS: ES⁺ 246.0 and 248.0.

### Step 2: 1-Methyl-2-oxo-5-(3-((4-phenethoxyphenyl)carbamoyl)phenyl)-1,2-dihydropyridine-3-carboxylic acid (Example 31)

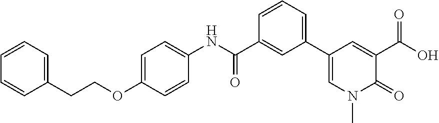 [SMILES: Cn1cc(-c2cccc(C(=O)Nc3ccc(OCCc4ccccc4)cc3)c2)cc(C(=O)O)c1=O]

Following the procedure of Example 1, using methyl 5-bromo-1-methyl-2-oxo-1,2-dihydropyridine-3-carboxylate in place of 5-bromo-2-hydroxybenzoic acid (CAS 89-55-4, Acros) and with reaction at 80° C. for 9 h, was obtained 1-methyl-2-oxo-5-(3-((4-phenethoxyphenyl)carbamoyl)phenyl)-1,2-dihydropyridine-3-carboxylic acid in 35% yield.

LCMS: Method B, 1.19 min, MS: ES⁺ 469.2; ¹H NMR (500 MHz, DMSO) δ ppm: 10.25 (s, 1H), 8.66 (s, 1H), 8.58 (s, 1H), 8.18 (s, 1H), 7.92 (d, J=7.8 Hz, 1H), 7.86 (d, J=7.8 Hz, 1H), 7.70-7.65 (m, 2H), 7.62 (t, J=7.8 Hz, 1H), 7.38-7.29 (m, 4H), 7.27-7.21 (m, 1H), 6.99-6.93 (m, 2H), 4.19 (t, J=6.9 Hz, 2H), 3.72 (s, 3H), 3.04 (t, J=6.9 Hz, 2H). 1H obscured/not observed. The isolated compound contained up to 1 mol. eq. of ammonia.

### Example 32

### 2-(Morpholinomethyl)-5-(3-((4-phenethoxyphenyl)carbamoyl)phenyl)nicotinic acid

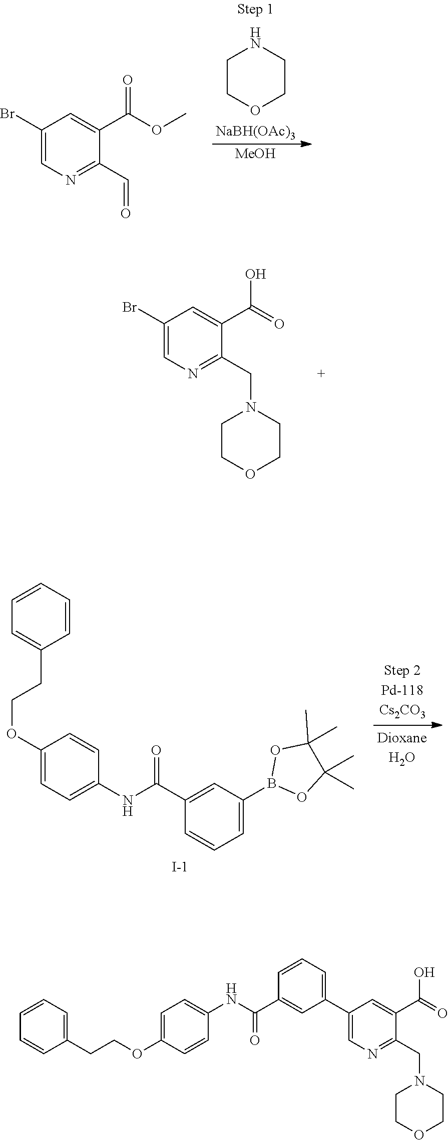

### Step 1: 5-Bromo-2-(morpholinomethyl)nicotinic acid

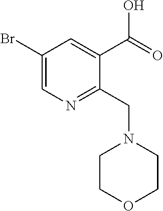 [SMILES: O=C(O)c1cc(Br)cnc1CN1CCOCC1]

To a solution of methyl 5-bromo-2-formylnicotinate (75 mg, 0.31 mmol) in DCM (0.75 mL), morpholine (0.27 mL, 3.1 mmol) was added. The mixture was stirred for 3 h then NaBH(OAc)₃ (195 mg, 0.92 mmol) was added. The mixture was stirred for a further 20 h then diluted with EtOAc (50 mL) and washed with sat. aq. NaHCO₃ (2×50 mL) and brine (2×50 mL). The organic phase was dried over Na₂SO₄ and concentrated under reduced pressure to give 5-bromo-2-(morpholinomethyl)nicotinic acid (50 mg, 32%).

LCMS: Method A, 0.23 min, MS: ES⁺ 301.0 and 303.0.

### Step 2: 2-(Morpholinomethyl)-5-(3-((4-phenethoxyphenyl)carbamoyl)phenyl)nicotinic acid (Example 32)

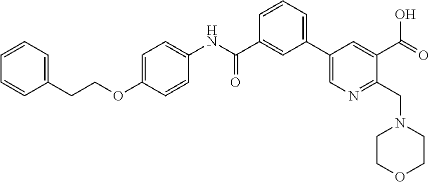 [SMILES: O=C(Nc1ccc(OCCc2ccccc2)cc1)c1cccc(-c2cnc(CN3CCOCC3)c(C(=O)O)c2)c1]

Following the procedure of Example 1, using 5-bromo-2-(morpholinomethyl)nicotinic acid in place of 5-bromo-2-hydroxybenzoic acid (CAS 89-55-4, Acros) and with reaction at 80° C. for 18 h, was obtained 2-(morpholinomethyl)-5-(3-((4-phenethoxyphenyl)carbamoyl)phenyl)nicotinic acid in 15% yield.

LCMS: Method A, 1.62 min, MS: ES⁺ 538.2; ¹H NMR (500 MHz, DMSO) δ ppm: 10.28 (s, 1H), 8.91-8.87 (m, 1H), 8.36-8.32 (m, 3H), 8.31-8.26 (m, 1H), 7.98 (d, J=7.9 Hz, 1H), 7.95 (d, J=7.9 Hz, 1H), 7.71-7.62 (m, 2H), 7.38-7.29 (m, 3H), 7.27-7.20 (m, 1H), 6.99-6.93 (m, 2H), 4.19 (t, J=6.9 Hz, 2H), 4.11 (s, 2H), 3.62-3.56 (m, 4H), 3.04 (d, J=6.9 Hz, 2H), 2.63-2.57 (m, 4H). 1H obscured/not observed. The isolated compound contained up to 1 mol. eq. of ammonia.

### Example 33

### 2-Morpholino-5-(3-((4-phenethoxyphenyl)carbamoyl)phenyl)nicotinic acid

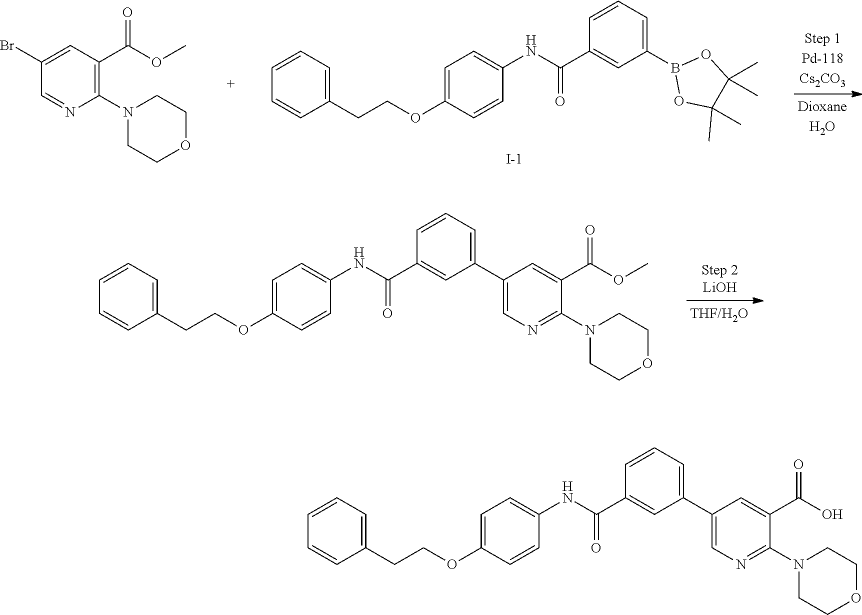 [SMILES: COC(=O)c1cc(Br)cnc1N1CCOCC1 ; CC1(C)OB(c2cccc(C(=O)Nc3ccc(OCCc4ccccc4)cc3)c2)OC1(C)C ; [I-] ; COC(=O)c1cc(-c2cccc(C(=O)Nc3ccc(OCCc4ccccc4)cc3)c2)cnc1N1CCOCC1 ; O=C(Nc1ccc(OCCc2ccccc2)cc1)c1cccc(-c2cnc(N3CCOCC3)c(C(=O)O)c2)c1]

### Step 1: Methyl 2-morpholino-5-(3-((4-phenethoxyphenyl)carbamoyl)phenyl)nicotinate

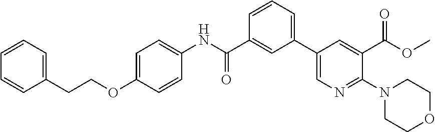 [SMILES: COC(=O)c1cc(-c2cccc(C(=O)Nc3ccc(OCCc4ccccc4)cc3)c2)cnc1N1CCOCC1]

Following the procedure of Example 1, using methyl 5-bromo-2-morpholinonicotinate (CAS 1017782-99-8, Key Organics) in place of 5-bromo-2-hydroxybenzoic acid (CAS 89-55-4, Acros), was obtained methyl 2-morpholino-5-(3-((4-phenethoxyphenyl)carbamoyl)phenyl)-nicotinate in 76% yield.

LCMS: Method A, 2.15 min, MS: ES⁺ 538.2.

### Step 2: 2-Morpholino-5-(3-((4-phenethoxyphenyl)carbamoyl)phenyl)nicotinic acid (Example 33)

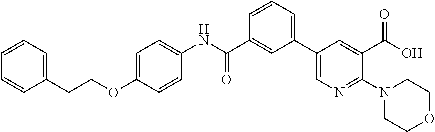 [SMILES: O=C(Nc1ccc(OCCc2ccccc2)cc1)c1cccc(-c2cnc(N3CCOCC3)c(C(=O)O)c2)c1]

To a solution of methyl 2-morpholino-5-(3-((4-phenethoxyphenyl)carbamoyl)phenyl) nicotinate (100 mg, 0.19 mmol) in THF (3 mL) was added LiOH (27 mg, 1.12 mmol) in water (2 mL) at rt. The mixture was stirred at 45° C. for 4 h, then cooled to rt, acidified with aq. HCl (1M, 5 mL) and extracted with EtOAc (3×20 mL). The combined organic phases were dried over Na₂SO₄ and concentrated under reduced pressure. The crude product was purified by RP flash chromatography (C18, 0-100% MeCN in 0.1% aq. NH₄OH) to afford 2-morpholino-5-(3-((4-phenethoxyphenyl)carbamoyl)phenyl)nicotinic acid (28.0 mg, 27%).

LCMS: Method B, 1.29 min, MS: ES⁺ 524.2; ¹H NMR (500 MHz, DMSO) δ ppm: 10.20 (s, 1H), 8.67 (s, 1H), 8.25 (s, 1H), 8.20 (s, 1H), 7.91-7.85 (m, 2H), 7.67 (d, J=9.0 Hz, 2H), 7.60 (t, J=7.7 Hz, 1H), 7.38-7.30 (m, 4H), 7.24 (t, J=6.8 Hz, 1H), 6.97-6.92 (m, 2H), 4.19 (t, J=6.9 Hz, 2H), 3.73-3.67 (m, 4H), 3.45-3.40 (m, 4H), 3.04 (t, J=6.9 Hz, 2H). 1H was obscured/not observed. The isolated compound contained up to 1 mol. eq. of ammonia.

### Example 34

### 2-(Methylamino)-5-(3-((4-phenethoxyphenyl)carbamoyl)phenyl)nicotinic acid

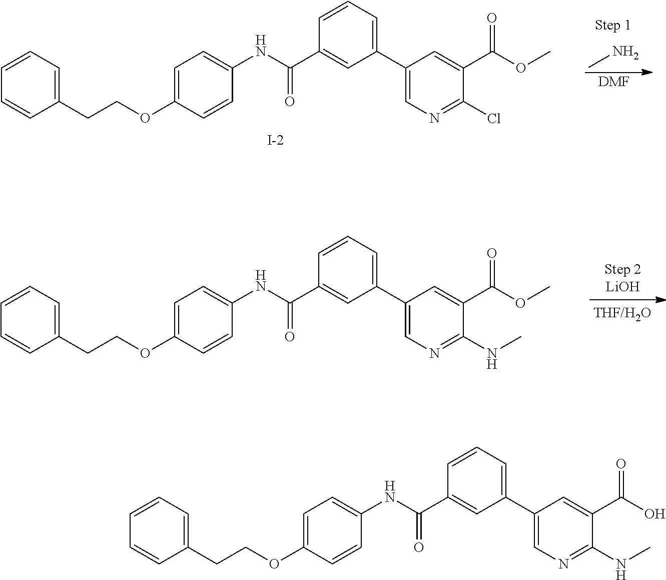

### Step 1: Methyl 2-(methylamino)-5-(3-((4-phenethoxyphenyl)carbamoyl)phenyl)nicotinate

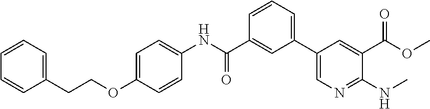 [SMILES: CNc1ncc(-c2cccc(C(=O)Nc3ccc(OCCc4ccccc4)cc3)c2)cc1C(=O)OC]

To a solution of methyl 2-chloro-5-(3-((4-phenethoxyphenyl)carbamoyl)phenyl)nicotinate (I-2) (50 mg, 0.10 μmol) in DMF (3 mL), methylamine (1M in MeOH, 2.05 mL, 2.05 mmol) was added. The mixture was stirred under microwave irradiation at 100° C. and stirred for 72 h, then cooled to rt, diluted with water (15 mL) and brine (15 mL) and extracted with EtOAc (3×30 mL). The combined organic phases were dried over Na₂SO₄ and concentrated under reduced pressure to give methyl 2-(methylamino)-5-(3-((4-phenethoxyphenyl)carbamoyl)phenyl)nicotinate (40 mg, 64%).

LCMS: Method A, 2.12 min, MS: ES⁺ 482.2.

### Step 2: 2-(Methylamino)-5-(3-((4-phenethoxyphenyl)carbamoyl)phenyl)nicotinic acid (Example 34)

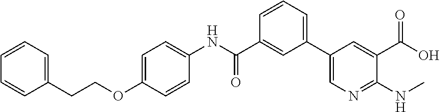 [SMILES: CNc1ncc(-c2cccc(C(=O)Nc3ccc(OCCc4ccccc4)cc3)c2)cc1C(=O)O]

Following the procedure of Example 33 Step 2, using methyl 2-(methylamino)-5-(3-((4-phenethoxyphenyl)carbamoyl)phenyl)nicotinate in place of methyl 2-morpholino-5-(3-((4-phenethoxyphenyl)carbamoyl)phenyl)nicotinate, was obtained 2-(methylamino)-5-(3-((4-phenethoxyphenyl)carbamoyl)phenyl)nicotinic acid in 61% yield.

LCMS: Method A, 1.70 min, MS: ES⁺ 468.2; ¹H NMR (500 MHz, DMSO) δ ppm: 10.20 (s, 1H), 8.71 (d, J=2.6 Hz, 1H), 8.41 (d, J=2.6 Hz, 1H), 8.15 (s, 1H), 7.86 (d, J=7.7 Hz, 1H), 7.83 (d, J=7.7 Hz, 1H), 7.71-7.65 (m, 2H), 7.58 (t, J=7.7 Hz, 1H), 7.38-7.29 (m, 4H), 7.27-7.20 (m, 1H), 6.98-6.93 (m, 2H), 4.19 (t, J=6.9 Hz, 2H), 3.04 (t, J=6.9 Hz, 2H), 3.02 (s, 3H). 2H were obscured/not observed. The isolated compound contained up to 1 mol. eq. of ammonia.

### Example 35

### 2-(Dimethylamino)-5-(3-((4-phenethoxyphenyl)carbamoyl)phenyl)nicotinic acid

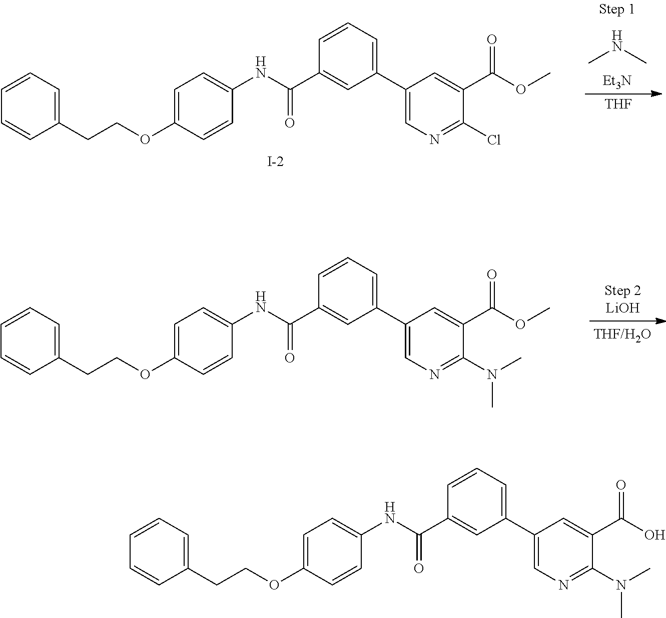

### Step 1: Methyl 2-(dimethylamino)-5-(3-((4-phenethoxyphenyl)carbamoyl)phenyl)nicotinate

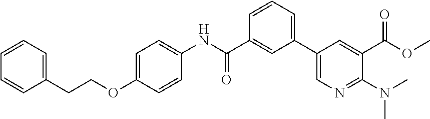 [SMILES: COC(=O)c1cc(-c2cccc(C(=O)Nc3ccc(OCCc4ccccc4)cc3)c2)cnc1N(C)C]

Following the procedure of Example 34 Step 1, using methanolic dimethylamine in place of methanolic methylamine, was obtained methyl 2-(dimethylamino)-5-(3-((4-phenethoxyphenyl)carbamoyl)phenyl)nicotinate in 61% yield.

LCMS: Method A, 2.15 min, MS: ES⁺ 496.2.

### Step 2: 2-(dimethylamino)-5-(3-((4-phenethoxyphenyl)carbamoyl)phenyl)nicotinic acid (Example 35)

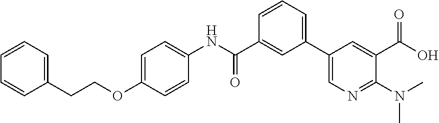 [SMILES: CN(C)c1ncc(-c2cccc(C(=O)Nc3ccc(OCCc4ccccc4)cc3)c2)cc1C(=O)O]

Following the procedure of Example 33 Step 2, using methyl 2-(dimethylamino)-5-(3-((4-phenethoxyphenyl)carbamoyl)phenyl)nicotinate in place of methyl 2-morpholino-5-(3-((4-phenethoxyphenyl)carbamoyl)phenyl)nicotinate, was obtained 2-(dimethylamino)-5-(3-((4-phenethoxyphenyl)carbamoyl)phenyl)nicotinic acid in 54% yield.

LCMS: Method A, 1.76 min, MS: ES⁺ 482.2; ¹H NMR (500 MHz, DMSO) δ ppm: 10.20 (s, 1H), 8.61 (s, 1H), 8.22-8.15 (m, 2H), 7.89-7.82 (m, 2H), 7.67 (d, J=9.0 Hz, 2H), 7.58 (t, J=7.7 Hz, 1H), 7.37-7.30 (m, 3H), 7.27-7.21 (m, 1H), 6.95 (d, J=9.0 Hz, 2H), 4.19 (t, J=6.9 Hz, 2H), 3.32 (s, 3H), 3.30 (s, 3H), 3.05 (t, J=6.9 Hz, 2H). 2H were obscured/not observed. The isolated compound contained up to 1 mol. eq. of ammonia.

### Example 36

### 2-((2-Hydroxyethyl)amino)-5-(3-((4-phenethoxyphenyl)carbamoyl)phenyl)nicotinic acid

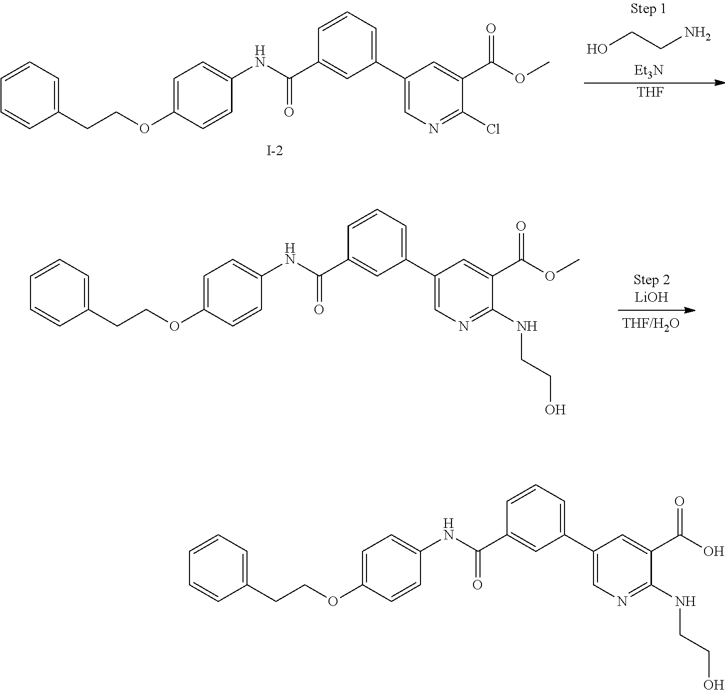

### Step 1: Methyl 2-((2-hydroxyethyl)amino)-5-(3-((4-phenethoxyphenyl)carbamoyl)phenyl) nicotinate

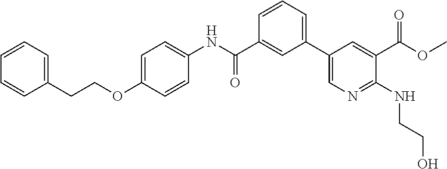 [SMILES: COC(=O)c1cc(-c2cccc(C(=O)Nc3ccc(OCCc4ccccc4)cc3)c2)cnc1NCCO]

To a solution of 2-aminoethan-1-ol (CAS 141-43-5, Merck, 5.0 mg, 0.08 mmol) and methyl 2-chloro-5-(3-((4-phenethoxyphenyl)carbamoyl)phenyl)nicotinate (I-2, 20 mg, 0.04 mmol) in THF (2 mL) was added triethylamine (0.02 mL, 0.12 mmol) and the mixture was then stirred at 40° C. for 72 h. The mixture was concentrated under reduced pressure to give methyl 2-((2-hydroxyethyl)amino)-5-(3-((4-phenethoxyphenyl)carbamoyl)phenyl)nicotinate (20 mg, 76%).

LCMS: Method A, 1.96 min, MS: ES⁺ 512.3.

### Step 2: 2-((2-Hydroxyethyl)amino)-5-(3-((4-phenethoxyphenyl)carbamoyl)phenyl)nicotinic acid (Example 36)

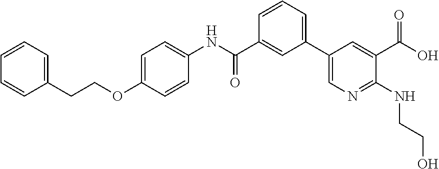 [SMILES: O=C(Nc1ccc(OCCc2ccccc2)cc1)c1cccc(-c2cnc(NCCO)c(C(=O)O)c2)c1]

To a solution of methyl 2-((2-hydroxyethyl)amino)-5-(3-((4-phenethoxyphenyl)-carbamoyl)phenyl)nicotinate (20 mg, 0.04 mmol) in THF (1 mL) was added NaOH (78 mg, 0.98 mmol) in water (1 mL) at rt. The mixture was stirred at 45° C. for 4 h, then cooled to rt, acidified with aq. HCl (2M, 1 mL) and extracted with EtOAc (3×10 mL). The combined organic phases were dried over Na₂SO₄ and concentrated under reduced pressure. The crude product was purified by RP flash chromatography (C18, 0-100% MeCN in 0.1% aq. NH₄OH) to afford 2-((2-hydroxyethyl)amino)-5-(3-((4-phenethoxyphenyl)carbamoyl)phenyl)-nicotinic acid (3 mg, 14%).

LCMS: Method A, 1.66 min, MS: ES⁺ 498.2; ¹H NMR (500 MHz, DMSO) δ ppm: 10.21 (s, 1H), 8.82-8.63 (m, 1H), 8.42 (d, J=2.6 Hz, 1H), 8.15 (s, 1H), 7.86 (d, J=7.7 Hz, 1H), 7.82 (d, J=7.7 Hz, 1H), 7.67 (dd, J=9.5, 3.2 Hz, 2H), 7.57 (t, J=7.7 Hz, 1H), 7.38-7.29 (m, 4H), 7.27-7.20 (m, 1H), 6.98-6.92 (m, 2H), 4.19 (t, J=6.9 Hz, 2H), 3.64-3.56 (m, 4H), 3.04 (t, J=6.9 Hz, 2H). 3H were obscured/not observed. The isolated compound contained up to 1 mol. eq. of ammonia.

### Example 37

### 2-((2-Methoxyethyl)amino)-5-(3-((4-phenethoxyphenyl)carbamoyl)phenyl)nicotinic acid

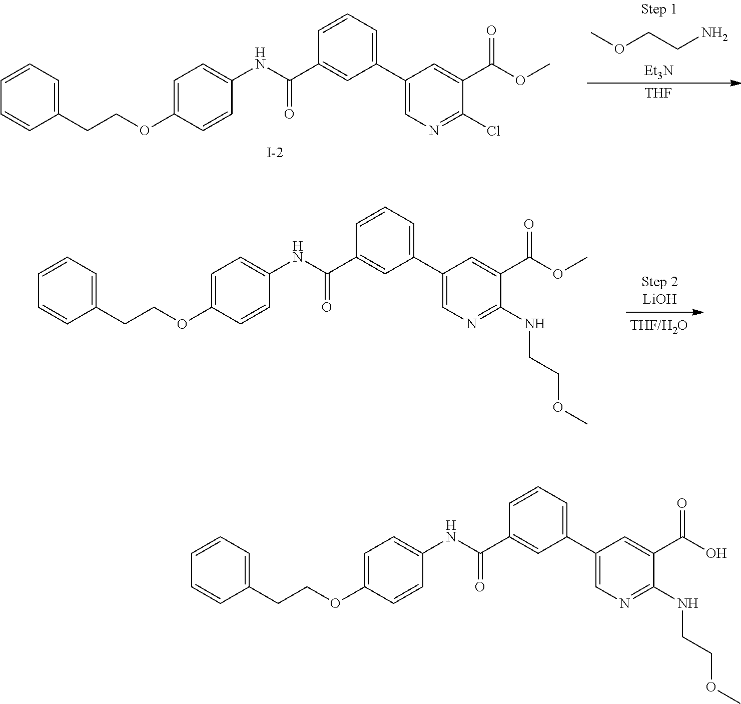

### Step 1: Methyl 2-((2-hydroxyethyl)amino)-5-(3-((4-phenethoxyphenyl)carbamoyl)phenyl) nicotinate

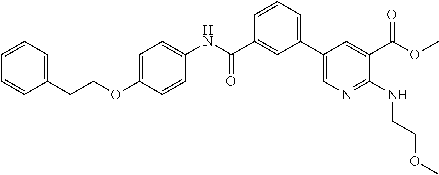 [SMILES: COCCNc1ncc(-c2cccc(C(=O)Nc3ccc(OCCc4ccccc4)cc3)c2)cc1C(=O)OC]

Following the procedure of Example 36 Step 1, using 2-methoxyethan-1-amine (CAS 109-85-3, Fluorochem) in place of 2-aminoethan-1-ol, was obtained methyl 2-((2-methoxyethyl)amino)-5-(3-((4-phenethoxyphenyl)carbamoyl)phenyl)nicotinate in 65% yield.

LCMS: Method A, 2.17 min, MS: ES⁺ 526.2.

### Step 2: 2-((2-Methoxyethyl)amino)-5-(3-((4-phenethoxyphenyl)carbamoyl)phenyl)nicotinic acid (Example 37)

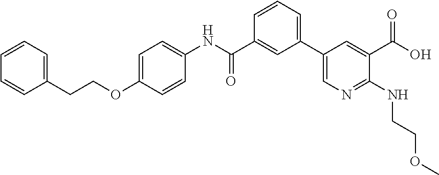 [SMILES: COCCNc1ncc(-c2cccc(C(=O)Nc3ccc(OCCc4ccccc4)cc3)c2)cc1C(=O)O]

Following the procedure of Example 36 Step 2, using methyl 2-((2-methoxyethyl)amino)-5-(3-((4-phenethoxyphenyl)carbamoyl)phenyl)nicotinate in place of methyl 2-((2-hydroxyethyl)amino)-5-(3-((4-phenethoxyphenyl)carbamoyl)phenyl)nicotinate, was obtained 2-((2-methoxyethyl)amino)-5-(3-((4-phenethoxyphenyl)carbamoyl)phenyl)nicotinic acid in 78% yield.

LCMS: Method A, 1.83 min, MS: ES⁺ 512.3; ¹H NMR (500 MHz, DMSO) δ ppm: 10.21 (s, 1H), 9.19 (s, 1H), 8.55 (s, 1H), 8.40 (d, J=2.4 Hz, 1H), 8.14 (d, J=2.4 Hz, 1H), 7.84 (d, J=7.7 Hz, 1H), 7.80 (d, J=7.7 Hz, 1H), 7.70-7.64 (m, 2H), 7.56 (t, J=7.7 Hz, 1H), 7.38-7.29 (m, 4H), 7.27-7.20 (m, 1H), 6.98-6.91 (m, 2H), 4.19 (t, J=6.9 Hz, 2H), 3.68-3.61 (m, 2H), 3.53 (t, J=5.6 Hz, 2H), 3.30 (s, 3H), 3.04 (t, J=6.9 Hz, 2H). 1H was obscured/not observed. The isolated compound contained up to 1 mol. eq. of ammonia.

### Example 38

### 2-(Cyclopropylamino)-5-(3-((4-phenethoxyphenyl)carbamoyl)phenyl)nicotinic acid

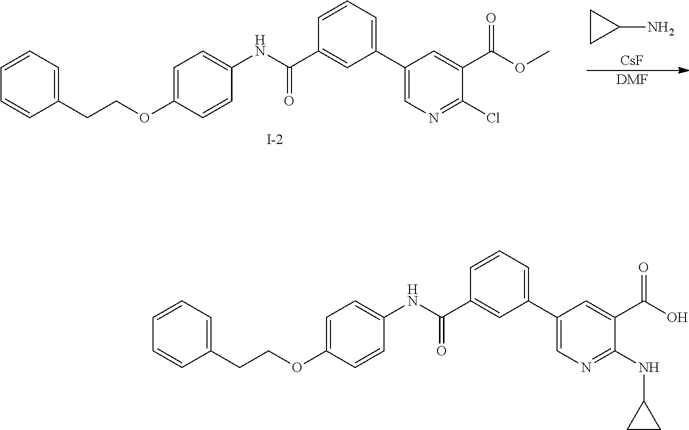

To a stirred solution of cyclopropanamine (CAS 765-30-0, Merck, 0.86 mL, 12.3 mmol) and methyl 2-chloro-5-(3-((4-phenethoxyphenyl)carbamoyl)phenyl)nicotinate (I-2, 20 mg, 0.041 mmol) in DMF (2 mL) at 0° C. was added CsF (13 mg, 0.082 mmol) and the mixture was then stirred at 50° C. for 24 h, cooled to rt, diluted with water (100 mL) and extracted with EtOAc (100 mL). The organic phase was washed with brine (2×100 mL), dried over MgSO₄ and concentrated under reduced pressure. The crude product was purified by preparative HPLC (Prep Method B, x=30, y=60) in 0.1% aq. NH₄OH) to afford 2-(cyclopropylamino)-5-(3-((4-phenethoxyphenyl)carbamoyl)phenyl)nicotinic acid (9 mg, 42%).

LCMS: Method A, 1.72 min, MS: ES⁺ 494.2; ¹H NMR (500 MHz, DMSO) δ ppm: 13.35 (s, 1H), 10.20 (s, 1H), 8.76 (d, J=2.6 Hz, 1H), 8.42 (d, J=2.6 Hz, 1H), 8.29 (s, 1H), 8.16 (s, 1H), 7.88 (d, J=7.7 Hz, 1H), 7.85 (d, J=7.8 Hz, 1H), 7.71-7.65 (m, 2H), 7.59 (t, J=7.7 Hz, 1H), 7.38-7.29 (m, 4H), 7.27-7.20 (m, 1H), 6.99-6.92 (m, 2H), 4.19 (t, J=6.9 Hz, 2H), 3.04 (t, J=6.9 Hz, 2H), 2.98-2.89 (m, 1H), 0.84-0.76 (m, 2H), 0.56-0.49 (m, 2H). The isolated compound contained up to 1 mol. eq. of ammonia.

### Example 39

### 5-(3-((4-((Benzyloxy)methyl)phenyl)carbamoyl)phenyl)-2-methylnicotinic acid

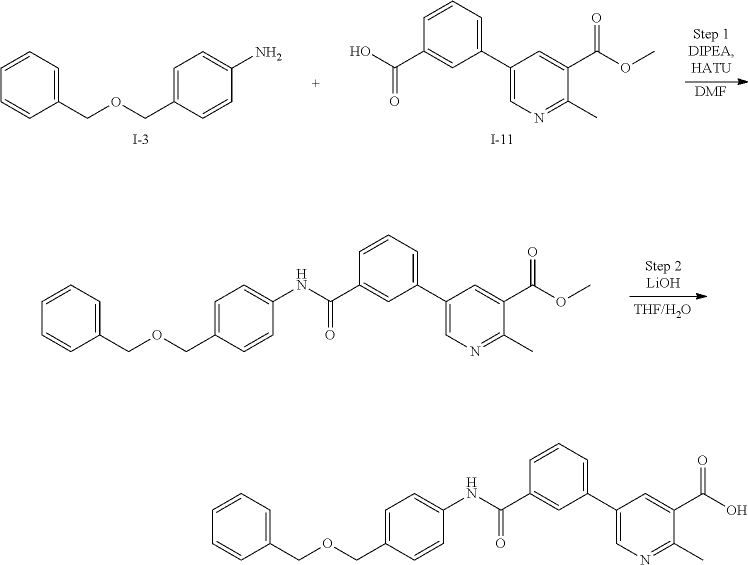

### Step 1: Methyl 5-(3-((4-((benzyloxy)methyl)phenyl)carbamoyl)phenyl)-2-methylnicotinate

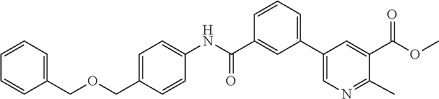 [SMILES: COC(=O)c1cc(-c2cccc(C(=O)Nc3ccc(COCc4ccccc4)cc3)c2)cnc1C]

To a stirred solution of DIPEA (0.096 mL, 0.55 mmol) and 3-(5-(methoxycarbonyl)-6-methylpyridin-3-yl)benzoic acid (I-11, 30 mg, 0.11 mmol) in DMF (2 mL), was added HATU (84 mg, 0.22 mmol) at rt. After 10 min, 4-((benzyloxy)methyl)aniline (I-3, 24 mg, 0.11 mmol) was added. The mixture was stirred at 45° C. for 18 h then cooled, poured into ice-water (30 mL) and extracted with EtOAc (2×15 mL). The combined organic phases were washed with brine (50 mL), dried over Na₂SO₄ and concentrated under reduced pressure to give methyl 5-(3-((4-((benzyloxy)methyl)phenyl)carbamoyl)phenyl)-2-methylnicotinate (45 mg, 45%).

LCMS: Method A, 2.05 min, MS: ES⁺ 467.2.

### Step 2: 5-(3-((4-((Benzyloxy)methyl)phenyl)carbamoyl)phenyl)-2-methylnicotinic acid (Example 39)

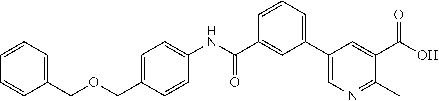 [SMILES: Cc1ncc(-c2cccc(C(=O)Nc3ccc(COCc4ccccc4)cc3)c2)cc1C(=O)O]

To a solution of methyl 5-(3-((4-((benzyloxy)methyl)phenyl)carbamoyl)phenyl)-2-methylnicotinate (45 mg, 0.10 mmol) in MeOH (5 mL) and THF (5 mL) was added LiOH (23 mg, 0.97 mmol) in water (2 mL) at rt. The mixture was stirred at 45° C. for 4 h, then cooled to rt, acidified with aq. HCl (2M, 0.9 mL) and extracted with EtOAc (3×20 mL). The combined organic phases were dried over Na₂SO₄ and concentrated under reduced pressure. The crude product was purified by RP flash chromatography (C18, 0-100% of 0.1% HCO₂H/MeCN in 0.1% aq. HCO₂H) to afford 5-(3-((4-((benzyloxy)methyl)phenyl)carbamoyl)phenyl)-2-methylnicotinic acid (35 mg, 76%).

LCMS: Method A, 1.68 min, MS: ES⁺ 453.2. ¹H NMR (500 MHz, DMSO) δ ppm: 13.45 (s, 1H), 10.39 (s, 1H), 9.01 (d, J=2.4 Hz, 1H), 8.48 (d, J=2.4 Hz, 1H), 8.29 (s, 1H), 7.99 (t, J=8.2 Hz, 2H), 7.81-7.76 (m, 2H), 7.67 (t, J=7.7 Hz, 1H), 7.39-7.34 (m, 6H), 7.34-7.26 (m, 1H), 4.53 (s, 2H), 4.51 (s, 2H), 2.77 (s, 3H).

### Example 40

### 5-(3-((3-((Benzyloxy)methyl)phenyl)carbamoyl)phenyl)-2-methylnicotinic acid

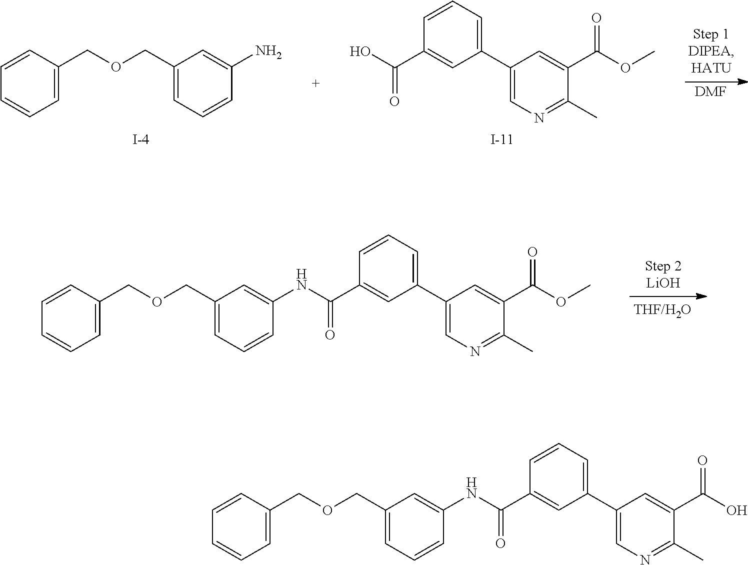

### Step 1: Methyl 5-(3-((3-((benzyloxy)methyl)phenyl)carbamoyl)phenyl)-2-methylnicotinate

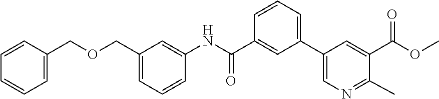 [SMILES: COC(=O)c1cc(-c2cccc(C(=O)Nc3cccc(COCc4ccccc4)c3)c2)cnc1C]

Following the procedure of Example 39 Step 1, using 3-((benzyloxy)methyl)aniline (I-4) in place of 4-((benzyloxy)methyl)aniline (I-3), was obtained methyl 5-(3-((3-((benzyloxy)methyl)phenyl)carbamoyl)phenyl)-2-methylnicotinate in 42% yield.

LCMS: Method A, 2.06 min, MS: ES⁺ 467.2.

### Step 2: 5-(3-((3-((Benzyloxy)methyl)phenyl)carbamoyl)phenyl)-2-methylnicotinic acid (Example 40)

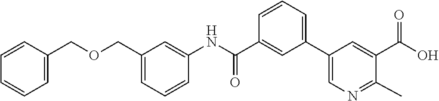 [SMILES: Cc1ncc(-c2cccc(C(=O)Nc3cccc(COCc4ccccc4)c3)c2)cc1C(=O)O]

Following the procedure of Example 39 Step 2, using methyl 5-(3-((3-((benzyloxy)methyl)phenyl)carbamoyl)phenyl)-2-methylnicotinate in place of methyl 5-(3-((4-((benzyloxy)methyl)phenyl)carbamoyl)phenyl)-2-methylnicotinate, was obtained 5-(3-((3-((benzyloxy)methyl)phenyl)carbamoyl)phenyl)-2-methylnicotinic acid in 61% yield.

LCMS: Method A, 1.72 min, MS: ES⁺ 453.1. ¹H NMR (500 MHz, DMSO) δ ppm: 13.67 (s, 1H), 10.45 (s, 1H), 9.15 (s, 1H), 8.68 (s, 1H), 8.39-8.34 (m, 1H), 8.07-8.01 (m, 2H), 7.85-7.81 (m, 1H), 7.81-7.75 (m, 1H), 7.70 (t, J=7.7 Hz, 1H), 7.42-7.34 (m, 5H), 7.34-7.27 (m, 1H), 7.11 (d, J=7.7 Hz, 1H), 4.59-4.54 (m, 4H), 2.84 (s, 3H).

### Example 41

### 5-(3-((4-(Cyclopropylmethoxy)phenyl)carbamoyl)phenyl)-2-methylnicotinic acid

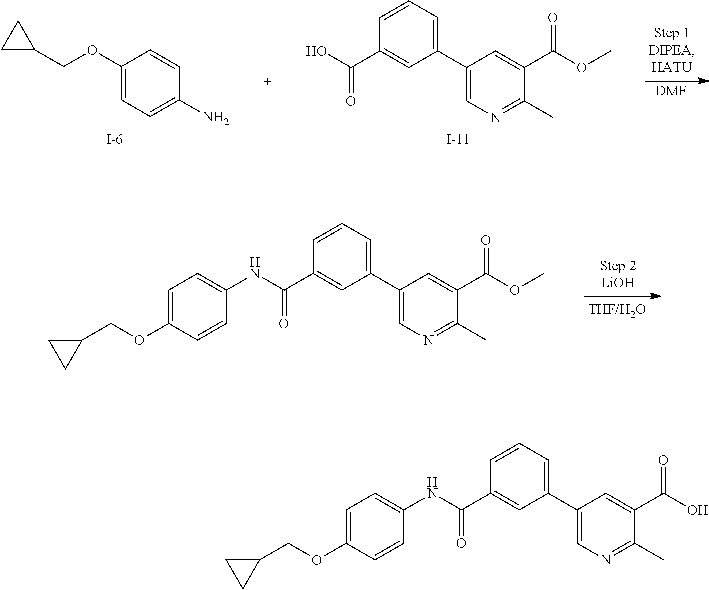

### Step 1: Methyl 5-(3-((4-(cyclopropylmethoxy)phenyl)carbamoyl)phenyl)-2-methylnicotinate

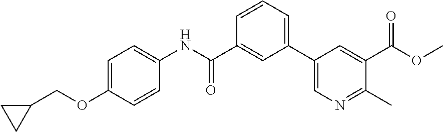 [SMILES: COC(=O)c1cc(-c2cccc(C(=O)Nc3ccc(OCC4CC4)cc3)c2)cnc1C]

Following the procedure of Example 39 Step 1, using 4-(cyclopropylmethoxy)aniline (I-6) in place of 4-((benzyloxy)methyl)aniline (I-3), was obtained methyl 5-(3-((4-(cyclopropylmethoxy)phenyl)carbamoyl)phenyl)-2-methylnicotinate in 43% yield.

LCMS: Method A, 1.90 min, MS: ES⁺ 417.2.

### Step 2: 5-(3-((4-(Cyclopropylmethoxy)phenyl)carbamoyl)phenyl)-2-methylnicotinic acid (Example 41)

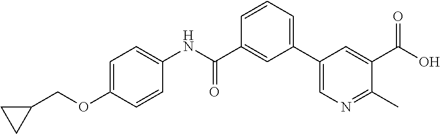 [SMILES: Cc1ncc(-c2cccc(C(=O)Nc3ccc(OCC4CC4)cc3)c2)cc1C(=O)O]

Following the procedure of Example 39 Step 2, using methyl 5-(3-((4-(cyclopropylmethoxy)phenyl)carbamoyl)phenyl)-2-methylnicotinate in place of methyl 5-(3-((4-((benzyloxy)methyl)phenyl)carbamoyl)phenyl)-2-methylnicotinate, was obtained 5-(3-((4-(cyclopropylmethoxy)phenyl)carbamoyl)phenyl)-2-methylnicotinic acid in 35% yield.

LCMS: Method A, 1.50 min, MS: ES⁺ 403.2. ¹H NMR (500 MHz, DMSO) δ ppm: 13.45 (s, 1H), 10.24 (s, 1H), 9.02 (d, J=2.4 Hz, 1H), 8.49 (d, J=2.4 Hz, 1H), 8.31-8.26 (m, 1H), 8.01-7.95 (m, 2H), 7.70-7.63 (m, 3H), 6.97-6.90 (m, 2H), 3.81 (d, J=6.9 Hz, 2H), 2.78 (s, 3H), 1.27-1.18 (m, 1H), 0.61-0.53 (m, 2H), 0.36-0.30 (m, 2H).

### Example 42

### 5-(3-((4-(Cyclopropoxymethyl)phenyl)carbamoyl)phenyl)-2-methylnicotinic acid

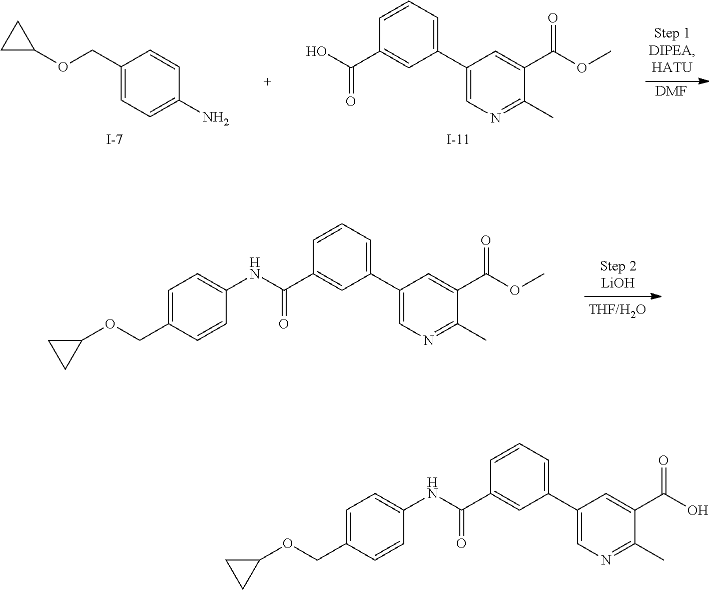

### Step 1: Methyl 5-(3-((4-(cyclopropoxymethyl)phenyl)carbamoyl)phenyl)-2-methylnicotinate

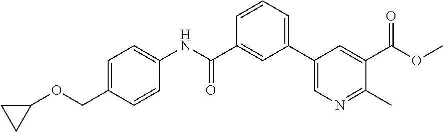 [SMILES: COC(=O)c1cc(-c2cccc(C(=O)Nc3ccc(COC4CC4)cc3)c2)cnc1C]

Following the procedure of Example 39 Step 1, using 4-(cyclopropoxymethyl)aniline (I-7) in place of 4-((benzyloxy)methyl)aniline (I-3), was obtained methyl 5-(3-((4-(cyclopropoxymethyl)phenyl)carbamoyl)phenyl)-2-methylnicotinate in 41% yield.

LCMS: Method A, 1.86 min, MS: ES⁺ 417.2.

### Step 2: 5-(3-((4-(Cyclopropoxymethyl)phenyl)carbamoyl)phenyl)-2-methylnicotinic acid (Example 42)

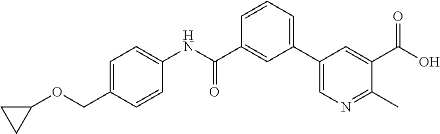 [SMILES: Cc1ncc(-c2cccc(C(=O)Nc3ccc(COC4CC4)cc3)c2)cc1C(=O)O]

Following the procedure of Example 39 Step 2, using methyl 5-(3-((4-(cyclopropoxymethyl)phenyl)carbamoyl)phenyl)-2-methylnicotinate in place of methyl 5-(3-((4-((benzyloxy)methyl)phenyl)carbamoyl)phenyl)-2-methylnicotinate, was obtained 5-(3-((4-(cyclopropoxymethyl)phenyl)carbamoyl)phenyl)-2-methylnicotinic acid in 39% yield.

LCMS: Method A, 1.45 min, MS: ES⁺ 403.2. ¹H NMR (500 MHz, DMSO) δ ppm: 13.45-13.35 (s, 1H), 10.38 (s, 1H), 9.03 (d, J=2.4 Hz, 1H), 8.50 (d, J=2.4 Hz, 1H), 8.30 (s, 1H), 8.00 (t, J=7.2 Hz, 2H), 7.7 (d, J=8.4 Hz, 2H), 7.68 (t, J=7.7 Hz, 1H), 7.33 (d, J=8.3 Hz, 2H), 4.48 (s, 2H), 3.37-3.32 (m, 1H), 2.78 (s, 3H), 0.57-0.53 (m, 2H), 0.49-0.43 (m, 2H).

### Example 43

### 5-(3-((4-((Benzylsulfonyl)methyl)phenyl)carbamoyl)phenyl)-2-methylnicotinic acid

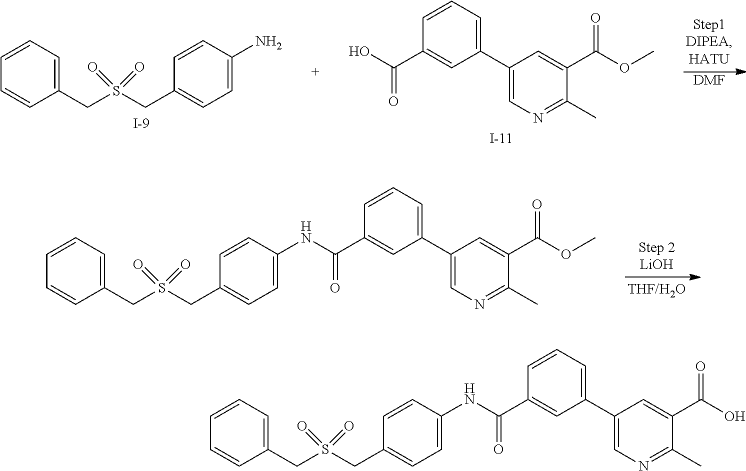

### Step 1: Methyl 5-(3-((4-((benzylsulfonyl)methyl)phenyl)carbamoyl)phenyl)-2-methylnicotinate

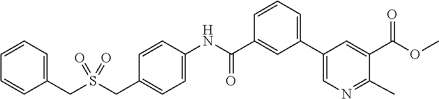 [SMILES: COC(=O)c1cc(-c2cccc(C(=O)Nc3ccc(CS(=O)(=O)Cc4ccccc4)cc3)c2)cnc1C]

Following the procedure of Example 39 Step 1, using 4-((benzylsulfonyl)methyl)aniline (I-9) in place of 4-((benzyloxy)methyl)aniline (I-3), was obtained methyl 5-(3-((4-((benzylsulfonyl)methyl)phenyl)carbamoyl)phenyl)-2-methylnicotinate in 61% yield.

LCMS: Method A, 1.82 min, MS: ES⁺ 515.1.

### Step 2: 5-(3-((4-((Benzylsulfonyl)methyl)phenyl)carbamoyl)phenyl)-2-methylnicotinic acid (Example 43)

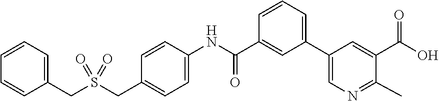 [SMILES: Cc1ncc(-c2cccc(C(=O)Nc3ccc(CS(=O)(=O)Cc4ccccc4)cc3)c2)cc1C(=O)O]

Following the procedure of Example 39 Step 2, using methyl 5-(3-((4-((benzylsulfonyl)-methyl)phenyl)carbamoyl)phenyl)-2-methylnicotinate in place of methyl 5-(3-((4-((benzyloxy)methyl)phenyl)carbamoyl)phenyl)-2-methylnicotinate, and purifying with basic modifier, was obtained 5-(3-((4-((benzylsulfonyl)methyl)phenyl)carbamoyl)phenyl)-2-methylnicotinic acid in 21% yield.

LCMS: Method A, 1.45 min, MS: ES⁺ 501.1. ¹H NMR (500 MHz, DMSO) δ ppm: 13.52-13.24 (m, 1H), 10.49 (s, 1H), 9.00 (d, J=2.8 Hz, 1H), 8.47 (d, J=2.8 Hz, 1H), 8.30 (s, 1H), 8.00 (t, J=8.2 Hz, 2H), 7.83 (d, J=8.2 Hz, 2H), 7.68 (t, J=7.7 Hz, 1H), 7.45-7.34 (m, 7H), 4.54-4.39 (m, 4H), 2.77 (s, 3H). The isolated compound contained up to 1 mol. eq. of ammonia.

### Example 44

### 5-(3-((4-((Cyclopropylmethoxy)methyl)phenyl)carbamoyl)phenyl)-2-methylnicotinic acid

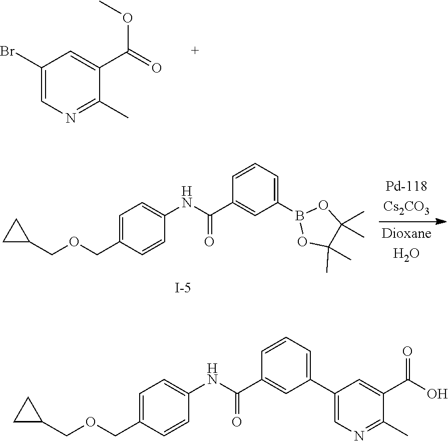

Following the procedure of Example 1, using N-(4-((cyclopropylmethoxy)methyl)phenyl)-3-(4,4,5,5-tetramethyl-1,3,2-dioxaborolan-2-yl)benzamide (I-5) in place of N-(4-phenethoxyphenyl)-3-(4,4,5,5-tetramethyl-1,3,2-dioxaborolan-2-yl)benzamide (I-1) and 5-bromo-2-methylnicotinate (CAS 1215916-40-7, BLD) in place of 5-bromo-2-hydroxybenzoic acid (CAS 89-55-4, Acros), was obtained 5-(3-((4-((cyclopropylmethoxy)methyl)phenyl)carbamoyl)phenyl)-2-methylnicotinic acid in 33% yield.

LCMS: Method A, 1.47 min, MS: ES⁺ 417.2. ¹H NMR (500 MHz, DMSO) δ ppm 10.23 (s, 1H), 8.61 (s, 1H), 8.11-8.06 (m, 2H), 7.79 (d, J=7.7 Hz, 1H), 7.74 (d, J=7.7 Hz, 1H), 7.62-7.57 (m, 2H), 7.47 (t, J=7.7 Hz, 1H), 7.15 (d, J=8.3 Hz, 2H), 4.28 (s, 2H), 3.11 (d, J=6.7 Hz, 2H), 2.52 (s, 3H), 0.92-0.81 (m, 1H), 0.34-0.27 (m, 2H), 0.11-−0.06 (m, 2H). 1 H was obscured/not observed. The isolated compound contained up to 1 mol. eq. of ammonia.

### Example 45

### 2-Chloro-5-(3-((4-((cyclopropylmethoxy)methyl)phenyl)carbamoyl)phenyl)nicotinic acid

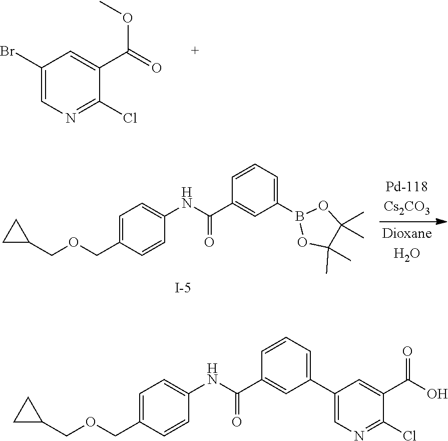

Following the procedure of Example 44, using methyl 5-bromo-2-chloronicotinate (CAS 78686-79-0, Fluorochem) in place of 5-bromo-2-methylnicotinate (CAS 1215916-40-7, BLD), was obtained 2-chloro-5-(3-((4-((cyclopropylmethoxy)methyl)phenyl)carbamoyl)-phenyl)nicotinic acid in 21% yield.

LCMS: Method A, 1.75 min, MS: ES⁺ 437.2. ¹H NMR (500 MHz, DMSO) δ ppm 10.22 (s, 1H), 8.55 (d, J=2.6 Hz, 1H), 8.14-8.09 (m, 1H), 8.07 (d, J=2.6 Hz, 1H), 7.84-7.79 (m, 1H), 7.79-7.75 (m, 1H), 7.61-7.55 (m, 2H), 7.47 (t, J=7.7 Hz, 1H), 7.14 (d, J=8.3 Hz, 2H), 4.27 (s, 2H), 3.10 (d, J=6.7 Hz, 2H), 0.92-0.80 (m, 1H), 0.33-0.25 (m, 2H), 0.04-−0.07 (m, 2H). 1H was obscured/not observed. The isolated compound contained up to 1 mol. eq. of ammonia.

### Example 46

### 6-(3-((6-Phenethoxypyridin-3-yl)carbamoyl)phenyl)picolinic acid

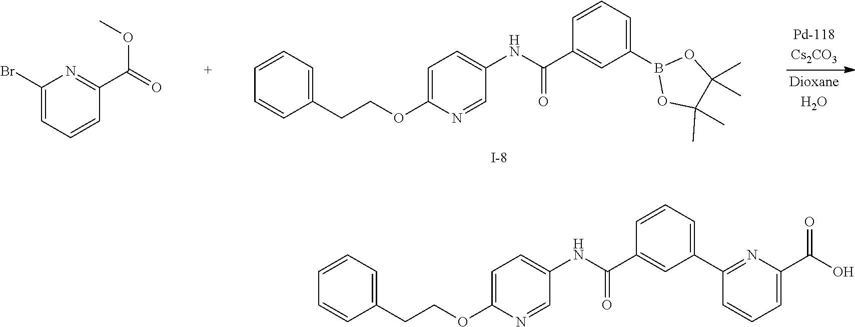

Following the procedure of Example 1, using N-(6-phenethoxypyridin-3-yl)-3-(4,4,5,5-tetramethyl-1,3,2-dioxaborolan-2-yl)benzamide (I-8) in place of N-(4-phenethoxyphenyl)-3-(4,4,5,5-tetramethyl-1,3,2-dioxaborolan-2-yl)benzamide (I-1) and methyl 6-bromopicolinate in place of 5-bromo-2-hydroxybenzoic acid (CAS 89-55-4, Acros), was obtained 6-(3-((6-phenethoxypyridin-3-yl)carbamoyl)phenyl)picolinic acid in 17% yield.

LCMS: Method A, 1.86 min, MS: ES⁺ 440.2. ¹H NMR (500 MHz, DMSO) δ ppm 10.49 (s, 1H), 8.67 (s, 1H), 8.55 (d, J=2.7 Hz, 1H), 8.34 (d, J=7.8 Hz, 1H), 8.12-8.05 (m, 2H), 8.01 (d, J=7.7 Hz, 1H), 7.96 (t, J=7.7 Hz, 1H), 7.88 (d, J=7.6 Hz, 1H), 7.67 (t, J=7.7 Hz 1H), 7.32 (d, J=4.3 Hz, 4H), 7.24 (h, J=4.2 Hz, 1H), 6.84 (d, J=8.9 Hz, 1H), 4.47 (t, J=6.9 Hz, 2H), 3.05 (t, J=6.9 Hz, 2H). 1H was obscured/not observed. The isolated compound contained up to 1 mol. eq. of ammonia.

### Example 47

### 5-(3-((4-((Benzyloxy)methyl)-2-fluorophenyl)carbamoyl)phenyl)-2-methylnicotinic acid

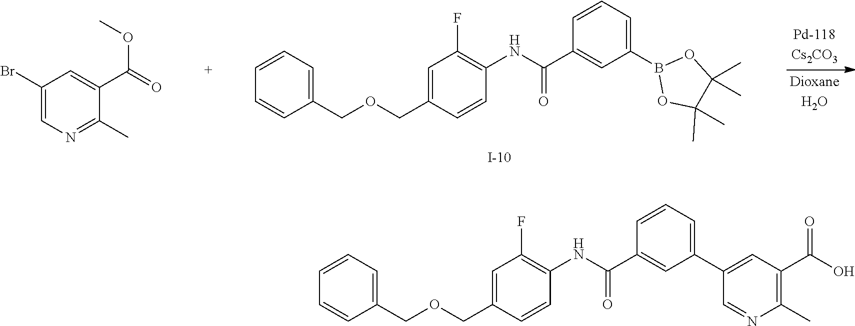

Following the procedure of Example 1, using N-(4-((benzyloxy)methyl)-2-fluorophenyl)-3-(4,4,5,5-tetramethyl-1,3,2-dioxaborolan-2-yl)benzamide (I-10) in place of N-(4-phenethoxyphenyl)-3-(4,4,5,5-tetramethyl-1,3,2-dioxaborolan-2-yl)benzamide (I-1) and methyl 5-bromo-2-methylnicotinate (CAS 1215916-40-7, BLD) in place of 5-bromo-2-hydroxybenzoic acid (CAS 89-55-4, Acros), was obtained 5-(3-((4-((benzyloxy)methyl)-2-fluorophenyl)carbamoyl)phenyl)-2-methylnicotinic acid in 17% yield.

LCMS: Method A, 1.66 min, MS: ES⁺ 471.2. ¹H NMR (500 MHz, DMSO) δ ppm 13.50 (s, 1H), 10.30 (s, 1H), 9.00 (d, J=2.5 Hz, 1H), 8.48 (d, J=2.5 Hz, 1H), 8.35 (s, 1H), 8.04-7.97 (m, 2H), 7.68 (t, J=7.7 Hz, 1H), 7.62 (t, J=8.0 Hz, 1H), 7.41-7.36 (m, 4H), 7.34-7.28 (m, 2H), 7.26-7.22 (m, 1H), 4.60-4.54 (m, 4H), 2.77 (s, 3H). The isolated compound contained up to 1 mol. eq. of ammonia.

### Example 48

### 6-(3-(4-(4-Phenylbutoxy)benzamido)phenyl)picolinic acid

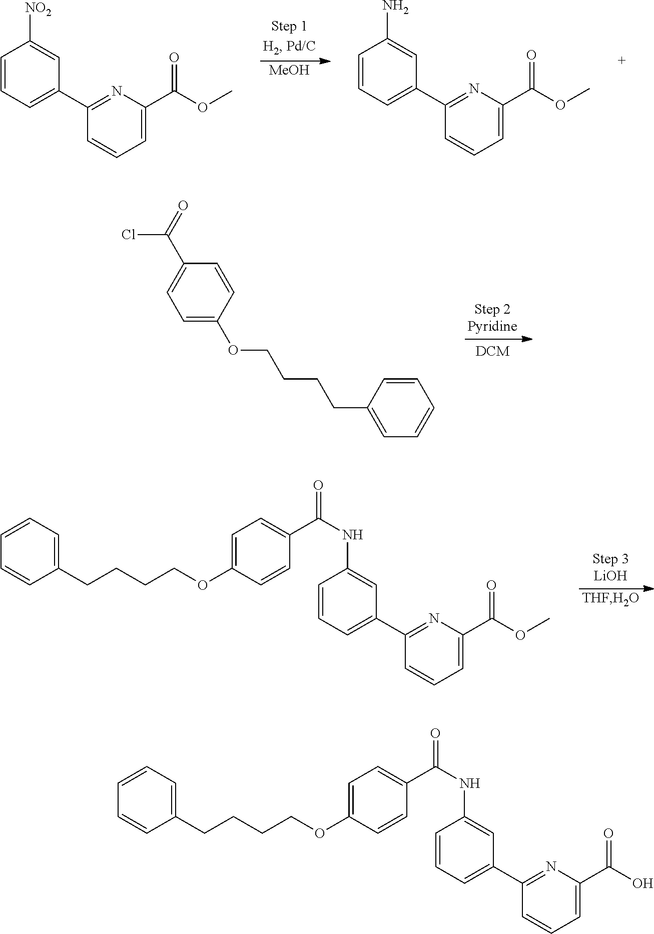

### Step 1: Methyl 6-(3-aminophenyl)picolinate

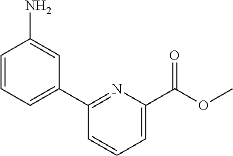 [SMILES: COC(=O)c1cccc(-c2cccc(N)c2)n1]

A solution of methyl 6-(3-nitrophenyl)picolinate (CAS 252921-23-6, Combi-Blocks, 200 mg, 0.78 mmol) in MeOH (15 mL) was passed once through a 10% Pd/C cartridge under hydrogen at 30° C. at 1 bar (H-Cube®). The reaction mixture was concentrated under reduced pressure to give methyl 6-(3-aminophenyl)picolinate (150 mg, 82%).

LCMS: Method A, 0.85 min, MS: ES⁺ 229.2.

### Step 2: Methyl 6-(3-(4-(4-phenylbutoxy)benzamido)phenyl)picolinate

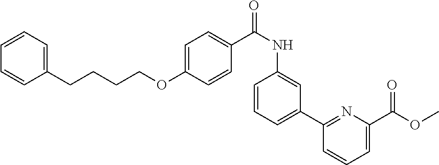 [SMILES: COC(=O)c1cccc(-c2cccc(NC(=O)c3ccc(OCCCCc4ccccc4)cc3)c2)n1]

To a solution of methyl 6-(3-aminophenyl)picolinate (60 mg, 0.26 mmol) in DCM (5 mL) was added 4-(4-phenylbutoxy)benzoyl chloride (CAS 108807-05-2, SAGEM, 98.7 mg, 0.34 mmol) then pyridine (0.03 mL, 0.37 mmol) was added dropwise at rt. The mixture was stirred at rt for 18 h, then diluted with sat. aq. NH₄Cl (10 mL) and extracted with DCM (3×20 mL). The combined organic phases were dried using a phase separator cartridge and concentrated under reduced pressure. The crude product was purified by flash chromatography (silica gel, 0-100% EtOAc in isohexane) to afford methyl 6-(3-(4-(4-phenylbutoxy)benzamido)phenyl)picolinate (60 mg, 47%) LCMS: Method A, 2.24 min, MS: ES⁺ 481.2.

### Step 3: 6-(3-(4-(4-Phenylbutoxy)benzamido)phenyl)picolinic acid (Example 48)

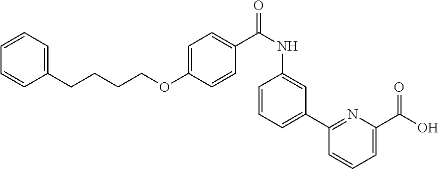 [SMILES: O=C(Nc1cccc(-c2cccc(C(=O)O)n2)c1)c1ccc(OCCCCc2ccccc2)cc1]

To a solution of methyl 6-(3-(4-(4-phenylbutoxy)benzamido)phenyl)picolinate (25 mg, 0.05 mmol) in THF (1 mL) was added LiOH (4 mg, 0.16 mmol) in water (1 mL) at rt. The reaction mixture was stirred at rt for 5 min, then acidified with HCl (1M, 0.18 mL) diluted with water (5 mL) and extracted with DCM (3×5 mL). The combined organic phases were dried over Na₂SO₄ and concentrated under reduced pressure. The crude product was triturated with acetonitrile then Et₂O to give 6-(3-(4-(4-phenylbutoxy)benzamido)phenyl)picolinic acid (18 mg, 73%).

LCMS: Method A, 2.12 min, MS: ES⁺ 467.2; ¹H NMR (500 MHz, DMSO) δ ppm: 10.29 (s, 1H), 8.50-8.46 (m, 1H), 8.10-8.02 (m, 2H), 8.02-7.95 (m, 3H), 7.97-7.92 (m, 1H), 7.85 (d, J=7.8 Hz, 1H), 7.49 (t, J=7.9 Hz, 1H), 7.30 (t, J=7.5 Hz, 2H), 7.26-7.21 (m, 2H), 7.19 (t, J=7.3 Hz, 1H), 7.09-7.04 (m, 2H), 4.12-4.06 (m, 2H), 2.70-2.64 (m, 2H), 1.79-1.70 (m, 4H). 1H was obscured/not observed. The isolated compound contained up to 1 mol. eq. of ammonia.

### Example 49

### 5-(5-((4-((Benzyloxy)methyl)phenyl)carbamoyl)thiazol-2-yl)-2-methylnicotinic acid

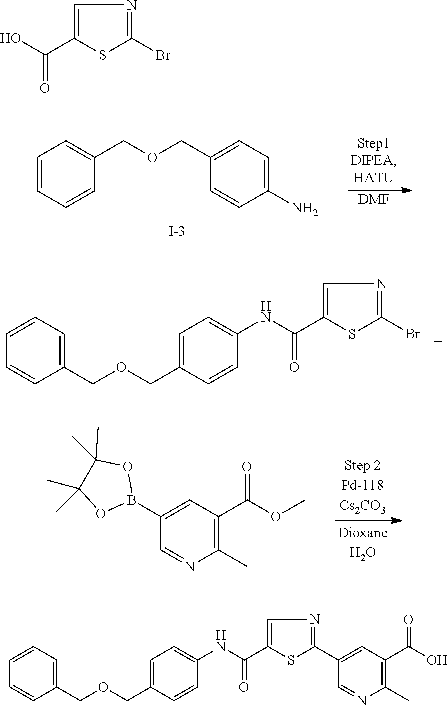

### Step 1: N-(4-((Benzyloxy)methyl)phenyl)-2-bromothiazole-5-carboxamide

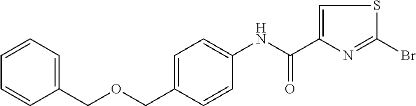 [SMILES: O=C(Nc1ccc(COCc2ccccc2)cc1)c1csc(Br)n1]

Following the procedure of Example 39 Step 1, using 2-bromothiazole-5-carboxylic acid (CAS 54045-76-0, BLD) in place of 3-(5-(methoxycarbonyl)-6-methylpyridin-3-yl)benzoic acid, was obtained N-(4-((benzyloxy)methyl)phenyl)-2-bromothiazole-5-carboxamide in 61% yield.

LCMS: Method A, 2.00 min, MS: ES⁺ 402.9/404.9.

### Step 2: 5-(5-((4-((Benzyloxy)methyl)phenyl)carbamoyl)thiazol-2-yl)-2-methylnicotinic acid (Example 49)

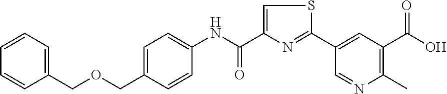 [SMILES: Cc1ncc(-c2nc(C(=O)Nc3ccc(COCc4ccccc4)cc3)cs2)cc1C(=O)O]

To a stirred solution of methyl 2-methyl-5-(4,4,5,5-tetramethyl-1,3,2-dioxaborolan-2-yl)nicotinate (89 mg, 0.32 mmol), and N-(4-((benzyloxy)methyl)phenyl)-2-bromothiazole-5-carboxamide (100 mg, 0.25 mmol) in 1,4-dioxane (4 mL) and water (1 mL) was added Cs₂CO₃ (323 mg, 0.99 mmol) and the reaction mixture was degassed with a flow of nitrogen at rt for 5 min. Pd-118 (0.03 g, 0.05 mmol) was added and the mixture degassed for a further 2 min. The reaction mixture was stirred at 80° C. for 18 h then cooled to rt, acidified with aq. HCl (1M, 5 mL) and extracted with EtOAc (3×10 mL). The combined organic phases were dried over Na₂SO₄ and concentrated under reduced pressure. The crude product was purified by RP flash chromatography (C18, 0-100% of 0.1% HCO₂H/MeCN in 0.1% aq. HCO₂H) to afford 5-(5-((4-((benzyloxy)methyl)phenyl)carbamoyl)thiazol-2-yl)-2-methylnicotinic acid (8 mg, 6% yield).

LCMS: Method A, 1.81 min, MS: ES⁺ 460.1; ¹H NMR (500 MHz, DMSO) δ ppm: 10.56 (s, 1H), 9.15 (s, 1H), 8.75 (s, 1H), 8.62 (s, 1H), 7.73 (d, J=8.2 Hz, 2H), 7.41-7.32 (m, 6H), 7.34-7.28 (m, 1H), 4.54 (s, 2H), 4.52 (s, 2H), 2.78 (s, 3H). 1H was obscured/not observed.

### Example 50

### 5-(4-((4-((Benzyloxy)methyl)phenyl)carbamoyl)thiazol-2-yl)-2-methylnicotinic acid

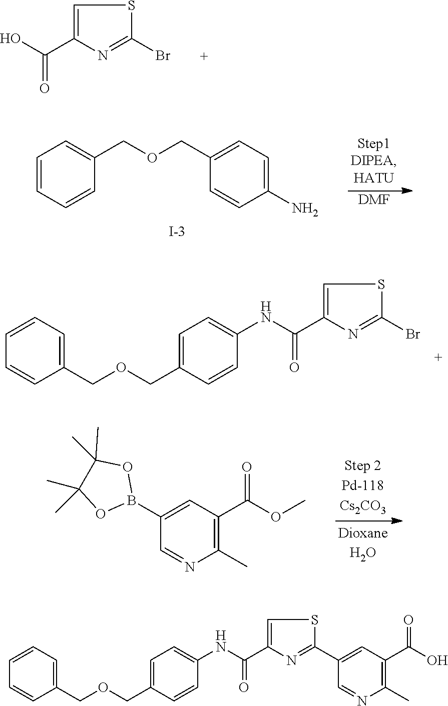

### Step 1: N-(4-((Benzyloxy)methyl)phenyl)-2-bromothiazole-4-carboxamide

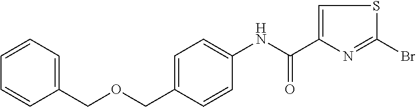 [SMILES: O=C(Nc1ccc(COCc2ccccc2)cc1)c1csc(Br)n1]

Following the procedure of Example 39 Step 1, using 2-bromothiazole-4-carboxylic acid (CAS 5198-88-9, BLD) in place of 3-(5-(methoxycarbonyl)-6-methylpyridin-3-yl)benzoic acid, was thus obtained N-(4-((benzyloxy)methyl)phenyl)-2-bromothiazole-4-carboxamide in 71% yield.

LCMS: Method A, 2.07 min, MS: ES⁺ 402.9/404.9.

### Step 2: 5-(4-((4-((Benzyloxy)methyl)phenyl)carbamoyl)thiazol-2-yl)-2-methylnicotinic acid (Example 50)

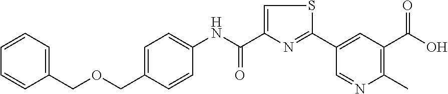 [SMILES: Cc1ncc(-c2nc(C(=O)Nc3ccc(COCc4ccccc4)cc3)cs2)cc1C(=O)O]

Following the procedure of Example 49 Step 2, using N-(4-((benzyloxy)methyl)phenyl)-2-bromothiazole-4-carboxamide in place of N-(4-((benzyloxy)methyl)phenyl)-2-bromothiazole-5-carboxamide, was obtained 5-(4-((4-((benzyloxy)methyl)phenyl)carbamoyl)thiazol-2-yl)-2-methylnicotinic acid in 38% yield.

LCMS: Method A, 1.87 min, MS: ES⁺ 460.1; ¹H NMR (500 MHz, DMSO) δ ppm: 13.62 (s, 1H), 10.38 (s, 1H), 9.36 (d, J=2.4 Hz, 1H), 8.77 (d, J=2.4 Hz, 1H), 8.57 (s, 1H), 7.86 (d, J=8.4 Hz, 2H), 7.38 (d, J=4.6 Hz, 6H), 7.34-7.29 (m, 1H), 4.55 (s, 2H), 4.53 (s, 2H), 2.81 (s, 3H).

### Example 51

### 6-Methyl-5′-((4-phenethoxyphenyl)carbamoyl)-[3,3′-bipyridine]-5-carboxylic acid

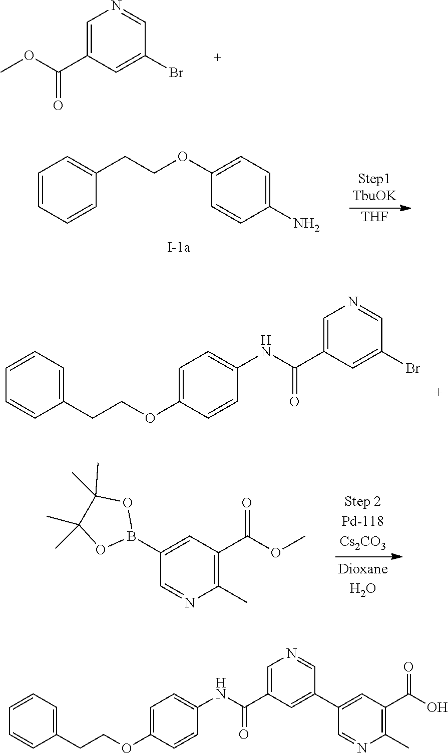

Step 1: 5-Bromo-N-(4-phenenethoxyphenyl)nicotinamide

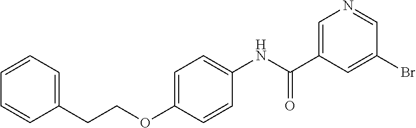 [SMILES: O=C(Nc1ccc(OCCc2ccccc2)cc1)c1cncc(Br)c1]

To methyl 5-bromopyridine-3-carboxylate (CAS 29681-44-5, Fluorochem, 166 mg, 0.77 mmol) and 4-phenethoxyaniline (I-1a, 200 mg, 0.77 mmol) in THF (5.00 mL) was added tBuOK (173 mg, 1.54 mmol). The mixture was stirred at rt for 4 h, quenched with water (0.1 mL) and concentrated under reduced pressure. The crude product was purified by flash chromatography (silica gel, 0-100% EtOAc in isohexane), followed by trituration with EtOAc to give 5-bromo-N-(4-phenethoxyphenyl)nicotinamide (50.0 mg, 12%).

¹H NMR (500 MHz, DMSO) δ ppm: 10.38 (s, 1H), 9.06 (d, J=2.0 Hz, 1H), 8.90 (d, J=2.0 Hz, 1H), 8.53 (s, 1H), 7.65 (d, J=8.9 Hz, 2H), 7.39-7.28 (m, 4H), 7.27-7.17 (m, 1H), 6.96 (d, J=8.9 Hz, 2H), 4.19 (t, J=6.9 Hz, 2H), 3.04 (t, J=6.9 Hz, 2H).

### Step 2: 6-Methyl-5′-((4-phenethoxyphenyl)carbamoyl)-[3,3′-bipyridine]-5-carboxylic acid (Example 51)

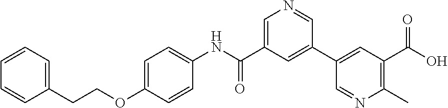 [SMILES: Cc1ncc(-c2cncc(C(=O)Nc3ccc(OCCc4ccccc4)cc3)c2)cc1C(=O)O]

Following the procedure of Example 49 Step 2, using 5-bromo-N-(4-phenethoxyphenyl)nicotinamide in place of N-(4-((benzyloxy)methyl)phenyl)-2-bromothiazole-5-carboxamide, was obtained 5′-((4-((benzyloxy)methyl)phenyl)carbamoyl)-6-methyl-[3,3′-bipyridine]-5-carboxylic acid in 29% yield.

LCMS: Method A, 1.65 min, MS: ES⁺ 454.2; ¹H NMR (500 MHz, DMSO) δ ppm: 10.33 (s, 1H), 9.08 (d, J=2.2 Hz, 1H), 9.03 (d, J=2.2 Hz, 1H), 9.00 (s, 1H), 8.56 (t, J=2.2 Hz, 1H), 8.48 (s, 1H), 7.60 (d, J=9.0 Hz, 2H), 7.31-7.22 (m, 4H), 7.19-7.13 (m, 1H), 6.90 (d, J=9.0 Hz, 2H), 4.12 (t, J=6.9 Hz, 2H), 2.97 (t, J=6.9 Hz, 2H), 2.72 (s, 3H). 1H was obscured/not observed.

### Example 52

### 5-(3-((4-((Benzyloxy)methyl)phenyl)carbamoyl)-4-fluorophenyl)-2-methylnicotinic acid

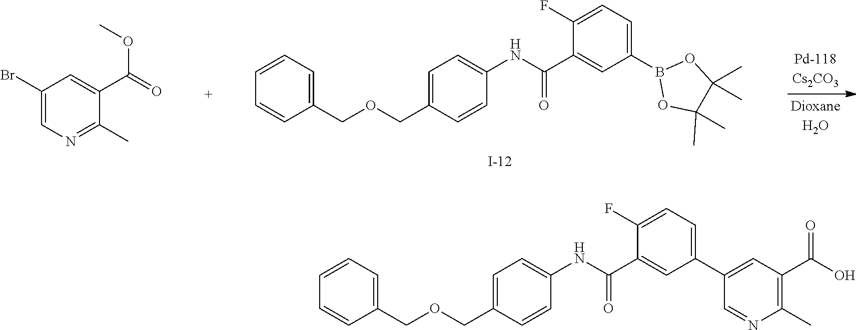

Following the procedure of Example 1, using N-(4-((benzyloxy)methyl)phenyl)-2-fluoro-5-(4,4,5,5-tetramethyl-1,3,2-dioxaborolan-2-yl)benzamide (I-12) in place of N-(4-phenethoxyphenyl)-3-(4,4,5,5-tetramethyl-1,3,2-dioxaborolan-2-yl)benzamide (I-1) and methyl 5-bromo-2-methylnicotinate (CAS 1215916-40-7, BLD) in place of 5-bromo-2-hydroxybenzoic acid (CAS 89-55-4, Acros), was obtained 5-(3-((4-((benzyloxy)methyl)-phenyl)carbamoyl)-4-fluorophenyl)-2-methylnicotinic acid in 46% yield.

LCMS: Method A, 1.70 min, MS: ES⁺ 471.2. ¹H NMR (500 MHz, DMSO) δ ppm 13.43 (s, 1H), 10.55 (s, 1H), 8.97 (d, J=2.4 Hz, 1H), 8.44 (d, J=2.5 Hz, 1H), 8.04 (dd, J=6.6, 2.5 Hz, 1H), 8.01-7.92 (m, 1H), 7.74 (d, J=8.2 Hz, 2H), 7.49 (t, J=9.2 Hz, 1H), 7.45-7.22 (m, 7H), 4.53 (s, 2H), 4.52 (s, 2H), 2.76 (s, 3H). The isolated compound contained up to 1 mol. eq. of ammonia.

### Example 53

### 6′-Methyl-4-((4-phenethoxyphenyl)carbamoyl)-[2,3′-bipyridine]-5′-carboxylic acid

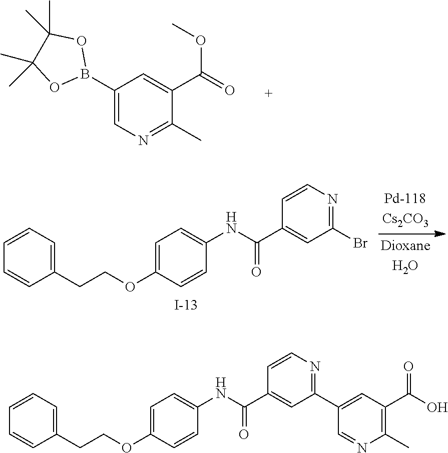

Following the procedure of Example 49 Step 2, using 2-bromo-N-(4-phenethoxyphenyl)isonicotinamide (I-13) in place of N-(4-((benzyloxy)methyl)phenyl)-2-bromothiazole-5-carboxamide, and purifying with basic modifier, was obtained 6′-methyl-4-((4-phenethoxyphenyl)-carbamoyl)-[2,3′-bipyridine]-5′-carboxylic acid in 13% yield.

LCMS: Method A, 1.70 min, MS: ES⁺ 454.2. ¹H NMR (500 MHz, DMSO) δ ppm 13.51 (br s, 1H), 10.48 (s, 1H), 9.35 (d, J=2.4 Hz, 1H), 8.89 (d, J=5.0 Hz, 1H), 8.87 (d, J=2.4 Hz, 1H), 8.52 (s, 1H), 7.86 (dd, J=5.0, 1.6 Hz, 1H), 7.69 (d, J=8.8 Hz, 2H), 7.38-7.29 (m, 4H), 7.27-7.22 (m, 1H), 6.99 (d, J=8.8 Hz, 2H), 4.20 (t, J=6.9 Hz, 2H), 3.05 (t, J=6.9 Hz, 2H), 2.80 (s, 3H). The isolated compound contained up to 1 mol. eq. of ammonia.

### Example 54

### 2-Methyl-5-(6-((4-phenethoxyphenyl)carbamoyl)pyrazin-2-yl)nicotinic acid

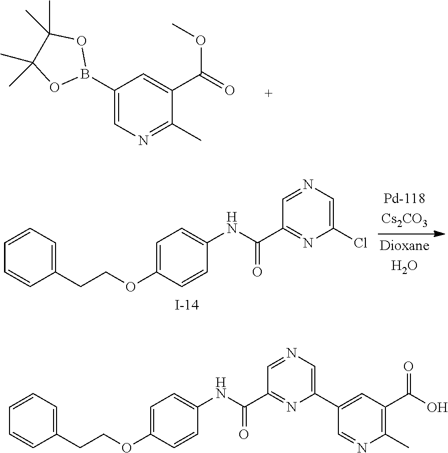

Following the procedure of Example 49 Step 2, using 6-chloro-N-(4-phenethoxyphenyl)-pyrazine-2-carboxamide (I-14) in place of N-(4-((benzyloxy)methyl)phenyl)-2-bromothiazole-5-carboxamide, and purifying with basic modifier, was obtained 2-methyl-5-(6-((4-phenethoxyphenyl)-carbamoyl)pyrazin-2-yl)nicotinic acid in 13% yield.

LCMS: Method A, 1.78 min, MS: ES⁺ 455.2. ¹H NMR (500 MHz, DMSO) δ ppm 10.60 (s, 1H), 9.50 (s, 1H), 9.40 (s, 1H), 9.20 (s, 1H), 8.74 (s, 1H), 7.75 (d, J=9.0 Hz, 2H), 7.42-7.29 (m, 4H), 7.28-7.19 (m, 1H), 6.98 (d, J=9.0 Hz, 2H), 4.20 (t, J=6.9 Hz, 2H), 3.05 (t, J=6.9 Hz, 2H), 2.71 (s, 3H). 1H was obscured/not observed. The isolated compound contained up to 1 mol. eq. of ammonia.

### Example 55

### 2-Methyl-5-(4-((4-phenethoxyphenyl)carbamoyl)pyrimidin-2-yl)nicotinic acid

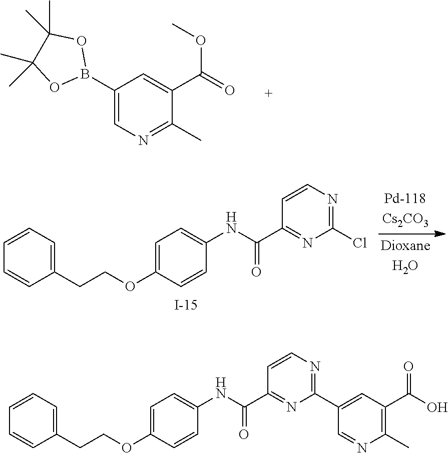

Following the procedure of Example 49 Step 2, using 2-chloro-N-(4-phenethoxyphenyl)pyrimidine-4-carboxamide (I-15) in place of N-(4-((benzyloxy)methyl)-phenyl)-2-bromothiazole-5-carboxamide, and purifying with basic modifier, was obtained 2-methyl-5-(4-((4-phenethoxy-phenyl)carbamoyl)pyrimidin-2-yl)nicotinic acid in 13% yield.

LCMS: Method A, 1.84 min, MS: ES⁺ 455.2. ¹H NMR (500 MHz, DMSO) δ ppm 13.48 (br s, 1H), 10.74 (s, 1H), 9.90 (d, J=2.3 Hz, 1H), 9.34-9.11 (m, 2H), 8.07 (d, J=5.0 Hz, 1H), 7.77 (d, J=9.0 Hz, 2H), 7.40-7.30 (m, 4H), 7.26-7.21 (m, 1H), 7.00 (d, J=9.0 Hz, 2H), 4.21 (t, J=6.9 Hz, 2H), 3.05 (t, J=6.9 Hz, 2H), 2.83 (s, 3H). The isolated compound contained up to 1 mol. eq. of ammonia.

### Examples 56-95

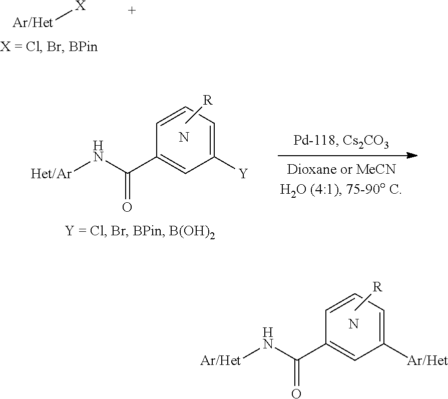

The following Examples 56-95 were prepared analogously to Example 1, substituting 5-bromo-2-hydroxybenzoic acid (CAS 89-55-4) and N-(4-phenethoxyphenyl)-3-(4,4,5,5-tetramethyl-1,3,2-dioxaborolan-2-yl)benzamide (I-1) with the appropriate starting materials, using Pd-118 (0.1-0.2 eq), MeCN or dioxane at 75-90° C. for 18-72 h, and with any minor modifications described. In some cases, an ester was used in place of a carboxylic acid, and under the reaction conditions partial or complete hydrolysis to the acid product occurred. All compounds may contain up to 1 eq ammonia.

### Example 56

### 5-[3-[[4-(Benzyloxymethyl)-3-fluoro-phenyl]carbamoyl]phenyl]-2-methyl-pyridine-3-carboxylic acid

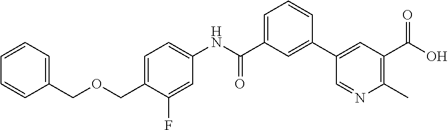 [SMILES: Cc1ncc(-c2cccc(C(=O)Nc3ccc(COCc4ccccc4)c(F)c3)c2)cc1C(=O)O]

Using N-(4-((benzyloxy)methyl)-3-fluorophenyl)-3-(4,4,5,5-tetramethyl-1,3,2-dioxaborolan-2-yl)benzamide (I-16) and methyl 5-bromo-2-methylnicotinate (CAS 1215916-40-7, BLD) was obtained 5-[3-[[4-(benzyloxymethyl)-3-fluoro-phenyl]carbamoyl]phenyl]-2-methyl-pyridine-3-carboxylic acid in 23% yield.

LCMS: Method A: 1.75 min, MS: ES⁺ 471.2; ¹H NMR (500 MHz, DMSO) δ ppm 10.58 (s, 1H), 8.93 (d, J=2.5 Hz, 1H), 8.40 (d, J=2.5 Hz, 1H), 8.30-8.26 (m, 1H), 8.01-7.95 (m, 2H), 7.80 (dd, J=12.5, 2.0 Hz, 1H), 7.68 (t, J=7.8 Hz, 1H), 7.62-7.56 (m, 1H), 7.47 (t, J=8.4 Hz, 1H), 7.41-7.35 (m, 4H), 7.35-7.27 (m, 1H), 4.58-4.54 (m, 4H), 2.75 (s, 3H). 1H obscured/not observed.

### Example 57

### 2-Methyl-5-[3-[[1-(2-phenylethyl)pyrazol-4-yl]carbamoyl]phenyl]pyridine-3-carboxylic acid

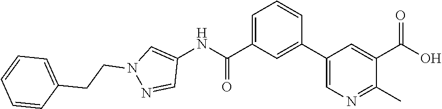 [SMILES: Cc1ncc(-c2cccc(C(=O)Nc3cnn(CCc4ccccc4)c3)c2)cc1C(=O)O]

Using 3-bromo-N-(1-phenethyl-1H-pyrazol-4-yl)benzamide (I-49) and methyl 2-methyl-5-(4,4,5,5-tetramethyl-1,3,2-dioxaborolan-2-yl)nicotinate (CAS 2291165-13-2, Combi-Blocks), also K₃PO₄ (3 eq) in place of Cs₂CO₃, and after 18 h at 80° C., stirring at 40° C. with LiOH (2 eq) for 3 h was obtained 2-methyl-5-(3-((1-phenethyl-1H-pyrazol-4-yl)carbamoyl)phenyl)-nicotinic acid in 74% yield.

LCMS: Method A: 1.37 min, MS: ES⁺ 427.2; ¹H NMR (400 MHz, DMSO) δ ppm 10.58 (s, 1H), 8.72 (d, J=2.5 Hz, 1H), 8.23 (t, J=1.9 Hz, 1H), 8.20 (d, J=2.5 Hz, 1H), 8.02 (s, 1H), 7.96-7.92 (m, 1H), 7.90-7.85 (m, 1H), 7.65-7.58 (m, 2H), 7.31-7.25 (m, 2H), 7.21 (d, J=7.3 Hz, 3H), 4.37-4.31 (m, 2H), 3.11 (t, J=7.3 Hz, 2H), 2.67 (s, 3H). 1H obscured/not observed.

### Example 58

### 2-Methyl-5-[3-[[4-(p-tolylsulfonylmethyl)phenyl]carbamoyl]phenyl]pyridine-3-carboxylic acid

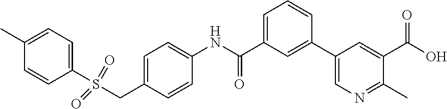 [SMILES: Cc1ccc(S(=O)(=O)Cc2ccc(NC(=O)c3cccc(-c4cnc(C)c(C(=O)O)c4)c3)cc2)cc1]

Using 3-bromo-N-(4-(tosylmethyl)phenyl)benzamide (I-50) and methyl 2-methyl-5-(4,4,5,5-tetramethyl-1,3,2-dioxaborolan-2-yl)nicotinate (CAS 2291165-13-2, Combi-Blocks), also K₃PO₄ (3 eq) in place of Cs₂CO₃, and after 18 h at 80° C., stirring at 40° C. with LiOH (2 eq) for 3 h was obtained 2-methyl-5-[3-[[4-(p-tolylsulfonylmethyl)phenyl]carbamoyl]phenyl]pyridine-3-carboxylic acid in 69% yield.

LCMS: Method A: 1.48 min, MS: ES⁺ 501.1; ¹H NMR (400 MHz, DMSO) δ ppm 10.47 (s, 1H), 8.63 (d, J=2.5 Hz, 1H), 8.23 (t, J=1.9 Hz, 1H), 8.12 (d, J=2.5 Hz, 1H), 7.95-7.90 (m, 1H), 7.87 (d, J=7.5 Hz, 1H), 7.73-7.67 (m, 2H), 7.65-7.55 (m, 3H), 7.40 (d, J=8.0 Hz, 2H), 7.14-7.08 (m, 2H), 4.60 (s, 2H), 2.64 (s, 3H), 2.40 (s, 3H). 1H obscured/not observed.

### Example 59

### 2-Methyl-5-[3-[[4-(1-phenylethoxymethyl)phenyl]carbamoyl]phenyl]pyridine-3-carboxylic acid

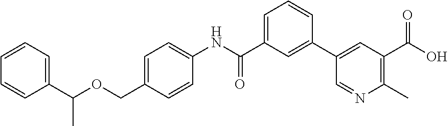 [SMILES: Cc1ncc(-c2cccc(C(=O)Nc3ccc(COC(C)c4ccccc4)cc3)c2)cc1C(=O)O]

Using N-(4-((1-phenylethoxy)methyl)phenyl)-3-(4,4,5,5-tetramethyl-1,3,2-dioxaborolan-2-yl)benzamide (I-19) and methyl 5-bromo-2-methylnicotinate (CAS 1215916-40-7, BLD) was obtained 2-methyl-5-[3-[[4-(1-phenylethoxymethyl)phenyl]carbamoyl]phenyl]pyridine-3-carboxylic acid in 8% yield.

LCMS: Method A: 1.76 min, MS: ES⁺ 467.2; ¹H NMR (500 MHz, DMSO) δ ppm 10.39 (s, 1H), 8.98 (s, 1H), 8.45 (s, 1H), 8.29 (s, 1H), 8.04-7.92 (m, 2H), 7.76 (d, J=8.5 Hz, 2H), 7.67 (t, J=7.8 Hz, 1H), 7.41-7.36 (m, 4H), 7.33-7.28 (m, 3H), 4.59-4.51 (m, 1H), 4.37-4.25 (m, 2H), 2.77 (s, 3H), 1.40 (d, J=6.4 Hz, 3H). 1H obscured/not observed.

### Example 60

### 5-[3-[[4-(1-Cyclopropylethoxy)phenyl]carbamoyl]phenyl]-2-methyl-pyridine-3-carboxylic acid

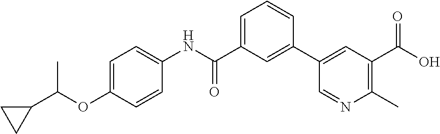 [SMILES: Cc1ncc(-c2cccc(C(=O)Nc3ccc(OC(C)C4CC4)cc3)c2)cc1C(=O)O]

Using N-(4-(1-cyclopropylethoxy)phenyl)-3-(4,4,5,5-tetramethyl-1,3,2-dioxaborolan-2-yl)benzamide (I-20) and methyl 5-bromo-2-methylnicotinate (CAS 1215916-40-7, BLD) was obtained 5-[3-[[4-(1-cyclopropylethoxy)phenyl]carbamoyl]phenyl]-2-methyl-pyridine-3-carboxylic acid in 52% yield.

LCMS: Method A: 1.61 min, MS: ES⁺ 417.2; ¹H NMR (500 MHz, DMSO) δ ppm 10.25 (s, 1H), 8.89 (d, J=2.6 Hz, 1H), 8.36 (s, 1H), 8.26 (s, 1H), 7.95 (dd, J=17.6, 7.7 Hz, 2H), 7.65 (d, J=8.3 Hz, 3H), 6.93 (d, J=8.9 Hz, 2H), 3.97-3.84 (m, 1H), 2.73 (s, 3H), 1.28 (d, J=6.0 Hz, 3H), 1.13-0.99 (m, 1H), 0.49 (dd, J=8.4, 2.5 Hz, 2H), 0.39-0.23 (m, 2H). 1H obscured/not observed.

### Example 61

### 5-[3-[[4-(Benzyloxymethyl)-2-methoxy-phenyl]carbamoyl]phenyl]-2-methyl-pyridine-3-carboxylic acid

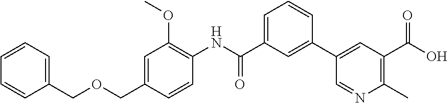 [SMILES: COc1cc(COCc2ccccc2)ccc1NC(=O)c1cccc(-c2cnc(C)c(C(=O)O)c2)c1]

Using N-(4-((benzyloxy)methyl)-2-methoxyphenyl)-3-(4,4,5,5-tetramethyl-1,3,2-dioxaborolan-2-yl)benzamide (I-21) and 5-bromo-2-methylnicotinic acid (CAS 351003-02-6, Combi-Blocks) was obtained 5-[3-[[4-(benzyloxymethyl)-2-methoxy-phenyl]carbamoyl]phenyl]-2-methyl-pyridine-3-carboxylic acid in 17% yield.

LCMS: Method A: 1.83 min, MS: ES⁺ 483.2; ¹H NMR (400 MHz, DMSO) δ ppm 9.70 (s, 1H), 9.03 (d, J=2.5 Hz, 1H), 8.50 (d, J=2.4 Hz, 1H), 8.33 (s, 1H), 7.99 (dt, J=7.9, 1.8 Hz, 2H), 7.71-7.62 (m, 2H), 7.42-7.34 (m, 4H), 7.34-7.27 (m, 1H), 7.09 (d, J=1.7 Hz, 1H), 6.98 (dd, J=8.1, 1.7 Hz, 1H), 4.56 (s, 2H), 4.55 (s, 2H), 3.84 (s, 3H), 2.78 (s, 3H). 1H obscured/not observed.

### Example 62

### 5-[3-[[3-Fluoro-4-(2-pyridylmethoxymethyl)phenyl]carbamoyl]phenyl]-2-methyl-pyridine-3-carboxylic acid

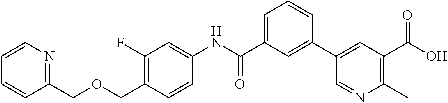 [SMILES: Cc1ncc(-c2cccc(C(=O)Nc3ccc(COCc4ccccn4)c(F)c3)c2)cc1C(=O)O]

Using N-(3-fluoro-4-((pyridin-2-ylmethoxy)methyl)phenyl)-3-(4,4,5,5-tetramethyl-1,3,2-dioxaborolan-2-yl)benzamide (I-22) and 5-bromo-2-methylnicotinic acid (CAS 351003-02-6, Combi-Blocks) was obtained 5-[3-[[3-fluoro-4-(2-pyridylmethoxymethyl)phenyl]carbamoyl]phenyl]-2-methyl-pyridine-3-carboxylic acid in 15% yield.

LCMS: Method A: 0.80 min, MS: ES⁺ 472.2; ¹H NMR (500 MHz, DMSO) δ ppm 10.60 (s, 1H), 8.86 (s, 1H), 8.59-8.51 (m, 1H), 8.33 (s, 1H), 8.27 (t, J=1.9 Hz, 1H), 8.03-7.91 (m, 2H), 7.87-7.77 (m, 2H), 7.67 (t, J=7.8 Hz, 1H), 7.60 (dd, J=8.4, 2.1 Hz, 1H), 7.55-7.44 (m, 2H), 7.36-7.27 (m, 1H), 4.66-4.62 (m, 4H), 2.72 (s, 3H). 1H obscured/not observed.

### Example 63

### 5-[3-[[3-Fluoro-4-[(4-methoxyphenyl)methoxymethyl]phenyl]carbamoyl]phenyl]-2-methyl-pyridine-3-carboxylic acid

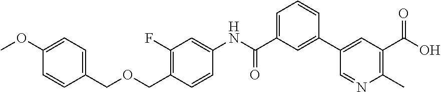 [SMILES: COc1ccc(COCc2ccc(NC(=O)c3cccc(-c4cnc(C)c(C(=O)O)c4)c3)cc2F)cc1]

Using N-(3-fluoro-4-(((4-methoxybenzyl)oxy)methyl)phenyl)-3-(4,4,5,5-tetramethyl-1,3,2-dioxaborolan-2-yl)benzamide (I-23) and 5-bromo-2-methylnicotinic acid (CAS 351003-02-6, Combi-Blocks) was obtained 5-[3-[[3-fluoro-4-[(4-methoxyphenyl)methoxymethyl]phenyl]carbamoyl]phenyl]-2-methyl-pyridine-3-carboxylic acid in 40% yield.

LCMS: Method A: 1.41 min, MS: ES⁺ 501.2; ¹H NMR (500 MHz, DMSO) δ ppm 10.60 (s, 1H), 8.89 (s, 1H), 8.36 (s, 1H), 8.28 (s, 1H), 8.00-7.91 (m, 2H), 7.79 (dd, J=12.5, 2.0 Hz, 1H), 7.66 (t, J=7.7 Hz, 1H), 7.58 (dd, J=8.3, 2.0 Hz, 1H), 7.44 (t, J=8.4 Hz, 1H), 7.29 (d, J=8.6 Hz, 2H), 6.93 (d, J=8.5 Hz, 2H), 4.51 (s, 2H), 4.47 (s, 2H), 3.76 (s, 3H), 2.73 (s, 3H). 1H obscured/not observed.

### Example 64

### 5-[3-[[3-Fluoro-4-[(3-methoxyphenyl)methoxymethyl]phenyl]carbamoyl]phenyl]-2-methyl-pyridine-3-carboxylic acid

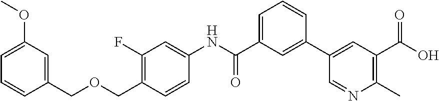 [SMILES: COc1cccc(COCc2ccc(NC(=O)c3cccc(-c4cnc(C)c(C(=O)O)c4)c3)cc2F)c1]

Using N-(3-fluoro-4-(((3-methoxybenzyl)oxy)methyl)phenyl)-3-(4,4,5,5-tetramethyl-1,3,2-dioxaborolan-2-yl)benzamide (I-24) and 5-bromo-2-methylnicotinic acid (CAS 351003-02-6, Combi-Blocks) was obtained 5-[3-[[3-fluoro-4-[(3-methoxyphenyl)methoxymethyl]phenyl]-carbamoyl]phenyl]-2-methyl-pyridine-3-carboxylic acid in 22% yield.

LCMS: Method A: 1.43 min, MS: ES⁺ 501.2; ¹H NMR (500 MHz, DMSO) δ ppm 13.43 (s, 1H), 10.57 (s, 1H), 9.01 (d, J=2.4 Hz, 1H), 8.47 (d, J=2.4 Hz, 1H), 8.29 (s, 1H), 8.00 (dd, J=7.8, 1.8 Hz, 2H), 7.80 (dd, J=12.5, 2.1 Hz, 1H), 7.69 (t, J=7.8 Hz, 1H), 7.59 (dd, J=8.4, 2.1 Hz, 1H), 7.47 (t, J=8.4 Hz, 1H), 7.28 (t, J=7.8 Hz, 1H), 6.97-6.89 (m, 2H), 6.87 (dd, J=8.0, 2.8 Hz, 1H), 4.55 (s, 2H), 4.53 (s, 2H), 3.76 (s, 3H), 2.78 (s, 3H).

### Examples 65 and 66

### 6-Fluoro-5-[3-[[4-(2-phenylethoxy)phenyl]carbamoyl]phenyl]pyridine-3-carboxylic acid and 6-hydroxy-5-[3-[[4-(2-phenylethoxy)phenyl]carbamoyl]-phenyl]pyridine-3-carboxylic acid

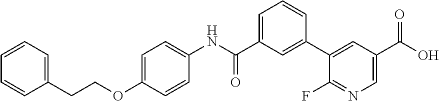 [SMILES: O=C(O)c1cnc(F)c(-c2cccc(C(=O)Nc3ccc(OCCc4ccccc4)cc3)c2)c1]

Using N-(4-phenethoxyphenyl)-3-(4,4,5,5-tetramethyl-1,3,2-dioxaborolan-2-yl)benzamide (I-1) and 5-bromo-6-fluoronicotinic acid (CAS 29241-63-2, Manchester Organics) was obtained 6-fluoro-5-[3-[[4-(2-phenylethoxy)phenyl]carbamoyl]phenyl]pyridine-3-carboxylic acid in 42% yield.

LCMS: Method A: 1.96 min, MS: ES⁺ 457.2; ¹H NMR (500 MHz, DMSO) δ ppm 10.19 (s, 1H), 8.21 (s, 1H), 8.09 (d, J=2.5 Hz, 1H), 8.00 (d, J=2.5 Hz, 1H), 7.94 (d, J=7.7 Hz, 1H), 7.90 (d, J=7.7 Hz, 1H), 7.69-7.64 (m, 2H), 7.55 (t, J=7.7 Hz, 1H), 7.38-7.29 (m, 4H), 7.27-7.20 (m, 1H), 6.98-6.91 (m, 2H), 4.19 (t, J=6.9 Hz, 2H), 3.04 (t, J=6.9 Hz, 2H). 1H obscured/not observed.

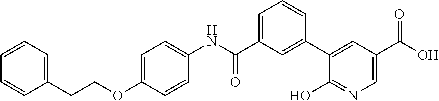 [SMILES: O=C(O)c1cnc(O)c(-c2cccc(C(=O)Nc3ccc(OCCc4ccccc4)cc3)c2)c1]

Also isolated was 6-hydroxy-5-[3-[[4-(2-phenylethoxy)phenyl]carbamoyl]phenyl]pyridine-3-carboxylic acid in 20% yield.

LCMS: Method A: 1.66 min, MS: ES⁺ 455.1; ¹H NMR (500 MHz, DMSO) δ ppm 10.19 (s, 1H), 8.21 (s, 1H), 8.09 (d, J=2.5 Hz, 1H), 8.00 (d, J=2.5 Hz, 1H), 7.94 (d, J=7.7 Hz, 1H), 7.90 (d, J=7.7 Hz, 1H), 7.69-7.64 (m, 2H), 7.55 (t, J=7.7 Hz, 1H), 7.38-7.29 (m, 4H), 7.27-7.20 (m, 1H), 6.98-6.91 (m, 2H), 4.19 (t, J=6.9 Hz, 2H), 3.04 (t, J=6.9 Hz, 2H). 2H obscured/not observed.

### Example 67

### 6-[3-[[4-(Benzyloxymethyl)phenyl]carbamoyl]phenyl]pyridazine-4-carboxylic acid

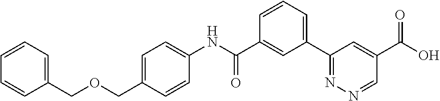 [SMILES: O=C(O)c1cnnc(-c2cccc(C(=O)Nc3ccc(COCc4ccccc4)cc3)c2)c1]

Using N-(4-((benzyloxy)methyl)phenyl)-3-(4,4,5,5-tetramethyl-1,3,2-dioxaborolan-2-yl)benzamide (I-25) and 6-chloropyridazine-4-carboxylic acid (CAS 1256794-24-7, BLD) was obtained 6-[3-[[4-(benzyloxymethyl)phenyl]carbamoyl]phenyl]pyridazine-4-carboxylic acid in 14% yield.

LCMS: Method A: 1.44 min, MS: ES⁺ 440.2; ¹H NMR (500 MHz, DMSO) δ ppm 10.54 (s, 1H), 9.41 (d, J=1.7 Hz, 1H), 8.71 (s, 1H), 8.42 (d, J=1.8 Hz, 1H), 8.39 (d, J=1.5 Hz, 1H), 8.09 (d, J=7.8 Hz, 1H), 7.80 (d, J=8.5 Hz, 2H), 7.71 (t, J=7.8 Hz, 1H), 7.40-7.33 (m, 6H), 7.33-7.26 (m, 1H), 4.53 (s, 2H), 4.52 (s, 2H). 1H obscured/not observed.

### Example 68

### 6-[3-[[4-(Benzyloxymethyl)phenyl]carbamoyl]phenyl]-3-methyl-pyrazine-2-carboxylic acid

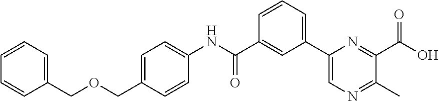 [SMILES: Cc1ncc(-c2cccc(C(=O)Nc3ccc(COCc4ccccc4)cc3)c2)nc1C(=O)O]

Using N-(4-((benzyloxy)methyl)phenyl)-3-(4,4,5,5-tetramethyl-1,3,2-dioxaborolan-2-yl)benzamide (I-25) and methyl 6-chloro-3-methylpyrazine-2-carboxylate (CAS 1166831-45-3, Combi-Blocks) was obtained 6-[3-[[4-(benzyloxymethyl)phenyl]carbamoyl]phenyl]-3-methyl-pyrazine-2-carboxylic acid in 40% yield.

LCMS: Method A: 1.53 min, MS: ES⁺ 454.2; ¹H NMR (500 MHz, DMSO) δ ppm 10.43 (s, 1H), 9.11 (s, 1H), 8.63 (s, 1H), 8.30 (d, J=7.9 Hz, 1H), 8.08-7.93 (m, 1H), 7.80 (d, J=8.5 Hz, 2H), 7.67 (t, J=7.7 Hz, 1H), 7.41-7.34 (m, 6H), 7.33-7.28 (m, 1H), 4.53 (s, 2H), 4.51 (s, 2H), 2.58 (s, 3H). 1H obscured/not observed.

### Example 69

### 2-Methyl-5-[6-[[4-(2-phenylethoxy)phenyl]carbamoyl]pyrimidin-4-yl]pyridine-3-carboxylic acid

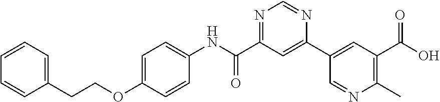 [SMILES: Cc1ncc(-c2cc(C(=O)Nc3ccc(OCCc4ccccc4)cc3)ncn2)cc1C(=O)O]

Using 6-chloro-N-(4-phenethoxyphenyl)pyrimidine-4-carboxamide (I-26) and methyl 2-methyl-5-(4,4,5,5-tetramethyl-1,3,2-dioxaborolan-2-yl)nicotinate (CAS 2291165-13-2, Combi-Blocks) was obtained 2-methyl-5-[6-[[4-(2-phenylethoxy)phenyl]carbamoyl]pyrimidin-4-yl]pyridine-3-carboxylic acid in 20% yield.

LCMS: Method A: 1.89 min, MS: ES⁺ 455.1; ¹H NMR (500 MHz, DMSO) δ ppm 10.79 (s, 1H), 9.47 (d, J=1.3 Hz, 1H), 9.37 (d, J=2.4 Hz, 1H), 8.86 (d, J=2.4 Hz, 1H), 8.66 (d, J=1.3 Hz, 1H), 7.84 (d, J=9.1 Hz, 2H), 7.43-7.28 (m, 4H), 7.28-7.12 (m, 1H), 6.98 (d, J=9.1 Hz, 2H), 4.21 (t, J=6.9 Hz, 2H), 3.05 (t, J=6.9 Hz, 2H), 2.80 (s, 3H). 1H obscured/not observed.

### Example 70

### 2-Methyl-5-[2-[[4-(2-phenylethoxy)phenyl]carbamoyl]-4-pyridyl]pyridine-3-carboxylic acid

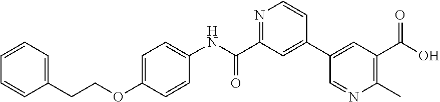 [SMILES: Cc1ncc(-c2ccnc(C(=O)Nc3ccc(OCCc4ccccc4)cc3)c2)cc1C(=O)O]

Using 4-bromo-N-(4-phenethoxyphenyl)picolinamide (I-27) and methyl 2-methyl-5-(4,4,5,5-tetramethyl-1,3,2-dioxaborolan-2-yl)nicotinate (CAS 2291165-13-2, Combi-Blocks) was obtained 2-methyl-5-[2-[[4-(2-phenylethoxy)phenyl]carbamoyl]-4-pyridyl]pyridine-3-carboxylic acid in 45% yield.

LCMS: Method A: 1.89 min, MS: ES⁺ 454.2; ¹H NMR (500 MHz, DMSO) δ ppm 10.59 (s, 1H), 8.83-8.72 (m, 2H), 8.37 (s, 1H), 8.29-8.20 (m, 1H), 8.01 (dd, J=5.1, 1.9 Hz, 1H), 7.83 (d, J=9.0 Hz, 2H), 7.37-7.28 (m, 4H), 7.25-7.20 (m, 1H), 6.95 (d, J=9.0 Hz, 2H), 4.19 (t, J=6.9 Hz, 2H), 3.04 (t, J=6.9 Hz, 2H), 2.71 (s, 3H). 1H obscured/not observed.

### Example 71

### 2-Methyl-5-[5-[[4-(2-phenylethoxy)phenyl]carbamoyl]pyridazin-3-yl]pyridine-3-carboxylic acid

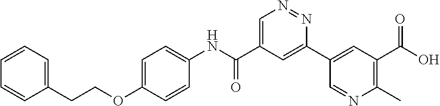 [SMILES: Cc1ncc(-c2cc(C(=O)Nc3ccc(OCCc4ccccc4)cc3)cnn2)cc1C(=O)O]

Using 6-chloro-N-(4-phenethoxyphenyl)pyridazine-4-carboxamide (I-28) and methyl 2-methyl-5-(4,4,5,5-tetramethyl-1,3,2-dioxaborolan-2-yl)nicotinate (CAS 2291165-13-2, Combi-Blocks) was obtained 2-methyl-5-[5-[[4-(2-phenylethoxy)phenyl]carbamoyl]pyridazin-3-yl]pyridine-3-carboxylic acid in 22% yield.

LCMS: Method A: 1.68 min, MS: ES⁺ 455.2; ¹H NMR (400 MHz, DMSO) δ ppm 11.04 (s, 1H), 9.54 (d, J=2.0 Hz, 1H), 9.11 (d, J=2.4 Hz, 1H), 8.77 (d, J=2.0 Hz, 1H), 8.60 (d, J=2.4 Hz, 1H), 7.71 (d, J=9.0 Hz, 2H), 7.42-7.27 (m, 4H), 7.27-7.13 (m, 1H), 6.98 (d, J=9.1 Hz, 2H), 4.20 (t, J=6.9 Hz, 2H), 3.04 (t, J=6.9 Hz, 2H), 2.69 (s, 3H). 1H obscured/not observed.

### Example 72

### 5-[4-[[4-(Cyclopropylmethoxy)phenyl]carbamoyl]-2-pyridyl]-2-methyl-pyridine-3-carboxylic acid

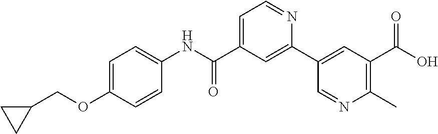 [SMILES: Cc1ncc(-c2cc(C(=O)Nc3ccc(OCC4CC4)cc3)ccn2)cc1C(=O)O]

Using 2-bromo-N-(4-(cyclopropylmethoxy)phenyl)isonicotinamide (I-29) and methyl 2-methyl-5-(4,4,5,5-tetramethyl-1,3,2-dioxaborolan-2-yl)nicotinate (CAS 2291165-13-2, Combi-Blocks) was obtained 5-[4-[[4-(cyclopropylmethoxy)phenyl]carbamoyl]-2-pyridyl]-2-methyl-pyridine-3-carboxylic acid in 67% yield.

LCMS: Method A: 1.45 min, MS: ES⁺ 404.2; ¹H NMR (500 MHz, DMSO) δ ppm 10.53 (s, 1H), 9.14 (s, 1H), 8.86 (d, J=5.0 Hz, 1H), 8.64 (d, J=2.4 Hz, 1H), 8.46 (s, 1H), 7.82 (d, J=5.0 Hz, 1H), 7.68 (d, J=8.9 Hz, 2H), 6.96 (d, J=8.6 Hz, 2H), 3.82 (d, J=6.9 Hz, 2H), 2.72 (s, 3H), 1.44-1.10 (m, 1H), 0.73-0.49 (m, 2H), 0.33 (d, J=4.9 Hz, 2H). 1H obscured/not observed.

### Example 73

### 5-[4-[[4-(Cyclopropylmethoxy)phenyl]carbamoyl]-5-fluoro-2-pyridyl]-2-methyl-pyridine-3-carboxylic acid

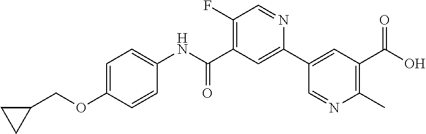 [SMILES: Cc1ncc(-c2cc(C(=O)Nc3ccc(OCC4CC4)cc3)c(F)cn2)cc1C(=O)O]

Using 2-bromo-N-(4-(cyclopropylmethoxy)phenyl)-5-fluoroisonicotinamide (I-30) and methyl 2-methyl-5-(4,4,5,5-tetramethyl-1,3,2-dioxaborolan-2-yl)nicotinate (CAS 2291165-13-2, Combi-Blocks) was obtained 5-[4-[[4-(cyclopropylmethoxy)phenyl]carbamoyl]-5-fluoro-2-pyridyl]-2-methyl-pyridine-3-carboxylic acid in 31% yield.

LCMS: Method A: 1.52 min, MS: ES⁺ 422.1; ¹H NMR (500 MHz, DMSO) δ ppm 10.65 (s, 1H), 9.14 (s, 1H), 8.82 (s, 1H), 8.65 (s, 1H), 8.32 (d, J=5.2 Hz, 1H), 7.63 (d, J=8.7 Hz, 2H), 6.94 (d, J=8.8 Hz, 2H), 3.81 (d, J=6.9 Hz, 2H), 2.72 (s, 3H), 1.33-1.14 (m, 1H), 0.67-0.51 (m, 2H), 0.39-0.24 (m, 2H). 1H obscured/not observed.

### Example 74

### 5-[4-[[4-(Benzyloxymethyl)phenyl]carbamoyl]-2-pyridyl]-2-methyl-pyridine-3-carboxylic acid

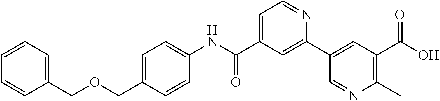 [SMILES: Cc1ncc(-c2cc(C(=O)Nc3ccc(COCc4ccccc4)cc3)ccn2)cc1C(=O)O]

Using N-(4-((benzyloxy)methyl)phenyl)-2-bromoisonicotinamide (I-31) and methyl 2-methyl-5-(4,4,5,5-tetramethyl-1,3,2-dioxaborolan-2-yl)nicotinate (CAS 2291165-13-2, Combi-Blocks) was obtained 5-[4-[[4-(benzyloxymethyl)phenyl]carbamoyl]-2-pyridyl]-2-methyl-pyridine-3-carboxylic acid in 41% yield.

LCMS: Method A: 1.73 min, MS: ES⁺ 454.2; ¹H NMR (500 MHz, DMSO) δ ppm 10.70 (s, 1H), 9.11 (d, J=2.4 Hz, 1H), 8.86 (d, J=5.0 Hz, 1H), 8.61 (d, J=2.4 Hz, 1H), 8.47 (s, 1H), 7.82 (dd, J=5.0, 1.6 Hz, 1H), 7.79 (d, J=8.4 Hz, 2H), 7.41-7.35 (m, 6H), 7.30 (q, J=4.3 Hz, 1H), 4.53 (s, 2H), 4.52 (s, 2H), 2.70 (s, 3H). 1H obscured/not observed.

### Example 75

### 5-[4-[[4-(Benzyloxymethyl)phenyl]carbamoyl]-6-methyl-2-pyridyl]-2-methyl-pyridine-3-carboxylic acid

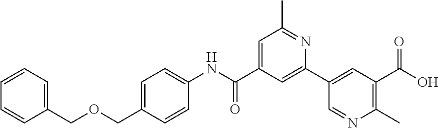 [SMILES: Cc1cc(C(=O)Nc2ccc(COCc3ccccc3)cc2)cc(-c2cnc(C)c(C(=O)O)c2)n1]

Using N-(4-((benzyloxy)methyl)phenyl)-2-chloro-6-methylisonicotinamide (I-32) and methyl 2-methyl-5-(4,4,5,5-tetramethyl-1,3,2-dioxaborolan-2-yl)nicotinate (CAS 2291165-13-2, Combi-Blocks) was obtained 5-[4-[[4-(benzyloxymethyl)phenyl]carbamoyl]-6-methyl-2-pyridyl]-2-methyl-pyridine-3-carboxylic acid in 36% yield.

LCMS: Method A: 1.40 min, MS: ES⁺ 468.2; ¹H NMR (500 MHz, DMSO) δ ppm 10.61 (s, 1H), 9.12 (s, 1H), 8.63 (s, 1H), 8.27 (s, 1H), 7.79 (d, J=8.5 Hz, 2H), 7.70 (s, 1H), 7.41-7.36 (m, 6H), 7.34-7.28 (m, 1H), 4.55 (s, 2H), 4.53 (s, 2H), 2.71 (s, 3H), 2.66 (s, 3H). 1H obscured/not observed.

### Example 76

### 5-[4-[[4-(Benzyloxymethyl)phenyl]carbamoyl]-5-fluoro-2-pyridyl]-2-methyl-pyridine-3-carboxylic acid

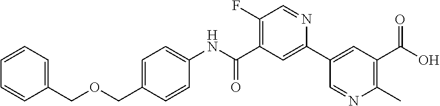 [SMILES: Cc1ncc(-c2cc(C(=O)Nc3ccc(COCc4ccccc4)cc3)c(F)cn2)cc1C(=O)O]

Using N-(4-((benzyloxy)methyl)phenyl)-2-bromo-5-fluoroisonicotinamide (I-33) and methyl 2-methyl-5-(4,4,5,5-tetramethyl-1,3,2-dioxaborolan-2-yl)nicotinate (CAS 2291165-13-2, Combi-Blocks) was obtained 5-[4-[[4-(benzyloxymethyl)phenyl]carbamoyl]-5-fluoro-2-pyridyl]-2-methyl-pyridine-3-carboxylic acid in 26% yield.

LCMS: Method A: 1.70 min, MS: ES⁺ 472.2; ¹H NMR (500 MHz, DMSO) δ ppm 10.81 (s, 1H), 9.11 (s, 1H), 8.84 (s, 1H), 8.61 (s, 1H), 8.34 (d, J=5.2 Hz, 1H), 7.74 (d, J=8.1 Hz, 2H), 7.42-7.35 (m, 6H), 7.33-7.28 (m, 1H), 4.54 (s, 2H), 4.53 (s, 2H), 2.71 (s, 3H). 1H obscured/not observed.

### Example 77

### 5-[4-[[4-(Benzyloxymethyl)-3-fluoro-phenyl]carbamoyl]-2-pyridyl]-2-methyl-pyridine-3-carboxylic acid

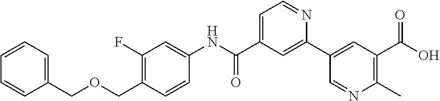 [SMILES: Cc1ncc(-c2cc(C(=O)Nc3ccc(COCc4ccccc4)c(F)c3)ccn2)cc1C(=O)O]

Using N-(4-((benzyloxy)methyl)-3-fluorophenyl)-2-bromoisonicotinamide (I-34) and methyl 2-methyl-5-(4,4,5,5-tetramethyl-1,3,2-dioxaborolan-2-yl)nicotinate (CAS 2291165-13-2, Combi-Blocks) was obtained 5-[4-[[4-(benzyloxymethyl)-3-fluoro-phenyl]carbamoyl]-2-pyridyl]-2-methyl-pyridine-3-carboxylic acid in 39% yield.

LCMS: Method A: 1.67 min, MS: ES⁺ 472.2; ¹H NMR (500 MHz, DMSO) δ ppm 10.90 (s, 1H), 9.20 (d, J=2.4 Hz, 1H), 8.89 (d, J=5.1 Hz, 1H), 8.71 (d, J=2.5 Hz, 1H), 8.50 (s, 1H), 7.84 (dd, J=5.0, 1.5 Hz, 1H), 7.79 (dd, J=12.3, 2.0 Hz, 1H), 7.60 (dd, J=8.2, 2.1 Hz, 1H), 7.50 (t, J=8.4 Hz, 1H), 7.41-7.35 (m, 4H), 7.35-7.27 (m, 1H), 4.58-4.54 (m, 4H), 2.73 (s, 3H). 1H obscured/not observed.

### Example 78

### 5-[4-[[4-(Cyclopropoxymethyl)-3-fluoro-phenyl]carbamoyl]-2-pyridyl]-2-methyl-pyridine-3-carboxylic acid

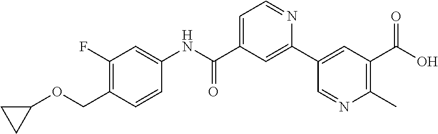 [SMILES: Cc1ncc(-c2cc(C(=O)Nc3ccc(COC4CC4)c(F)c3)ccn2)cc1C(=O)O]

Using 2-bromo-N-(4-(cyclopropoxymethyl)-3-fluorophenyl)isonicotinamide (I-35) and methyl 2-methyl-5-(4,4,5,5-tetramethyl-1,3,2-dioxaborolan-2-yl)nicotinate (CAS 2291165-13-2, Combi-Blocks) was obtained 5-[4-[[4-(cyclopropoxymethyl)-3-fluoro-phenyl]carbamoyl]-2-pyridyl]-2-methyl-pyridine-3-carboxylic acid in 36% yield.

LCMS: Method A: 1.49 min, MS: ES⁺ 422.2; ¹H NMR (500 MHz, DMSO) δ ppm 10.78 (s, 1H), 9.36 (d, J=2.4 Hz, 1H), 8.94-8.86 (m, 2H), 8.53 (s, 1H), 7.87 (d, J=5.0 Hz, 1H), 7.77 (d, J=12.3 Hz, 1H), 7.59-7.53 (m, 1H), 7.45 (t, J=8.4 Hz, 1H), 4.53 (s, 2H), 3.36 (dt, J=6.1, 3.2 Hz, 1H), 2.80 (s, 3H), 0.53 (q, J=4.6 Hz, 2H), 0.48 (h, J=5.3 Hz, 2H). 1H obscured/not observed.

### Example 79

### 5-[4-[[4-(Cyclopropylmethoxy)-3-fluoro-phenyl]carbamoyl]-2-pyridyl]-2-methyl-pyridine-3-carboxylic acid

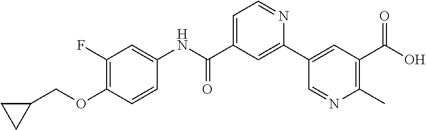 [SMILES: Cc1ncc(-c2cc(C(=O)Nc3ccc(OCC4CC4)c(F)c3)ccn2)cc1C(=O)O]

Using 2-bromo-N-(4-(cyclopropylmethoxy)-3-fluorophenyl)isonicotinamide (I-36) and methyl 2-methyl-5-(4,4,5,5-tetramethyl-1,3,2-dioxaborolan-2-yl)nicotinate (CAS 2291165-13-2, Combi-Blocks) was obtained 5-[4-[[4-(cyclopropylmethoxy)-3-fluoro-phenyl]carbamoyl]-2-pyridyl]-2-methyl-pyridine-3-carboxylic acid in 37% yield.

LCMS: Method A: 1.54 min, MS: ES⁺ 422.2; ¹H NMR (500 MHz, DMSO) δ ppm 13.37 (s, 1H), 10.53 (s, 1H), 9.33-9.29 (m, 1H), 8.86-8.81 (m, 2H), 8.45 (s, 1H), 7.79 (d, J=5.0 Hz, 1H), 7.71-7.64 (m, 1H), 7.41 (d, J=8.8 Hz, 1H), 7.11 (t, J=9.3 Hz, 1H), 3.83 (d, J=7.0 Hz, 2H), 2.74 (s, 3H), 1.22-1.13 (m, 1H), 0.55-0.48 (m, 2H), 0.30-0.24 (m, 2H).

### Example 80

### 5-[3-[[4-(Benzyloxymethyl)phenyl]carbamoyl]-4-methoxy-phenyl]-2-methyl-pyridine-3-carboxylic acid

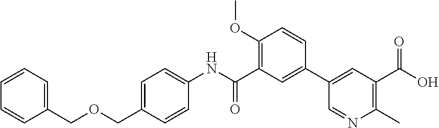 [SMILES: COc1ccc(-c2cnc(C)c(C(=O)O)c2)cc1C(=O)Nc1ccc(COCc2ccccc2)cc1]

Using (3-((4-((benzyloxy)methyl)phenyl)carbamoyl)-4-methoxyphenyl)boronic acid (I-37) and 5-bromo-2-methylnicotinic acid (CAS 351003-02-6, Combi-Blocks) was obtained 5-[3-[[4-(benzyloxymethyl)phenyl]carbamoyl]-4-methoxy-phenyl]-2-methyl-pyridine-3-carboxylic acid in 20% yield.

LCMS: Method A: 1.66 min, MS: ES⁺ 483.2; ¹H NMR (500 MHz, DMSO) δ ppm 13.36 (s, 1H), 10.24 (s, 1H), 8.94 (d, J=2.5 Hz, 1H), 8.38 (d, J=2.5 Hz, 1H), 7.96 (d, J=2.6 Hz, 1H), 7.91 (dd, J=8.6, 2.5 Hz, 1H), 7.75 (d, J=8.3 Hz, 2H), 7.42-7.24 (m, 8H), 4.53 (s, 2H), 4.51 (s, 2H), 3.95 (s, 3H), 2.75 (s, 3H).

### Example 81

### 5-[5-[[4-(Benzyloxymethyl)phenyl]carbamoyl]-2-methoxy-phenyl]-2-methyl-pyridine-3-carboxylic acid

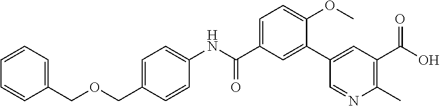 [SMILES: COc1ccc(C(=O)Nc2ccc(COCc3ccccc3)cc2)cc1-c1cnc(C)c(C(=O)O)c1]

Using N-(4-((benzyloxy)methyl)phenyl)-4-methoxy-3-(4,4,5,5-tetramethyl-1,3,2-dioxaborolan-2-yl)benzamide (I-38) and 5-bromo-2-methylnicotinic acid (CAS 351003-02-6, Combi-Blocks) was obtained 5-[5-[[4-(benzyloxymethyl)phenyl]carbamoyl]-2-methoxy-phenyl]-2-methyl-pyridine-3-carboxylic acid in 31% yield.

LCMS: Method A: 1.63 min, MS: ES⁺ 483.2; ¹H NMR (400 MHz, DMSO) δ ppm 13.29 (s, 1H), 10.18 (s, 1H), 8.79 (d, J=2.3 Hz, 1H), 8.32 (d, J=2.3 Hz, 1H), 8.12-7.99 (m, 2H), 7.79-7.70 (m, 2H), 7.43-7.20 (m, 8H), 4.52 (s, 2H), 4.50 (s, 2H), 3.89 (s, 3H), 2.77 (s, 3H).

### Example 82

### 5-[3-[[4-(Benzyloxymethyl)phenyl]carbamoyl]-4-methyl-phenyl]-2-methyl-pyridine-3-carboxylic acid

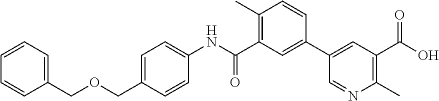 [SMILES: Cc1ccc(-c2cnc(C)c(C(=O)O)c2)cc1C(=O)Nc1ccc(COCc2ccccc2)cc1]

Using (3-((4-((benzyloxy)methyl)phenyl)carbamoyl)-4-methylphenyl)boronic acid (I-39) and 5-bromo-2-methylnicotinic acid (CAS 351003-02-6, Combi-Blocks) was obtained 5-[3-[[4-(benzyloxymethyl)phenyl]carbamoyl]-4-methyl-phenyl]-2-methyl-pyridine-3-carboxylic acid in 39% yield.

LCMS: Method A: 1.68 min, MS: ES⁺ 467.2; ¹H NMR (500 MHz, DMSO) δ ppm 13.38 (s, 1H), 10.42 (s, 1H), 8.98 (d, J=2.5 Hz, 1H), 8.44 (d, J=2.5 Hz, 1H), 7.85 (d, J=2.1 Hz, 1H), 7.82-7.73 (m, 3H), 7.45 (d, J=8.0 Hz, 1H), 7.41-7.33 (m, 6H), 7.32-7.27 (m, 1H), 4.53 (s, 2H), 4.51 (s, 2H), 2.76 (s, 3H), 2.44 (s, 3H).

### Example 83

### 5-[5-[[4-(Benzyloxymethyl)phenyl]carbamoyl]-2-methyl-phenyl]-2-methyl-pyridine-3-carboxylic acid

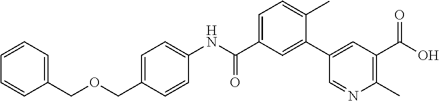 [SMILES: Cc1ccc(C(=O)Nc2ccc(COCc3ccccc3)cc2)cc1-c1cnc(C)c(C(=O)O)c1]

Using N-(4-((benzyloxy)methyl)phenyl)-4-methyl-3-(4,4,5,5-tetramethyl-1,3,2-dioxaborolan-2-yl)benzamide (I-40) and 5-bromo-2-methylnicotinic acid (CAS 351003-02-6, Combi-Blocks) was obtained 5-[5-[[4-(benzyloxymethyl)phenyl]carbamoyl]-2-methyl-phenyl]-2-methyl-pyridine-3-carboxylic acid in 15% yield.

LCMS: Method A: 1.73 min, MS: ES⁺ 467.2; ¹H NMR (500 MHz, DMSO) δ ppm 13.35 (s, 1H), 10.24 (s, 1H), 8.70 (s, 1H), 8.19 (s, 1H), 7.94 (d, J=7.9 Hz, 1H), 7.90 (s, 1H), 7.76 (d, J=8.1 Hz, 2H), 7.52 (d, J=7.9 Hz, 1H), 7.44-7.23 (m, 7H), 4.53 (s, 2H), 4.51 (s, 2H), 2.80 (s, 3H), 2.33 (s, 3H).

### Example 84

### 5-[3-[[4-(Cyclopropylmethoxy)phenyl]carbamoyl]-4-fluoro-phenyl]-2-methyl-pyridine-3-carboxylic acid

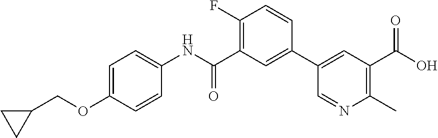 [SMILES: Cc1ncc(-c2ccc(F)c(C(=O)Nc3ccc(OCC4CC4)cc3)c2)cc1C(=O)O]

Using N-(4-(cyclopropylmethoxy)phenyl)-2-fluoro-5-(4,4,5,5-tetramethyl-1,3,2-dioxaborolan-2-yl)benzamide (I-41) and 5-bromo-2-methylnicotinic acid (CAS 351003-02-6, Combi-Blocks) was obtained 5-[3-[[4-(cyclopropylmethoxy)phenyl]carbamoyl]-4-fluoro-phenyl]-2-methyl-pyridine-3-carboxylic acid in 36% yield.

LCMS: Method A: 1.53 min, MS: ES⁺ 421.1; ¹H NMR (500 MHz, DMSO) δ ppm 13.40 (s, 1H), 10.35 (s, 1H), 8.98 (d, J=2.4 Hz, 1H), 8.44 (d, J=2.5 Hz, 1H), 8.02 (dd, J=6.6, 2.5 Hz, 1H), 7.99-7.92 (m, 1H), 7.63 (d, J=9.1 Hz, 2H), 7.48 (t, J=9.2 Hz, 1H), 6.93 (d, J=9.0 Hz, 2H), 3.81 (d, J=7.0 Hz, 2H), 2.77 (s, 3H), 1.33-1.06 (m, 1H), 0.65-0.46 (m, 2H), 0.41-0.14 (m, 2H).

### Example 85

### 5-[5-[[4-(Benzyloxymethyl)phenyl]carbamoyl]-2-fluoro-phenyl]-2-methyl-pyridine-3-carboxylic acid

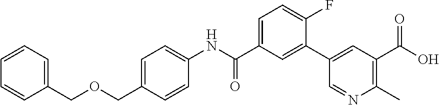 [SMILES: Cc1ncc(-c2cc(C(=O)Nc3ccc(COCc4ccccc4)cc3)ccc2F)cc1C(=O)O]

Using (5-((4-((benzyloxy)methyl)phenyl)carbamoyl)-2-fluorophenyl)boronic acid (I-42) and methyl 5-bromo-2-methylnicotinate (CAS 1215916-40-7, BLD) was obtained 5-[5-[[4-(benzyloxymethyl)phenyl]carbamoyl]-2-fluoro-phenyl]-2-methyl-pyridine-3-carboxylic acid in 12% yield.

LCMS: Method A: 1.76 min, MS: ES⁺ 471.2; ¹H NMR (500 MHz, DMSO) δ ppm 13.49 (s, 1H), 10.37 (s, 1H), 8.88 (s, 1H), 8.40 (s, 1H), 8.24 (dd, J=7.5, 2.4 Hz, 1H), 8.10-8.05 (m, 1H), 7.77 (d, J=8.4 Hz, 2H), 7.55 (dd, J=10.4, 8.6 Hz, 1H), 7.39-7.34 (m, 6H), 7.33-7.28 (m, 1H), 4.54 (s, 2H), 4.52 (s, 2H), 2.79 (s, 3H).

### Example 86

### 5-[3-[[4-(Benzyloxymethyl)phenyl]carbamoyl]-2-fluoro-phenyl]-2-methyl-pyridine-3-carboxylic acid

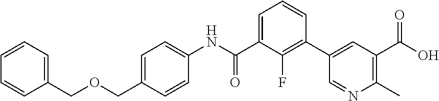 [SMILES: Cc1ncc(-c2cccc(C(=O)Nc3ccc(COCc4ccccc4)cc3)c2F)cc1C(=O)O]

Using (3-((4-((benzyloxy)methyl)phenyl)carbamoyl)-2-fluorophenyl)boronic acid (I-43) and 5-bromo-2-methylnicotinic acid (CAS 351003-02-6, Combi-Blocks) was obtained 5-[3-[[4-(benzyloxymethyl)phenyl]carbamoyl]-2-fluoro-phenyl]-2-methyl-pyridine-3-carboxylic acid in 40% yield.

LCMS: Method A: 1.73 min, MS: ES⁺ 471.2; ¹H NMR (500 MHz, DMSO) δ ppm 13.45 (s, 1H), 10.54 (s, 1H), 8.84 (d, J=2.1 Hz, 1H), 8.39 (s, 1H), 7.83-7.77 (m, 1H), 7.75-7.69 (m, 3H), 7.47 (t, J=7.6 Hz, 1H), 7.41-7.34 (m, 6H), 7.33-7.28 (m, 1H), 4.53 (s, 2H), 4.51 (s, 2H), 2.79 (s, 3H).

### Example 87

### 5-[3-[[4-(Benzyloxymethyl)phenyl]carbamoyl]-2,4-difluoro-phenyl]-2-methyl-pyridine-3-carboxylic acid

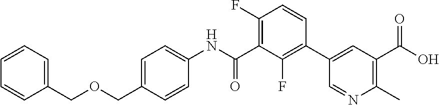 [SMILES: Cc1ncc(-c2ccc(F)c(C(=O)Nc3ccc(COCc4ccccc4)cc3)c2F)cc1C(=O)O]

Using (3-((4-((benzyloxy)methyl)phenyl)carbamoyl)-2,4-difluorophenyl)boronic acid (I-44) and 5-bromo-2-methylnicotinic acid (CAS 351003-02-6, Combi-Blocks) was obtained 5-[3-[[4-(benzyloxymethyl)phenyl]carbamoyl]-2,4-difluoro-phenyl]-2-methyl-pyridine-3-carboxylic acid in 30% yield.

LCMS: Method A: 1.75 min, MS: ES⁺ 489.1; ¹H NMR (500 MHz, DMSO) δ ppm 10.91 (s, 1H), 8.63 (s, 1H), 8.15 (s, 1H), 7.78 (s, 1H), 7.73-7.62 (m, 2H), 7.47-7.24 (m, 8H), 4.54-4.49 (m, 4H), 2.72 (s, 3H). 1H obscured/not observed.

### Example 88

### 5-[3-[[4-(Cyclopropylmethoxymethyl)-3-fluoro-phenyl]carbamoyl]-4-fluoro-phenyl]-2-methyl-pyridine-3-carboxylic acid

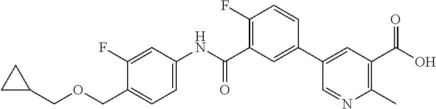 [SMILES: Cc1ncc(-c2ccc(F)c(C(=O)Nc3ccc(COCC4CC4)c(F)c3)c2)cc1C(=O)O]

Using N-(4-((cyclopropylmethoxy)methyl)-3-fluorophenyl)-2-fluoro-5-(4,4,5,5-tetramethyl-1,3,2-dioxaborolan-2-yl)benzamide (I-45) and 5-bromo-2-methylnicotinic acid (CAS 351003-02-6, Combi-Blocks) was obtained 5-[3-[[4-(cyclopropylmethoxymethyl)-3-fluoro-phenyl]carbamoyl]-4-fluoro-phenyl]-2-methyl-pyridine-3-carboxylic acid in 66% yield.

LCMS: Method A: 1.61 min, MS: ES⁺ 453.2; ¹H NMR (500 MHz, DMSO) δ ppm 10.65 (s, 1H), 8.61 (d, J=2.6 Hz, 1H), 8.06 (d, J=2.6 Hz, 1H), 7.82 (dd, J=6.7, 2.6 Hz, 1H), 7.77-7.70 (m, 1H), 7.58-7.52 (m, 1H), 7.35-7.26 (m, 2H), 7.28-7.21 (m, 1H), 4.32 (s, 2H), 3.12 (d, J=6.8 Hz, 2H), 2.52 (s, 3H), 0.92-0.81 (m, 1H), 0.34-0.25 (m, 2H), 0.08-−0.04 (m, 2H). 1H obscured/not observed.

### Example 89

### 5-[3-[[4-(Cyclopropylmethoxy)-3-fluoro-phenyl]carbamoyl]-4-fluoro-phenyl]-2-methyl-pyridine-3-carboxylic acid

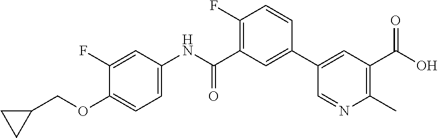 [SMILES: Cc1ncc(-c2ccc(F)c(C(=O)Nc3ccc(OCC4CC4)c(F)c3)c2)cc1C(=O)O]

Using N-(4-(cyclopropylmethoxy)-3-fluorophenyl)-2-fluoro-5-(4,4,5,5-tetramethyl-1,3,2-dioxaborolan-2-yl)benzamide (I-46) and 5-bromo-2-methylnicotinic acid (CAS 351003-02-6, Combi-Blocks) was obtained 5-[3-[[4-(cyclopropylmethoxy)-3-fluoro-phenyl]carbamoyl]-4-fluoro-phenyl]-2-methyl-pyridine-3-carboxylic acid in 50% yield.

LCMS: Method A: 1.60 min, MS: ES⁺ 439.1; ¹H NMR (500 MHz, DMSO) δ ppm 10.36 (s, 1H), 8.45 (d, J=2.6 Hz, 1H), 7.91 (d, J=2.6 Hz, 1H), 7.69 (dd, J=6.7, 2.6 Hz, 1H), 7.66-7.59 (m, 1H), 7.47 (dd, J=13.5, 2.6 Hz, 1H), 7.23-7.14 (m, 2H), 6.89 (t, J=9.2 Hz, 1H), 3.63 (d, J=7.0 Hz, 2H), 2.42 (s, 3H), 1.04-0.93 (m, 1H), 0.37-0.30 (m, 2H), 0.12-0.05 (m, 2H). 1H obscured/not observed.

### Example 90

### 5-[3-[[4-(Benzyloxymethyl)-3-fluoro-phenyl]carbamoyl]-4-fluoro-phenyl]-2-methyl-pyridine-3-carboxylic acid

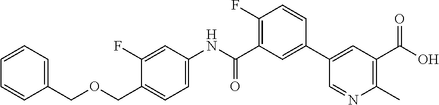 [SMILES: Cc1ncc(-c2ccc(F)c(C(=O)Nc3ccc(COCc4ccccc4)c(F)c3)c2)cc1C(=O)O]

Using N-(4-((benzyloxy)methyl)-3-fluorophenyl)-2-fluoro-5-(4,4,5,5-tetramethyl-1,3,2-dioxaborolan-2-yl)benzamide (I-47) and 5-bromo-2-methylnicotinic acid (CAS 351003-02-6, Combi-Blocks) was obtained 5-[3-[[4-(benzyloxymethyl)-3-fluoro-phenyl]carbamoyl]-4-fluoro-phenyl]-2-methyl-pyridine-3-carboxylic acid in 33% yield.

LCMS: Method A: 1.79 min, MS: ES⁺ 489.2; ¹H NMR (500 MHz, DMSO) δ ppm 10.77 (s, 1H), 8.85 (d, J=2.5 Hz, 1H), 8.31 (d, J=2.6 Hz, 1H), 8.02 (dd, J=6.6, 2.6 Hz, 1H), 7.98-7.91 (m, 1H), 7.77-7.70 (m, 1H), 7.52-7.45 (m, 3H), 7.39-7.33 (m, 4H), 7.33-7.27 (m, 1H), 4.67-4.43 (m, 4H), 2.71 (s, 3H). 1H obscured/not observed.

### Example 91

### 5-[3-[[4-(Cyclopropoxymethyl)-3-fluoro-phenyl]carbamoyl]-4-fluoro-phenyl]-2-methyl-pyridine-3-carboxylic acid

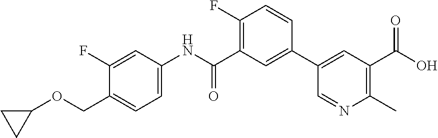 [SMILES: Cc1ncc(-c2ccc(F)c(C(=O)Nc3ccc(COC4CC4)c(F)c3)c2)cc1C(=O)O]

Using N-(4-(cyclopropoxymethyl)-3-fluorophenyl)-2-fluoro-5-(4,4,5,5-tetramethyl-1,3,2-dioxaborolan-2-yl)benzamide (I-48) and 5-bromo-2-methylnicotinic acid (CAS 351003-02-6, Combi-Blocks) was obtained 5-[3-[[4-(cyclopropoxymethyl)-3-fluoro-phenyl]carbamoyl]-4-fluoro-phenyl]-2-methyl-pyridine-3-carboxylic acid in 16% yield.

LCMS: Method A: 1.55 min, MS: ES⁺ 439.2; ¹H NMR (500 MHz, DMSO) δ ppm 10.75 (s, 1H), 8.89-8.85 (m, 1H), 8.34 (d, J=2.5 Hz, 1H), 8.05-8.00 (m, 1H), 7.98-7.92 (m, 1H), 7.75-7.68 (m, 1H), 7.52-7.45 (m, 2H), 7.41 (t, J=8.3 Hz, 1H), 4.51 (s, 2H), 3.38-3.30 (m, 1H), 2.73 (s, 3H), 0.56-0.48 (m, 2H), 0.51-0.43 (m, 2H). 1H obscured/not observed.

### Example 92

### 5-[3-[[4-(1-Cyclopropylethoxy)-3-fluoro-phenyl]carbamoyl]-4-fluoro-phenyl]-2-methyl-pyridine-3-carboxylic acid

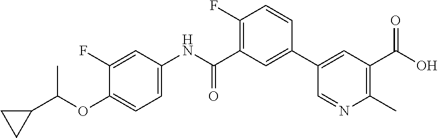 [SMILES: Cc1ncc(-c2ccc(F)c(C(=O)Nc3ccc(OC(C)C4CC4)c(F)c3)c2)cc1C(=O)O]

Using N-(4-(1-cyclopropylethoxy)-3-fluorophenyl)-2-fluoro-5-(4,4,5,5-tetramethyl-1,3,2-dioxaborolan-2-yl)benzamide (I-17) and 5-bromo-2-methylnicotinic acid (CAS 351003-02-6, Combi-Blocks) was obtained 5-[3-[[4-(1-cyclopropylethoxy)-3-fluoro-phenyl]carbamoyl]-4-fluoro-phenyl]-2-methyl-pyridine-3-carboxylic acid in 37% yield.

LCMS: Method A: 1.79 min, MS: ES⁺ 453.2; ¹H NMR (400 MHz, DMSO) δ ppm 10.55 (s, 1H), 8.89 (d, J=2.5 Hz, 1H), 8.35 (d, J=2.5 Hz, 1H), 8.01 (dd, J=6.6, 2.5 Hz, 1H), 7.98-7.90 (m, 1H), 7.70 (dd, J=13.4, 2.5 Hz, 1H), 7.52-7.43 (m, 1H), 7.42-7.34 (m, 1H), 7.16 (t, J=9.2 Hz, 1H), 3.87-3.76 (m, 1H), 2.73 (s, 3H), 1.30 (d, J=6.1 Hz, 3H), 1.10-1.00 (m, 1H), 0.53-0.43 (m, 2H), 0.33-0.20 (m, 2H). 1H obscured/not observed.

### Example 93

### 5-[3-[[3-Cyano-4-(1-cyclopropylethoxy)phenyl]carbamoyl]-4-fluoro-phenyl]-2-methyl-pyridine-3-carboxylic acid

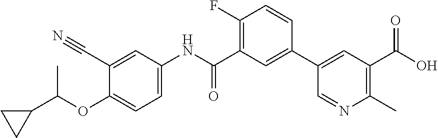 [SMILES: Cc1ncc(-c2ccc(F)c(C(=O)Nc3ccc(OC(C)C4CC4)c(C#N)c3)c2)cc1C(=O)O]

Using iN-(3-cyano-4-(1-cyclopropylethoxy)phenyl)-2-fluoro-5-(4,4,5,5-tetramethyl-1,3,2-dioxaborolan-2-yl)benzamide (I-18) and 5-bromo-2-methylnicotinic acid (CAS 351003-02-6, Combi-Blocks) was obtained 5-[3-[[3-cyano-4-(1-cyclopropylethoxy)phenyl]carbamoyl]-4-fluoro-phenyl]-2-methyl-pyridine-3-carboxylic acid in 51% yield.

LCMS: Method A: 1.74 min, MS: ES⁺ 460.2; ¹H NMR (400 MHz, DMSO) δ ppm 10.64 (s, 1H), 8.93 (d, J=2.5 Hz, 1H), 8.39 (d, J=2.4 Hz, 1H), 8.09-8.01 (m, 2H), 8.01-7.93 (m, 1H), 7.87 (dd, J=9.2, 2.7 Hz, 1H), 7.49 (dd, J=9.9, 8.6 Hz, 1H), 7.31 (d, J=9.3 Hz, 1H), 4.19-4.08 (m, 1H), 2.74 (s, 3H), 1.34 (d, J=6.1 Hz, 3H), 1.17-1.07 (m, 1H), 0.58-0.46 (m, 2H), 0.43-0.34 (m, 1H), 0.34-0.26 (m, 1H). 1H obscured/not observed.

### Example 94

### 2-Methyl-5-[2-methylsulfanyl-5-[[4-(2-phenylethoxy)phenyl]carbamoyl]-3-pyridyl]pyridine-3-carboxylic acid

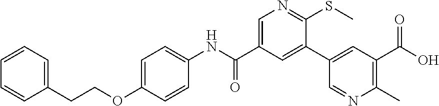 [SMILES: CSc1ncc(C(=O)Nc2ccc(OCCc3ccccc3)cc2)cc1-c1cnc(C)c(C(=O)O)c1]

Using 5-bromo-6-(methylthio)-N-(4-phenethoxyphenyl)nicotinamide (I-59) and methyl 2-methyl-5-(4,4,5,5-tetramethyl-1,3,2-dioxaborolan-2-yl)nicotinate (CAS 2291165-13-2, Combi-Blocks) was obtained 2-methyl-5-[2-methylsulfanyl-5-[[4-(2-phenylethoxy)phenyl]carbamoyl]-3-pyridyl]pyridine-3-carboxylic acid in 58% yield.

LCMS: Method A: 1.58 min, MS: ES⁺ 500.2; ¹H NMR (500 MHz, DMSO) δ ppm 10.24 (s, 1H), 8.99 (d, J=2.3 Hz, 1H), 8.40 (d, J=2.5 Hz, 1H), 8.07 (d, J=2.3 Hz, 1H), 7.94 (d, J=2.4 Hz, 1H), 7.64 (d, J=9.1 Hz, 2H), 7.40-7.27 (m, 4H), 7.26-7.17 (m, 1H), 6.94 (d, J=9.1 Hz, 2H), 4.18 (t, J=6.9 Hz, 2H), 3.03 (t, J=6.9 Hz, 2H), 2.68 (s, 3H), 2.52 (s, 3H). 1H obscured/not observed.

### Example 95

### 5-[2-(2-Methoxyethoxy)-5-[[4-(2-phenylethoxy)phenyl]carbamoyl]-3-pyridyl]-2-methyl-pyridine-3-carboxylic acid

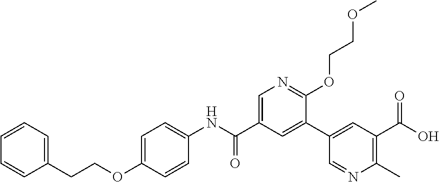 [SMILES: COCCOc1ncc(C(=O)Nc2ccc(OCCc3ccccc3)cc2)cc1-c1cnc(C)c(C(=O)O)c1]

Using 5-bromo-6-(2-methoxyethoxy)-N-(4-phenethoxyphenyl)nicotinamide (I-60) and methyl 2-methyl-5-(4,4,5,5-tetramethyl-1,3,2-dioxaborolan-2-yl)nicotinate (CAS 2291165-13-2, Combi-Blocks) was obtained 5-[2-(2-methoxyethoxy)-5-[[4-(2-phenylethoxy)phenyl]carbamoyl]-3-pyridyl]-2-methyl-pyridine-3-carboxylic acid in 16% yield.

LCMS: Method A: 1.58 min, MS: ES⁺ 528.1; ¹H NMR (500 MHz, DMSO) δ ppm 10.21 (s, 1H), 8.74 (d, J=2.4 Hz, 1H), 8.70 (s, 1H), 8.37 (d, J=2.4 Hz, 1H), 8.22 (s, 1H), 7.64 (d, J=9.1 Hz, 2H), 7.40-7.28 (m, 4H), 7.26-7.20 (m, 1H), 6.95 (d, J=9.1 Hz, 2H), 4.63-4.50 (m, 2H), 4.19 (t, J=6.9 Hz, 2H), 3.81-3.58 (m, 2H), 3.28 (s, 3H), 3.04 (t, J=6.9 Hz, 2H), 2.70 (s, 3H). 1H obscured/not observed.

### General Method—Examples 96-101

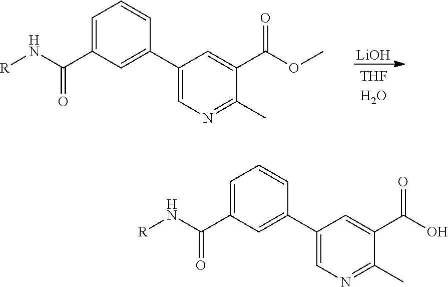

Examples 96-101 were prepared as follows:

To the appropriate starting material in THF (2 mL) was added LiOH (6 eq) in water (2 mL). The mixture was stirred at 40° C. for 2 h then concentrated to ~50% volume under reduced pressure. The remaining mixture was diluted with water and acidified to ~pH 2 with aq. HCl (1M). The solid was collected and triturated with acetonitrile (20 mL) and diethyl ether (15 mL) to afford desired product.

### Example 96

### 5-[3-[[4-[(4-Chlorophenyl)sulfonylmethyl]phenyl]carbamoyl]phenyl]-2-methyl-pyridine-3-carboxylic acid

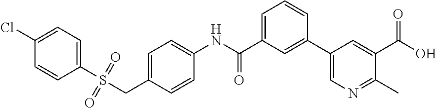 [SMILES: Cc1ncc(-c2cccc(C(=O)Nc3ccc(CS(=O)(=O)c4ccc(Cl)cc4)cc3)c2)cc1C(=O)O]

Using methyl 5-(3-((4-(((4-chlorophenyl)sulfonyl)methyl)phenyl)carbamoyl)phenyl)-2-methylnicotinate (I-53) was obtained 5-[3-[[4-[(4-chlorophenyl)sulfonylmethyl]phenyl]-carbamoyl]phenyl]-2-methyl-pyridine-3-carboxylic acid in 71% yield.

LCMS: Method A: 1.54 min, MS: ES⁺ 521.1; ¹H NMR (500 MHz, DMSO) δ ppm 10.44 (s, 1H), 9.07 (d, J=2.4 Hz, 1H), 8.56 (s, 1H), 8.31 (s, 1H), 8.00 (d, J=7.6 Hz, 2H), 7.75-7.65 (m, 7H), 7.14 (d, J=8.2 Hz, 2H), 4.70 (s, 2H), 2.80 (s, 3H). 1H obscured/not observed.

### Example 97

### 5-[3-[[4-[(3-Chlorophenyl)sulfonylmethyl]phenyl]carbamoyl]phenyl]-2-methyl-pyridine-3-carboxylic acid

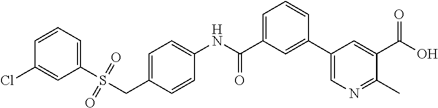 [SMILES: Cc1ncc(-c2cccc(C(=O)Nc3ccc(CS(=O)(=O)c4cccc(Cl)c4)cc3)c2)cc1C(=O)O]

Using methyl 5-(3-((4-(((3-chlorophenyl)sulfonyl)methyl)phenyl)carbamoyl)phenyl)-2-methylnicotinate (I-54) was obtained 5-[3-[[4-[(3-chlorophenyl)sulfonylmethyl]phenyl]-carbamoyl]phenyl]-2-methyl-pyridine-3-carboxylic acid in 81% yield.

LCMS: Method A: 1.21 min, MS: ES⁺ 521.1; ¹H NMR (500 MHz, DMSO) δ ppm 10.50 (s, 1H), 9.11 (d, J=2.4 Hz, 1H), 8.60 (d, J=2.4 Hz, 1H), 8.34 (t, J=1.9 Hz, 1H), 8.01 (dd, J=7.8, 1.8 Hz, 2H), 7.84-7.79 (m, 2H), 7.77-7.73 (m, 2H), 7.70-7.61 (m, 3H), 7.18-7.14 (m, 2H), 4.75 (s, 2H), 2.81 (s, 3H). 1H obscured/not observed.

### Example 98

### 5-[3-[[4-[(2-Chlorophenyl)sulfonylmethyl]phenyl]carbamoyl]phenyl]-2-methyl-pyridine-3-carboxylic acid

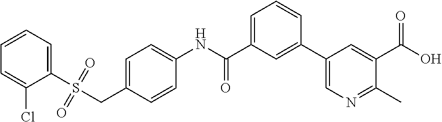 [SMILES: Cc1ncc(-c2cccc(C(=O)Nc3ccc(CS(=O)(=O)c4ccccc4Cl)cc3)c2)cc1C(=O)O]

Using methyl 5-(3-((4-(((2-chlorophenyl)sulfonyl)methyl)phenyl)carbamoyl)phenyl)-2-methylnicotinate (I-55) was obtained 5-[3-[[4-[(2-chlorophenyl)sulfonylmethyl]phenyl]-carbamoyl]phenyl]-2-methyl-pyridine-3-carboxylic acid in 60% yield.

LCMS: Method A: 1.17 min, MS: ES⁺ 521.1; ¹H NMR (500 MHz, DMSO) δ ppm 10.45 (s, 1H), 9.09 (d, J=2.5 Hz, 1H), 8.58 (d, J=2.4 Hz, 1H), 8.34-8.29 (m, 1H), 8.03-7.97 (m, 2H), 7.80-7.77 (m, 1H), 7.76-7.74 (m, 1H), 7.74-7.73 (m, 1H), 7.73-7.70 (m, 2H), 7.70-7.66 (m, 1H), 7.53-7.49 (m, 1H), 7.21-7.16 (m, 2H), 4.84 (s, 2H), 2.78 (s, 3H). 1H obscured/not observed.

### Example 99

### 5-[3-[[4-[(4-Methoxyphenyl)sulfonylmethyl]phenyl]carbamoyl]phenyl]-2-methyl-pyridine-3-carboxylic acid

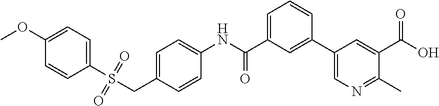 [SMILES: COc1ccc(S(=O)(=O)Cc2ccc(NC(=O)c3cccc(-c4cnc(C)c(C(=O)O)c4)c3)cc2)cc1]

Using methyl 5-(3-((4-(((4-methoxyphenyl)sulfonyl)methyl)phenyl)carbamoyl)phenyl)-2-methylnicotinate (I-56) was obtained 5-[3-[[4-[(4-methoxyphenyl)sulfonylmethyl]phenyl-]carbamoyl]phenyl]-2-methyl-pyridine-3-carboxylic acid in 49% yield.

LCMS: Method A: 1.11 min, MS: ES⁺ 517.1; ¹H NMR (500 MHz, DMSO) δ ppm 10.47 (s, 1H), 9.10 (d, J=2.4 Hz, 1H), 8.58 (d, J=2.4 Hz, 1H), 8.32 (d, J=2.0 Hz, 1H), 8.00 (m, 2H), 7.75-7.65 (m, 3H), 7.64-7.59 (m, 2H), 7.14-7.06 (m, 4H), 4.58 (s, 2H), 3.84 (s, 3H), 2.81 (s, 3H). 1H obscured/not observed.

### Example 100

### 5-[3-[[4-[(3-Methoxyphenyl)sulfonylmethyl]phenyl]carbamoyl]phenyl]-2-methyl-pyridine-3-carboxylic acid

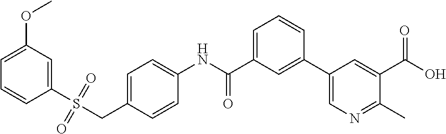 [SMILES: COc1cccc(S(=O)(=O)Cc2ccc(NC(=O)c3cccc(-c4cnc(C)c(C(=O)O)c4)c3)cc2)c1]

Using methyl 5-(3-((4-(((3-methoxyphenyl)sulfonyl)methyl)phenyl)carbamoyl)phenyl)-2-methylnicotinate (I-57) was obtained 5-[3-[[4-[(3-methoxyphenyl)sulfonylmethyl]phenyl]-carbamoyl]phenyl]-2-methyl-pyridine-3-carboxylic acid in 14% yield.

LCMS: Method A: 1.28 min, MS: ES⁺ 517.0; ¹H NMR (500 MHz, DMSO) δ ppm 10.40 (s, 1H), 9.01 (d, J=2.4 Hz, 1H), 8.47 (d, J=2.4 Hz, 1H), 8.28 (t, J=1.9 Hz, 1H), 8.01-7.94 (m, 2H), 7.72-7.64 (m, 3H), 7.51 (t, J=8.0 Hz, 1H), 7.31-7.25 (m, 2H), 7.24-7.20 (m, 1H), 7.18-7.13 (m, 2H), 4.66 (s, 2H), 3.80 (s, 3H), 2.77 (s, 3H). 1H obscured/not observed.

### Example 101

### 2-Methyl-5-[3-[[4-[(6-methyl-3-pyridyl)sulfonylmethyl]phenyl]carbamoyl]phenyl]pyridine-3-carboxylic acid

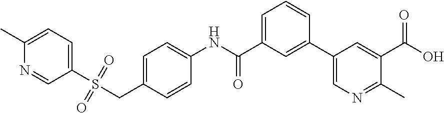 [SMILES: Cc1ccc(S(=O)(=O)Cc2ccc(NC(=O)c3cccc(-c4cnc(C)c(C(=O)O)c4)c3)cc2)cn1]

Using methyl 2-methyl-5-(3-((4-(((6-methylpyridin-3-yl)sulfonyl)methyl)phenyl)carbamoyl)-phenyl)nicotinate (I-58) was obtained 2-methyl-5-[3-[[4-[(6-methyl-3-pyridyl)sulfonylmethyl]-phenyl]carbamoyl]phenyl]pyridine-3-carboxylic acid in 9% yield.

LCMS: Method A: 1.10 min, MS: ES⁺ 502.1; ¹H NMR (500 MHz, DMSO) δ ppm 13.46 (s, 1H), 10.42 (s, 1H), 8.97 (d, J=2.4 Hz, 1H), 8.64 (d, J=2.4 Hz, 1H), 8.44 (d, J=2.4 Hz, 1H), 8.27 (t, J=1.9 Hz, 1H), 7.99-7.96 (m, 2H), 7.74-7.69 (m, 2H), 7.66 (t, J=7.8 Hz, 1H), 7.50 (d, J=8.2 Hz, 1H), 7.19-7.12 (m, 2H), 4.74 (s, 2H), 2.76 (s, 3H), 2.57 (s, 3H). 1H obscured/not observed.

### GPR35 Assay

The functional assay described measures the ability of GPR35 modulators to inhibit a GPR35 agonist-induced phospho-ERK signal. This is expressed as the concentration of modulator required to reduce the phospho-ERK signal by 50 percent, i.e. the IC₅₀; the signal window being defined as the difference between agonist plus modulator vehicle (no modulator) and agonist vehicle (no agonist) controls.

This assay is a cellular GPR35 phospho-ERK homogeneous time resolved fluorescence (HTRF) assay using the GPR35-CHO-K1-mt aequorin-Gα16 cell line. Upon activation of GPR35 with the agonist lodoxamide at EC₈₀ concentration, ERK 1/2 is phosphorylated, and after lysis of the cell membrane, phospho-ERK1/2 (Thr202/Tyr204) is detected in a sandwich assay using two different specific antibodies, one labelled with Eu³⁺-cryptate (donor) and the second with d2 (acceptor). When the dyes are in proximity, the excitation of the donor with a light source (laser or flash lamp) triggers a Fluorescence Resonance Energy Transfer (FRET) towards the acceptor, resulting in specific acceptor fluorescence (at 665 nm). The specific signal is directly proportional to the amount of phospho-ERK1/2 (Thr202/Tyr204) in the sample. Compounds that interfere with the levels of phospho-ERK1/2 (Thr202/Tyr204) will result in a low HTRF signal.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims

TABLE 1
Activity of selected compounds according to the invention

| Example | |
|---|---|
| Number | Activity Group |
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 19 | A |
| 20 | A |
| 21 | B |
| 22 | A |
| 23 | B |
| 24 | A |
| 25 | B |
| 26 | B |
| 27 | B |
| 28 | A |
| 29 | B |
| 30 | A |
| 31 | B |
| 32 | B |
| 33 | A |
| 34 | A |
| 35 | A |
| 36 | A |
| 37 | A |
| 38 | A |
| 39 | A |
| 40 | B |
| 41 | B |
| 42 | B |
| 43 | B |
| 44 | B |
| 45 | B |
| 46 | B |
| 47 | B |
| 48 | A |
| 49 | B |
| 50 | B |
| 51 | B |
| 52 | A |
| 53 | A |
| 54 | B |
| 55 | B |
| 56 | A |
| 57 | A |
| 58 | A |
| 59 | A |
| 60 | A |
| 61 | B |
| 62 | B |
| 63 | A |
| 64 | A |
| 65 | A |
| 66 | B |
| 67 | A |
| 68 | A |
| 69 | A |
| 70 | A |
| 71 | B |
| 72 | B |
| 73 | B |
| 74 | A |
| 75 | A |
| 76 | A |
| 77 | A |
| 78 | B |
| 79 | B |
| 80 | A |
| 81 | A |
| 82 | B |
| 83 | A |
| 84 | B |
| 85 | A |
| 86 | B |
| 87 | B |
| 88 | A |
| 89 | A |
| 90 | A |
| 91 | B |
| 92 | A |
| 93 | B |
| 94 | B |
| 95 | B |
| 96 | A |
| 97 | B |
| 98 | B |
| 99 | B |
| 100 | B |
| 101 | B |

A = IC₅₀ < 1 μM;
B = IC₅₀ > 1 μM and <10 μM

### REFERENCES

¹ Quon et al, ACS Pharmacology and Translational Science 3, 801-812 (2020)
² Kaya et al, Frontiers in Immunology 12: 717392 (2021)
³ Wang et al, J Biol Chem 281, 22021-22028 (2006)
⁴ Kapolka et al, PNAS 117, 13117-13126 (2020)
⁵ Giovanni et al, Cell 185, 815-830-e19 (2022)
⁶ Oka et al, Biochem & Biophys Res Comms 395, 232-237 (2010)
⁷ Deng et al. Scientific Reports 2, Article number: 373 (2012)
⁸ Maravillas-Montero et al, J Immunology 194, 29-33 (2015)
⁹ Lee et al Agricultural and Food Chem doi: 10.1021/acs.jafc.2c01251. Online ahead of print (2022)
¹⁰ Jenkins et al, Biochem J 432, 451-419 (2010)
¹¹ Yang et al, Pharmacology 86, 1-5. doi: 10.1159/000314164. Epub (2010)
¹² Taniguchi et al. FEBS Letters 580, 5003-5008 (2006)
¹³ MacKenzie et al, Molecular Pharmacology 85, 91-104 (2014)
¹⁴ Wei et al, J Med Chem 64, 2634-2647 (2021)
¹⁵ Abdalhameed et al. Bioorg Med Chem Lett 27, 612-615 (2017)
¹⁶ https://www.ncbi.nlm.nih.gov/gene?Db=gene&Cmd=DetailsSearch&Term=2859
¹⁷ https://www.proteinatlas.org/ENSG00000178623-GPR35/tissue
¹⁸ MacKenzie et al, Frontiers in Endocrinology (Lausanne) 2, 68, 1-10 (2011)
¹⁹ Imielinski et al, Nat Genet 41, 1335-1340 (2009)
²⁰ Ellinghaus et al, Hepatology 58, 1074-1083 (2013)
²¹ Schneditz et al, Sci Signal 12:eaau9408 (2019) doi: 10.1126/scisignal.aau9048
²² Pagano et al, Gut (2021) Gut. 2022 March; 71(3):509-520. doi: 10.1136/gutjnl-2020-323363. Epub 2021 Mar. 23
²³ Kaya et al, Cell Rep 32:107979 (2020)
²⁴ http://gepia.cancer-pku.cn/detail.php?gene=GPR35
²⁵ Okumura et al, Cancer Sci 95, 131-135 (2004)
²⁶ Ali et al, Tumor Biology 41 1-11 (2019)
²⁷ Mackiewicz at al, Pharmacological Rep doi: 10.1007/s43440-022-00371-2. Online ahead of print (2022)
²⁸ Olson et al, Front. Chem., 9 article 671483 (2021)
²⁹ Sun, Y. V. et al, (2008) Genet. Epidemiol. 32, 350-360 doi: 10.1002/gepi.20309
³⁰ Min, K. D. et al (2010) Biochem. Biophys. Res. Commun. 393, 55-60
³¹ Ohshiro, H. et al (2008) Biochem. Biophys. Res. Commun. 365, 344-348
³² Cosi, C. et al (2010). Neuropharmacology 60, 1227-1231
³³ US20070077602; Leonard et al
³⁴ Wang, J. et al (2006) J. Biol. Chem. 281, 22021-22028
³⁵ Fallarini, S. et al (2010) Biochem. Bio-phys. Res. Commun. 398, 420-425
³⁶ Barth, M. C. et al (2009) J. Biol. Chem. 284, 19189-19195
³⁷ Birring, S. S. et al (2017) Lancet Respir. Med. 5 (10), 806-815
³⁸ Yang, Y. et al (2010) Pharmacology; 86(1):1-5
³⁹ Smith J. A. (January 2015). Current Allergy and Asthma Reports. 15 (1): 489
⁴⁰ Shu C. et al (November 2022), Cell Death Disc, 444
⁴¹ Miyamoto, K. et al. Cell Reports, (2023), 42, 113005.
⁴² Song, Z. et al. Bioorg. Med. Chem. (2023). https://doi.org/10.1016/j.bmc.2023.117511
⁴³ Otkur, W. et al. Eur. J. Pharmacol, (2023), 949, 175719.
⁴⁴ Zheng, X. et al. Brain, Behaviour and Immunity, (2019), 79, 244.
⁴⁵ Boleij, A. et al. Communications Biology, (2021), 4:585.

1. A compound of formula (I), or a pharmaceutically acceptable salt or solvate thereof, 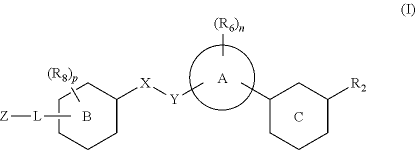
X is NR⁷ and Y is CO;
ring C is a 6-membered heteroaryl or aryl group, or a partially or fully unsaturated 6-membered heterocyclic group containing at least one N, and optionally comprising at least one CO group, and wherein said aryl, heteroaryl or heterocyclic group is optionally further substituted by one or more substituents selected from alkyl, haloalkyl, alkoxy, haloalkoxy, halo, cyano, NR₉SO₂R₁₁, NRCOR₁₂, NR₁₃R₁₄, OH, CO₂R₁₅, SO₂NR₁₆R₁₇, CONR₁₈R₁₉, cycloalkyl and (CH₂)_{q}-heterocycloalkyl;
ring A is a group: 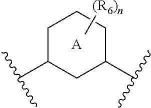
wherein A is a phenyl group or a 6-membered heteroaryl group;
or ring A is a group: 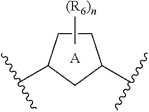
wherein A is a 5-membered heteroaryl group;
wherein in each case the wavy lines indicate the point of attachment to Y and ring C respectively;
ring B is a phenyl group or a 6-membered heteroaryl group;
R₂ is (CR₃₃R₃₄)ₘCOOH;
each R₆ is independently selected from alkyl, halo, haloalkyl, alkoxy, haloalkoxy, cyano, NR₁₀COR₂₀, NR₁₀SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl;
each R₈ is independently selected from alkyl, halo, haloalkyl, alkoxy, haloalkoxy, cyano, NR₁₀COR₂₀, NR₁₀—SO₂R₂₁, (CH₂)_{q}SR₂₂, (CH₂)_{q}SOR₂₃, (CH₂)_{q}SO₂R₂₄, SO₂NR₂₅R₂₆, (CH₂)_{q}OH, (CH₂)_{q}OR₂₇, NR₂₈R₂₉, CONR₃₀R₃₁, cycloalkyl and (CH₂)_{q}-heterocycloalkyl;
R₇ is selected from H and alkyl;
each R₉ is independently selected from H and alkyl;
each R₁₀ is independently selected from H and alkyl;
R₁₁-R₃₁ are each independently selected from H, alkyl, haloalkyl, aralkyl, alkoxy, alkoxyalkyl, hydroxyalkyl and cycloalkyl;
m, n and p are each independently an integer from 0 to 4;
each q is independently an integer from 0 to 4;
L is a direct bond or is a group selected from, —SO₂—, —O—SO₂—, —SO₂—O—, —O—, —NR₃₂—SO₂—, —NR₃₂—SO₂-alkylene, —SO₂—NR₃₂—, —SO₂—NR₃₂-alkylene, alkylene, heteroalkylene, cycloalkylene, heterocycloalkylene, alkylene-cycloalkylene, alkylene-SO₂—, —SO₂-alkylene, alkylene-SO—, —SO-alkylene, alkylene-SO₂-alkylene, alkylene-SO-alkylene, cycloalkylene-alkylene, alkylene-heterocycloalkylene, heterocycloalkylene-alkylene, heteroalkylene-heterocycloalkylene, heterocycloalkylene-heteroalkylene, heteroalkylene-cycloalkylene, cycloalkylene-heteroalkylene, wherein the alkylene moiety in the above groups is optionally substituted by one or more substituents selected from halo, alkyl, haloalkyl and cycloalkyl;
R₃₂, R₃₃ and R₃₄ are each independently selected from H and alkyl; and
Z is a group selected from alkyl, cycloalkyl, aryl, heteroaryl and heterocycloalkyl, each of which is optionally further substituted by one or more groups selected from CN, halo, alkyl, haloalkyl, alkenyl, alkynyl, alkoxy and haloalkoxy.

2. A compound according claim 1 which is of formula (Ie): 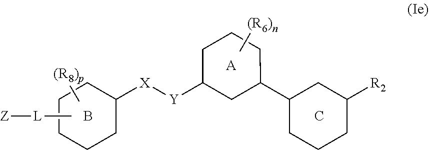
wherein ring A, ring B, ring C, X, Y, L, Z, R₂, R₆, R₈, n and p are as defined in claim 1.

3. A compound according to claim 2 wherein A is selected from phenyl, pyridinyl, pyradizinyl, pyrimidinyl and pyrazinyl, each of which is optionally substituted by one to four R₆ groups.

4. A compound according to claim 1 which is of formula (If): 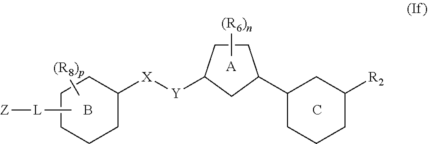
wherein ring A, ring B, ring C, X, Y, L, Z, R₂, R₆, R₈, n and p are as defined in claim 1, and n is preferably an integer from 0 to 3.

5. A compound according to claim 4 wherein A is selected from pyrrolyl, thiazolyl, oxazolyl, furanyl, thienyl and pyrazolyl, each of which is optionally substituted by one to three R₆ groups.

6. A compound according to any preceding claim wherein said compound is of formula (Ig): 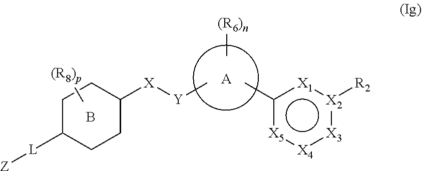
wherein X₁, X₂, X₃, X₄, X₅ form a 6-membered heteroaryl group containing at least one N, or a 6-membered aryl group, and wherein said heteroaryl or aryl group is optionally further substituted by one or more substituents selected from alkyl, haloalkyl, alkoxy, haloalkoxy, halo, NR₉SO₂R₁₁, NRCOR₁₂, NR₁₃R₁₄, OH, CO₂R₁₅, SO₂NR₁₆R₁₇, CONR₁₈R₁₉, cycloalkyl and (CH₂)_{q}-heterocycloalkyl; and
A, B, Z, L, X, Y, R₂, R₆, R₈, n and p are as defined in claim 1.

7. A compound according to any preceding claim wherein said compound is of formula (Ib): 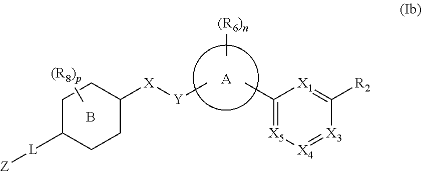
wherein:
X₁ is N or CR₁;
X₃ is N or CR₃;
X₄ is N or CR₄;
X₅ is N or CR₅;
R₁, R₃, R₄ and R₅ are each independently selected from H, alkyl, haloalkyl, alkoxy, haloalkoxy, halo, NR₁₃R₁₄, OH, NR₉SO₂-alkyl, CONR₁₈R₁₉, cycloalkyl and (CH₂)_{q}-heterocycloalkyl; and
wherein R₂, R₆, R₈, n, p, L, A, B, X, Y and Z are as defined in claim 1.

8. A compound according to any preceding claim wherein n is 0.

9. A compound according to any one of claims 6 to 8 wherein X₁, X₂, X₃, X₄ and X₅ form a 6-membered heteroaryl group comprising 1 or 2 nitrogens, or a phenyl group.

10. A compound according to claim 9 wherein X₁, X₂, X₃, X₄ and X₅ form a phenyl, pyridinyl, pyrimidinyl, pyradizinyl or pyrazinyl group.

11. A compound according to any preceding claim wherein R₂ is COOH.

12. A compound according to any one of claims 1 to 3 or 6 to 11 wherein said compound is of formula (Ic): 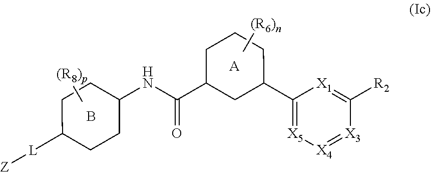
wherein:
X₁ is N or CR₁;
X₃ is N or CR₃;
X₄ is N or CR₄;
X₅ is N or CR₅;
R₁, R₃, R₄ and R₅ are each independently selected from H, alkyl, haloalkyl, alkoxy, haloalkoxy, halo, NR₁₃R₁₄, OH, NR₉SO₂-alkyl, CONR₁₈R₁₉, cycloalkyl and (CH₂)_{q}-heterocycloalkyl;
A is a phenyl or pyridinyl group, more preferably phenyl; and
R₂, R₆, R₈, n, p, L, B and Z are as defined in claim 1.

13. A compound according to claim 12 wherein R₁, R₃, R₄ and R₅ are each independently selected from H, C₁₋₆-alkyl, C₁₋₆-haloalkyl, C₁₋₆-alkoxy, C₁₋₆-haloalkoxy, Cl, F, NH₂, NH—C₁₋₆-alkyl, NH—C₃₋₆-cycloalkyl, N(C₁₋₆-alkyl)₂, OH, NHSO₂—C₁₋₆-alkyl, CONR₁₈R₁₉, C₃₋₆-cycloalkyl, NH-(hydroxy-C₁₋₆-alkyl), NH—(C₁₋₆-alkoxy), CH₂-heterocycloalkyl and heterocycloalkyl.

14. A compound according to claim 12 or claim 13 wherein R₁, R₃, R₄ and R₅ are each independently selected from H, Me, MeO, CF₃, Cl, F, NH₂, NH-Me, NH-cyclopropyl, NMe₂, OH, NHSO₂Me, CONH₂, cyclopropyl, NHCH₂CH₂OH, NHCH₂CH₂OMe, CH₂—N-morpholinyl and N-morpholinyl.

15. A compound according to any one of claims 12 to 14 wherein:
R₂ is COOH;
X₁ is CR₁;
X₃ is CR₃;
X₄ is N; and
X₅ is CR₅.

16. A compound according to any one of claims 12 to 14 wherein:
R₂ is COOH;
X₁ is N;
X₃ is CR₃;
X₄ is CR₄; and
X₅ is CR₅.

17. A compound according to any one of claims 12 to 14 wherein:
R₂ is COOH;
X₁ is N;
X₃ is CR₃;
X₄ is N;
X₅ is CR₅.

18. A compound according to any one of claims 12 to 17 wherein R₃ is selected from H, Me, MeO, CF₃, Cl, F, NH₂, NH-Me, NH-cyclopropyl, NMe₂, OH, NHSO₂Me, CONH₂, cyclopropyl, NHCH₂CH₂OH, NHCH₂CH₂OMe, CH₂—N-morpholinyl and N-morpholinyl; and R₁, R₄ and R₅ are H.

19. A compound according to claim 12 wherein:
X₁ is CH or N, more preferably CH;
X₃ is C-alkyl, more preferably Me;
X₄ is N; and
X₅ is CH.

20. A compound according to any one of claims 1 to 5 wherein ring C is a partially or fully unsaturated 6-membered heterocyclic group containing at least one N, and optionally comprising at least one CO group,

21. A compound according to claim 20 which is of formula (Id), 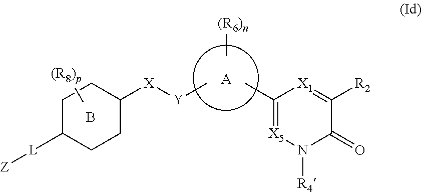
wherein X₁ is N or CR₁ and X₅ is N or CR₅;
R₁ and R₅ are each independently selected from H, alkyl, haloalkyl, alkoxy, haloalkoxy, halo, NR₁₃R₁₄, OH, NR₉SO₂-alkyl, CONR₁₈R₁₉, cycloalkyl and (CH₂)_{q}-heterocycloalkyl; and
R₄′ is selected from H, alkyl and cycloalkyl;
and where A, B, L, Z, X, Y, R₂, R₆, R₃, n and p are as defined in claim 1.

22. A compound according to claim 21 wherein X₁ and X₅ are both CH, and R₄′ is H or Me.

23. A compound according to any preceding claim wherein B is a phenyl or pyridinyl group, each of which is optionally substituted by one to four R₃ groups as defined in claim 1.

24. A compound according to any preceding claim wherein B is a phenyl group, optionally substituted by one or two halo groups.

25. A compound according to any preceding claim wherein Z is a group selected from C₁₋₆-alkyl, phenyl, C₃₋₆-cycloalkyl and a 5- or 6-membered heterocycloalkyl group, each of which is optionally further substituted by one or more groups selected from alkyl, halo, haloalkyl, CN, alkenyl, alkynyl and alkoxy.

26. A compound according to any preceding claim wherein Z is a group selected from phenyl, pyridinyl, piperidinyl, cyclopropyl and tetrahydropyranyl, more preferably phenyl, each of which is optionally further substituted by one or more groups selected from alkyl, halo, haloalkyl, CN, alkenyl, alkynyl and alkoxy.

27. A compound according to any preceding claim L is selected from —O—, —NH—SO₂—, —NH—SO₂—(CR′R″)ₐ—, —(CR′R″)ₐ—SO₂—NH—, —SO₂—NH—, —SO₂—NH—(CR′R″)ₐ—, —(CR′R″)ₐ—NH—SO₂—, —O—SO₂—, —SO₂—O—, —(CR′R″)ₐ—, —(CR′R″)ₐ—O—, O—(CR′R″)ₐ—, —(CR′R″)ₐ—O—(CR′R″)_{b}—, —(CR′R″)ₐ—S—(CR′R″)_{b}—, —(CR′R″)ₐ—SO—(CR′R″)_{b}—, —(CR′R″)ₐ—SO₂—, —SO₂—(CR′R″)ₐ—, —(CR′R″)ₐ—SO—, —SO—(CR′R″)ₐ—, —(CR′R″)ₐ—SO₂—(CR′R″)_{b}—, —(CR′R″)ₐ—S—(CR′R″)_{b}—O—, —O—(CR′R″)ₐ—S—(CR′R″)_{b}—, —(CR′R″)ₐ—O—(CR′R″)_{b}—S—, —S—(CR′R″)ₐ—O—(CR′R″)_{b}—, heterocycloalkylene-(CR′R″)ₐ—O—, —O—(CR′R″)ₐ-heterocycloalkylene-, —(CR′R″)ₐ—O-heterocycloalkylene, heterocycloalkylene-O—(CR′R″)ₐ—, —O-heterocycloalkylene-(CR′R″)ₐ— and —(CR′R″)ₐ-heterocycloalkylene-O—, wherein a and b are each independently an integer from 1 to 6, and each R′ and each R″ is independently selected from H and alkyl, more preferably where R′ and R″ are both H.

28. A compound according to any preceding claim wherein L is selected from —CH₂—, —CH₂CH₂CH₂—, —CH₂CH₂—, —CH₂O—, —OCH₂—, —CH(Me)OCH₂, —CH₂OCH(Me)-, —CH(CF₃)OCH₂, —CH₂OCH(CF₃)—, —CH₂CH₂O—, —OCH₂CH₂—, —CH₂CH₂CH₂O—, —OCH₂CH₂CH₂—, —CH₂SO₂CH₂—, —CH₂SOCH₂—, —CH₂SCH₂—, —NH—SO₂—, —NH—SO₂—CH₂—, —CH₂—SO₂—NH—, —SO₂—NH—, —SO₂—NH—CH₂—, —CH₂—NH—SO₂—, —CH₂CH₂CH₂CH₂O—, —OCH₂CH₂CH₂CH₂—, —CH₂SO₂—, —SO₂CH₂—, —CH₂SO—, —SOCH₂—, —CH₂OCH₂, —CH(Me)O—, —OCH(Me)-, —CH(CF₃)O—, —OCH(CF₃)—, —CH₂SCH₂O— and —OCH₂SCH₂—.

29. A compound according to any preceding claim wherein L-Z is —OCH₂CH₂Ph, —OCH₂Ph, —OCH₂CH₂CH(Me)₂, —OCH₂CH(Me)₂, —OSO₂-(4-methylphenyl), —CH₂SO₂CH₂-Ph, —CH₂SO₂-Ph, —OCH₂-cyclopropyl, —OCH₂CH₂CH₂CH₃, —OCH₂CH₂CH₂CH₂Ph, —CH₂OCH₂Ph, —OCH₂SCH₂Ph, —CH₂OCH₂Ph, —CH₂CH₂Ph, —CH₂SO₂-(4-methylphenyl), —CH₂SO₂-(4-methoxyphenyl), —CH₂SO₂-(4-chlorophenyl), —CH₂SO₂-(3-chlorophenyl), —CH₂SO₂-(2-chlorophenyl), —CH₂OCH(Me)-Ph, —CH₂OCH(CF₃)-Ph, —CH₂OCH₂-cyclopropyl, —CH₂O-cyclopropyl, —OCH(Me)-cyclopropyl, —CH(Me)O-cyclopropyl, —OCH(CF₃)-cyclopropyl, —CH(CF₃)O-cyclopropyl, CH₂OCH₂-(pyridin-2-yl), —CH₂OCH₂(4-methoxyphenyl), CH₂OCH₂(3-methoxyphenyl), —CH₂SO₂-(4-methylpyridin-3-yl)-chlorophenyl),

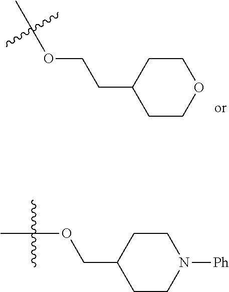 [SMILES: *OCCC1CCOCC1 ; *OCC1CCN(c2ccccc2)CC1]

30. A compound according to any preceding claim wherein L-Z is —OCH₂CH₂Ph, —OCH₂CH₂CH₂CH₂Ph or —CH₂OCH₂Ph, more preferably —OCH₂CH₂Ph.

31. A compound which is selected from the following: 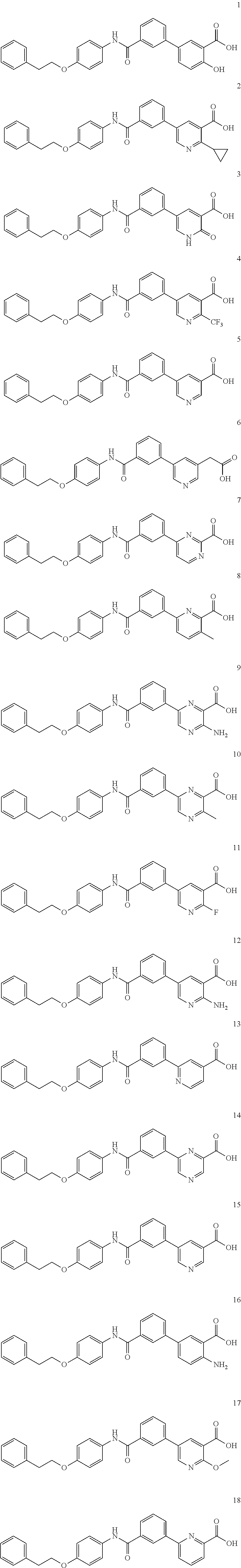 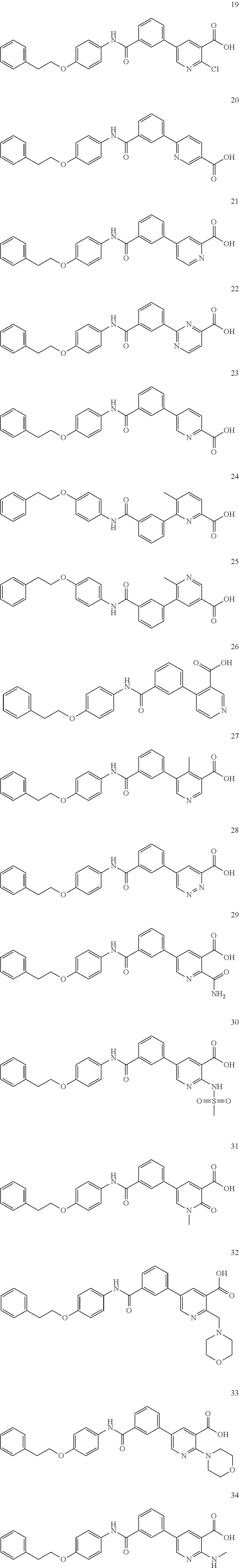 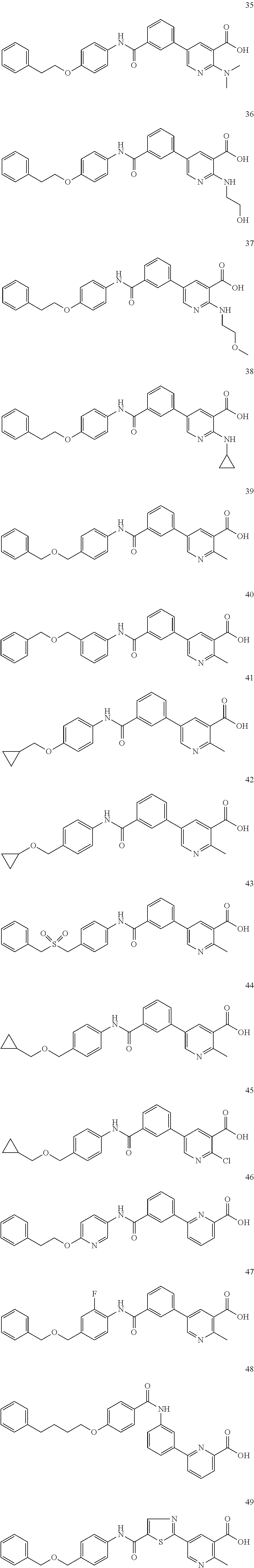 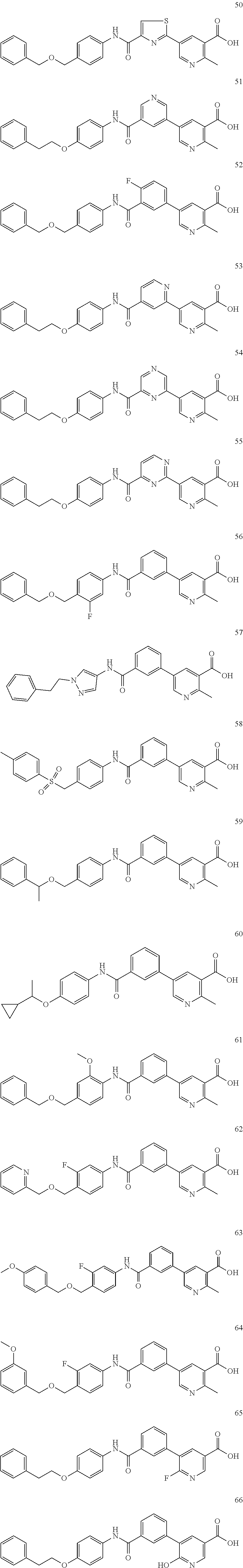 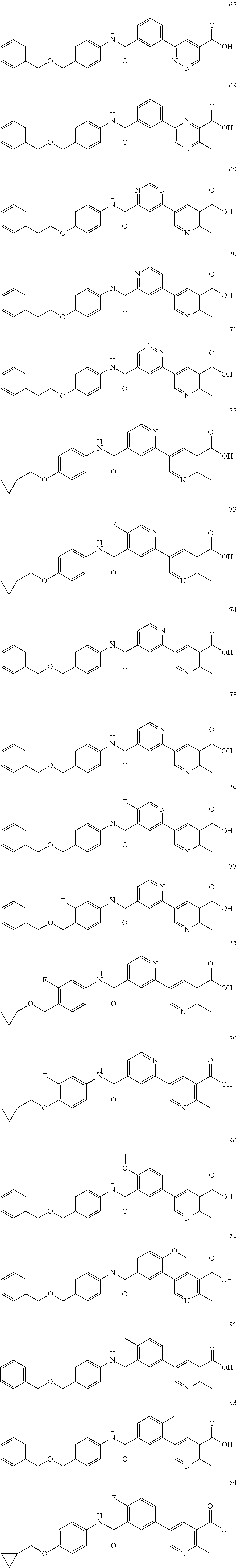 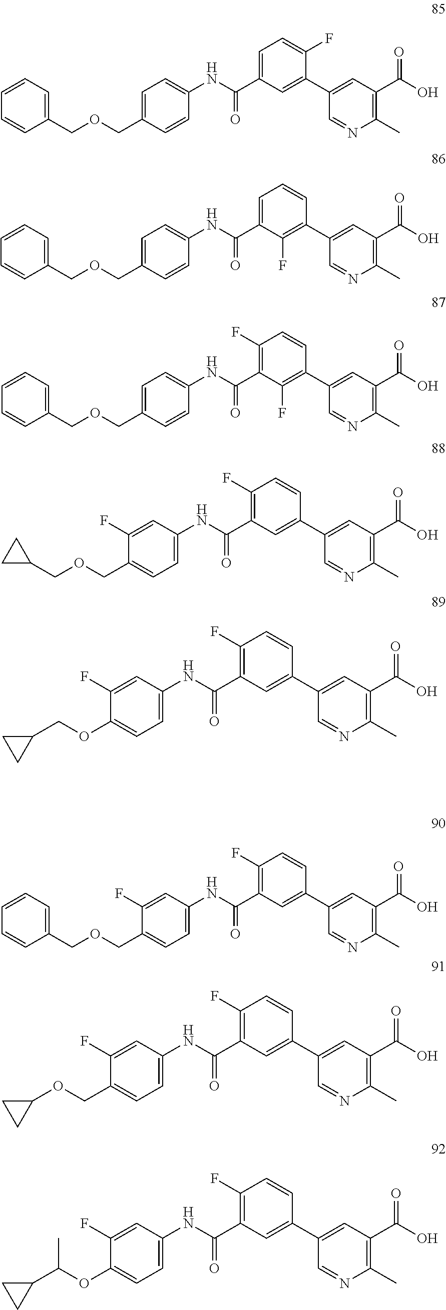 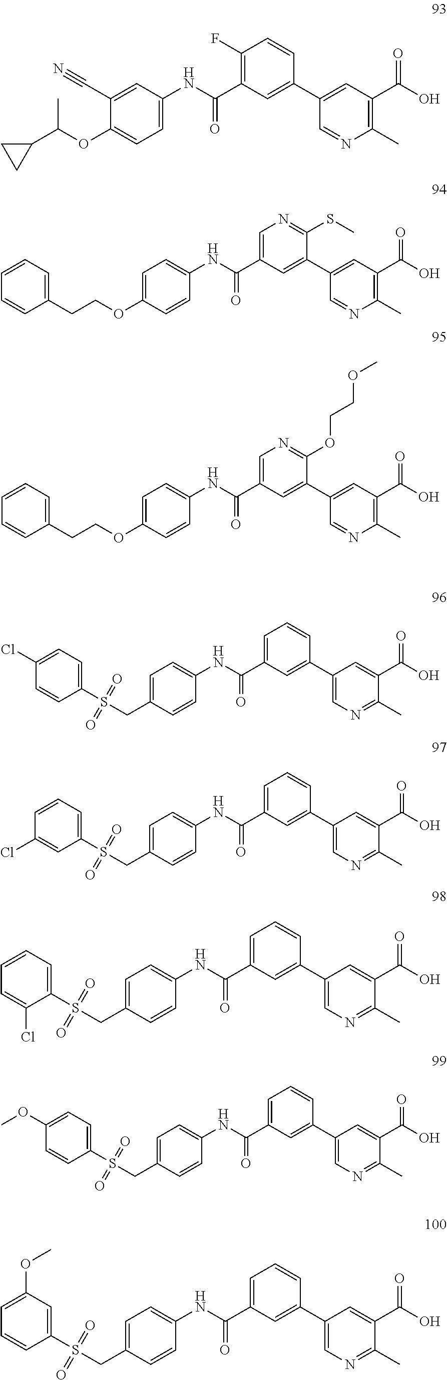 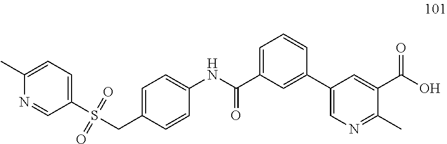 [SMILES: Cc1ccc(S(=O)(=O)Cc2ccc(NC(=O)c3cccc(-c4cnc(C)c(C(=O)O)c4)c3)cc2)cn1]
and pharmaceutically acceptable salts and solvates thereof.

32. A pharmaceutical composition comprising a compound according to any of claims 1 to 31, and a pharmaceutically acceptable diluent, excipient, or carrier.

33. A compound according to any one of claims 1 to 31, or a pharmaceutical composition according to claim 32, for use as a medicament.

34. A compound according to any one of claims 1 to 31, or a pharmaceutical composition according to claim 32, for use in treating or preventing a disorder selected from a proliferative disorder, a fibrotic disorder, a gastrointestinal disorder, an inflammatory disorder, an immune disorder, and a cardiovascular disease.

35. A compound or pharmaceutical composition for use according to claim 34, wherein the disorder is a proliferative disorder, preferably a cancer or leukemia.

36. A compound or pharmaceutical composition for use according to claim 35, wherein the cancer is selected from colon, colorectal, rectum, stomach, oesophagus, pancreas, gall bladder, bile duct, liver, lung, kidney, gynaecological, breast, testicular, skin, prostate, central nervous system and brain cancer.

37. A compound or pharmaceutical composition for use according to claim 34, wherein the disorder is a gastrointestinal disorder, preferably selected from inflammatory bowel disease, ulcerative colitis, primary sclerosing cholangitis and Crohn's disease.

38. A compound or pharmaceutical composition for use according to claim 34 wherein the disorder is a cardiovascular disease, preferably selected from hypertension, heart failure, atherosclerosis, peripheral vascular disease and stroke.

39. A compound or pharmaceutical composition for use according to any one of claims 34 to 38, wherein the use comprises modulating GPR35, preferably wherein the use comprises inhibiting GPR35 signalling.

40. A method of treating a disorder as defined in any of claims 34 to 38, comprising administering to a subject a compound as defined in any of claims 1 to 31, or a pharmaceutical composition as defined in claim 32.

41. A compound as defined in any one of claims 1 to 31, or a pharmaceutically acceptable salt or solvate thereof, or a pharmaceutical composition according to claim 32, for use in treating or preventing a GPR35-associated disease or disorder.

42. Use of a compound as defined in any one of claims 1 to 31, or a pharmaceutically acceptable salt or solvate thereof, in the preparation of a medicament for treating or preventing a GPR35-associated disease or disorder in a subject.

43. Use of a compound as defined in any one of claims 1 to 31, or a pharmaceutically acceptable salt or solvate thereof, in the preparation of a medicament for treating or preventing a disorder selected from a proliferative disorder, a gastrointestinal disorder, an inflammatory disorder, an immune disorder, a fibrotic disorder and a cardiovascular disease.
